(19) United States
(12) Patent Application Publication
(10) Pub. No.: US 2025/0239349 A1
(43) Pub. Date: Jul. 24, 2025

(54) SYSTEMS AND METHODS FOR DETERMINING T-CELL CROSS-REACTIVITY BETWEEN ANTIGENS

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Vinod Balachandran, New York, NY (US); Zachary Sethna, New York, NY (US); Luis Rojas, New York, NY (US); Benjamin D. Greenbaum, New York, NY (US)

(21) Appl. No.: 18/833,363

(22) PCT Filed: Jan. 26, 2023
(86) PCT No.: PCT/US2023/011643

§ 371 Date: Jul. 25, 2024

Related U.S. Application Data
(60) Provisional application No. 63/303,500, filed on Jan. 26, 2022.

(51) Int. Cl. G16H 20/17; C12Q 1/6881; G16B 20/10; G16B 20/20

(52) U.S. Cl. CPC G16H 20/17; C12Q 1/6881; G16B 20/10; G16B 20/20; C12Q 2600/106

(57) ABSTRACT

A neoantigen model discrimination method to determine if the immune system can discriminate it from “self” by estimating if a neoantigen has sufficient antigenic distance from its wild-type peptide to differentially bind the MHC or activate a T cell. In one embodiment, a model can be integrated to estimate the fitness of tumor clones as the aggregate of the cost due to T cells recognizing high quality neoantigens offset by the gain from mutations in canonical oncogenes. In an additional embodiment, a model can be used to rationally identify antigens that either elicit desired, or prevent undesired responses of any immunologically based medicine.

This application is an International Application which claims priority from U.S. provisional patent application No. 63/303,500, filed on Jan. 26, 2022, the entire contents of which are incorporated herein by reference.

For countries that permit incorporation by reference, all patents, patent applications and publications cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted being prior art.

### GOVERNMENT INTERESTS

This invention was made with Government support under grant no. U01 CA224175 awarded by National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to a method for determining within a computing system a likelihood that a human subject afflicted with a cancer will be responsive to a treatment regimen that comprises administering a checkpoint blockade immunotherapy directed to the cancer of the subject.

### BACKGROUND OF THE INVENTION

In 1957, Burnet and Thomas proposed that the immune system in multicellular organisms must eliminate transformed cells as an evolutionary necessity to maintain tissue homeostasis. This theory of ‘cancer immunosurveillance’ was later redefined more broadly as ‘cancer immunoediting’—as a consequence of the immune system protecting the host from cancer, the immune system must also sculpt developing cancers. When cancers develop, they accumulate mutations, some of which generate new protein sequences (neoantigens). As neoantigens are mostly absent from the human proteome, they can escape T cell central tolerance in the thymus to become antigens in cancers. However, neoantigens typically arise in passenger mutations, and therefore distribute heterogeneously in cancer cell clones with variable immunogenicity. Thus, T cells selectively ‘edit’ clones with more immunogenic neoantigens, inducing less immunogenic clones to outgrow in cancers.

### SUMMARY OF THE INVENTION

Aspects of the invention are directed to a method for determining within a computing system a likelihood that a human subject afflicted with a cancer will be responsive to a treatment regimen that comprises administering a checkpoint blockade immunotherapy directed to the cancer of the subject. In embodiments, the computing system has one or more programmable processors, memory, and a plurality of instructions stored on the memory that are executable by the one or programmable processors. In embodiments, the method comprises: a) obtaining a plurality of sequencing reads from one or more samples from the human cancer subject that is representative of the cancer; b) determining a human leukocyte antigen (HLA) type of the human subject; c) computing for a plurality of clones, and for each respective clone a in the plurality of clones, an initial frequency Xa of the respective clone a in the one or more samples; d) for each respective clone a in the plurality of clones, computing a corresponding clone fitness score of the respective clone, thereby computing a plurality of clone fitness scores, each corresponding clone fitness score computed for a respective clone a by a first procedure comprising: i. identifying a plurality of neoantigens in the respective clone a; ii. computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and iii. determining the corresponding clone fitness score of the respective clone a as an aggregate of the neoantigen recognition potentials across the plurality of neoantigens in the respective clone a; and e) computing a total fitness for the one or more samples as a sum of the clone fitness scores across the plurality of clones, wherein each clone fitness score is weighted by the initial frequency Xa of the corresponding clone a, the total fitness quantifies the likelihood that the human subject afflicted with the cancer will be responsive to the treatment regimen. In embodiments, the method further comprises administering to the human subject the checkpoint blockade immunotherapy if the human subject is likely to be responsive to the treatment regimen. In embodiments, the checkpoint blockade immunotherapy comprises an antibody, fragment, or derivative thereof. In embodiments, the antibody is specific for CTLA4, PD1, PD-L1, LAG3, TIM-3, GITR, OX40, CD40, TIGIT, 4-1BB, B7-H3, B7-H4, or BTLA. In embodiments, the checkpoint blockade immunotherapy comprises ipilimumab or tremelimumab. In embodiments, the cancer is a carcinoma, a melanoma, a lymphoma/leukaemia, a sarcoma, or a neuro-glial tumor. In embodiments, the cancer is lung cancer, pancreatic cancer, colon cancer, stomach cancer, esophagus cancer, breast cancer, ovary cancer, prostate cancer, or liver cancer. In embodiments, each clone a in the plurality of clones is uniquely defined by a unique set of somatic mutations, and the plurality of clones is determined by a variant allele frequency of each respective somatic mutation in a plurality of somatic mutations determined from the plurality of sequencing reads. In embodiments, the somatic mutation is a single nucleotide variant or an indel. In embodiments, the plurality of clones is determined by identifying a plurality of inferred copy number variations using the plurality of sequencing reads. In embodiments, each clone a in the plurality of clones is uniquely defined by a unique set of somatic mutations, and the plurality of clones is determined by a combination of (i) a variant allele frequency of each respective somatic mutation in the plurality of somatic mutations determined from the plurality of sequencing reads and (ii) an identification of a plurality of inferred copy number variations using the whole-genome sequencing data. In embodiments, the plurality of sequencing reads exhibits an average read depth of less than 40. In embodiments, the plurality of sequencing reads exhibits an average read depth of between 25 and 60. In embodiments, each neoantigen in the plurality of neoantigens of a clone in the plurality of clones is a nonamer peptide. In embodiments, each neoantigen in the plurality of neoantigens of a clone in the plurality of clones is a peptide comprising eight, nine, ten, or eleven residues in length. In embodiments, the HLA type of the human subject is determined from the plurality of sequencing reads. In embodiments, the HLA type of the human subject is determined using a polymerase chain reaction using a biological sample from the human subject. In embodiments, the plurality of clones comprises two clones. In embodiments, the plurality of clones comprises between two clones and ten clones. In embodiments, the plurality of clones comprises greater than ten clones. In embodiments, the initial frequency Xa of the respective clone a in the one or more samples is determined using the plurality of sequencing reads from the one or more samples from the human subject.

Aspects of the invention are directed towards a system for determining a likelihood that a human subject afflicted with a cancer will be responsive to a treatment regimen that comprises administering a checkpoint blockade immunotherapy directed to the cancer of the subject. In embodiments, the system comprises: memory; one or more processors; and one or more modules stored in memory and configured for execution by the one or more processors, the modules comprising instructions for: (A) obtaining a plurality of sequencing reads from one or more samples from the human subject that is representative of the cancer; (B) determining a human leukocyte antigen (HLA) type of the human subject; (C) determining a plurality of clones, and for each respective clone a in the plurality of clones, an initial frequency Xa of the respective clone a in the one or more samples; (D) for each respective clone a in the plurality of clones, computing a corresponding clone fitness score of the respective clone, thereby computing a plurality of clone fitness scores, each corresponding clone fitness score computed for a respective clone a by a first procedure comprising: (a) identifying a plurality of neoantigens in the respective clone a; (b) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (c) determining the corresponding clone fitness score of the respective clone a as an aggregate of the neoantigen recognition potentials across the plurality of neoantigens in the respective clone a; and (E) computing a total fitness for the one or more samples as a sum of the clone fitness scores across the plurality of clones, wherein each clone fitness score is weighted by the initial frequency Xa of the corresponding clone a, and the total fitness quantifies the likelihood that the human subject afflicted with the cancer will be responsive to the treatment regimen.

Aspects of the invention are directed towards a system for determining a likelihood that a human subject afflicted with a cancer will be responsive to a treatment regimen that comprises administering a checkpoint blockade immunotherapy directed to the cancer to the subject. In embodiments, the system comprises: memory; one or more processors; and one or more modules stored in memory and configured for execution by the one or more processors, the modules comprising instructions for carrying out the method of any one of the aspects or embodiments described herein.

Aspects of the invention are directed towards a non-transitory computer readable storage medium for determining a likelihood that a human subject afflicted with a cancer will be responsive to a treatment regimen that comprises administering a checkpoint blockade immunotherapy directed to the cancer to the subject. In embodiments, the non-transitory computer readable storage medium stores one or more programs for execution by one or more processors of a computer system, the one or more computer programs comprising instructions for: a. obtaining a plurality of sequencing reads from one or more samples from the human subject that is representative of the cancer; b. determining a human leukocyte antigen (HLA) type of the human subject; c. determining a plurality of clones, and for each respective clone a in the plurality of clones, an initial frequency Xa of the respective clone a in the one or more samples; d. for each respective clone a in the plurality of clones, computing a corresponding clone fitness score of the respective clone, thereby computing a plurality of clone fitness scores, each corresponding clone fitness score computed for a respective clone a by a first procedure comprising: i. identifying a plurality of neoantigens in the respective clone a; ii. computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and iii. determining the corresponding clone fitness score of the respective clone a as an aggregate of the neoantigen recognition potentials across the plurality of neoantigens in the respective clone a; and e. computing a total fitness for the one or more samples as a sum of the clone fitness scores across the plurality of clones, wherein each clone fitness score is weighted by the initial frequency Xa of the corresponding clone a, and the total fitness quantifies the likelihood that the human subject afflicted with the cancer will be responsive to the treatment regimen.

Aspects of the invention are directed towards a non-transitory computer readable storage medium for determining a likelihood that a human subject afflicted with a cancer will be responsive to a treatment regimen that comprises administering a checkpoint blockade immunotherapy directed to the cancer to the subject. In embodiments, the non-transitory computer readable storage medium stores one or more programs for execution by one or more processors of a computer system, the one or more computer programs comprising instructions for carrying out the method of any one of the aspects or embodiments described herein.

Aspects of the invention are directed towards a method for identifying within a computing system a neoantigen to target as an immunotherapy for a cancer. In embodiments, the computing system comprises one or more programmable processors, memory, and a plurality of instructions stored on the memory that are executable by the one or programmable processors. In embodiments, the method comprises: a) obtaining a plurality of sequencing reads from one or more samples from a human cancer subject that is representative of the cancer; b) determining a human leukocyte antigen (HLA) type of the human subject from the plurality of sequencing reads; c) determining a plurality of clones, and for each respective clone a in the plurality of clones, an initial frequency Xa of the respective clone a in the one or more samples from the plurality of sequencing reads; for each respective clone a in the plurality of clones, computing a corresponding clone fitness score of the respective clone, thereby computing a plurality of clone fitness scores, each corresponding clone fitness score computed for a respective clone a by a first procedure comprising: i. identifying a plurality of neoantigens in the respective clone a; ii. computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and iii. determining the corresponding clone fitness score of the respective clone a as an aggregate of the neoantigen recognition potentials across the plurality of neoantigens in the respective clone a; and d) selecting at least a first neoantigen from a plurality of neoantigens for a respective clone a in the plurality of respective clones based upon the recognition potential of the first neoantigen to target as an immunotherapy for the cancer. In embodiments, the obtaining (a), the determining (b), the determining (c), and the computing (d) are repeated for a plurality of human subjects across a plurality of HLA types. In embodiments, the first neoantigen is selected on the basis of the recognition potential of the first neoantigen across the plurality of HLA types. In embodiments, the obtaining (a), the determining (b), the determining (c), and the computing (d) are repeated for a plurality of human subjects. In embodiments, the first neoantigen is selected on the basis of the recognition potential of the first neoantigen across the plurality of human subject. In embodiments, wherein the cancer is a carcinoma, a melanoma, a lymphoma/leukemia, a sarcoma, or a neuro-glial tumor. In embodiments, the cancer is lung cancer, pancreatic cancer, colon cancer, stomach cancer, esophagus cancer, breast cancer, ovary cancer, prostate cancer, or liver cancer. In embodiments, each clone a in the plurality of clones is uniquely defined by a unique set of somatic mutations, and wherein the plurality of clones is determined by a variant allele frequency of each respective somatic mutation in a plurality of somatic mutations determined from the whole-genome sequencing data. In further embodiments, the somatic mutation is a single nucleotide variant or an indel. In embodiments, the plurality of clones is determined by identifying a plurality of inferred copy number variations using the whole-genome sequencing data. In embodiments, each clone a in the plurality of clones is uniquely defined by a unique set of somatic mutations, and the plurality of clones is determined by a combination of (i) a variant allele frequency of each respective somatic mutation in the plurality of somatic mutations determined from the whole-genome sequencing data and (ii) an identification of a plurality of inferred copy number variations using the whole-genome sequencing data. In embodiments, the plurality of sequencing reads exhibits an average read depth of less than 40. In embodiments, the plurality of sequencing reads exhibits an average read depth of between 25 and 60. In embodiments, each neoantigen in the plurality of neoantigens of a clone in the plurality of clones is a nonamer peptide. In embodiments, each neoantigen in the plurality of neoantigens of a clone in the plurality of clones is a peptide comprising eight, nine, ten, or eleven residues in length. In embodiments, the determining the HLA type of the human subject is determined from the plurality of sequencing reads. In embodiments, the determining the HLA type of the human subject is determined using a polymerase chain reaction using a biological sample from the cancer subject. In embodiments, the plurality of clones comprises two clones. In embodiments, the plurality of clones comprises between two clones and ten clones. In embodiments, the initial frequency Xa of the respective clone a in the one or more samples is determined using the plurality of sequencing reads from the one or more samples from the human subject.

Aspects of the invention are directed towards a system for identifying an immunotherapy for a cancer. In embodiments, the system comprises: memory; one or more processors; and one or more modules stored in memory and configured for execution by the one or more processors, the modules comprising instructions for: (A) obtaining a plurality of sequencing reads from one or more samples from the human cancer subject that is representative of the cancer; (B) determining a human leukocyte antigen (HLA) type of the human subject from the plurality of sequencing reads; (C) determining a plurality of clones, and for each respective clone a in the plurality of clones, an initial frequency Xa of the respective clone a in the one or more samples from the plurality of sequencing reads; (D) for each respective clone a in the plurality of clones, computing a corresponding clone fitness score of the respective clone, thereby computing a plurality of clone fitness scores, each corresponding clone fitness score computed for a respective clone a by a first procedure comprising: (a) identifying a plurality of neoantigens in the respective clone a; (b) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (c) determining the corresponding clone fitness score of the respective clone a as an aggregate of the neoantigen recognition potentials across the plurality of neoantigens in the respective clone a; and (E) selecting at least a first neoantigen from a plurality of neoantigens for a respective clone a in the plurality of respective clones based upon the recognition potential of the first neoantigen as the immunotherapy for the cancer.

Aspects of the invention are directed towards a system for identifying an immunotherapy for a cancer. In embodiments, the system comprises: memory; one or more processors; and one or more modules stored in memory and configured for execution by the one or more processors, the modules comprising instructions for executing the method of any one of the aspects or embodiments described herein.

Aspects of the invention are directed towards a non-transitory computer readable storage medium for identifying an immunotherapy for a cancer. In embodiments, the non-transitory computer readable storage medium stores one or more programs for execution by one or more processors of a computer system, the one or more computer programs comprising instructions for: (A) obtaining a plurality of sequencing reads from one or more samples from a human subject that is representative of the cancer; (B) determining a human leukocyte antigen (HLA) type of the human subject from the plurality of sequencing reads; (C) determining a plurality of clones, and for each respective clone a in the plurality of clones, an initial frequency Xa of the respective clone a in the one or more samples from the plurality of sequencing reads; (D) for each respective clone a in the plurality of clones, computing a corresponding clone fitness score of the respective clone, thereby computing a plurality of clone fitness scores, each corresponding clone fitness score computed for a respective clone a by a first procedure comprising: i. identifying a plurality of neoantigens in the respective clone a; ii. computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and iii. determining the corresponding clone fitness score of the respective clone a as an aggregate of the neoantigen recognition potentials across the plurality of neoantigens in the respective clone a; and (E) selecting at least a first neoantigen from a plurality of neoantigens for a respective clone a in the plurality of respective clones based upon the recognition potential of the first neoantigen as the immunotherapy for the cancer.

Aspects of the invention are directed towards a non-transitory computer readable storage medium for identifying an immunotherapy for a cancer. In embodiments, the non-transitory computer readable storage medium stores one or more programs for execution by one or more processors of a computer system, the one or more computer programs comprising instructions for executing the method of any one of the aspects or embodiments described herein.

Aspects of the invention are directed towards a method of treating a human subject afflicted with a cancer with a checkpoint blockade immunotherapy directed to the cancer. In embodiments, the method comprises: (1) determining within a computing system that the human subject is likely to be responsive to the checkpoint blockade immunotherapy, wherein the computing system has one or more programmable processors, memory, and a plurality of instructions stored on the memory that are executable by the one or programmable processors; and (2) administering the checkpoint block immunotherapy when it is determined that the human subject is likely to be responsive to the checkpoint blockade immunotherapy; wherein the determination that the human subject will be responsive to the checkpoint blockade immunotherapy comprises: (A) obtaining a plurality of sequencing reads from one or more samples from the human cancer subject that is representative of the cancer; (B) determining a human leukocyte antigen (HLA) type of the human subject; (C) determining a plurality of clones, and for each respective clone a in the plurality of clones, an initial frequency Xa of the respective clone a in the one or more samples; (D) for each respective clone a in the plurality of clones, computing a corresponding clone fitness score of the respective clone, thereby computing a plurality of clone fitness scores, each corresponding clone fitness score computed for a respective clone a by a first procedure comprising: (a) identifying a plurality of neoantigens in the respective clone a; (b) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (c) determining the corresponding clone fitness score of the respective clone a as an aggregate of the neoantigen recognition potentials across the plurality of neoantigens in the respective clone a; and (E) computing a total fitness for the one or more samples as a sum of the clone fitness scores across the plurality of clones, wherein each clone fitness score is weighted by the initial frequency Xa of the corresponding clone a, and the total fitness quantifies the likelihood that the human subject afflicted with the cancer will be responsive to the checkpoint blockade immunotherapy. In embodiments, the checkpoint blockade immunotherapy comprises an antibody, fragment, or derivative thereof. In embodiments, the antibody is specific for CTLA4, PD1, PD-L1, LAG3, TIM-3, GITR, OX40, CD40, TIGIT, 4-1BB, B7-H3, B7-H4, or BTLA. In further embodiments, the checkpoint blockade immunotherapy comprises ipilimumab or tremelimumab. In embodiments, the cancer is a carcinoma, a melanoma, a lymphoma/leukemia, a sarcoma, or a neuro-glial tumor. In embodiments, the cancer is lung cancer, pancreatic cancer, colon cancer, stomach cancer, esophagus cancer, breast cancer, ovary cancer, prostate cancer, or liver cancer.

Aspects of the invention are directed towards a method for determining T-cell cross-reactivity between antigens. In embodiments, the method comprises: (1) determining within a computing system that the human subject is likely to be responsive to the checkpoint blockade immunotherapy, wherein the computing system has one or more programmable processors, memory, and a plurality of instructions stored on the memory that are executable by the one or programmable processors; and (2) administering the checkpoint block immunotherapy when it is determined that the human subject is likely to be responsive to the checkpoint blockade immunotherapy; wherein the determination that the human subject will be responsive to the checkpoint blockade immunotherapy comprises: (A) obtaining a plurality of sequencing reads from one or more samples from the human cancer subject that is representative of the cancer; (B) determining a human leukocyte antigen (HLA) type of the human subject; (C) determining a plurality of clones, and for each respective clone a in the plurality of clones, an initial frequency Xa of the respective clone a in the one or more samples; (D) for each respective clone a in the plurality of clones, computing a corresponding clone fitness score of the respective clone, thereby computing a plurality of clone fitness scores, each corresponding clone fitness score computed for a respective clone a by a first procedure comprising: (a) identifying a plurality of neoantigens in the respective clone a; (b) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (c) determining the corresponding clone fitness score of the respective clone a as an aggregate of the neoantigen recognition potentials across the plurality of neoantigens in the respective clone a; and (E) computing a total fitness for the one or more samples as a sum of the clone fitness scores across the plurality of clones, wherein each clone fitness score is weighted by the initial frequency Xa of the corresponding clone a, and the total fitness quantifies the likelihood that the human subject afflicted with the cancer will be responsive to the checkpoint blockade immunotherapy. In embodiments, the checkpoint blockade immunotherapy comprises an antibody, fragment, or derivative thereof. In embodiments, the antibody is specific for CTLA4, PD1, PD-L1, LAG3, TIM-3, GITR, OX40, CD40, TIGIT, 4-1BB, B7-H3, B7-H4, or BTLA. In further embodiments, the checkpoint blockade immunotherapy comprises ipilimumab or tremelimumab. In embodiments, the cancer is a carcinoma, a melanoma, a lymphoma/leukemia, a sarcoma, or a neuro-glial tumor. In embodiments, the cancer is lung cancer, pancreatic cancer, colon cancer, stomach cancer, esophagus cancer, breast cancer, ovary cancer, prostate cancer, or liver cancer.

Other objects and advantages of this invention will become readily apparent from the ensuing description.

### BRIEF DESCRIPTION OF THE FIGURES

FIG. 1 shows that long-term pancreatic ductal adenocarcinomas (PDAC) survivors evolve tumors with distinct recurrence time, multiplicity, and tissue-tropism, as described in the Examples. Panel A shows the experimental design and samples used. Panel B shows overall survival and Panel C shows disease-free survival of PDAC patients for short-term survivors (STS) and long-term survivors (LTS). Panel D shows number of recurrent PDACs, Panel E shows correlation with overall survival of recurrent PDACs, Panel F shows patterns of recurrent PDACs, and Panel G shows sites of recurrent PDAC (STS other=omentum, mesentery, aorta, diaphragm, perirectum; LTS other=pericardium, inferior vena cava, adrenal, kidney, liver. n=individual patients for Panels B-F, or recurrent tumors for Panel G. Horizontal bars=median. P values by log-rank test (Mantel-Cox) for Panels B and C, two-tailed Mann-Whitney test for Panel D, two-tailed Pearson correlation for Panel E, and Chi-square test for Panel F.

FIG. 2 shows that long-term PDAC survivors evolve tumors with fewer neoantigens, as described in the Examples. Panel A shows Shannon entropy (S, left), and difference in Shannon entropy in recurrent (S_{rec}) to primary (Sₚᵣᵢₘ) PDACs (right). Panel B shows tumor mutational burden (TMB) and neoantigen number (NA) in primary and recurrent PDACs. Panel C shows difference in TMB and NA between recurrent to primary PDACs for STS and LTS. Panel D shows difference in number of mutations that generate neoantigens (NA Mut) between recurrent to primary PDACs. n=individual tumors. Prim—Primary; Rec—Recurrent. Horizontal bars=median. P values by two-tailed Mann-Whitney test.

FIG. 3 shows that high quality neoantigens are immunoedited in long-term PDAC survivors, as described in the Examples. Panel A shows the neoantigen quality model. Panel B shows model and experimental approach to estimate cross reactivity distance C. Panel C shows measured (top) and fitted (bottom) p^{MT}-T cell receptor (TCR) activation curves (A.A [AA] position 4) for strong and weak p^{WT}-TCR pairs. Panel D shows measured (top) and fitted (bottom) p^{MT}-TCR activation heat maps (all AA positions) for strong and weak p^{WT}-TCR pairs. Panel E shows composite p^{MT}-TCR EC₅₀s of all strong and weak p^{WT}-TCR pairs. Panel F shows p^{MT}-TCR activation heat map and observed versus modeled C(p^{WT} p^{MT}) for HLA*B27:05 restricted p^{WT}-TCR pair (n=number of single AA-substituted peptide pairs). Panel G shows cross-reactivity distance C. Panel H shows observed AA substitution frequency versus matrix M defined substitution distance in primary and recurrent STS and LTS PDACs (M distance=matrix M (from Panel G) defined AA distance; n number of substitutions). Panel I shows cumulative probability distributions of log C and D (n=number of neoantigens) for STS and LTS. Red rectangles in heat maps=AA in p^{WT}. Green line=linear regression fit. Heat maps ordered by C defined nearest AA neighbor. P values by two-tailed Pearson correlation (Panels F and H) and two-sided Kolmogorov-Smirnov test (Panel I)

FIG. 4 shows top ranked T cells in LTS tumors have more similar CDR3β sequences. (Panel A) T cell receptor (TCR) CDR3β sequence dissimilarity (TCR dissimilarity index) in STS and LTS primary and recurrent PDACs. TCR dissimilarity index calculated using the Restricted Boltzmann Machine model 18. n=individual tumors. Horizontal bars=median. (Panel B) Trend of P value of TCR dissimilarity index between STS and LTS PDACs (as in left panel) with number of clones in the sample. n=17 tumors. Blue line indicates a P value of 0.05; circle=mean P value; error bars=standard error of the mean. (Panel C) TCR dissimilarity index based on T cell clone size (Supplementary Methods) and immune fitness cost F̅₁. Green line=linear regression fit. P value by

FIG. 5 shows tumor mutational features in short and long-term PDAC survivors, as described in the Examples. Panel A shows whole-exome sequencing depth in primary and recurrent PDACs from STS and LTS. Panel B shows number of synonymous mutations in primary and recurrent PDACs from STS and LTS. Panel C shows oncoprints of driver mutation frequencies in primary and recurrent PDACs (frequencies=percentage of patients in each cohort that harbor corresponding driver gene mutations). Panel D shows frequency of primary (left) and recurrent (right) PDACs with mutations in ≥3 oncogenes. Panel E shows number of nonsynonymous mutations (TMB) versus number of mutations in oncogenes in primary and recurrent PDACs (n=individual tumors; horizontal bars=median). P value by two-tailed Mann-Whitney test.

FIG. 6 shows tumor evolutionary trees in short and long-term PDAC survivors, as described in the Examples. Panels A and B shows tumor clone phylogenies and frequencies in primary and recurrent PDACs from STS (Panel A, n=6) and LTS (Panel B, n=9).

FIG. 7 shows TCR transduction and antigen specificity, as described in the Examples. Panel A shows experimental schema to transduce and measure p^{WT}-specific TCR activation (hVα,ρ=human α and β variable regions; mCα,β=mouse α and β constant regions). Panel B shows representative gating strategy to detect transduced TCR activation and specificity. Panels C-E show sequences of model p^{WT}s and p^{WT}-specific TCRs, and TCR activation across varied p^{WT} concentrations.

FIG. 8 shows that T cell activation is variably degenerate to single A.A substitutions, as described in the Examples. Panels A-C show T cell activation to model p^{WT}s (black curves) and single A.A substituted p^{MT}s (color curves).

FIG. 9 shows that T cell activation to degenerate substitutions follows a sigmoidal function. Panel A-C show fitted T cell activation curves to model p^{WT}s (black curves) and single A.A substituted p^{MT}s (color curves).

FIG. 10 shows cross-reactivity distance C model. Panels A and B show A.A position dependent factor (Panel A) and substitution matrix (Panel B) of cross-reactivity model based on TCR cross-reactivity to strong (CMV) and weak (gp100) p^{WT}s and single A.A substituted p^{MT}s (FIG. 3, Panels D and E). Panel C shows correlation of substitution-induced differential MHC-I binding (log(A)=K_{d}^{WT}/K_{d}^{MT}) and substitution induced differential TCR activation (log(C)=EC₅₀^{MT}/EC₅₀^{WT}) for all model p^{WT}-TCR pairs and single A.A substituted p^{MT}s. K_{d}^{WT} and K_{d}^{MT} determined through computational predictions of p^{WT} and p^{MT} binding to HLA-A*02:01 (CMV, gp100 peptides) and HLA-B*27:05 (tumor neopeptide) with Net MHC 3.4. EC₅₀^{MT} and EC₅₀^{WT}measured experimentally through p^{WT} and p^{MT} reactivity to TCRs. n=individual peptide-TCR measurements. P values by two-tailed Pearson correlation.

FIG. 11 shows that LTS and STS PDACs have equivalent genetic changes in HLA class-I pathway genes. Panel A shows number of mutations (synonymous and non-synonymous), homozygous deletions, heterozygous deletions, and copy number neutral loss of heterozygosity (LOH) changes in primary and recurrent PDACs. Panel B shows mRNA expression in HLA class-I pathway genes by bulk RNA sequencing (ICGC, TCGA cohorts) and transcriptional analysis (Affymetrix, Memorial Sloan Kettering Cancer Center [MSKCC] cohort) in primary PDAC tumors. Panel C shows representative multiplexed immunohistochemical images (left) and ratio (right) of MHC-I+ tumor cells (CK19+) and MHC-I+ non-tumor cells (CK19−) in STS and LTS primary PDACs. n=individual tumors. Horizontal bars=median. Horizontal bars on violin plots show median and quartiles. P value by Wald's test adjusted for multiple comparison testing.

FIG. 12 shows evaluation of clone fitness model predictions. The log-likelihood score (eq. (31)) is shown for the STS and LTS cohorts to estimate the statistical information gain of fitness models and the amount of evidence of the selective pressures captured by each of the models. The orange bars show the aggregated log-likelihood scores, ΔL^{STS}(F, F_{N}) and ΔL^{LTS}(F, F_{N}), of the two-component fitness model, F, with parameters σₗ, σ_{P} optimized for each recurrent tumor sample, as compared to the null model, F_{N}, standing for neutral clone evolution, with zero fitness and parameters σₗ=0, σ_{P}=0. The red bars present the corresponding aggregated log-likelihood scores ΔL^{STS}(F_{P}, F_{N}) and ΔL^{LTS}(F_{P}, F_{N}) for the driver-gene only fitness model, F_{P}, which accounts for positive selection on driver genes but disregards the effect of immune selection, with parameter σₗ=0, σ_{P} optimized for each recurrent tumor sample. Finally, the blue bars present the corresponding aggregated log-likelihood scores ΔL^{STS}(Fₗ, F_{N} and ΔL^{LTS}(Fₗ, F_{N}) for the immune-only fitness model, F_{I}, with parameter σ_{P}=0 and σₗ optimized for each recurrent tumour sample.

FIG. 13 shows neoantigen quality fitness models.

FIG. 14 shows the neoantigen quality fitness model identifies edited clones to predict the clonal composition of recurrent tumors. Panel A shows recurrent tumour clone composition prediction based on the primary tumour composition and the fitness model. Panel B shows model fitted \hat{X}_{rec}^{∝}/Xₚᵣᵢₘ^{∝} and observed X_{rec}^{∝}/X_{rec}^{∝} clone frequency changes for the STS (left) and LTS (right) cohorts. Frequency ratios below the sampling threshold were evaluated with pseudocounts. Panels C-E show the immune fitness cost F̅_{I} of recurrent tumors (Panel C), new clones (Panel E), and the percentage of new neoantigens in recurrent tumors (Panel D). Panel F shows TCR dissimilarity index and immune fitness cost F̅_{I} in tumors. n indicates the number of tumors. The green line is a linear regression fit. The horizontal bars show the median values. P values were determined using two-tailed Spearman correlation (Panel B), two-tailed Pearson correlation (Panel F) and two-tailed Mann-Whitney U-tests (Panels C-E).

FIG. 15 shows a flowchart corresponding to a method performed by software components of a system for providing systems and methods for determining T-cell cross-reactivity between antigens 1500 according to embodiments of the present invention.

FIG. 16 shows a flowchart corresponding to a method performed by software components of a system for providing systems and methods for determining T-cell cross-reactivity between antigens according to embodiments of the present invention.

FIG. 17 shows a flowchart corresponding to a method performed by software components of a system for providing systems and methods for determining T-cell cross-reactivity between antigens according to embodiments of the present invention.

FIG. 18 shows a flowchart corresponding to a method performed by software components for computing each corresponding clone fitness score computed for a respective clone a according to embodiments of the present invention.

FIG. 19 shows a flowchart corresponding to a method performed by software components providing a method for treating a human subject afflicted with a cancer with a checkpoint blockade immunotherapy directed to the cancer according to embodiments of the present invention.

FIG. 20 shows is a flowchart corresponding to a method performed by software components for the determination that the human subject will be responsive to the checkpoint blockade immunotherapy according to embodiments of the present invention.

FIG. 21 shows a flowchart corresponding to a method performed by software components for providing a corresponding clone fitness score computed for a respective clone a according to embodiments of the present invention.

FIG. 22 shows a computing system of software components for providing a method for providing systems and methods for determining T-cell cross-reactivity between antigens according to embodiments of the present invention.

FIG. 23 shows a computer system 2300 adapted according to certain embodiments of the server and/or the user interface device utilized to execute the software component modules disclosed herein.

FIG. 24 shows clinicopathologic characteristics of the matched primary and recurrent PDAC cohort.

FIG. 25 shows a comprehensive list of all neopeptide sequences and HLA alleles with predicted neopeptide binding.

FIG. 26 shows a comprehensive list of all human infectious derived, class-I restricted peptide sequences with positive immune assays derived from the Immune Epitope Database used in this study.

### DETAILED DESCRIPTION OF THE INVENTION

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” can refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” or “approximately” can refer to within an acceptable error range for the particular value as determined by one of ordinary skill in the art, which will depend in part on how the value is measured or determined, i.e., the limitations of the measurement system. For example, “about” can refer to within 3 or more than 3 standard deviations, per the practice in the art. Alternatively, “about” can refer to a range of up to 20%, e.g., up to 10%, up to 5%, or up to 1% of a given value. Alternatively, particularly with respect to biological systems or processes, the term can refer to within an order of magnitude, e.g., within 5-fold, or within 2-fold, of a value.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

The “median” value can refer to the median value obtained from a population of subjects having a cancer. The median values can be previously determined reference values or can be contemporaneously determined values.

It will be understood that, although the terms first, second, etc. can be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

Unless defined otherwise, all technical and scientific terms used herein have the meaning commonly understood by a person skilled in the art to which this invention belongs. The following references provide one of ordinary skill in the art with a general definition of many of the terms used herein: Singleton et al, Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991); Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al, ed., 1994); “A Practical Guide to Molecular Cloning” (Perbal Bernard V., 1988); “Phage Display: A Laboratory Manual” (Barbas et al, 2001). The contents of these references and other references containing standard protocols, widely known to and relied upon by those of skill in the art, including manufacturers' instructions are hereby incorporated by reference as part of the presently disclosed subject matter.

Aspects of the invention provide strategies for improving cancer therapy (e.g., immunotherapy) by selection of particular subjects to receive therapy based on the neoantigen portfolio of the subjects. Aspects of the invention also provides methods for predicting responsiveness of cancer patients to immunotherapy based on the subject's neoantigen portfolio.

Aspects of the invention are drawn towards a method for determining within a computing system a likelihood that a human subject afflicted with a cancer will be responsive to a treatment regimen that comprises administering a checkpoint blockade immunotherapy directed to the cancer of the subject.

The terms “cancer” and “cancerous” can refer to or describe, for example, the physiological condition in mammals that is characterized by unregulated cell growth. Examples of cancer include but are not limited to, carcinoma, melanoma, lymphoma/leukemia, neuro-glial tumor, chronic lymphocytic leukemia, non-small cell lung cancer, clear cell renal carcinoma, mesothelioma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, stomach cancer, esophageal cancer, colorectal cancer, endometrial or uterine carcinoma, salivary gland carcinoma, kidney cancer, liver cancer, prostate cancer, pancreatic cancer, vulval cancer, thyroid cancer, hepatic carcinoma and various types of head and neck cancer. For example, the cancer is renal cell carcinoma, such as ccRCC. Exemplary cancers can be benign or malignant and can be one that is influenced by the immune system.

The phrase “computing system” can refer to a single computer or a collection of computers or similar devices that communicate together to perform one or more functions of the computer. For example, the computing system can comprise one or more programmable processors, memory, and/or instructions. For example, the computing system can comprise a plurality of instructions stored on the memory that are executable by the one or programmable processors.

In embodiments, the computing system can comprise a data collection device that receives sequencing reads (e.g., whole genome sequencing reads, exome sequencing reads, targeted sequencing reads, etc.) originating from one or more biological samples (e.g., biopsies) of a subject. In embodiments, the data collection device can receive such data directly from nucleic acid sequencers. For instance, in embodiments the data collection device receives this data wirelessly through radio-frequency signals. In embodiments such signals are in accordance with an 802.11 (WiFi), Bluetooth, or ZigBee standard. In embodiments, the data collection device receives such data directly. In embodiments the data collection device receives this data across a communications networks.

Examples of networks include, but are not limited to, the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.1 lac, IEEE 802.1 lax, IEEE 802. 1 ib, IEEE 802.1 lg and/or IEEE 802.1 In), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of the present disclosure.

Of course, other topologies of the system are possible. For instance, rather than relying on a communications network, information may be sent directly to the data collection device. Further, the data collection device may constitute a portable electronic device, a server computer, or in fact constitute several computers that are linked together in a network or be a virtual machine in a cloud computing context.

In embodiments, the data collection device can comprise one or more computers. Thus, in embodiments, methods described herein can be spread across any number of networked computers and/or resides on each of several networked computers and/or is hosted on one or more virtual machines at a remote location accessible across the communications network. One of skill in the art will appreciate that any of a wide array of different computer topologies can be used for the application and all such topologies are within the scope of the present disclosure.

In embodiments, the data collection device can comprise one or more processing units (CPU's), a network or other communications interface, a memory (e.g., random access memory), one or more magnetic disk storage and/or persistent devices optionally accessed by one or more controllers, one or more communication busses for interconnecting the aforementioned components, a user interface, the user interface including a display and input (e.g., keyboard, keypad, touch screen), and a power supply for powering the aforementioned components. In embodiments, the input is a touch-sensitive display, such as a touch-sensitive surface. In embodiments, the user interface includes one or more soft keyboard embodiments. The soft keyboard embodiments may include standard (QWERTY) and/or non-standard configurations of symbols on the displayed icons. In embodiments, data in memory is seamlessly shared with non-volatile memory using known computing techniques such as caching. In embodiments, memory and/or memory includes mass storage that is remotely located with respect to the central processing unit(s). In other words, some data stored in memory and/or memory may in fact be hosted on computers that are external to the data collection device but that can be electronically accessed by the data collection device over an Internet, intranet, or other form of network or electronic cable using network interface.

In embodiments, the memory of the data collection device can store an operating system that includes procedures for handling various basic system services; a subject assessment module; whole genome sequencing reads for the subject, the whole genome sequencing reads comprising, for each biological sample from the subject, a plurality of sequence reads; a human leukocyte antigen (HLA) type of the subject; a plurality of clones associated with a subject, and, for each respective clone in the plurality of clones, an initial frequency Xa of the clone, a clone fitness score, and a plurality of neoantigens of the clone, each such neoantigen including a neoantigen recognition potential, an amplitude, an optional MHC affinity, and optional MHC affinity of the wildtype sequence corresponding to the neoantigen, and a probability of T-cell receptor recognition; and a total fitness of the cancer.

In embodiments, the subject assessment module is accessible within any browser (phone, tablet, laptop/desktop). In embodiments the subject assessment module runs on native device frameworks and is available for download onto the data collection device running an operating system such as Android or iOS.

In embodiments, one or more of the data elements or modules of the data collection device described herein can be stored in one or more of the previously described memory devices and correspond to a set of instructions for performing a function described above. The above-identified data, modules, or programs (e.g., sets of instructions) need not be implemented as separate software programs, procedures or modules, and thus various subsets of these modules may be combined or otherwise re-arranged in various implementations. In implementations, the memory and/or optionally stores a subset of the modules and data structures identified above. Furthermore, in embodiments the memory and/or stores additional modules and data structures not described above. Further still, in embodiments, the data collection device stores data using a neoantigen fitness model for two or more subjects, five or more subjects, one hundred or more subjects, or 1000 or more subjects.

In embodiments, a data collection device is a smart phone (e.g., an iPhone), laptop, tablet computer, desktop computer, or other form of electronic device (e.g., a gaming console). In embodiments, the data collection device is not mobile. In embodiments, the data collection device is mobile.

It should be appreciated that the data collection device optionally has a different configuration or arrangement of the components. For example, the various components can be implemented in hardware, software, firmware, or a combination thereof, including one or more signal processing and/or application specific integrated circuits.

RF (radio frequency) circuitry of network interface can receive and send RF signals, also called electromagnetic signal. In embodiments, the sequencing reads and HLA type and/or other data is received using this RF circuitry from one or more devices such as nucleic acid sequencers. In embodiments, the RF circuitry converts electrical signals to/from electromagnetic signals and communicates with communications networks and other communications devices via the electromagnetic signals. The RF circuitry optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry optionally communicates with the communication network 106. In some embodiments, the circuitry does not include RF circuitry and, in fact, is connected to the network through one or more hard wires (e.g., an optical cable, a coaxial cable, or the like).

In embodiments, the power supply optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management, and distribution of power in portable devices.

The data collection device can comprise an operating system that includes procedures for handling various basic system services. The operating system (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components.

In embodiments the data collection device is a smart phone. In other embodiments, the data collection device is not a smart phone but rather is a tablet computer, desktop computer, emergency vehicle computer, or other form or wired or wireless networked device.

While the system can work standalone, in some embodiments it can also be linked with electronic medical records to exchange information in any way.

In embodiments, the method can comprise (a) obtaining a plurality of sequencing reads from one or more samples from the human cancer subject that is representative of the cancer; (b) determining a human leukocyte antigen (HLA) type of the human subject; (c) computing for a plurality of clones, and for each respective clone a in the plurality of clones, an initial frequency Xa of the respective clone a in the one or more samples; (d) for each respective clone a in the plurality of clones, computing a corresponding clone fitness score of the respective clone, thereby computing a plurality of clone fitness scores; and (e) computing a total fitness for the one or more samples as a sum of the clone fitness scores across the plurality of clones, wherein each clone fitness score is weighted by the initial frequency Xa of the corresponding clone a, the total fitness quantifies the likelihood that the human subject afflicted with the cancer will be responsive to the treatment regimen

In embodiments, each corresponding clone fitness score for a respective clone a can be computed by a first procedure comprising: identifying a plurality of neoantigens in the respective clone a; computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and determining the corresponding clone fitness score of the respective clone a as an aggregate of the neoantigen recognition potentials across the plurality of neoantigens in the respective clone a.

Embodiments can further comprise administering to the subject the checkpoint blockade immunotherapy if the subject is likely to be responsive to the treatment regimen.

The term “subject” can refer to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested). In embodiments, the subject is a human.

The term “sample” can refer to a biological sample obtained or derived from a source of interest, as described herein. In embodiments, a source of interest comprises an organism, such as an animal or human. In embodiments, a biological sample is a biological tissue or fluid. Non-limiting biological samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom, etc.

In embodiments, the sample can comprise a population of cells. The term “cell population” can refer to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, or at least about 1000 cells expressing similar or different phenotypes.

The term “immunotherapy” can refer to a treatment that involves the activation of a specific immune response and/or an immune effector function. Immunotherapy can function to remove cells that express antigens (e.g., neoantigens) from a patient. Such elimination may take place as a result of the improvement or induction of an immune response and/or immune effector function in a patient specific for an antigen or a cell expressing an antigen.

Immunotherapies that boost the ability of endogenous T cells to destroy cancer cells have demonstrated therapeutic efficacy in a variety of human malignancies. However, some cancer patients have resistance to certain immunotherapies. The presently disclosed subject matter provides methods for identifying cancer patients who would be candidates and/or who would likely to respond to an immunotherapy.

Non-limiting examples of immunotherapies include therapies comprising one or more immune checkpoint blocking antibody, adoptive T cell therapies, non-checkpoint blocking antibody-based immunotherapies, small molecule inhibitors, cancer vaccines, and combinations thereof.

The phrase “checkpoint blockade therapy” can refer to the use of agents that inhibit immune checkpoint nucleic acids and/or proteins. Immune checkpoints share the common function of providing inhibitory signals that suppress immune response and inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof, as well as RNA interference, antisense, nucleic acid aptamers, etc. that can downregulate the expression and/or activity of immune checkpoint nucleic acids, or fragments thereto.

Non-limiting examples of immune checkpoint blocking antibodies include antibodies against CTLA cytotoxic T-lymphocyte antigen 4 (anti-CTLA4 antibodies), antibodies against programmed death 1 (anti-PD-1 antibodies), antibodies against Programmed death-ligand 1 (anti-PD-L1 antibodies), antibodies against lymphocyte activation gene-3 (anti-LAG3 antibodies), antibodies against T cell immunoglobulin and mucin domain-containing protein 3 (anti-TIM-3 antibodies), antibodies against glucocorticoid-induced TNFR-related protein (GITR), antibodies against OX40, antibodies against CD40, antibodies against T cell immunoreceptor with Ig and ITIM domains (TIGIT), antibodies against 4-1BB, antibodies against B7 homolog 3 (anti-B7-H3 antibodies), antibodies against B7 homolog 4 (anti-B7-H4 antibodies), and antibodies against B- and T-lymphocyte attenuator (anti-BTLA antibodies).

Adoptive T cell therapy involves the isolation and ex vivo expansion of tumor specific T cells to achieve greater number of T cells. The tumor specific T cells are infused into cancer patients to give their immune system the ability to overwhelm remaining tumor via T cells which can attack and kill cancer. Non-limiting examples of adoptive T cell therapy include tumor-infiltrating lymphocyte (TIL) cell therapies, therapies comprising engineered or modified T cells, e.g., T cells engineered or modified with T cell receptor (TCR-transduced T cells), or T cells engineered or modified with chimeric antigen receptor (CAR-transduced T cells). These engineered or modified T cells recognize specific antigens associated with the cancers and attack cancers.

In embodiments, the checkpoint blockade immunotherapy can be an antibody, fragment, or derivative thereof. An “antibody” or “antibodies” can refer to antigen-binding proteins of the immune system. An “antibody” can refer to whole, full-length antibodies having an antigen-binding region, and any fragment thereof in which the “antigen-binding portion” or “antigen-binding region” is retained, or single chains, for example, single chain variable fragment (scFv), thereof. The term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments that lack the Fc fragment of intact antibody, clear more rapidly from the circulation, and can have less non-specific tissue binding of an intact antibody (Wahl et al, J. Nucl. Med. 24:316-325 (1983). In certain embodiments, an antibody is a glycoprotein comprising at least two heavy (H) chains and two light (L) chains interconnected by disulfide bonds. Each heavy chain is comprised of a heavy chain variable region (abbreviated herein as VH) and a heavy chain constant (CH) region. The heavy chain constant region is comprised of three domains, CH 1, CH 2 and CH 3. Each light chain is comprised of a light chain variable region (abbreviated herein as VL) and a light chain constant CL region. The light chain constant region is comprised of one domain, CL. The VH and VL regions can be further sub-divided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antigen-binding portion”, “antigen-binding fragment”, or “antigen-binding region” of an antibody can refer to that region or portion of an antibody that binds to the antigen and which confers antigen specificity to the antibody; fragments of antigen-binding proteins. The antigen-binding function of an antibody can be performed by fragments of a full-length antibody. Examples of antigen-binding portions encompassed within the term “antibody fragments” of an antibody include a Fab fragment, a monovalent fragment consisting of the VL, VH, CL and CHT domains; a F(ab)2 fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; a dAb fragment (Ward et al., 1989 Nature 341:544-546), which consists of a VH domain; and an isolated complementarity determining region (CDR).

In embodiments, the antibody, fragment, or derivative thereof can be an anti-cancer antibody. For example, the antibody can be specific for CTLA4, PD1, PD-L1, LAG3, TIM-3, GITR, OX40, CD40, TIGIT, 4-1BB, B7-H3, B7-H4, or BTLA. For example, the checkpoint blockade immunotherapy comprises ipilimumab or tremelimumab. Ipilimumab is a monoclonal antibody medication that works to activate the immune system by targeting CTLA-4, a protein receptor that downregulates the immune system. Tremelimumab is a fully human monoclonal antibody against CTLA-4 that is an immune checkpoint blocker.

In embodiments, the disclosed subject matter provides methods of predicting the responsiveness of subjects (e.g., cancer subjects) to an immunotherapy (e.g., “the responsiveness prediction method”). The term “response” or “responsiveness” can refer to an alteration in a subject's condition that occurs as a result of or correlates with treatment. In embodiments, a response is a beneficial response. In embodiments, a beneficial response can include stabilization of the condition (e.g., prevention or delay of deterioration expected or typically observed to occur absent the treatment), amelioration (e.g., reduction in frequency and/or intensity) of one or more symptoms of the condition, and/or improvement in the prospects for cure of the condition, etc. In embodiments, “response” can refer to response of an organism, an organ, a tissue, a cell, or a cell component or in vitro system. In embodiments, a response is a clinical response. In embodiments, presence, extent, and/or nature of response can be measured and/or characterized according to particular criteria. In embodiments, such criteria can include clinical criteria and/or objective criteria. In embodiments, techniques for assessing response can include, but are not limited to, clinical examination, positron emission tomography, chest X-ray CT scan, MRI, ultrasound, endoscopy, laparoscopy, presence or level of a particular marker in a sample, cytology, and/or histology. Where a response of interest is a response of a tumor to a therapy, ones skilled in the art will be aware of a variety of established techniques for assessing such response, including, for example, for determining tumor burden, tumor size, tumor stage, etc. The likelihood of a subject having predictive features identified herein exhibiting a particular response is relative to a similarly situated second subject (e.g., a second subject having the same type of cancer, optionally the same type of cancer with similar characteristics (e.g., stage and/or location/distribution)) or group of subjects that lack one or more predictive feature.

In embodiments, the disclosed subject matter provides methods of identifying subjects (e.g., cancer subjects) as candidates for treatment with a treatment regimen (hereinafter “the patient selection method”). A “treatment regimen” can refer to the combination of doses, frequency of administration, or duration of treatment, with or without the addition of a second pharmaceutical agent.

Embodiments as described herein also provide methods for determining T-cell cross-reactivity between antigens. For example, term “cross-reactivity” can refer to the ability of an antigen binding protein, such as a T-cell or an antibody, that is specific for one antigen to react with a second antigen. For example, cross-reactivity can refer to the association between two different antigenic substances. Thus, a T-cell or antibody can be considered cross-reactive when it binds to an epitope other than that which induces its formation. Cross-reactive epitopes generally include many of the same complementary structural features as the inducing epitope and in some cases may actually fit better than the original.

In embodiments, each clone a in the plurality of clones can be uniquely defined by a unique set of somatic mutations, and the plurality of clones can be determined by a variant allele frequency of each respective somatic mutation in a plurality of somatic mutations determined from the plurality of sequencing reads.

The term “mutation” can refer to permanent change in the DNA sequence that makes up a gene. In embodiments, mutations can range in size from a single DNA building block (DNA base) to a large segment of a chromosome. In embodiments, mutations can include missense mutations, frameshift mutations, duplications, insertions, nonsense mutation, deletions and repeat expansions. In embodiments, a missense mutation is a change in one DNA base pair that results in the substitution of one amino acid for another in the protein made by a gene. In embodiments, a nonsense mutation is also a change in one DNA base pair. Instead of substituting one amino acid for another, however, the altered DNA sequence prematurely signals the cell to stop building a protein. In embodiments, an insertion changes the number of DNA bases in a gene by adding a piece of DNA. In embodiments, a deletion changes the number of DNA bases by removing a piece of DNA. In embodiments, small deletions can remove one or a few base pairs within a gene, while larger deletions can remove an entire gene or several neighboring genes. In embodiments, a duplication consists of a piece of DNA that is abnormally copied one or more times. In embodiments, frameshift mutations occur when the addition or loss of DNA bases changes a gene's reading frame. A reading frame consists of groups of 3 bases that each code for one amino acid. In embodiments, a frameshift mutation shifts the grouping of these bases and changes the code for amino acids. In embodiments, insertions, deletions, and duplications can all be frameshift mutations. In embodiments, a repeat expansion is another type of mutation. In embodiments, nucleotide repeats are short DNA sequences that are repeated a number of times in a row. For example, a trinucleotide repeat is made up of 3-base-pair sequences, and a tetranucleotide repeat is made up of 4-base-pair sequences. In certain embodiments, a repeat expansion is a mutation that increases the number of times that the short DNA sequence is repeated

In embodiments, the tumor-specific mutation that results in a neoantigen can be a somatic mutation. A “somatic mutation” can refer to a mutation that comprises DNA alterations in non-germline cells. Somatic mutations commonly occur in cancer cells. Somatic mutations in cancer cells can result in the expression of neoantigens, that in embodiments, transform a stretch of amino acids from being recognized as “self to “non-self Human tumors without a viral etiology can accumulate tens to hundreds of fold somatic mutations in tumor genes during neoplastic transformation, and some of these somatic mutations can occur in protein-coding regions and result in the formation of neoantigens. The exome is the protein-encoding part of the genome. Based on the mutations present within the protein-encoding part of the genome (i.e., the exome) of an individual tumor, potential neoantigens can be predicted for the individual tumor. In embodiments, whole exome sequencing is performed in a biological sample (e.g., a tumor sample) obtained from a subject having cancer.

In embodiments, the somatic mutation can be a single nucleotide variant or an indel. A “single nucleotide variant” (SNV) can refer to a nucleic acid sequence in which sequence variability is present in a single nucleotide. An “indel” can refer to the insertion or deletion of one or more nucleotide bases in a target DNA sequence in a chromosome. In coding regions of a nucleic acid sequence, unless the length of an indel is a multiple of 3, it will produce a frameshift when the sequence is translated.

Cancers can be screened to detect neoantigens using any of a variety of known technologies. In embodiments, neoantigens or expression thereof can be detected at the nucleic acid level (e.g., in DNA or RNA). In embodiments, neoantigens or expression thereof can be detected at the protein level (e.g., in a sample comprising polypeptides from cancer cells, which sample can be or comprise polypeptide complexes or other higher order structures including but not limited to cells, tissues, or organs).

In embodiments, a neoantigen can be detected by the method selected from the group consisting of whole exome sequencing, immunoassay, microarray, genome sequencing, RNA sequencing, ELISA, Western Transfer, DNA or RNA sequencing, mass spectrometry, and combinations thereof. In certain embodiments, one or more neoantigen is detected by whole exome sequencing. In embodiments, one or more neoantigen can be detected by the immunogenicity analysis method of somatic mutations described in Snyder et al. Engl J Med 371, 2189-2199 (2014). In embodiments, one or more neoantigen can be detected by the pVAC-Seq method described in Hndal et al., Genome Med (2016); 8: 11. In embodiments, one or more neoantigen can be detected by the in silico neoantigen prediction pipeline method described in Rizvi et al, Science 348, 124-128 (2015). In embodiments, one or more neoantigen can be detected by any of the methods described in WO2015/103037, WO2016/081947, and WO 2018136664, each of which are incorporated herein by reference in their entireties.

In embodiments, a plurality of sequencing reads can be obtained from one or more samples from the human subject that is representative of the cancer. For example, the plurality of clones can be determined by identifying a plurality of inferred copy number variations using the plurality of sequencing reads.

In embodiments, the raw sequence reads can be processed in order to identify the plurality of clones. For instance, in embodiments, raw sequence data reads can be aligned to a reference human genome (e.g., hgl9) using an alignment tool such as the Burrows-Wheeler Alignment tool. Base-quality score recalibration, and duplicate-read removal is performed. In embodiments, this recalibration excludes germline variants, annotation of mutations, and indels as described in Snyder et al, 2014, “Genetic Basis for Clinical Response to CTLA-4 Blockade in Melanoma,” N. Engl. J. Med. 371, 2189-2199, which is hereby incorporated by reference. In embodiments, local realignment and quality score recalibration is conducted using the Genome Analysis Toolkit (GATK) according to GATK best practices. See, DePristo et al, 2011, “A framework for variation discovery and genotyping using next-generation DNA sequencing data,” Nature Genet. 43, pp. 491-498; and Van der Auwera et al, 2013, “From FastQ Data to High-Confidence Variant Calls: The Genome Analysis Toolkit Best Practices Pipeline,” Curr. Prot. in Bioinformatics 43, 11.10.1-11.10.33 each of which is hereby incorporated by reference. Further, sequence alignment and mutation identification can be performed. In embodiments, sequence alignment and mutation identification can be performed using FASTQ files that are processed to remove any adapter sequences at the end of the reads. In some embodiments, adapter sequences are removed using cutadapt (vl.6). See Martin, 2011, “Cutadapt removes adapter sequences from high-throughput sequencing reads,” EMBnet.journal 17, pp. 10-12, which is hereby incorporated by reference. Then, resulting files can be mapped using a mapping software such as the BWA mapper (bwa mem vO.7.12), (see Li and Durbin, 2009, “Fast and accurate short read alignment with Burrows-Wheeler Transform,” Bioinformatics 25, pp. 1754-1760, which is hereby incorporated by reference). In such embodiments, the resulting files (e.g. SAM files) can be sorted, and read group tags added using the PICARD tools. After sorting in coordinate order, the BAMs can be processed with a tool such as PICARD MarkDuplicates. Realignment and recalibration is then carried out in some embodiments, (e.g., with a first realignment using the InDel realigner followed by base quality value recalibration with the BaseQRecalibrator). Once realignment and recalibration have been performed, mutation callers can then be used to identify single nucleotide variants. Exemplary mutation callers that can be used include, but are not limited to, Mutect 1.1.4, Somatic Sniper 1.0.4, Varscan 2.3.7, and Strelka 1.013). See Wei et al, 2015, “MAC: identifying and correcting annotation for multi-nucleotide variations, BMC Genomics 16, p. 569; Snyder and Chan, 2015, “Immunogenic peptide discovery in cancer genomes,” Curr Opin Genet Dev 30, pp. 7-16; Nielsen et al., 2003, “Reliable prediction of T-cell epitopes using neural networks with novel sequence representations,” Protein Sci 12, pp. 1007-1017; and Shen and Seshan, 2016, “FACETS: allele-specific copy number and clonal heterogeneity analysis tool for high-throughput DNA sequencing,” Nucleic Acids Res. 44, el31, each of which is incorporated by reference.

In embodiments, SNVs with an allele read count of less than 4 or with corresponding normal coverage of less than 7 reads are filtered out. In embodiments, SNVs with an allele read count of less than 7, of less than 5, or of less than 3 or with corresponding normal coverage of less than 12 reads, less than 8 reads, or less than 5 reads are filtered out. See, for example, Riaz et al, 2016, “Recurrent SERPINB3 and SERPINB4 mutations in patients who respond to anti-CTLA4 immunotherapy,” Nat. Genet. 48, 1327-1329, which is hereby incorporated by reference.

In some embodiments, the assignment of a somatic mutation to a neoantigen can be estimated using a bioinformatics tool such as NASeek. See Snyder et al, 2014, “Genetic Basis for Clinical Response to CTLA-4 Blockade in Melanoma,” N. Engl. J. Med. 371, pp. 2189-2199, which is hereby incorporated by reference. NASeek is a computational algorithm that first translates all mutations in exomes to strings of 17 amino acids, for both the wildtype and mutated sequences, with the amino acid resulting from the mutation centrally situated. Secondly, it evaluates putative MHC Class I binding for both wildtype and mutant nonamers using a sliding window method using NetMHC3.4 (on the Internet cbs.dtu.dk/services/NetMHC-3.4/) {see Andreatta and Nielsen, 2016, “Gapped sequence alignment using artificial neural networks: application to the MHC class I system,” Bioinformatics 32, pp. 511-517, which is hereby incorporated by reference) for patient-specific HLA types, to generate predicted binding affinities for both peptides. NASeek finally assesses for similarity between nonamers that predicted to be presented by patient-specific MHC Class I. In some embodiments, all nonamers with binding scores below 500 nM are defined as neoantigens. In some embodiments, all nonamers with binding scores below 250 nM are defined as neoantigens. In some embodiments, all nonamers with binding scores below 800 nM are defined as neoantigens.

The phrase “sequencing reads” can refer to any information or data indicative of the order of nucleotide bases (e.g., adenine, guanine, cytosine, and thymine/uracil) in a molecule of DNA or RNA (e.g., a whole genome, a whole transcriptome, an exome (exome), an oligonucleotide, a polynucleotide, a fragment, etc.). It is to be understood that the present teachings contemplate sequence information obtained using all available various methods, platforms or techniques, including but not limited to: capillary electrophoresis, microarrays, ligation-based systems, polymerase-based systems, hybridization-based systems, direct or indirect nucleotide identification systems, pyrosequencing (pyrosequencing), ion- or pH-based detection systems, electronic signature-based systems, microwell-based (e.g., nanopore), visualization-based systems, and the like.

In embodiments, the plurality of sequencing reads (e.g., whole genome sequencing reads, exome sequencing reads, targeted sequencing reads, etc.) can exhibit an average read depth of less than 200, less than 100, less than 50, less than 40, or less than 20. In some embodiments, the plurality of sequencing reads are whole genome sequencing reads that collectively exhibit an average read depth of between 25 and 60.

In other embodiments, each clone a in the plurality of clones can be uniquely defined by a unique set of somatic mutations, and the plurality of clones can be determined by a combination of (i) a variant allele frequency of each respective somatic mutation in the plurality of somatic mutations determined from the plurality of sequencing reads and (ii) an identification of a plurality of inferred copy number variations using the whole-genome sequencing data.

The phrase “variant allele frequency” can refer to the relative frequency of an allele (variant of a gene) at a particular locus in a population (e.g., a fraction of all chromosomes in the population that carry a particular allele among a population of cells, a population of organisms, a population of subjects, or a population of molecules or a population of DNA molecules, among others.

The phrase “copy number variations” (CNV) can refer to variation in the number of copies of a nucleic acid sequence present in a sample (e.g., a nucleic acid sample isolated from, or derived from, or obtained from a subject) in comparison with the copy number of the nucleic acid sequence present in a reference sample (e.g., a nucleic acid sample isolated from, or derived from, or obtained from a reference subject exhibiting known genotypes). In some embodiments, the nucleic acid sequence is 1 kb or larger. In some embodiments, the nucleic acid sequence is a whole chromosome or significant portion thereof. In some embodiments, copy number differences are identified by comparison of a sequence of interest in a sample with an expected level of the sequence of interest. For example, the level of the sequence of interest in the sample is compared to that present in a reference sample. In some embodiments, copy number variation refers to a form of structural variation of the DNA of a genome that results in a cell having an abnormal or, for certain genes, a normal variation in the number of copies of one or more sections of the DNA.

In some embodiments, copy number variations (“CNVs”) can refer to relatively large regions of the genome that have been deleted (fewer than the normal number) or duplicated (more than the normal number) on certain chromosomes. For example, the chromosome that normally has sections in order as A-B-C-D-E might instead have sections A-B-C-C-D-E (a duplication of “C”) or A-B-D-E (a deletion of “C”). This variation accounts for roughly 12% of human genomic DNA and each variation may range from about 500 base pairs (500 nucleotide bases) to several megabases in size (e.g., between 5,000 to 5 million bases). In some embodiments, copy number variations refer to relatively small regions of the genome that have been deleted (e.g., micro-deletions) or duplicated on certain chromosomes. In some embodiments, copy number variations refer to genetic variants due to presence of single-nucleotide polymorphisms (SNPs), which affect only one single nucleotide base. In some embodiments, copy number variants/variations include deletions, including micro-deletions, insertions, including micro-insertions, duplications, multiplications, inversions, translocations and complex multi-site variants. In some embodiments, copy number variants/variations encompass chromosomal aneuploidies and partial aneuploidies.

The phrase “whole-genome sequencing” can refer to a process that determines the DNA sequence of each DNA strand in a sample. The resulting sequence may be referred to as “raw sequencing data” or “reads”. For example, whole-genome sequencing can be used to determine the complete DNA sequence of an organism's chromosomal DNA as well as DNA contained in other organelles such as mitochondria

In embodiments, each neoantigen in the plurality of neoantigens of a clone in the plurality of clones can be a nonamer peptide. In embodiments, each neoantigen in the plurality of neoantigens of a clone in the plurality of clones is a peptide that is eight, nine, ten, or eleven residues in length. In embodiments each neoantigen in the plurality of neoantigens of a clone in the plurality of clones is a peptide that is 3-30 amino acids, e.g., about 3-5, about 5-15 (e.g., about 8-11, about 5-10, or about 10-15), about 15-20, about 20-25, or about 20-30 amino acids, in length. In embodiments, each neoantigen in the plurality of neoantigens of a clone in the plurality of clones is a peptide that is about 8-11 amino acids in length. In embodiments, each neoantigen in the plurality of neoantigens of a clone in the plurality of clones is a peptide that is about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 amino acids in length. In certain embodiments, each neoantigen in the plurality of neoantigens of a clone in the plurality of clones is a peptide that is at least about 3, at least about 5, or at least about 8 amino acids in length. In certain embodiments, each neoantigen in the plurality of neoantigens of a clone in the plurality of clones is a peptide is less than about 30, less than about 20, less than about 15, or less than about 10 amino acids in length

Embodiments comprise determining a human leukocyte antigen (HLA) type of the subject. For example, the HLA type of the human subject can be determined from the plurality of sequencing reads. For example, the HLA type of the subject can be determined using a polymerase chain reaction using a biological sample from the subject. In embodiments, HLA typing can be performed using the sequence reads by either low to intermediate resolution polymerase chain reaction-sequence-specific primer (PCR-SSP) method or by high-resolution SeCore HLA sequence-based typing method (HLA-SBT) (INVITROGEN). In embodiments ATHLATES is used for HLA typing and confirmation. See Liu and Duffy et al., 2013, “ATHLATES: accurate typing of human leukocyte antigen through exome sequencing,” Nucleic Acids Res 41:el42, which is hereby incorporated by reference.

Embodiments comprise computing for a plurality of clones, and for each respective clone a in the plurality of clones, an initial frequency Xa of a respective clone a in the one or more sample. For example, the plurality of clones comprises two clones. For example, the plurality of clones comprises between two clones and ten clones. For example, the plurality of clones comprises greater than ten clones.

In embodiments, the initial frequency Xa of the respective clone a in the one or more samples can be determined using the plurality of sequencing reads from the one or more samples from the subject.

Aspects of the invention are also drawn towards a system for determining a likelihood that a subject afflicted with a cancer will be responsive to a treatment regimen that comprises administering a checkpoint blockade immunotherapy directed to the cancer of the subject. For example, the “system” can comprise memory; one or more processors; and one or more modules stored in memory and configured for execution by the one or more processors.

In embodiments, the modules can comprise instructions for (A) obtaining a plurality of sequencing reads from one or more samples from the human subject that is representative of the cancer; (B) determining a human leukocyte antigen (HLA) type of the human subject; (C) determining a plurality of clones, and for each respective clone a in the plurality of clones, an initial frequency Xa of the respective clone a in the one or more samples; (D) for each respective clone a in the plurality of clones, computing a corresponding clone fitness score of the respective clone, thereby computing a plurality of clone fitness scores, each corresponding clone fitness score computed for a respective clone a by a first procedure comprising: (a) identifying a plurality of neoantigens in the respective clone a; (b) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (c) determining the corresponding clone fitness score of the respective clone a as an aggregate of the neoantigen recognition potentials across the plurality of neoantigens in the respective clone a; and (E) computing a total fitness for the one or more samples as a sum of the clone fitness scores across the plurality of clones, wherein each clone fitness score is weighted by the initial frequency Xa of the corresponding clone a, and the total fitness quantifies the likelihood that the human subject afflicted with the cancer will be responsive to the treatment regimen.

Still further, aspects of the invention are drawn towards a system for determining a likelihood that a human subject afflicted with a cancer will be responsive to a treatment regimen that comprises administering a checkpoint blockade immunotherapy directed to the cancer to the subject.

In embodiments, the system comprises memory; one or more processors; and one or more modules stored in memory and configured for execution by the one or more processors, the modules comprising instructions for carrying out embodiments as described herein.

Also, aspects of the invention are drawn towards a non-transitory computer readable storage medium for determining a likelihood that a subject afflicted with a cancer will be responsive to a treatment regimen that comprises administering a checkpoint blockade immunotherapy directed to the cancer to the subject. In embodiments, the non-transitory computer readable storage medium can store one or more programs for execution by one or more processors of a computer system.

For example, the one or more computer programs comprises instructions for (a) obtaining a plurality of sequencing reads from one or more samples from the subject that is representative of the cancer; (b) determining a human leukocyte antigen (HLA) type of the subject; (c) determining a plurality of clones, and for each respective clone a in the plurality of clones, an initial frequency Xa of the respective clone a in the one or more samples; (d) for each respective clone a in the plurality of clones, computing a corresponding clone fitness score of the respective clone, thereby computing a plurality of clone fitness scores, each corresponding clone fitness score computed for a respective clone a by a first procedure comprising: (i) identifying a plurality of neoantigens in the respective clone a; (ii) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (iii) determining the corresponding clone fitness score of the respective clone a as an aggregate of the neoantigen recognition potentials across the plurality of neoantigens in the respective clone a; and (e) computing a total fitness for the one or more samples as a sum of the clone fitness scores across the plurality of clones, wherein each clone fitness score is weighted by the initial frequency Xa of the corresponding clone a, and the total fitness quantifies the likelihood that the human subject afflicted with the cancer will be responsive to the treatment regimen.

In embodiments, the non-transitory computer readable storage medium can store one or more programs for execution by one or more processors of a computer system, the one or more computer programs comprising instructions for carrying out embodiments as described herein.

Aspects of the invention are also drawn towards a method for identifying within a computing system a neoantigen to target as an immunotherapy for a cancer.

A “neoantigen”, “neoepitope” or “neopeptide” can refer to a tumor-specific antigen that arises from one or more tumor-specific mutation. In embodiments, the tumor-specific mutation can alter the amino acid sequence of genome encoded proteins. In embodiments, the neoantigen is a tumor-specific antigen that arises from a tumor-specific mutation. In certain embodiments, a neoantigen is not expressed by healthy cells (e.g., non-tumor cells or non-cancer cells) in a subject.

Many antigens expressed by cancer cells are self-antigens which are selectively expressed or overexpressed on the cancer cells. These self-antigens are difficult to target with immunotherapy because they require overcoming both central tolerance (whereby autoreactive T cells are deleted in the thymus during development) and peripheral tolerance (whereby mature T cells are suppressed by regulatory mechanisms). Targeting neoantigens can abrogate these tolerance mechanisms. In embodiments, a neoantigen can be recognized by cells of the immune system of a subject (e.g., T cells) as “non-self.

Neoantigens are not recognized as “self-antigens” by immune system. T cells that are capable of targeting neoantigens are not subject to central and peripheral tolerance mechanisms to the same extent as T cells which recognize self-antigens.

Neoantigens can vary in different subjects, e.g., different subjects can have different combinations of neoantigens, also referred to as “neoantigen signatures”. For example, each subject can have a unique neoantigen signature. In embodiments, a neoantigen signature can refer to a combination of one or more neoantigen identified by methods as described herein.

Neoantigens can be located in any genes in a subject.

Embodiments can comprise assessing neoantigen immunogenicity. Immunogenicity can refer to the ability of a particular substance, such as an antigen (e.g., a neoantigen), to induce or stimulate an immune response in the cells expressing such antigen.

In embodiments, a neoantigen can be a neoantigenic peptide comprising a tumor specific mutation. The neoantigenic peptide can be a peptide that is incorporated into a larger protein. In embodiments, a neoantigenic peptide can be a series of residues, typically L-amino acids, connected one to the other, typically by peptide bonds between the a-amino and carboxyl groups of adjacent amino acids. A neoantigenic peptide can be a variety of lengths, either in their neutral (uncharged) forms or in forms which are salts, and either free of modifications such as glycosylation, side chain oxidation, or phosphorylation.

In embodiments, the size of the neoantigenic peptide can be about 3-30 amino acids, e.g., about 3-5, about 5-15 (e.g., about 8-11, about 5-10, or about 10-15), about 15-20, about 20-25, or about 20-30 amino acids, in length. In embodiments, the neoantigenic peptide can be about 8-11 amino acids in length. In embodiments, the neoantigenic peptide can be about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 amino acids in length. In embodiments, the neoantigenic peptide can be at least about 3, at least about 5, or at least about 8 amino acids in length. In embodiments, the neoantigenic peptide can be less than about 30, less than about 20, less than about 15, or less than about 10 amino acids in length

The term “neoantigen number” or “neoantigen burden” can refer to the number of neoantigen(s) measured, detected or predicted in a sample (e.g., a biological sample from a subject). In embodiments, the neoantigen number can be measured by using whole exome sequencing and in silico prediction. In embodiments, neoantigen number can be measured by any methods for detecting neoantigens, e.g., those described herein. In embodiments, the neoantigen number can be measured by whole exome sequencing the biological sample. In embodiments, the neoantigen number can be measured by the immunogenicity analysis method of somatic mutations described in Snyder et al., 204, Engl J Med 371, 2189-2199. In embodiments, the neoantigen number can be measured by the pVAC-Seq method described in Hndal et al., 2016, Genome Med 8, 11. In embodiments, the neoantigen number can be measured by the in silico neoantigen prediction pipeline described in Rizvi et al., 2015 Science 348, 124-128. In embodiments, the neoantigen number can be measured by any of the methods described in WO2015/103037 and WO2016/081947.

The neoantigen number obtained from a population of subjects having the cancer can vary depending on the type of the cancer, therapy (e.g., immunotherapy, chemotherapy), the caner histology, and/or the site of disease (tumor).

In embodiments, the median neoantigen number obtained from a population of subjects having cancer can be between 10 and 10,000, between 10 and 50, between 30 and 50, between 30 and 40, between 50 and 100, between 100 and 200, between 200 and 300, between 300 and 400, between 400 and 500, between 500 and 600, between 600 and 700, between 700 and 800, between 800 and 900, between 900 and 1,000, between 1,000 and 2,000, between 2,000 and 3,000, between 3,000 and 4,000, between 4,000 and 5,000, between 5,000 and 6,000, between 6,000 and 7,000, between 7,000 and 8,000, between 8,000 and 9,000, or between 9,000 and 10,000. In embodiments, the median neoantigen number obtained from a population of subjects having cancer can be about 40. In embodiments, the median neoantigen number obtained from a population of subjects having cancer can be about 38.

In embodiments, the method can comprise (a) obtaining a plurality of sequencing reads from one or more samples from a human cancer subject that is representative of the cancer; (b) determining a human leukocyte antigen (HLA) type of the human subject from the plurality of sequencing reads; (c) determining a plurality of clones, and for each respective clone a in the plurality of clones, an initial frequency Xa of the respective clone a in the one or more samples from the plurality of sequencing reads; for each respective clone a in the plurality of clones, computing a corresponding clone fitness score of the respective clone, thereby computing a plurality of clone fitness scores, each corresponding clone fitness score computed for a respective clone a by a first procedure comprising: (i) identifying a plurality of neoantigens in the respective clone a; (ii) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (iii) determining the corresponding clone fitness score of the respective clone a as an aggregate of the neoantigen recognition potentials across the plurality of neoantigens in the respective clone a; and (d) selecting at least a first neoantigen from a plurality of neoantigens for a respective clone a in the plurality of respective clones based upon the recognition potential of the first neoantigen to target as an immunotherapy for the cancer.

In embodiments, the obtaining (a), the determining (b), the determining (c), and the computing (d) can be repeated for a plurality of human subjects across a plurality of HLA types. For example, the obtaining (a), the determining (b), the determining (c), and the computing (d) can be repeated for two subjects, four subjects, ten subjects, twenty-five subjects, fifty subjects, or greater than fifty subjects.

In embodiments, the subjects comprise a plurality of HLA types. “HLA” refers to human leukocyte antigen. HLA is a protein found on the surface of most cells in the body and plays an important part in the body's immune response to foreign substances. There are three general groups of HLA: HLA-A, HLA-B, and HLA-DR. There are many different specific HLA proteins within each of these three groups. For example, there are 59 different HLA-A proteins, 118 different HLA-B and 124 different HLA-DR). Each of these HLA has a different numerical designation (e.g., HLA-A1, HLA-A2).

In embodiments, the first neoantigen can be selected on the basis of the recognition potential of the first neoantigen across the plurality of HLA types.

In embodiments, the first neoantigen can be selected on the basis of the recognition potential of the first neoantigen across the plurality of subjects.

As described herein, each clone a in the plurality of clones can be uniquely defined by a unique set of somatic mutations (e.g., a SNV or indel), and the plurality of clones can be determined by a variant allele frequency of each respective somatic mutation in a plurality of somatic mutations determined from the whole-genome sequencing data.

As described herein, the plurality of clones can be determined by identifying a plurality of inferred copy number variations, such as by using the whole-genome sequencing data.

As described herein, each clone a in the plurality of clones can be uniquely defined by a unique set of somatic mutations, and the plurality of clones can be determined by a combination of (i) a variant allele frequency of each respective somatic mutation in the plurality of somatic mutations determined from the whole-genome sequencing data and (ii) an identification of a plurality of inferred copy number variations using the whole-genome sequencing data.

As described herein, the plurality of sequencing reads can exhibit an average read depth of less than 40. As described herein, the plurality of sequencing reads exhibits an average read depth of between 25 and 60. In embodiments, the plurality of sequencing reads (e.g., whole genome sequencing reads, exome sequencing reads, targeted sequencing reads, etc.) can exhibit an average read depth of less than 200, less than 100, less than 50, less than 40, or less than 20. In some embodiments, the plurality of sequencing reads are whole genome sequencing reads that collectively exhibit an average read depth of between 25 and 60.

As described herein, each neoantigen in the plurality of neoantigens of a clone in the plurality of clones is a nonamer peptide. For example, each neoantigen in the plurality of neoantigens of a clone in the plurality of clones is a peptide comprising eight, nine, ten, or eleven residues in length. In embodiments each neoantigen in the plurality of neoantigens of a clone in the plurality of clones is a peptide that is 3-30 amino acids, e.g., about 3-5, about 5-15 (e.g., about 8-11, about 5-10, or about 10-15), about 15-20, about 20-25, or about 20-30 amino acids, in length. In embodiments, each neoantigen in the plurality of neoantigens of a clone in the plurality of clones is a peptide that is about 8-11 amino acids in length. In embodiments, each neoantigen in the plurality of neoantigens of a clone in the plurality of clones is a peptide that is about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 amino acids in length. In certain embodiments, each neoantigen in the plurality of neoantigens of a clone in the plurality of clones is a peptide that is at least about 3, at least about 5, or at least about 8 amino acids in length. In certain embodiments, each neoantigen in the plurality of neoantigens of a clone in the plurality of clones is a peptide is less than about 30, less than about 20, less than about 15, or less than about 10 amino acids in length.

As described herein, the HLA type of the subject can be determined from the plurality of sequencing reads. There are three general groups of HLA: HLA-A, HLA-B, and HLA-DR. There are many different specific HLA proteins within each of these three groups. For example, there are 59 different HLA-A proteins, 118 different HLA-B and 124 different HLA-DR). Each of these HLA has a different numerical designation (e.g., HLA-A1, HLA-A2).

As described herein, the HLA type of the subject can be determined using a polymerase chain reaction using a biological sample from the cancer subject. In embodiments, HLA typing can be performed using the sequence reads by either low to intermediate resolution polymerase chain reaction-sequence-specific primer (PCR-SSP) method or by high-resolution SeCore HLA sequence-based typing method (HLA-SBT) (INVITROGEN). In embodiments ATHLATES is used for HLA typing and confirmation. See Liu and Duffy et al., 2013, “ATHLATES: accurate typing of human leukocyte antigen through exome sequencing,” Nucleic Acids Res 41:el42, which is hereby incorporated by reference.

As described herein, the plurality of clones comprises two clones. As described herein, the plurality of clones comprises between two clones and ten clones. As described herein, the plurality of clones comprises greater than ten clones.

As described herein, the initial frequency Xa of the respective clone a in the one or more samples can be determined using the plurality of sequencing reads from the one or more samples from the subject.

Aspects of the invention are also directed to a system for identifying an immunotherapy for a cancer. As described herein, systems can comprise memory; one or more processors; and one or more modules stored in memory and configured for execution by the one or more processors.

As described herein, the modules can comprise instructions for (A) obtaining a plurality of sequencing reads from one or more samples from the human cancer subject that is representative of the cancer; (B) determining a human leukocyte antigen (HLA) type of the human subject from the plurality of sequencing reads; (C) determining a plurality of clones, and for each respective clone a in the plurality of clones, an initial frequency Xa of the respective clone a in the one or more samples from the plurality of sequencing reads; (D) for each respective clone a in the plurality of clones, computing a corresponding clone fitness score of the respective clone, thereby computing a plurality of clone fitness scores, each corresponding clone fitness score computed for a respective clone a by a first procedure comprising: (a) identifying a plurality of neoantigens in the respective clone a; (b) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (c) determining the corresponding clone fitness score of the respective clone a as an aggregate of the neoantigen recognition potentials across the plurality of neoantigens in the respective clone a; and (E) selecting at least a first neoantigen from a plurality of neoantigens for a respective clone a in the plurality of respective clones based upon the recognition potential of the first neoantigen as the immunotherapy for the cancer.

Further, aspects of the invention are drawn to a system for identifying an immunotherapy for a cancer.

For example, embodiments comprise memory; one or more processors; and one or more modules stored in memory and configured for execution by the one or more processors. In embodiments, the modules comprise instructions for executing the method as described herein.

Aspects of the invention are also drawn towards a non-transitory computer readable storage medium for identifying an immunotherapy for a cancer. As described here, the term “immunotherapy” can refer to a treatment that involves the activation of a specific immune response and/or an immune effector function. Immunotherapy can function to remove cells that express antigens (e.g., neoantigens) from a patient. Such elimination may take place as a result of the improvement or induction of an immune response and/or immune effector function in a patient specific for an antigen or a cell expressing an antigen.

As described herein, the non-transitory computer readable storage medium can store one or more programs for execution by one or more processors of a computer system. The one or more computer programs can comprise instructions for: (A) obtaining a plurality of sequencing reads from one or more samples from a subject that is representative of the cancer; (B) determining a human leukocyte antigen (HLA) type of the subject from the plurality of sequencing reads; (C) determining a plurality of clones, and for each respective clone a in the plurality of clones, an initial frequency Xa of the respective clone a in the one or more samples from the plurality of sequencing reads; (D) for each respective clone a in the plurality of clones, computing a corresponding clone fitness score of the respective clone, thereby computing a plurality of clone fitness scores, each corresponding clone fitness score computed for a respective clone a by a first procedure comprising: (i) identifying a plurality of neoantigens in the respective clone a; computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (iii) determining the corresponding clone fitness score of the respective clone a as an aggregate of the neoantigen recognition potentials across the plurality of neoantigens in the respective clone a; and (E) selecting at least a first neoantigen from a plurality of neoantigens for a respective clone a in the plurality of respective clones based upon the recognition potential of the first neoantigen as the immunotherapy for the cancer.

Aspects of the invention are also drawn towards a non-transitory computer readable storage medium for identifying an immunotherapy for a cancer. As described herein, the non-transitory computer readable storage medium can store one or more programs for execution by one or more processors of a computer system. As described herein, the one or more computer programs can comprise instructions for executing embodiments as described herein.

Still further, aspects of the invention are drawn towards a method of treating a subject afflicted with a cancer.

The term “treating”, or “treatment” can refer to clinical intervention in an attempt to alter the disease course of the individual or cell being treated, and can be performed either for prophylaxis or during the course of clinical pathology.

Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

Embodiments can comprise (1) determining within a computing system that the subject is likely to be responsive to the checkpoint blockade immunotherapy, wherein the computing system has one or more programmable processors, memory, and a plurality of instructions stored on the memory that are executable by the one or programmable processors; and (2) administering the checkpoint block immunotherapy when it is determined that the human subject is likely to be responsive to the checkpoint blockade immunotherapy.

The term “administering” can refer to the physical introduction of an agent (e.g., an immunotherapy) into a subject using a variety of methods and delivery systems known to those of ordinary skill in the art. Exemplary routes of administration include, for example, intravenous, intramuscular, subcutaneous, intraperitoneal, spinal or other parenteral routes of administration by injection or infusion. The phrase “parenteral administration” as used herein refers to modes of administration other than enteral and topical administration, generally by injection, and includes, but is not limited to, intravenous, intramuscular, intraarterial, intrathecal, intralymphatic; intralesional, intracystic, intraorbital, intracardiac, intradermal, intraperitoneal, cervical, subcutaneous, subepidermal, intraarticular, subcapsular, subarachnoid, intrathecal, epidural and intrasternal injections and infusions, as well as in vivo including electroporation. In some embodiments, the formulation is administered via a parenteral route, eg, orally. Other parenteral routes include topical, epidermal or mucosal routes of administration, eg, intranasally, vaginally, rectally, sublingually or topically. Administration may be performed, for example, once, several times, and/or over one or more extended periods of time.

As described herein, the determination that the subject will be responsive to the checkpoint blockade immunotherapy comprises: (A) obtaining a plurality of sequencing reads from one or more samples from the subject that is representative of the cancer; (B) determining a human leukocyte antigen (HLA) type of the subject; (C) determining a plurality of clones, and for each respective clone a in the plurality of clones, an initial frequency Xa of the respective clone a in the one or more samples; (D) for each respective clone a in the plurality of clones, computing a corresponding clone fitness score of the respective clone, thereby computing a plurality of clone fitness scores, each corresponding clone fitness score computed for a respective clone a by a first procedure comprising: (a) identifying a plurality of neoantigens in the respective clone a; (b) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and (c) determining the corresponding clone fitness score of the respective clone a as an aggregate of the neoantigen recognition potentials across the plurality of neoantigens in the respective clone a; and (E) computing a total fitness for the one or more samples as a sum of the clone fitness scores across the plurality of clones, wherein each clone fitness score is weighted by the initial frequency Xa of the corresponding clone a, and the total fitness quantifies the likelihood that the human subject afflicted with the cancer will be responsive to the checkpoint blockade immunotherapy.

As described herein, the checkpoint blockade immunotherapy can refer to the use of agents that inhibit immune checkpoint nucleic acids and/or proteins. Immune checkpoints share the common function of providing inhibitory signals that suppress immune response and inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereto.

Non-limiting examples of immune checkpoint blocking antibodies include antibodies against CTLA cytotoxic T-lymphocyte antigen 4 (anti-CTLA4 antibodies), antibodies against programmed death 1 (anti-PD-1 antibodies), antibodies against Programmed death-ligand 1 (anti-PD-L1 antibodies), antibodies against lymphocyte activation gene-3 (anti-LAG3 antibodies), antibodies against T cell immunoglobulin and mucin domain-containing protein 3 (anti-TIM-3 antibodies), antibodies against glucocorticoid-induced TNFR-related protein (GITR), antibodies against OX40, antibodies against CD40, antibodies against T cell immunoreceptor with Ig and ITIM domains (TIGIT), antibodies against 4-1BB, antibodies against B7 homolog 3 (anti-B7-H3 antibodies), antibodies against B7 homolog 4 (anti-B7-H4 antibodies), and antibodies against B- and T-lymphocyte attenuator (anti-BTLA antibodies).

The presently disclosed subject matter provides identification of neoantigens in subjects having cancer. Accordingly, aspects of the invention can be drawn to methods for identifying within a computing system a neoantigen to be used as an anti-cancer vaccine, the computing system having one or more programmable processors, memory, and a plurality of instructions stored on the memory that are executable by the one or more programmable processors. The term “vaccine” can refer to a composition for generating immunity for the prophylaxis and/or treatment of diseases (e.g., neoplasia/tumor). In embodiments, vaccines are medicaments that comprise antigens and can be used in humans or animals for generating specific defense and protective substance by vaccination.

Neoantigen-based cancer vaccine can induce more robust and specific anti-tumor T-cell responses compared with conventional shared-antigen-targeted vaccine. Certain genetic loci, also known as immunogenic hotspots, can be enriched for neoantigens in specific tumors that display great T cell infiltration and adaptive immune activation. Vaccine targeting tumor-specific immunogenic hotspot generated neoantigen can induce robust and specific anti-tumor T cell response against the tumor cell. The vaccine can be used along or in combination with other cancer treatment, e.g., immunotherapy.

In one aspect, the present disclosure provides a vaccine comprising one or more of the presently disclosed neoantigens, or a polynucleotide encoding the neoantigen, or a protein or peptide comprising the neoantigen. In embodiments, the neoantigen can be selected based, at least in part, on predicted immunogenicity, for example, in silico. In embodiments, the predicted immunogenicity can be analyzed using computational algorithms for MHC class I and class II binding as well as use of tandem minigene libraries for class II epitope screening. In addition, in some embodiments, neoantigen specific T cell assays can be used to differentiate true immunogenic neoepitopes from putative ones {see Kvistborg et. al, 2016, J. ImmunoTherapy of Cancer 4:22 for detailed review). In embodiments, any methods and tools known in the art are used to predict immunogenicity of a neoantigen. For example, in some embodiments, the Immune Epitope Database (IEDB) T Cell Epitope-MHC Binding Prediction Tool disclosed in Brown et. al. 2010, Nucleic Acids Res. January; 38 (Database issue):D854-62) can be used to predict the binding of neoantigen to autologous HLA-A encoded MHC proteins. In certain embodiments, immunogenicity analysis strategy and tools disclosed in WO2015/103037 can be used in accordance with the disclosed subject matter, and the content of the forgoing patent is incorporated herein by reference in its entirety.

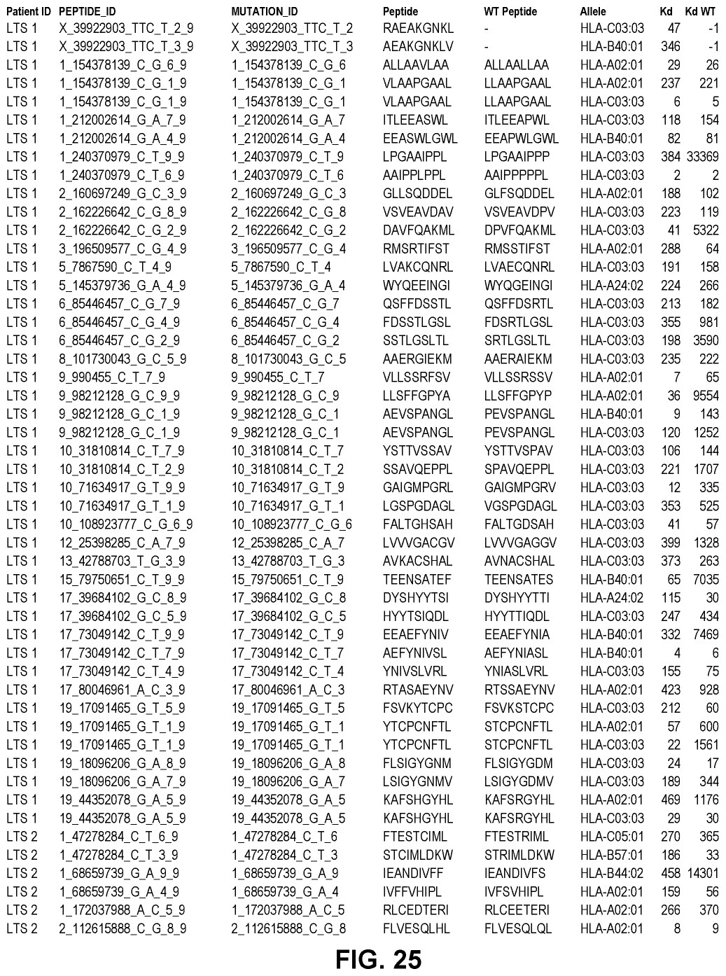

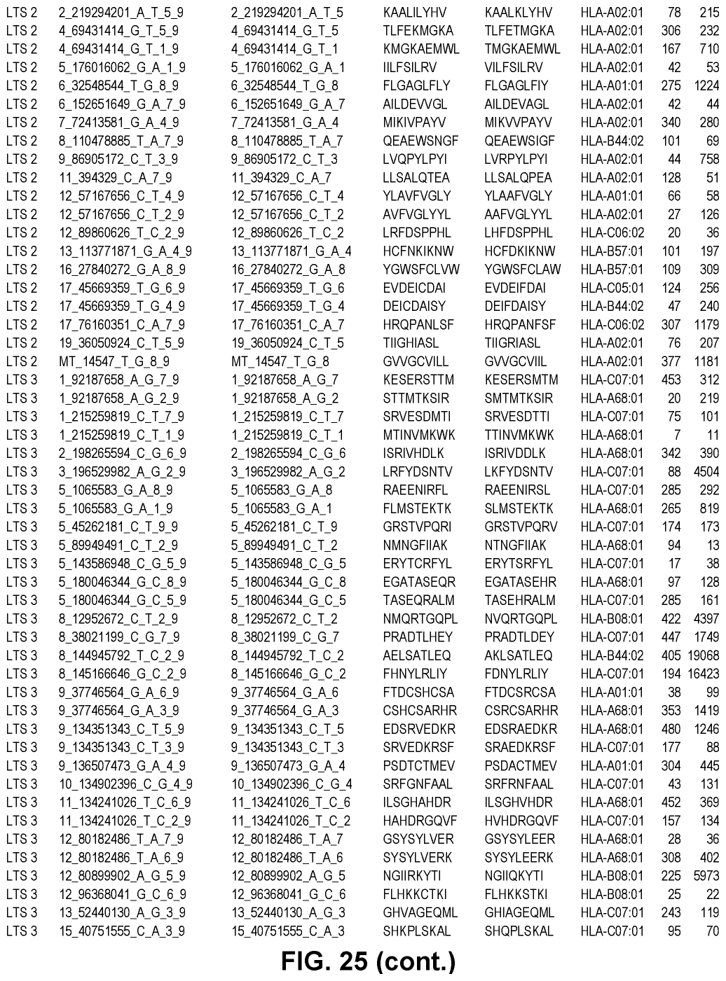

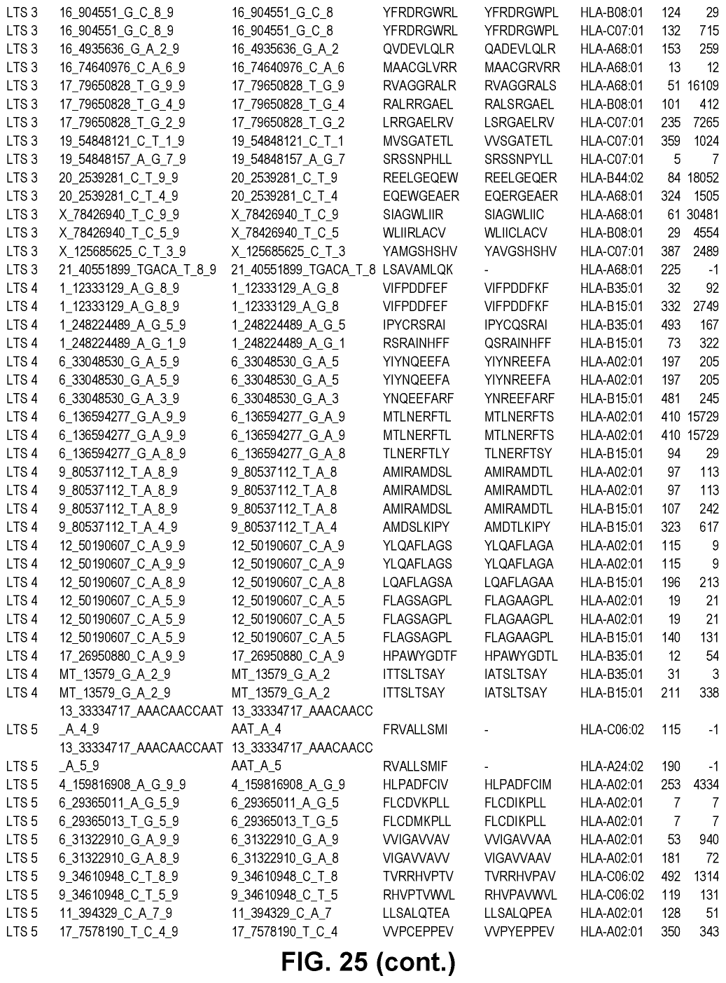

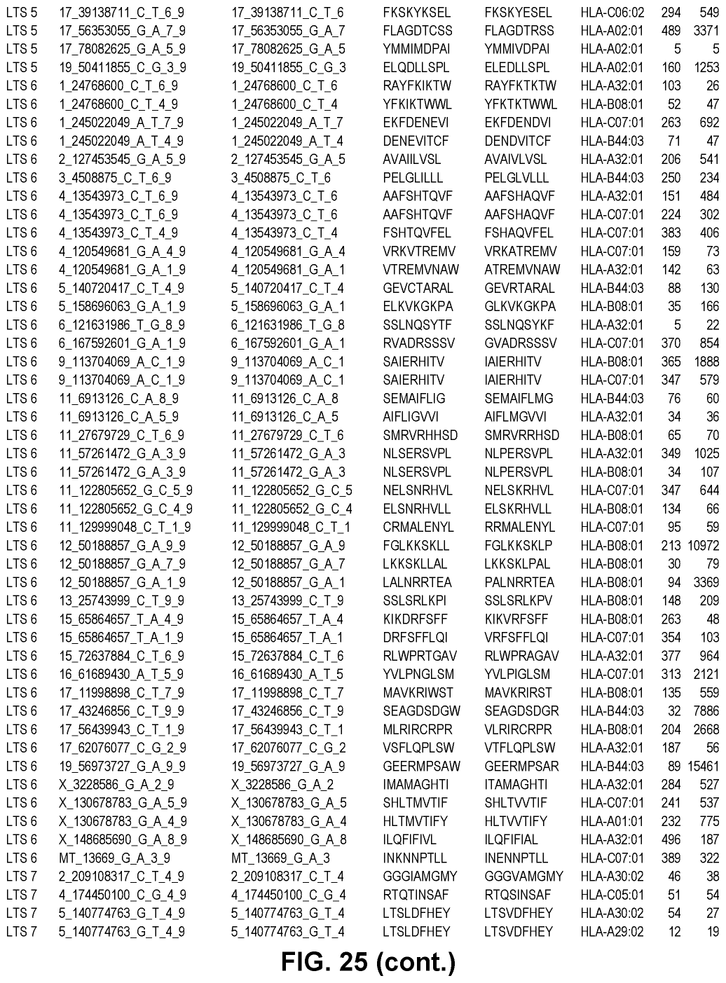

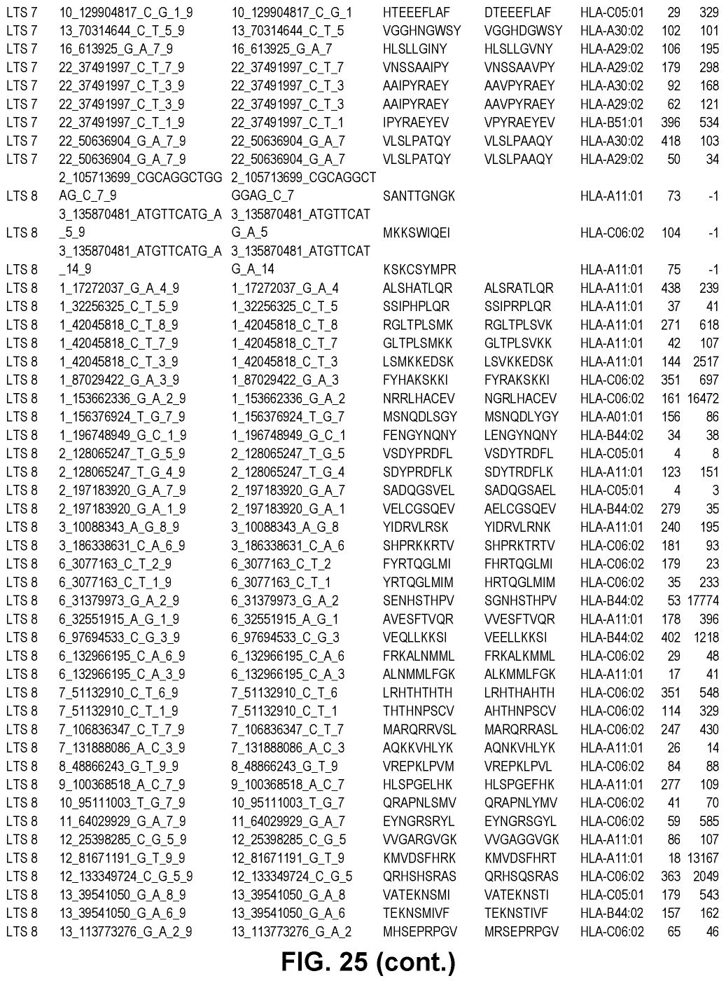

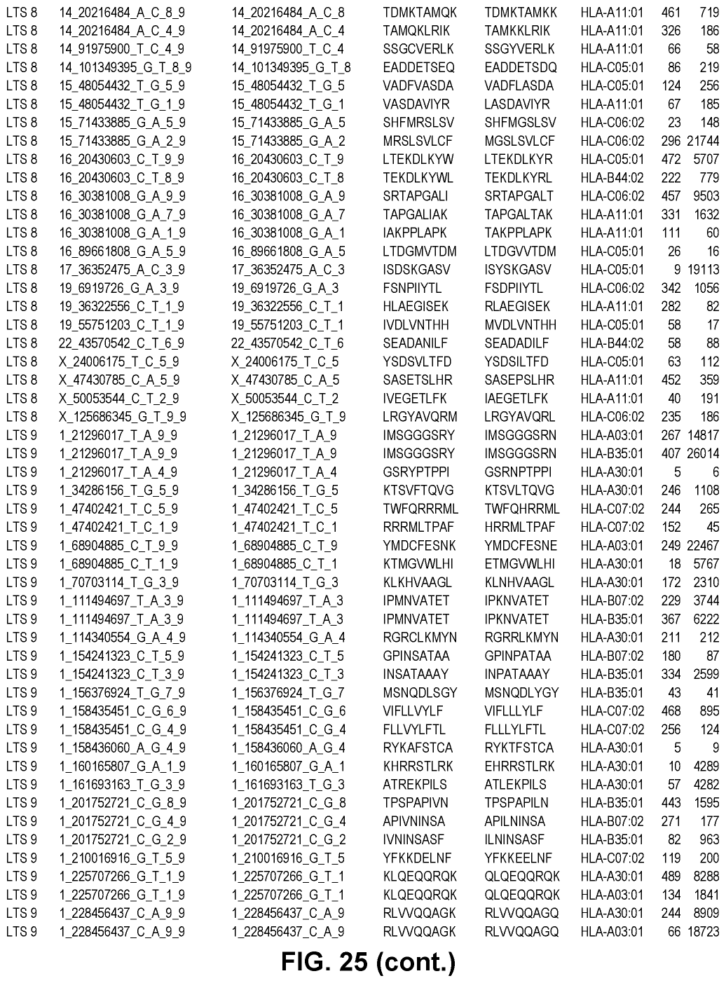

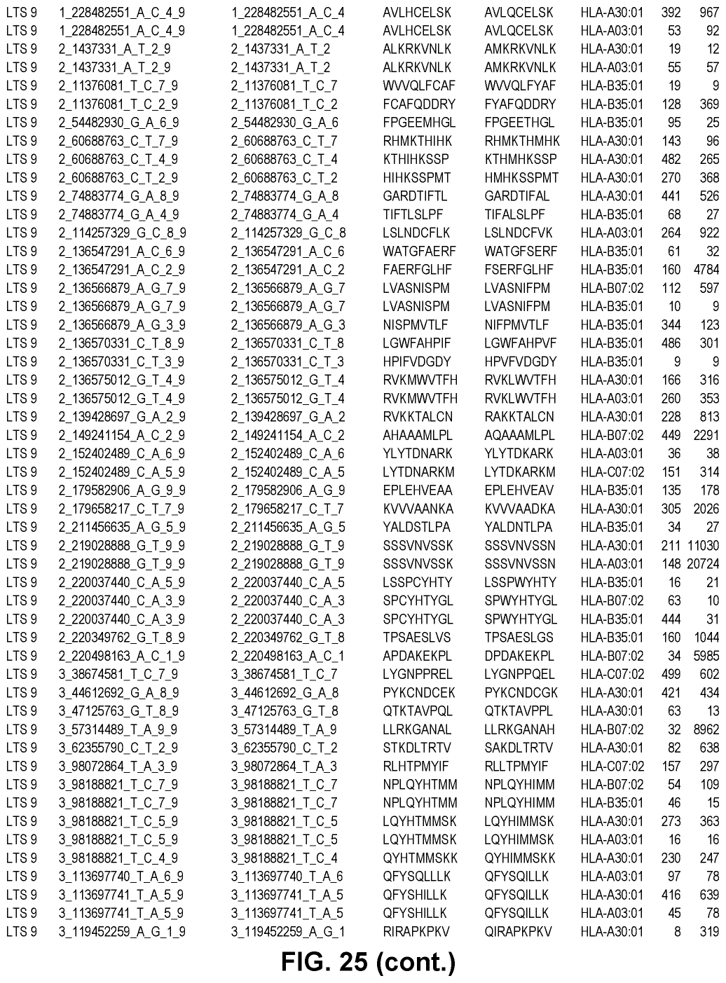

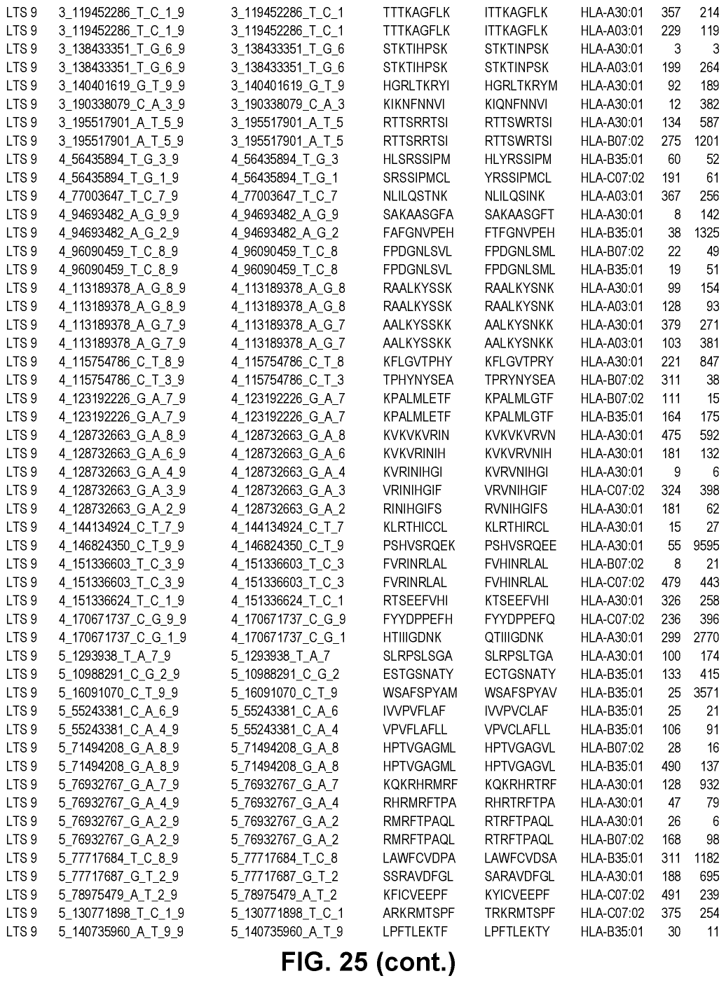

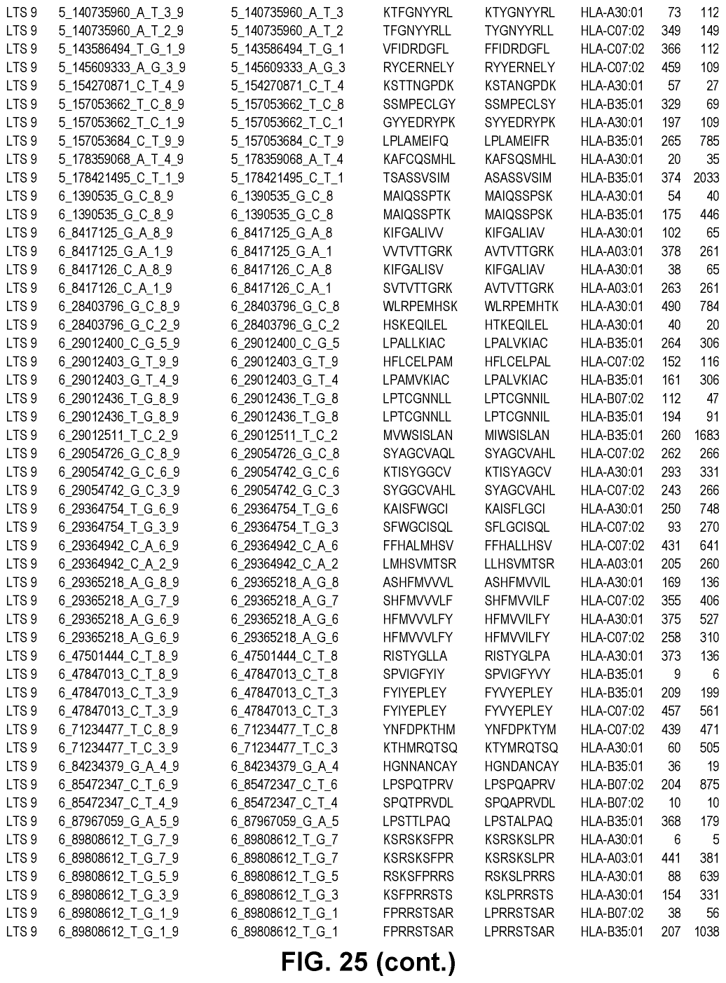

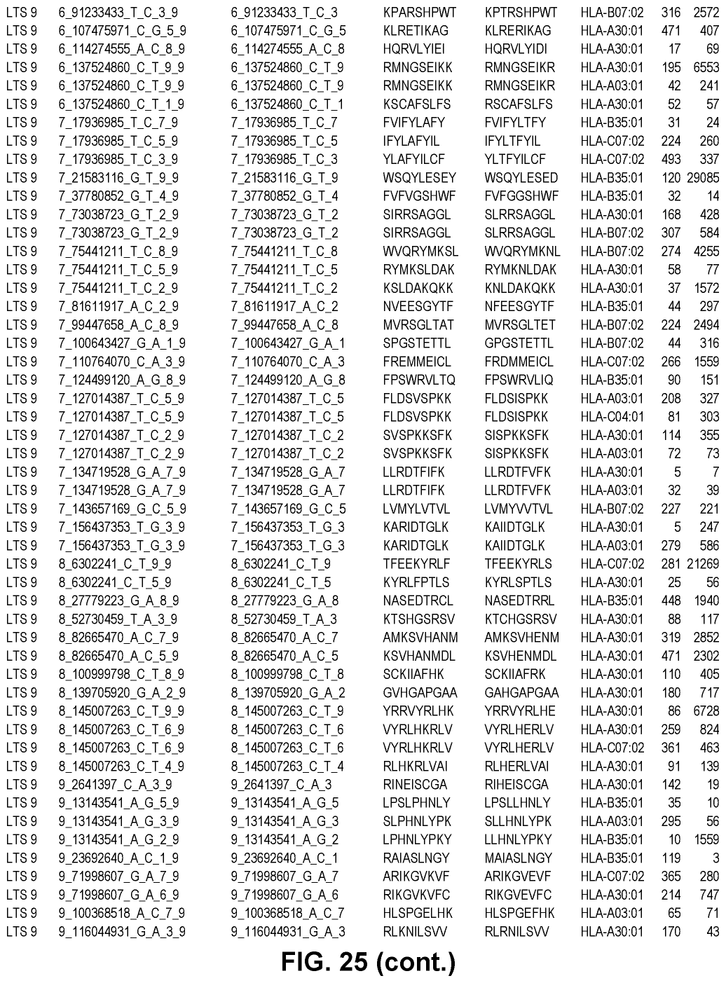

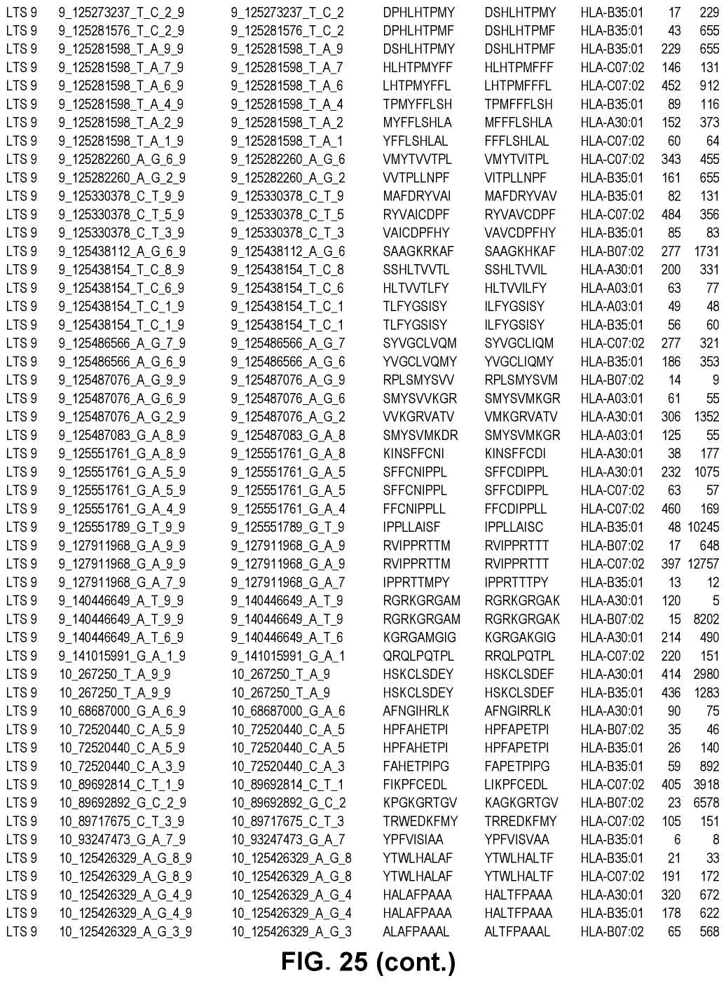

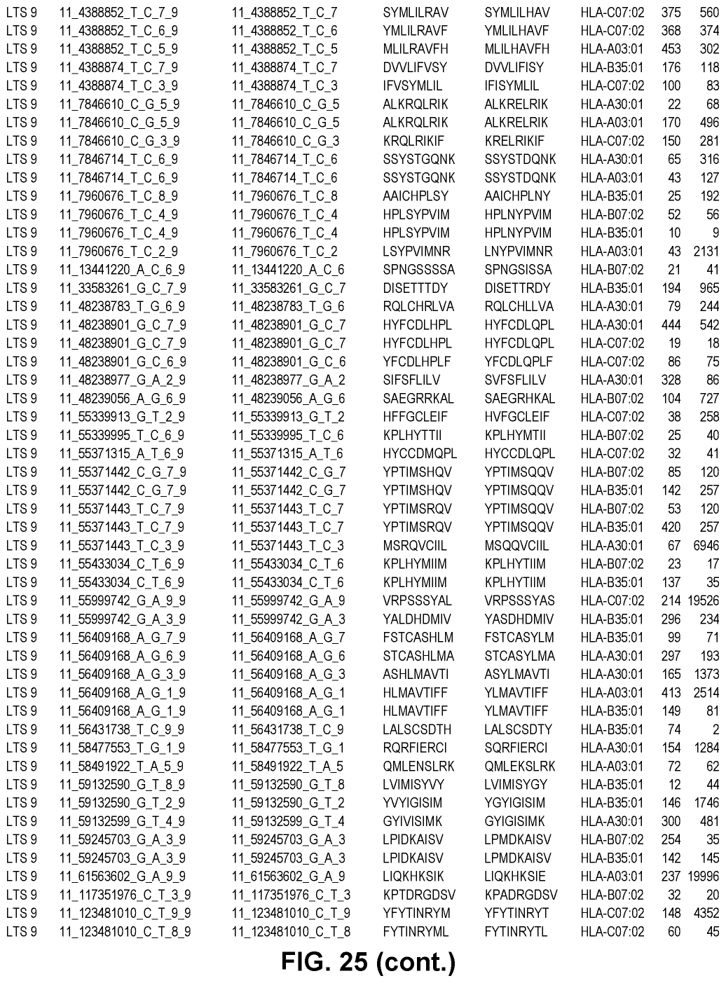

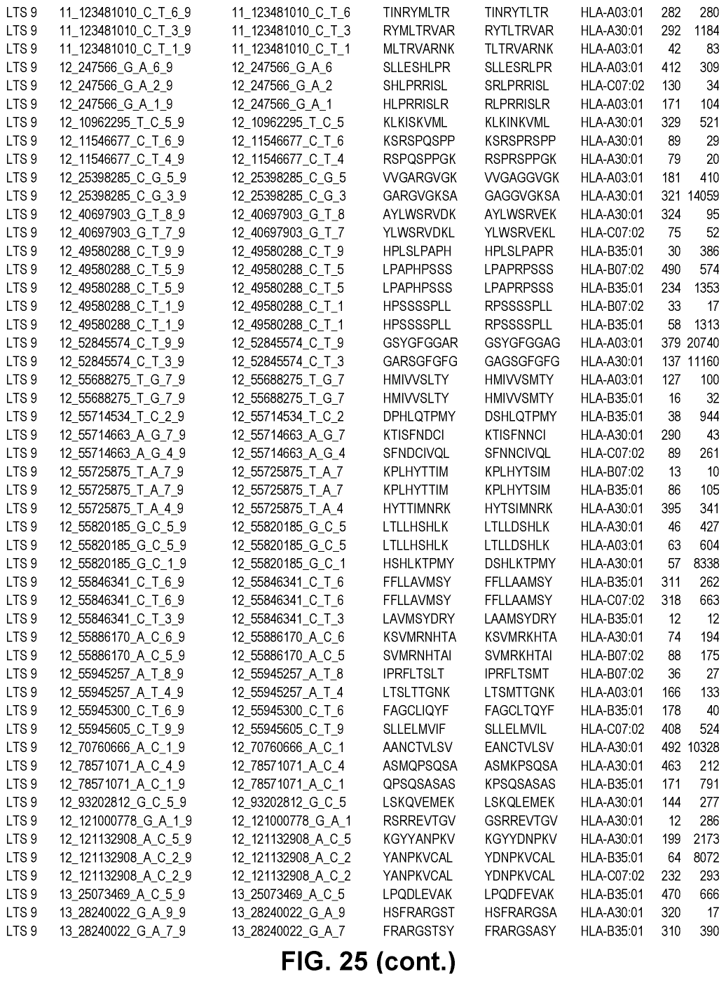

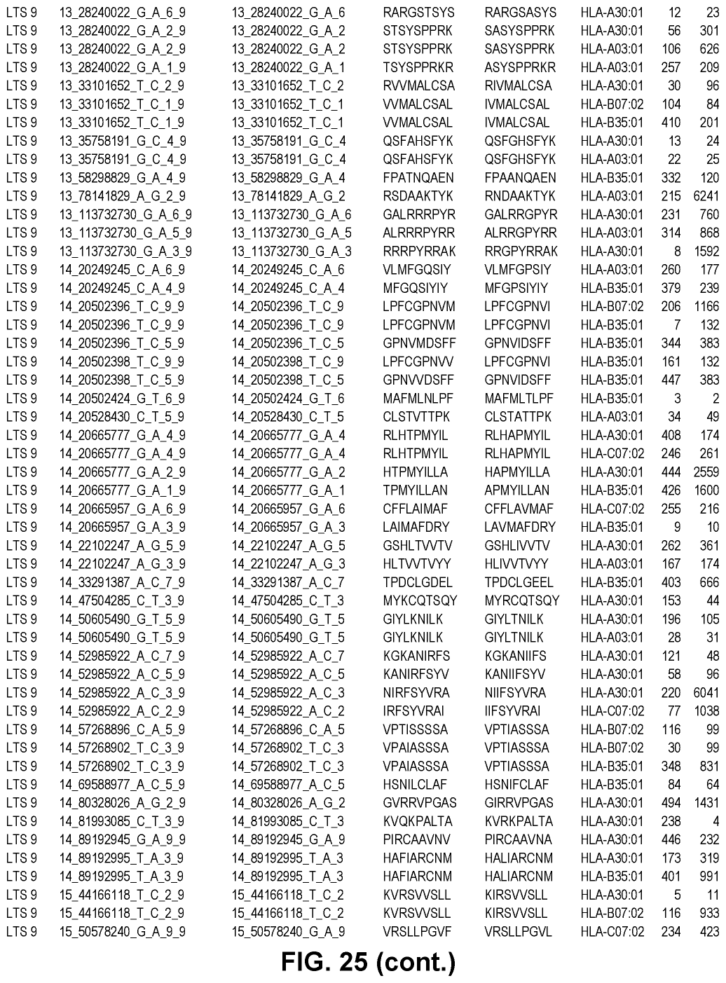

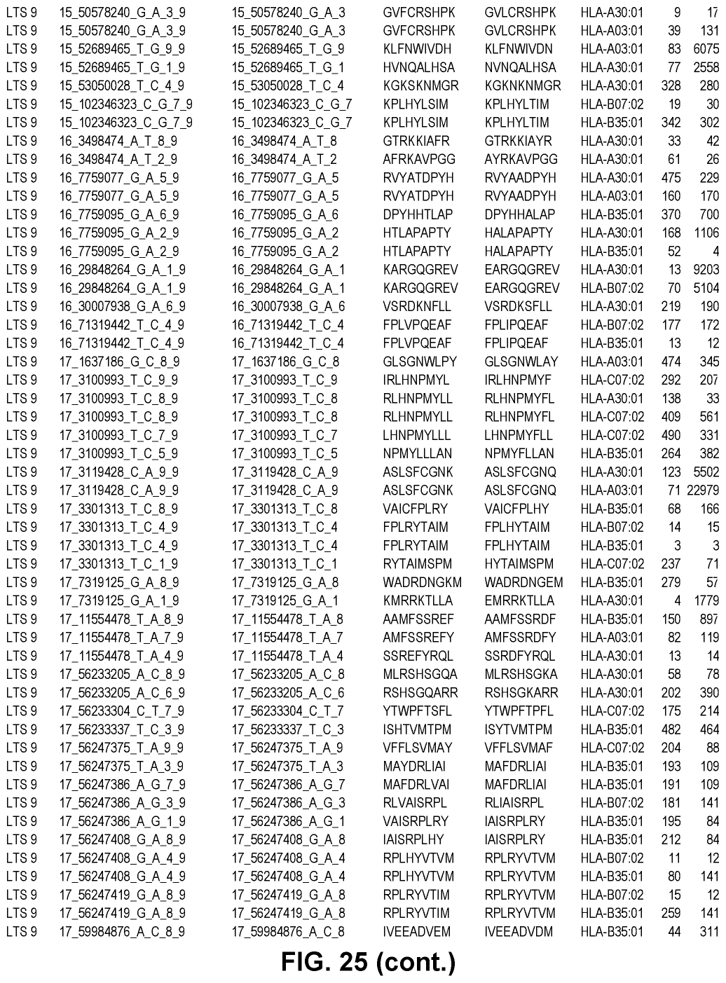

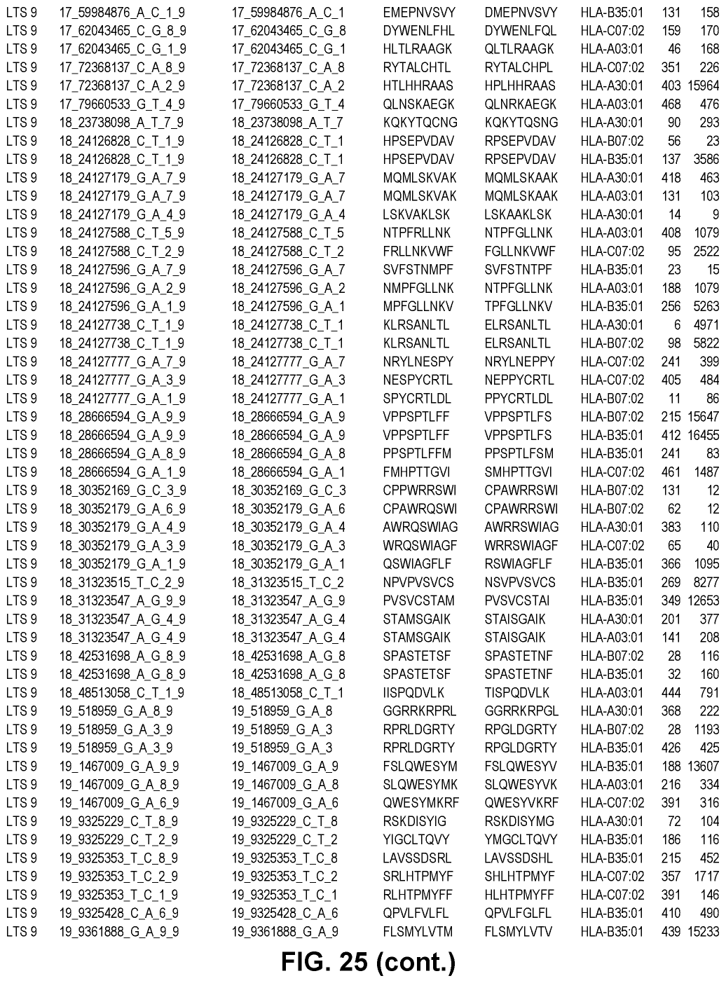

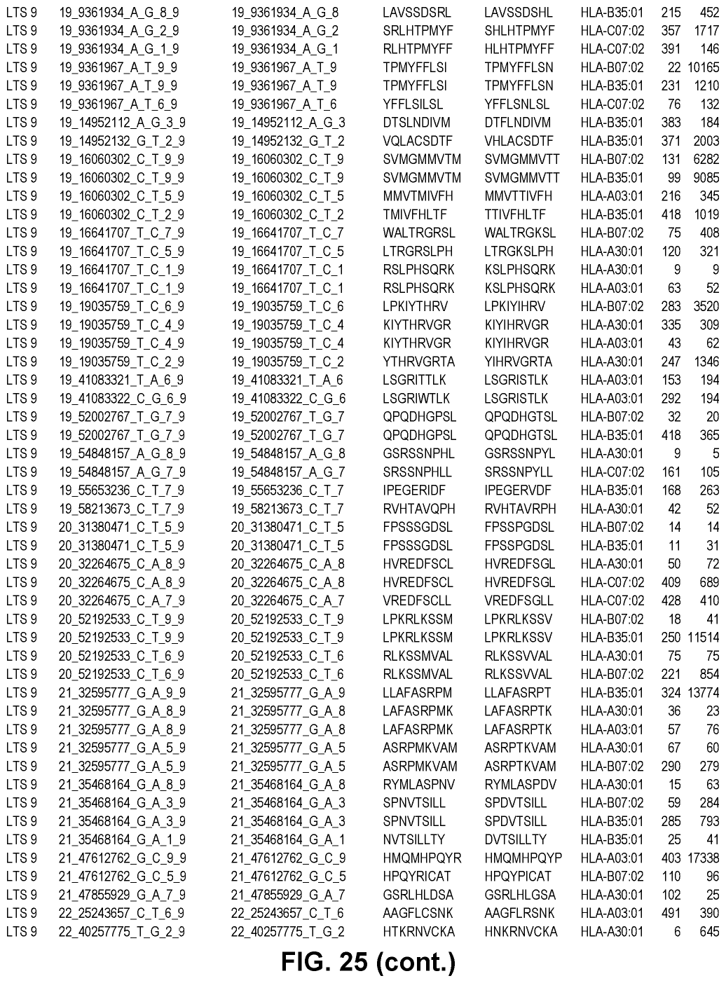

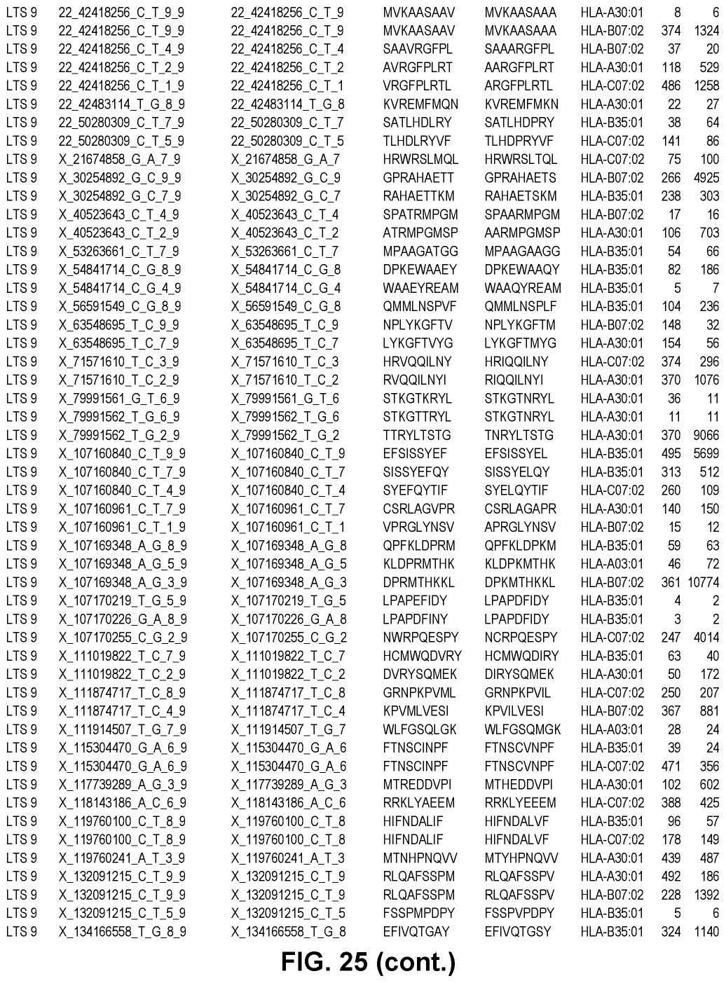

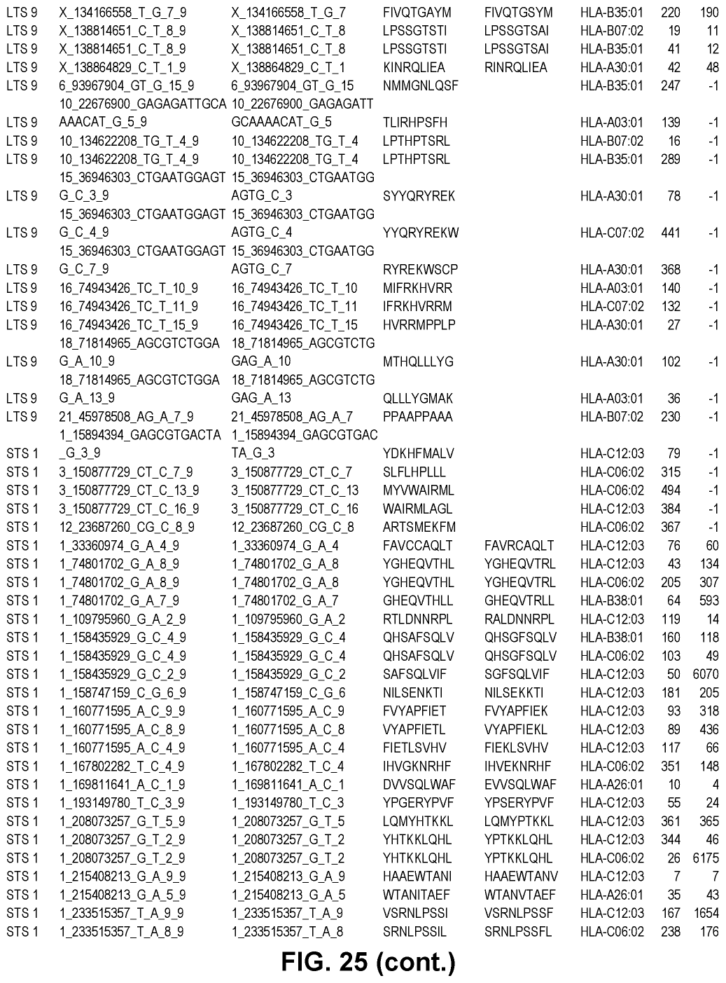

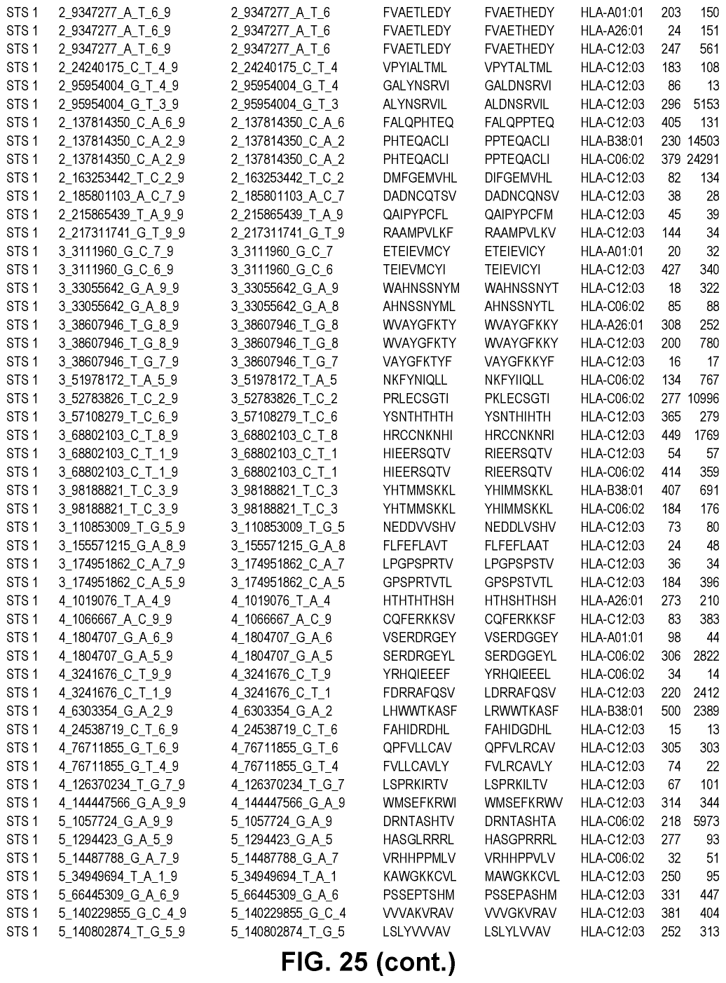

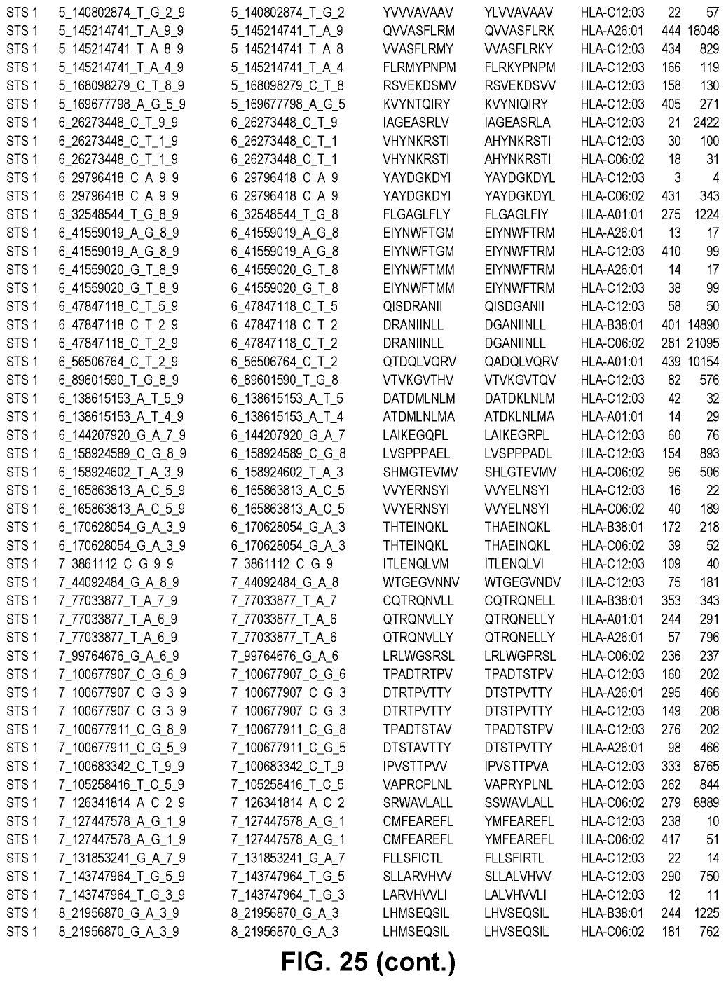

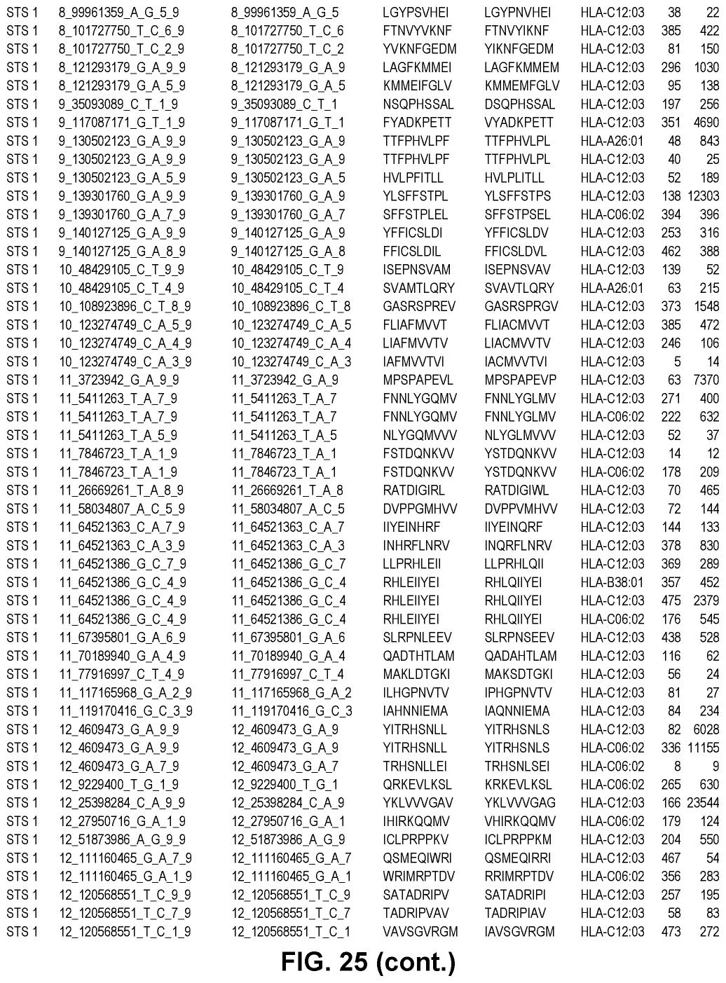

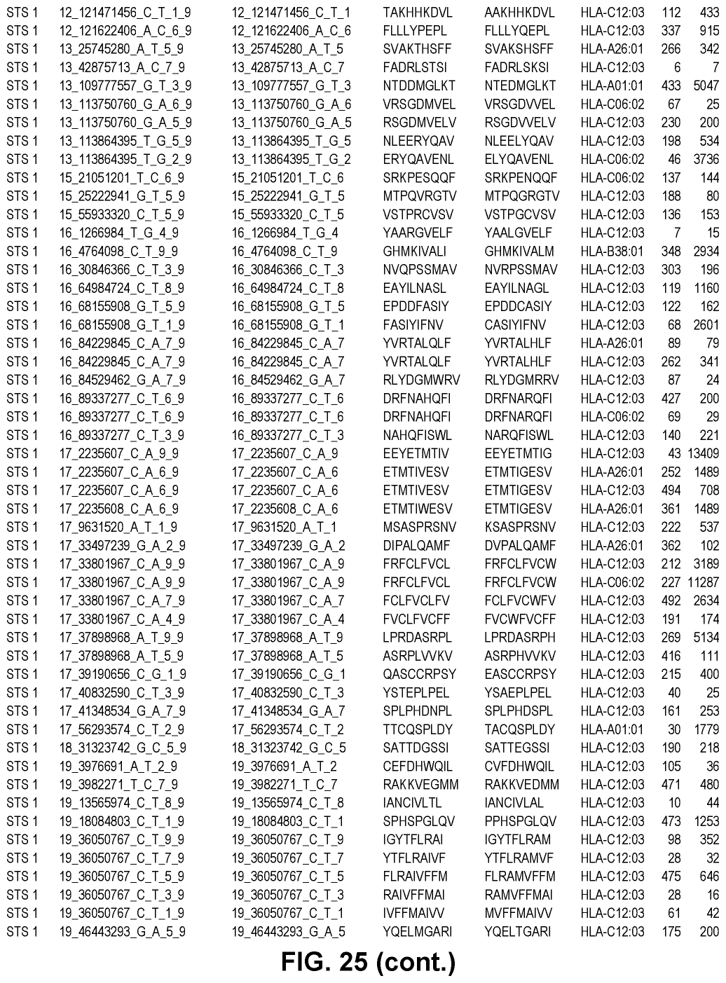

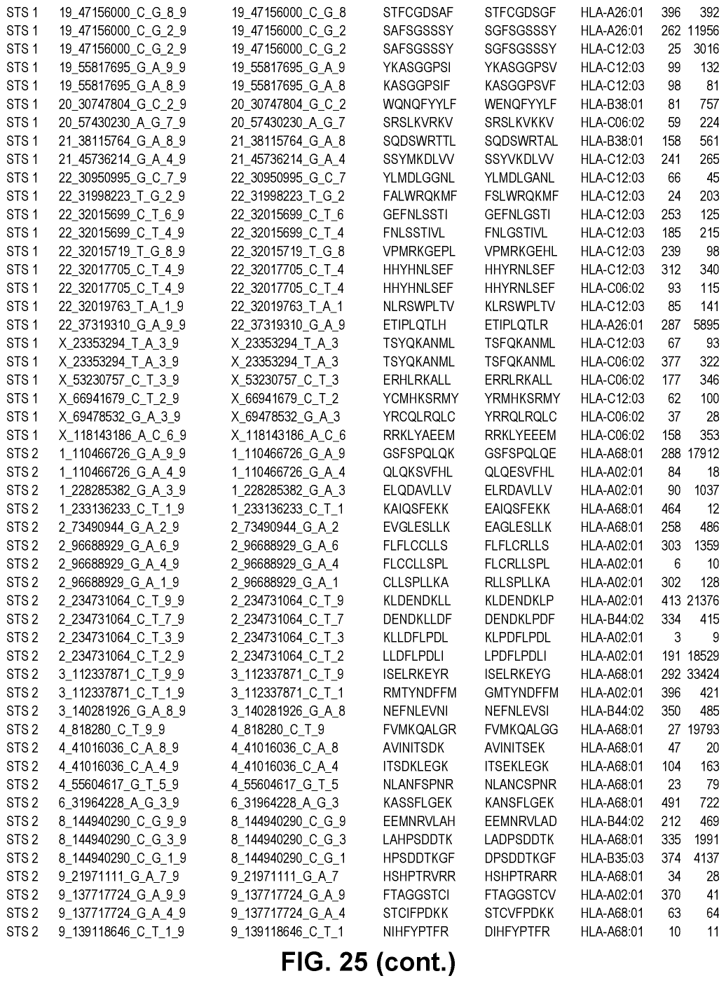

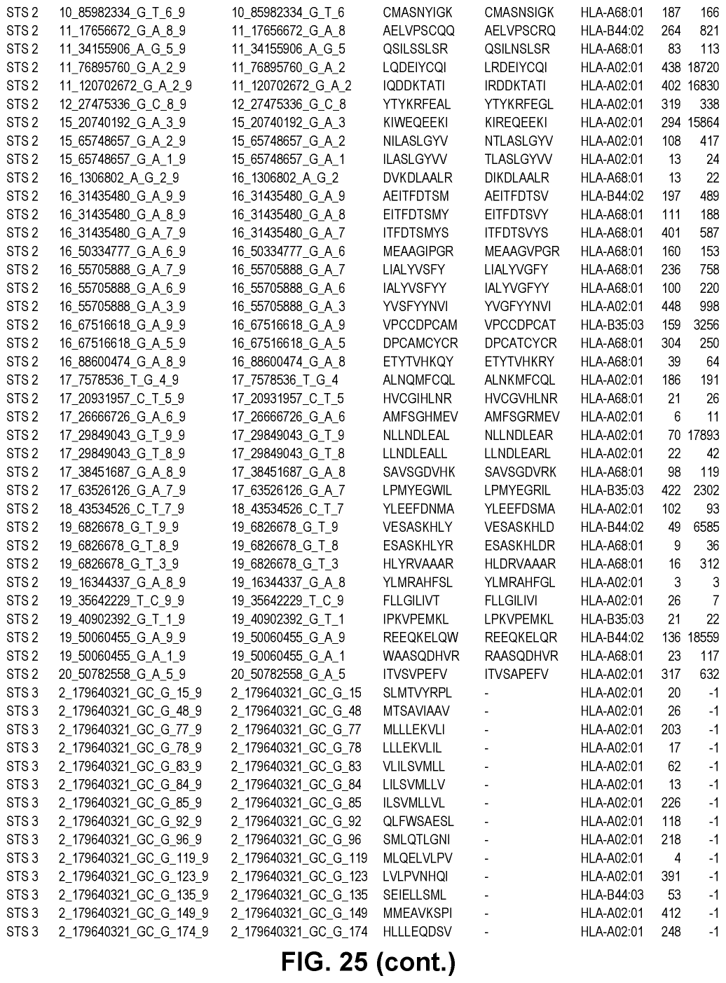

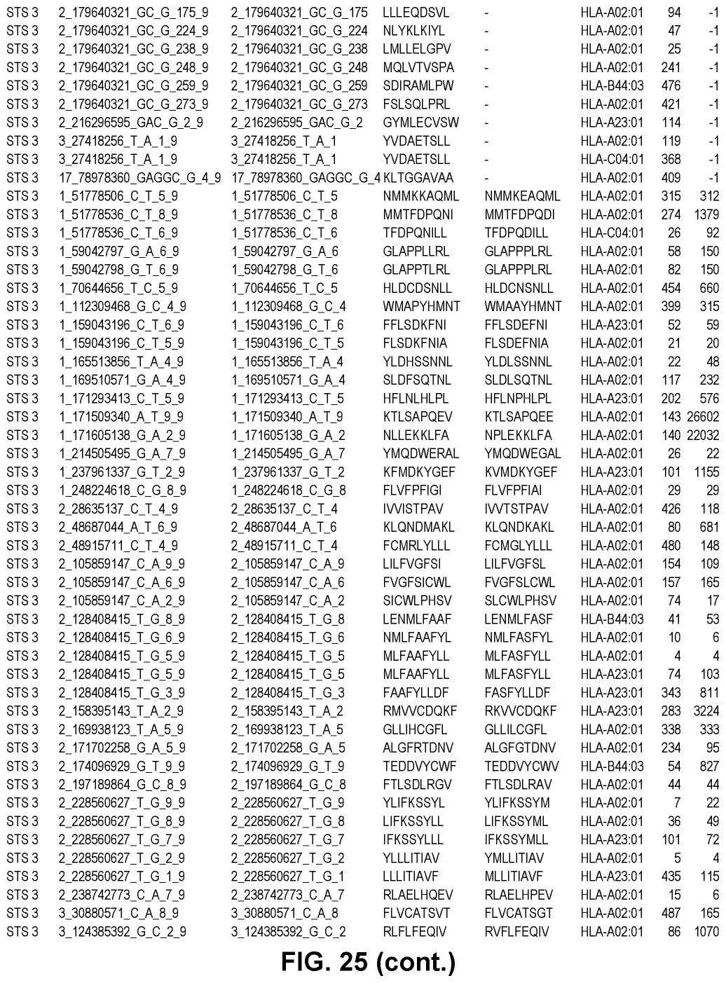

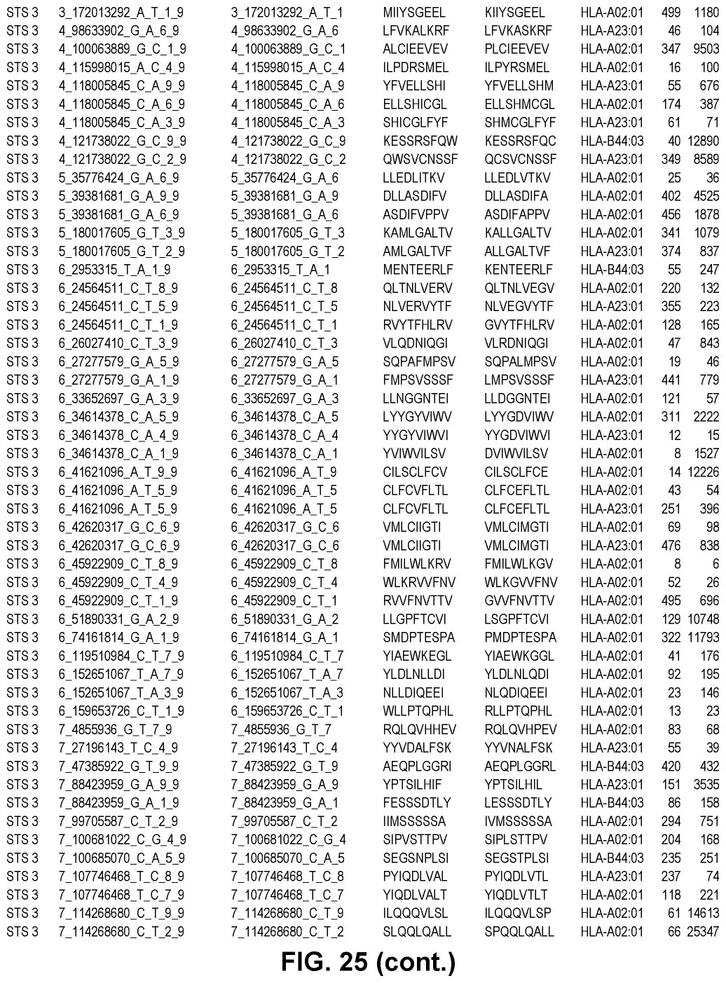

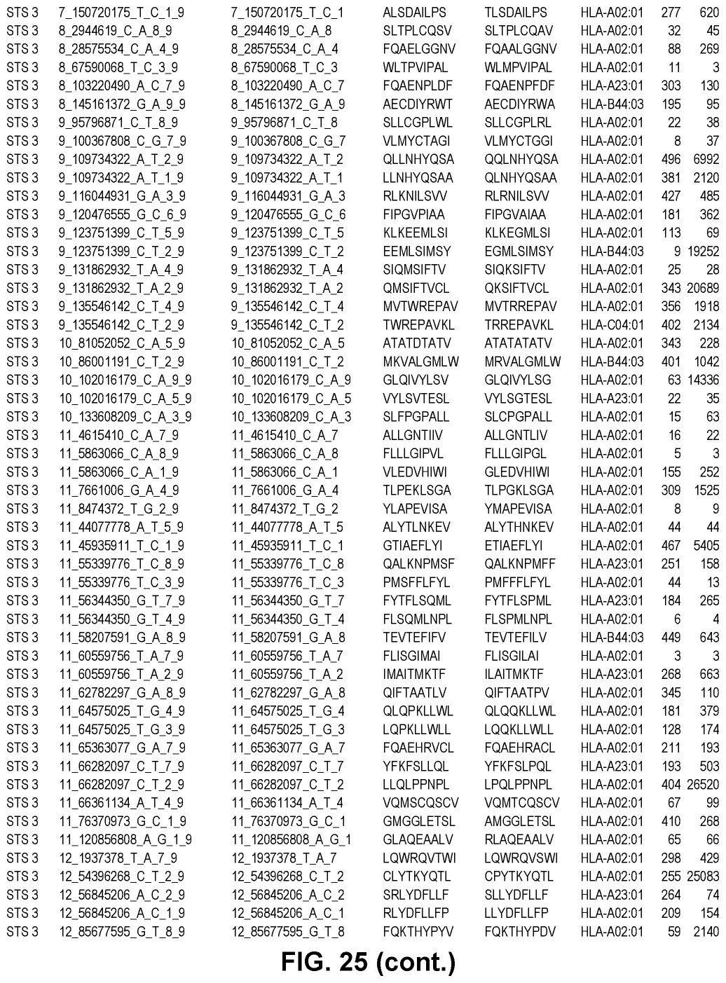

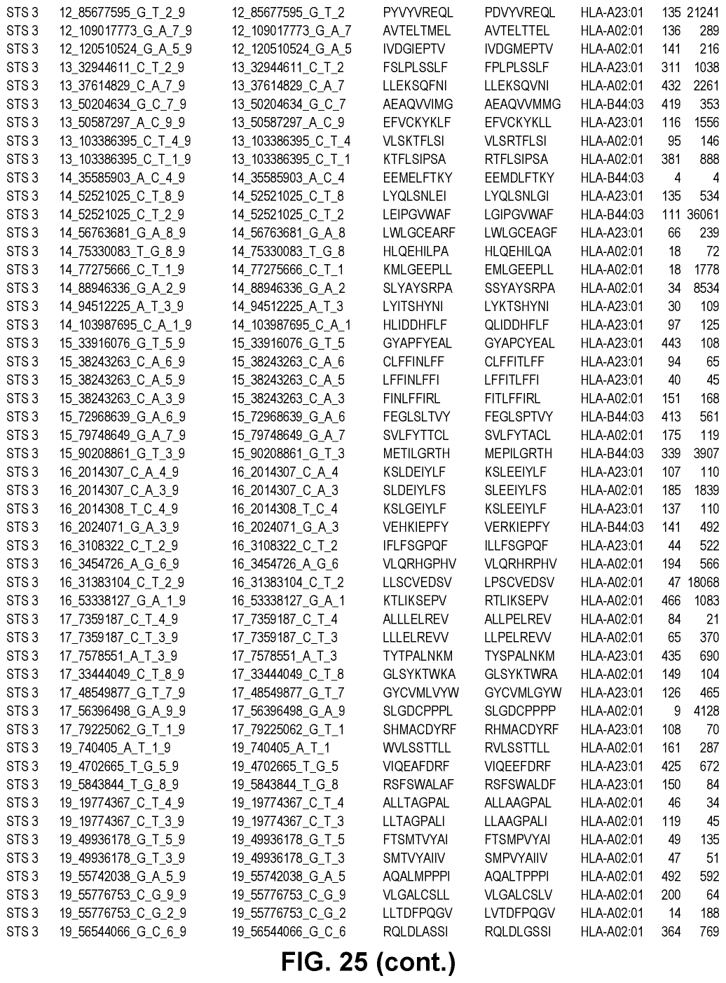

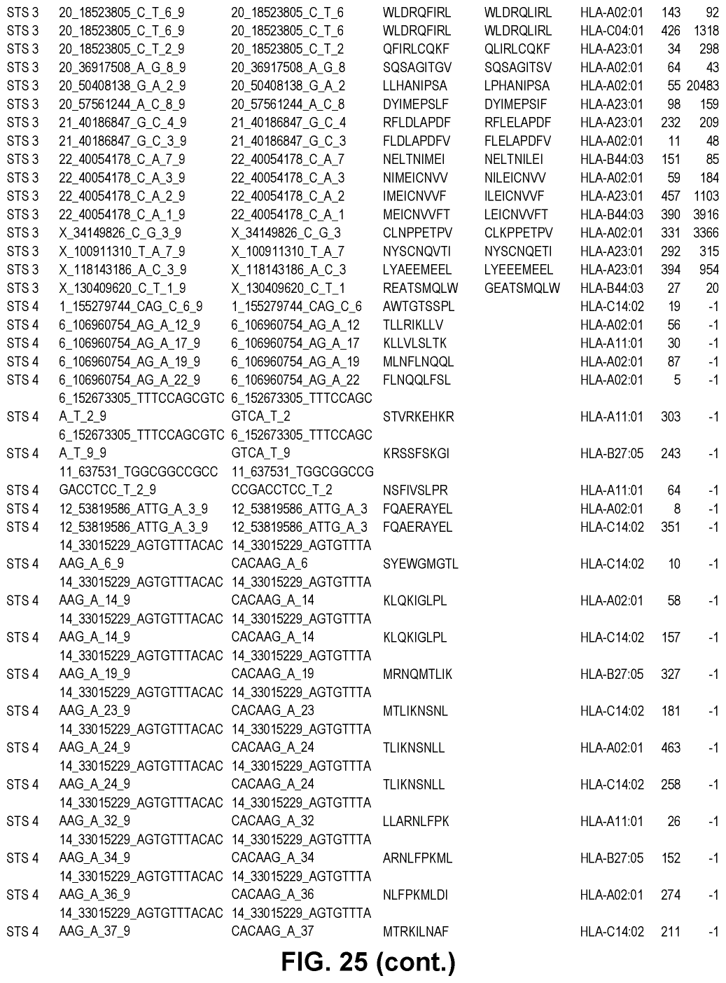

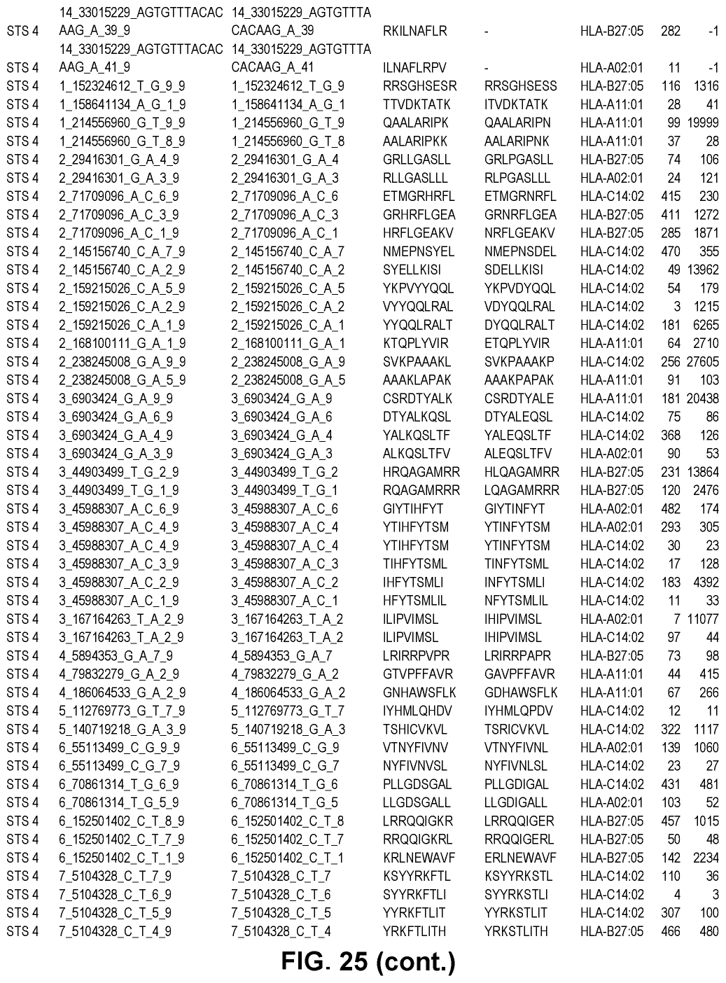

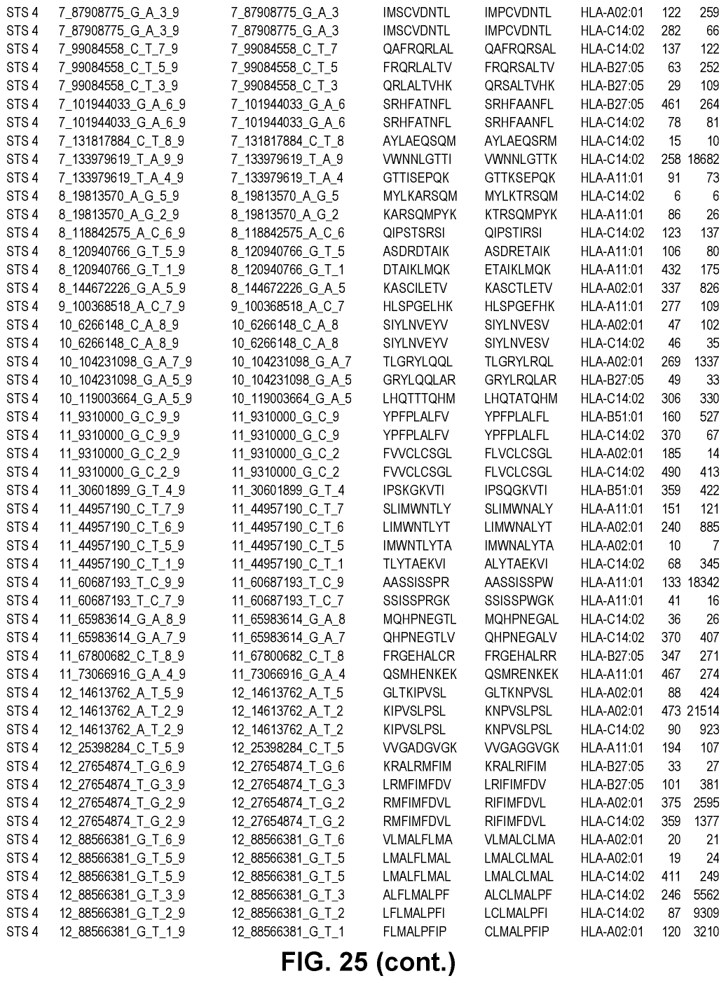

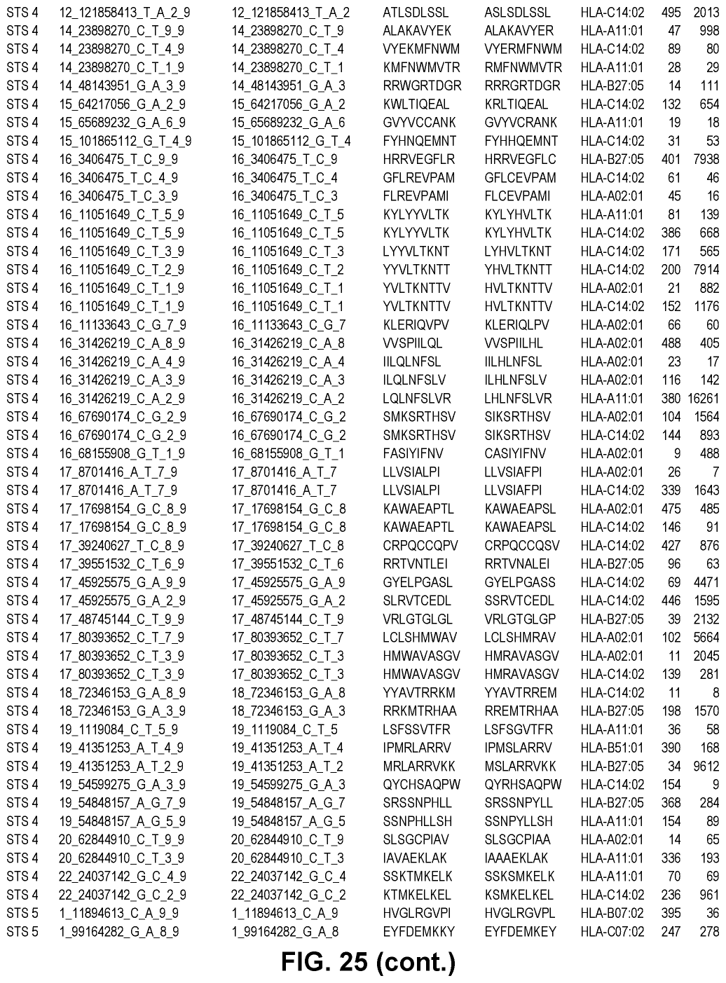

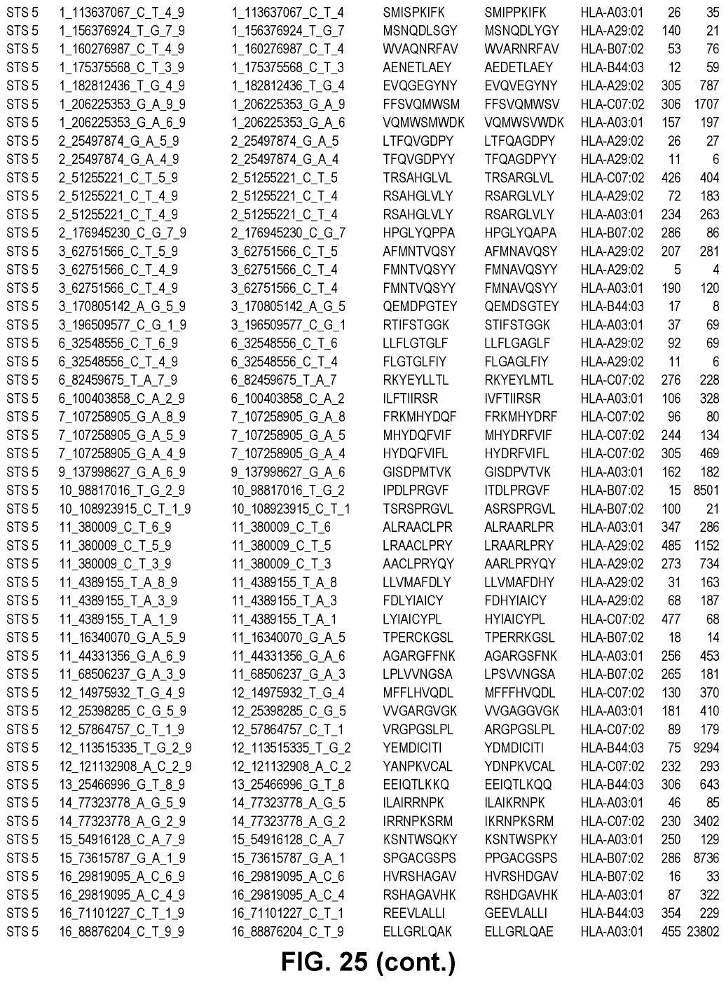

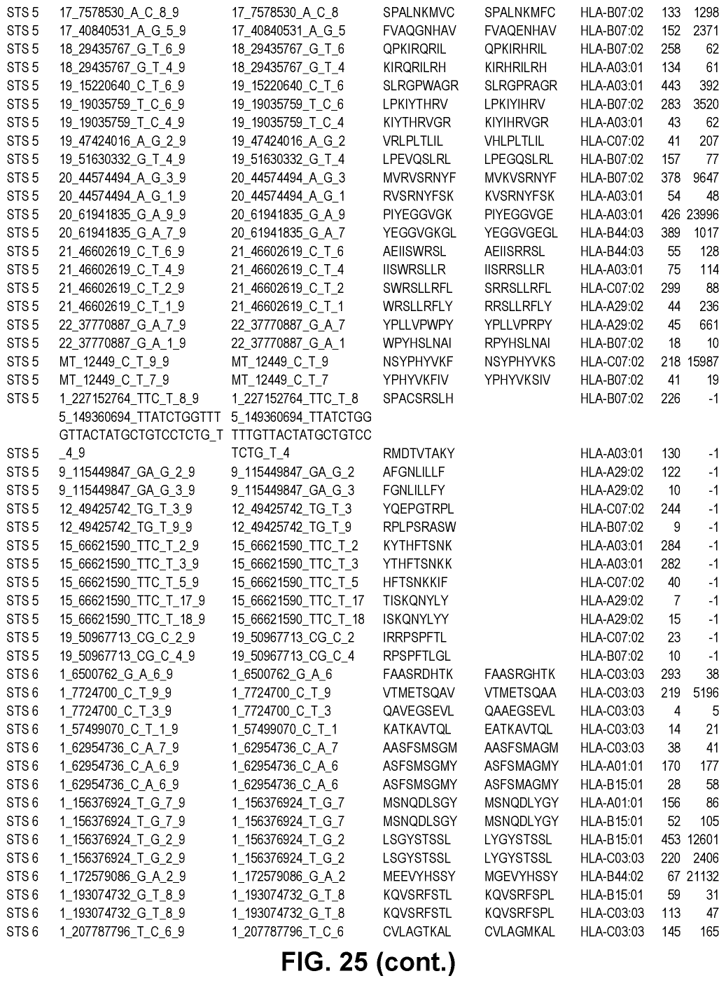

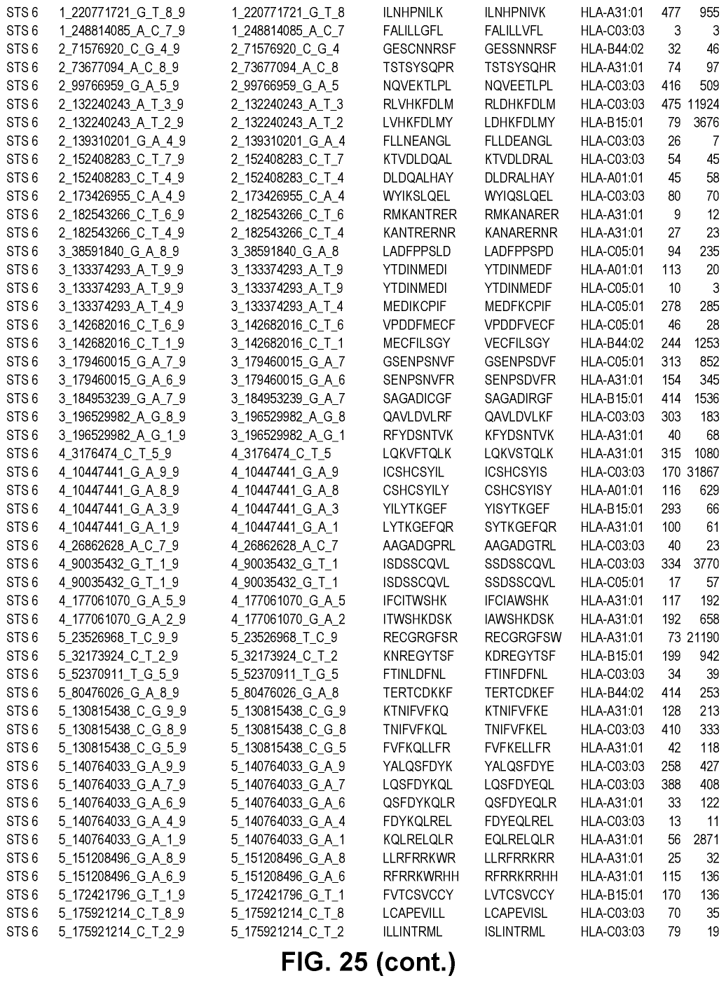

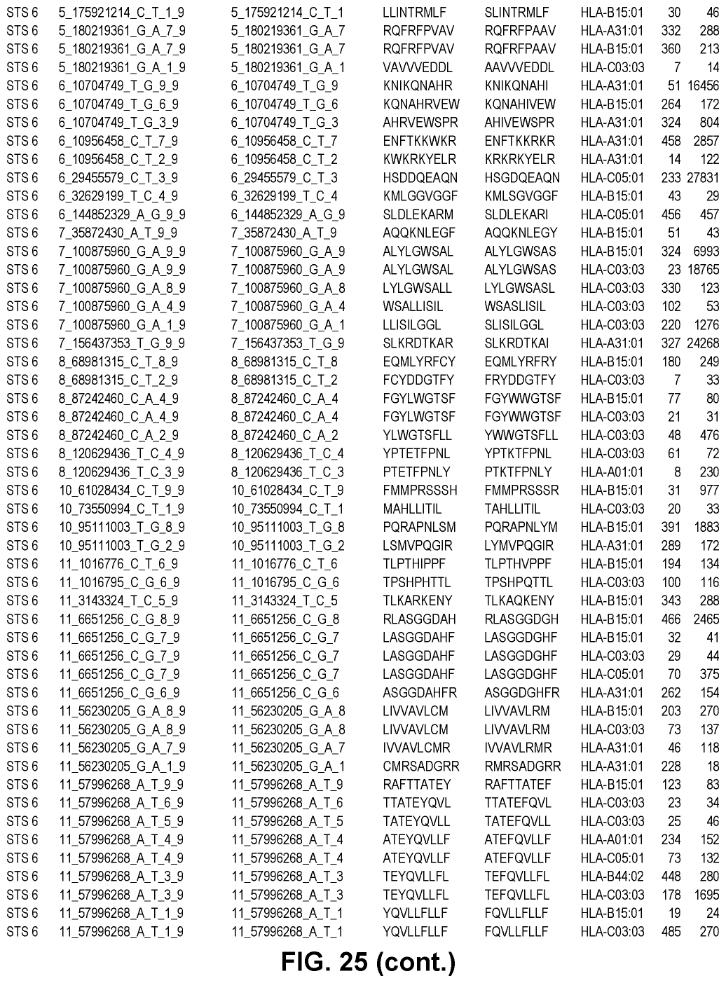

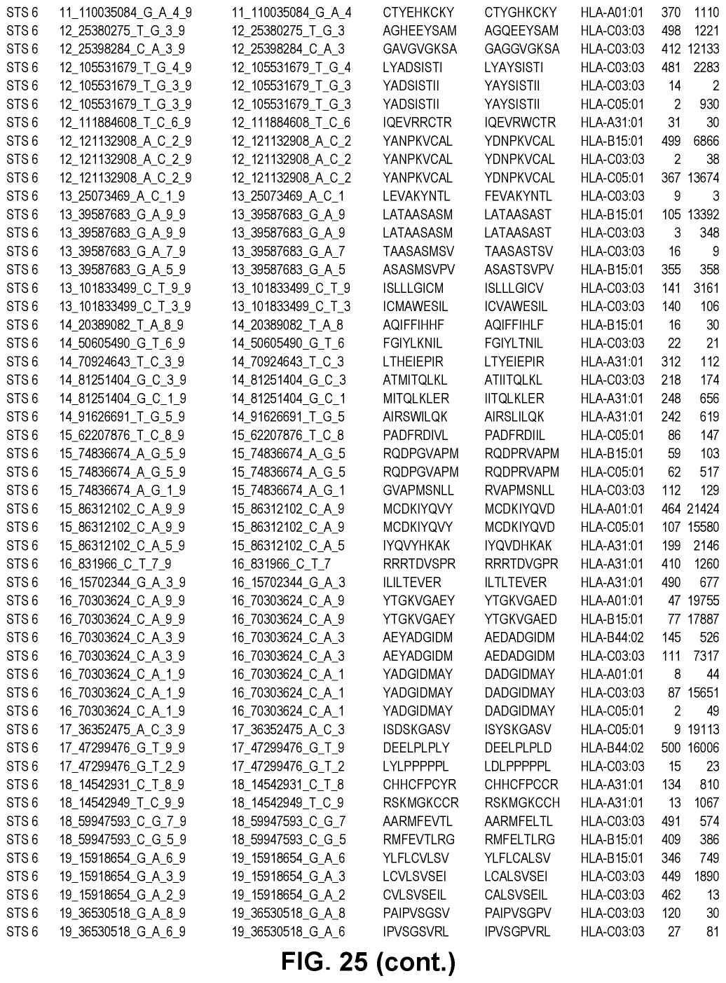

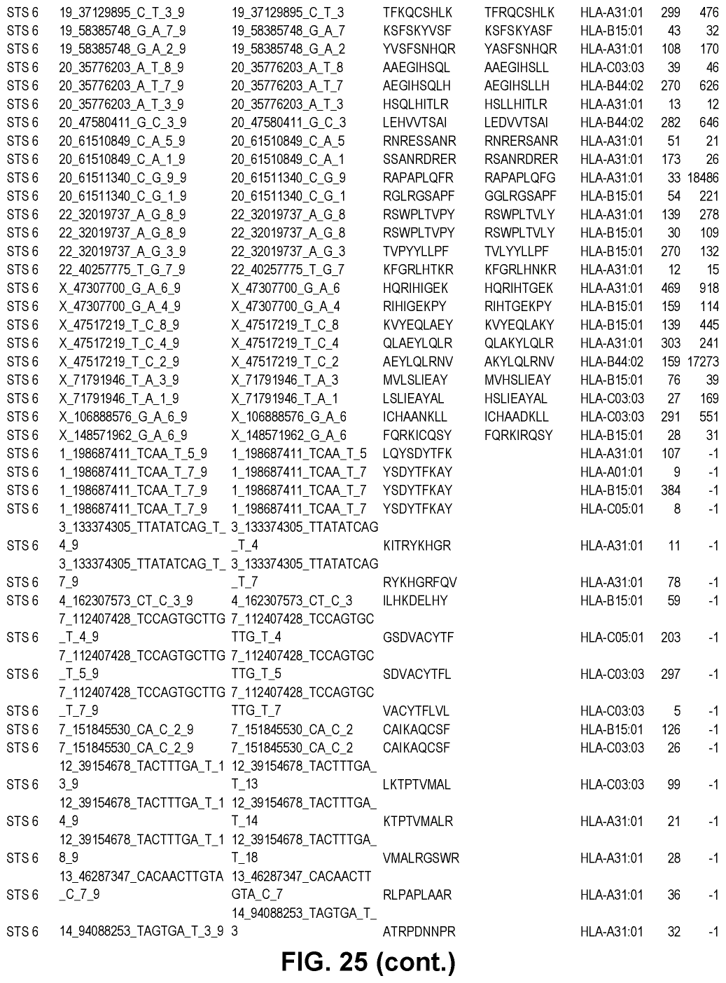

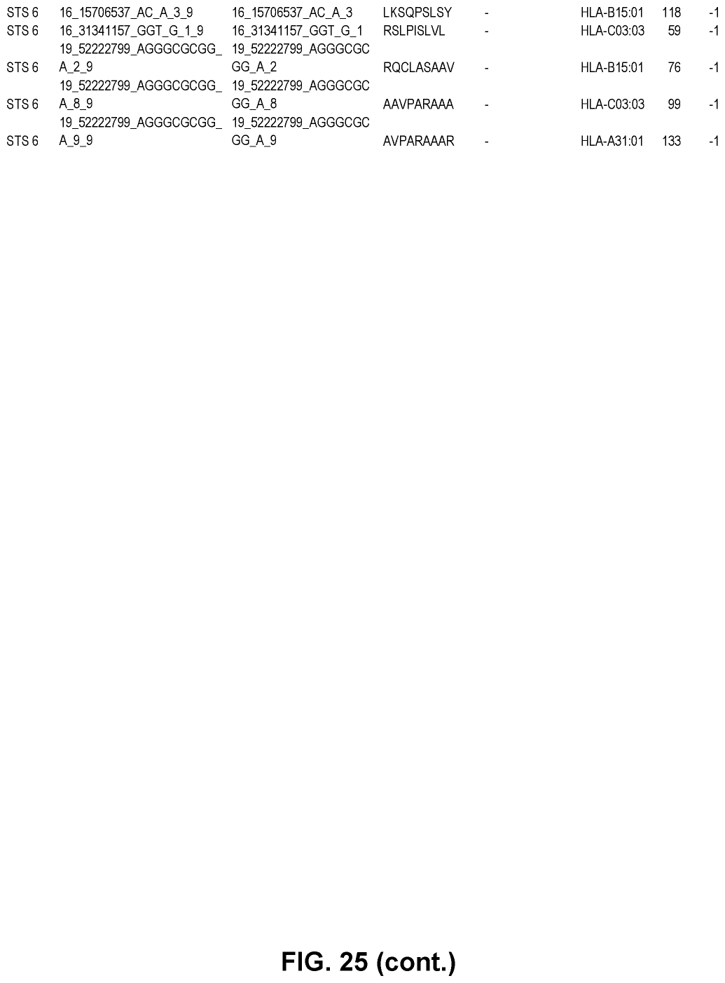

In embodiments, the vaccine comprises one or more neoantigen of according to FIG. 25 or a polynucleotide encoding said neoantigen or a protein or peptide comprising said neoantigen. In embodiments, the one or more neoantigens can be selected from the neoantigenic peptides listed in FIG. 25. In embodiments, the vaccine comprises one or more neoantigens associated with a cancer. In embodiments, the one or more neoantigens can correlate with a neoantigen-microbial homology that is higher than the median neoantigen-microbial homology occurring in subjects with the cancer, or a polynucleotide encoding the neoantigen or a protein or peptide comprising the neoantigen. In embodiments, the neoantigen can correlate with an activated T cell number that is higher than the median activated T cell number occurring in subjects with a cancer, or a polynucleotide encoding the neoantigen or a protein or peptide comprising the neoantigen.

In embodiments, the vaccine comprises a neoantigen that occurs in a subject with a cancer, where this subject has an activated T cell number that is higher than the median activated T cell number of a population of subjects with the cancer. In embodiments, the neoantigen less frequently occurs in subjects with the cancer and having activated T cell numbers at or less than the median activated T cell number.

In embodiments, the number of different neoantigens in the vaccine varies, for example, in some embodiments the vaccine comprises 2, 3, 4, 5, 6, 7, 8, 9, 10 or more different neoantigens. In embodiments, the neoantigens of a given vaccine are linked, e.g., by any biochemical strategy to link two proteins. In some embodiments, the neoantigens of a given vaccine are not linked.

In embodiments, the vaccine comprises one or more polynucleotides.

In embodiments, the one or more polynucleotides are RNA, DNA, or a mixture thereof. In certain embodiments, the vaccine is in the form of DNA or RNA vaccines relating to neoantigens. In embodiments, the one or more neoantigens are delivered via a bacterial or viral vector containing DNA or RNA sequences that encode one or more neoantigen.

Non-limiting examples of vaccines of the present disclosure include tumor cell vaccines, antigen vaccines, and dendritic cell vaccines, RNA vaccines, and DNA vaccines. Tumor cell vaccines are made from cancer cells removed from the patient. In some such embodiments, the cells are altered (and killed) to make them more likely to be attacked by the immune system and then injected back into the patient. In embodiments, the tumor cell vaccines are autologous, e.g., the vaccine is made from killed tumor cells taken from the same person who receives the vaccine. In embodiments, the tumor cell vaccines are allogeneic, e.g., the cells for the vaccine come from someone other than the patient being treated.

In embodiments, the vaccine is an antigen presenting cell vaccine, e.g., a dendritic cell vaccine. Dendritic cells are special immune cells in the body that help the immune system recognize cancer cells. They break down cancer cells into smaller pieces (including antigens), and then hold out these antigens so T cells can see them. The T cells then start an immune reaction against any cells in the body that contain these antigens.

In embodiments, the antigen presenting cell such as a dendritic cell is pulsed or loaded with the neoantigen, or genetically modified (via DNA or RNA transfer) to express one or more neoantigen (see, e.g., Butterfield, 2015, BMJ. 22, 350; and Palucka, 2013, Immunity 39, 38-48). In embodiments, the dendritic cell is genetically modified to express one or more neoantigen peptide. In embodiments, any suitable method known in the art is used for preparing dendritic cell vaccines of the present disclosure. For example, in embodiments, immune cells are removed from the patient's blood and exposed to cancer cells or cancer antigens, as well as to other chemicals that turn the immune cells into dendritic cells and help them grow. The dendritic cells are then injected back into the patient, where they can cause an immune response to cancer cells in the body.

Furthermore, aspects of the invention provide methods for treating cancer in a subject. In embodiments, the method comprises administering to the subject a vaccine comprising a neoantigen according to FIG. 25.

In embodiments, the vaccination is therapeutic vaccination, administered to a subject who has cancer. In embodiments, the vaccination is prophylactic vaccination, administered to a subject who can be at risk of developing cancer. In embodiments, the vaccine is administered to a subject who has previously had cancer and in whom there is a risk of the cancer recurring.

Vaccines can be administered in any suitable way as known in the art. In embodiments, the vaccine is delivered using a vector delivery system. In embodiments, the vector delivery system is viral, bacterial or makes use of liposomes. In embodiments, a listeria vaccine or electroporation is used to deliver the vaccine.

Aspects of the invention further provides a composition comprising a vaccine as described herein. In embodiments, the composition is a pharmaceutical composition. In embodiments, the pharmaceutical composition further comprises a pharmaceutically acceptable carrier, diluent or excipient.

In embodiments, the vaccine leads to generation of an immune response in a subject. In embodiments, the immune response is humoral and/or cell-mediated immunity, for example the stimulation of antibody production, or the stimulation of cytotoxic or killer cells, which can recognize and destroy (or otherwise eliminate) cells (e. g., tumor cells) expressing antigens (e.g., neoantigen) corresponding to the antigens in the vaccine on their surface. In embodiments, inducing or stimulating an immune response includes all types of immune responses and mechanisms for stimulating them. In embodiments, the induced immune response comprises expansion and/or activation of Cytotoxic T Lymphocytes (CTLs). In embodiments, the induced immune response comprises expansion and/or activation of CD8+ T cells. In embodiments, the induced immune response comprises expansion and/or activation of helper CD4+ T Cells. In some embodiments, the extent of an immune response is assessed by production of cytokines, including, but not limited to, IL-2, IFN-γ, and/or TNFa.

In embodiments, the neoantigens can be used in adoptive T cell therapy. For example, embodiments provide a population of T cells that target one or more of the neoantigens described in FIG. 25. In embodiments, the neoantigen are selected based, at least in part, on predicted immunogenicity, for example, in silico. In embodiments, the predicted immunogenicity is analyzed using computational algorithms for MHC class I and class II binding as well as use of tandem minigene libraries for class II epitope screening. In addition, or alternatively, in embodiments neoantigen specific T cell assays are used to differentiate true immunogenic neoepitopes from putative ones {see, Kvistborg et. al, 2016, J. ImmunoTherapy of Cancer 4:22 for a detailed review). In embodiments, any methods and tools known in the art are used to predict immunogenicity of a neoantigen. For example, in embodiments the Immune Epitope Database (IEDB) T Cell Epitope-MHC Binding Prediction Tool disclosed in Brown et. al, 2010, Nucleic Acids Res. 38 (Database issue): D854-62 is used to predict the binding of neoantigen to autologous HLA-A encoded MHC proteins. In embodiments, immunogenicity analysis strategy and tools disclosed in WO2015/103037 are used in accordance with the disclosed subject matter, and the content of the forgoing patent is incorporated herein by reference in its entirety. In certain embodiments, a neoantigen is selectively targeted based on one or more of the following: (i) homology to an epitope of a known pathogen or microbe; and/or (ii) ability to activate T cells, e.g. in an in vitro assay.

In embodiments, the population of T cells target one or more neoantigen of FIG. 25. In embodiments, the population of T cells target one or more neoantigen associated with a cancer. For example, the one or more neoantigen can correlate with a neoantigen-microbial homology that is higher than the median neoantigen-microbial homology occurring in subjects with the cancer. For example, the one or more neoantigen can correlate with an activated T cell number that is higher than the median activated T cell number occurring in subjects with the cancer. In embodiments, the neoantigen comprised in the vaccine occurs in a subject with a cancer, where the subject has an activated T cell number that is higher than the median activated T cell number of a population of subjects with the cancer. In embodiments, the neoantigen occurs less frequently in subjects with the cancer and having activated T cell numbers at or less than the median activated T cell number.

In embodiments, the T cells are selectively expanded to target the one or more neoantigen. T cells are lymphocytes that mature in the thymus and are chiefly responsible for cell-mediated immunity. T cells are involved in the adaptive immune system. In some embodiments, the T cells as described herein can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, regulatory T cells (also known as suppressor T cells), natural killer T cells, mucosal associated invariant T cells, and γδ T cells. Cytotoxic T cells (CTL or killer T cells) are a subset of T lymphocytes capable of inducing the death of infected somatic or tumor cells.

In embodiments, the T cells that specifically target one or more neoantigen are engineered or modified T cells. In embodiments, the engineered T cells comprise a recombinant antigen receptor that specifically targets or binds to one or more of the presently disclose neoantigens. In embodiments, the recombinant antigen receptor specifically targets one or more neoantigen as described in FIG. 25. In embodiments, the recombinant antigen receptor specifically targets one or more neoantigen associated with a cancer. For example, the one or more neoantigens can correlate with a neoantigen-microbial homology that is higher than the median neoantigen-microbial homology occurring in subjects with the cancer. For example, the one or more neoantigens can correlate with an activated T cell number that is higher than the median activated T cell number occurring in subjects with the cancer. In embodiments, the recombinant antigen receptor is a chimeric antigen receptor (CAR). In embodiments, the recombinant antigen receptor is a T cell receptor (TCR). In embodiments, the CAR comprises an extracellular antigen-binding domain that specifically binds to one or more neoantigen, a transmembrane domain, and an intracellular signaling domain. CARs can activate the T-cell in response to recognition by the extracellular antigen-binding domain of its target. When T cells express such a CAR, they recognize and kill cells that express one or more neoantigen.

Affinity-enhanced TCRs are generated by identifying a T cell clone from which the TCR a and β chains with the desired target specificity are cloned. The candidate TCR then undergoes PCR directed mutagenesis at the complimentary determining regions (“CDR”) of the α and β chains. The mutations in each CDR region are screened to select for mutants with enhanced affinity over the native TCR. Once completed, lead candidates are cloned into vectors to allow functional testing in T cells expressing the affinity enhanced TCR.

In embodiments, the T cell population is enriched with T cells that are specific to one or more neoantigen, e.g., having an increased number of T cells that target one or more neoantigen. Therefore, the T cell population differs from a naturally occurring T cell population, in that the percentage or proportion of T cells that target a neoantigen is increased.

In embodiments, the T cell population comprises at least about 10%, about 20%, about 30%, about 40%, about 50%, about 60%, about 70%, about 80%, about 90%, about 95%, or about 100% T cells that target one or more neoantigen. In certain embodiments, the T cell population comprises no more than about 5%, about 10%, about 15%, about 20%, about 25%, about 30%, about 35%, about 40%, about 45%, or about 50% T cells that do not target one or more neoantigen.

In embodiments, the T cell population is generated from T cells isolated from a subject with cancer. In one non-limiting example, the T cell population is generated from T cells in a biological sample isolated from a subject with cancer. In some embodiments, the biological sample is a tumor sample, a peripheral blood sample, or a sample from a tissue of the subject. In certain embodiments, the T cell population is generated from a biological sample in which the one or more neoantigen is identified or detected.

Aspects of the invention further provide a composition comprising such T cell populations as described herein. In embodiments, the composition is a pharmaceutical composition that comprises a pharmaceutically acceptable carrier. Furthermore, embodiments provide a method of treating cancer in a subject, comprising administering to the subject a composition comprising such T cell population as described herein. In embodiments, the cancer is any of the cancers enumerated herein. In embodiments, the composition comprises a population of T cells that target one or more neoantigen of FIG. 25.

In embodiments, the methods are used in vitro, ex vivo or in vivo, for example, either for in situ treatment or for ex vivo treatment followed by the administration of the treated cells to the subject. In embodiments, the T cell population or composition is reinfused into the subject, for example following T cell isolation and expansion to target the one or more neoantigens of FIG. 25.

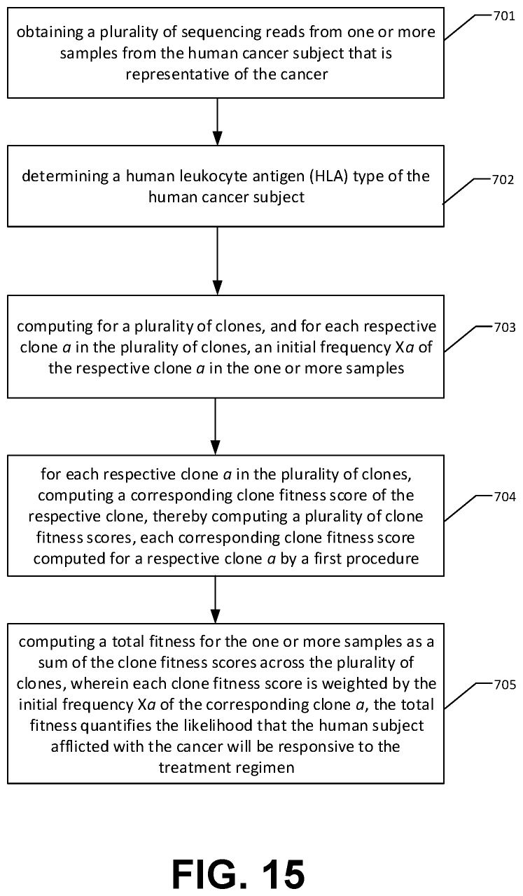

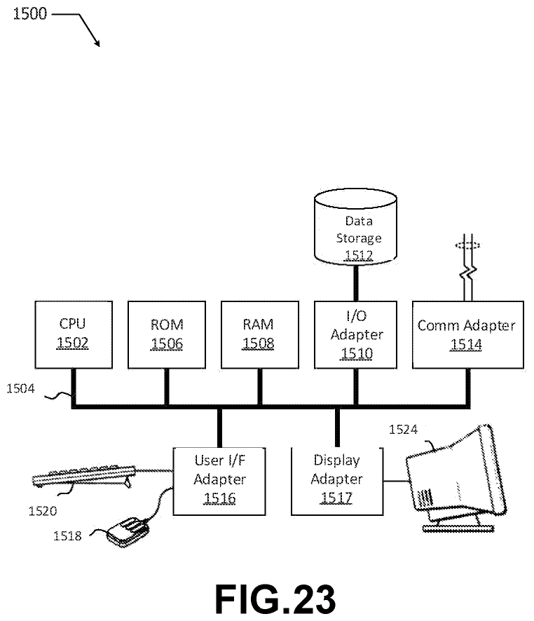

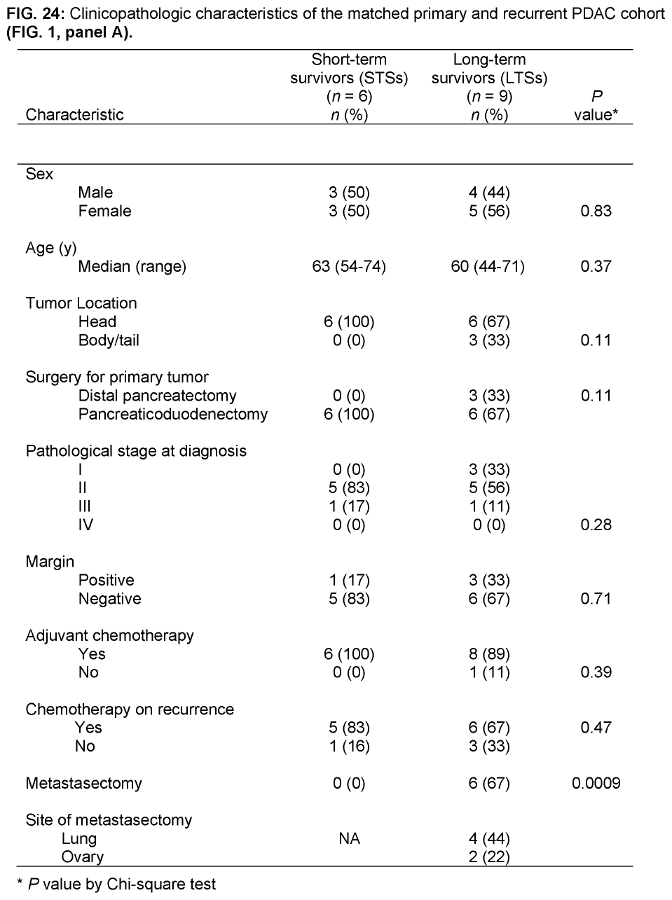

Aspects of the invention are also drawn to computer software and systems comprising the same. For example, the methods described herein can be implemented as a system and method performed by software components of a system determining T-cell cross-reactivity between antigens according to embodiments of the invention. The Determining the various values associated with determining T-cell cross-reactivity between antigens, determining a likelihood that a human subject afflicted with a cancer will be responsive to a treatment regimen that comprises administering a checkpoint blockade immunotherapy directed to the cancer, identifying a neoantigen to target as an immunotherapy for a cancer, and defining that treating a human subject afflicted with a cancer with a checkpoint blockade immunotherapy directed to the cancer can be a time-intensive activity in which large numbers of plurality of sequencing reads, human leukocyte antigen (HLA) types of the human cancer, a plurality of clones, and a plurality of neoantigens are to be considered, identified, and compared. The calculation of each of the clone fitness scores of the respective clones, the calculation of a recognition potential of each respective neoantigen in the plurality of neoantigens in the respective clone a, a calculation of a total fitness for the one or more samples using the clone fitness scores across the plurality of clones, and the subsequent use of these values in determining a preferred course of treatment for an individual's cancer provides an improved methodology for identifying a particular individual's cancer and determining a preferred course of treatment. These calculations and determinations may be performed using software modules that perform a sequence of operations illustrated in the various flowcharts shown in FIGS. 15-21. These sequences of operations may be executed on an example computing system shown in FIG. 23.

FIG. 15 illustrates a flowchart corresponding to a method performed by software components of a system for providing systems and methods for determining T-cell cross-reactivity between antigens 1500 according to embodiments of the present invention. For example, the method of FIG. 15 can be used for providing systems and methods for determining T-cell cross-reactivity between antigens 1500, such as cross-reactivity distance, C, according to Example 2.

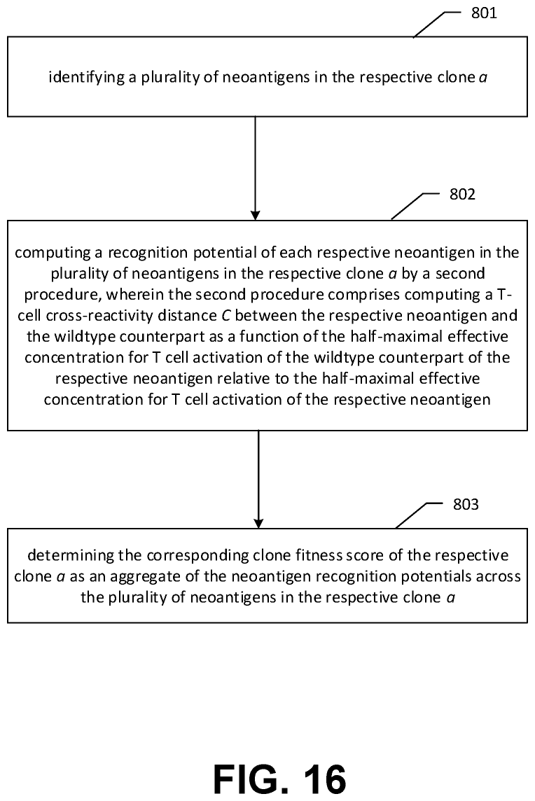

Method 1500 begins with obtaining a plurality of sequencing reads, in step 1501, from one or more samples from the human cancer subject that is representative of the cancer. In step 1502, an HLA type of the human cancer subject is determined. Method 1500 determines, in step 1503, a plurality of clones; and for each respective clone a in the plurality of clones, an initial frequency Xa of the respective clone a is computed in the one or more samples from the plurality of sequencing reads. For each respective clone a in the plurality of clones, computing a corresponding clone fitness score of the respective clone, thereby computes a plurality of clone fitness scores, each corresponding clone fitness score computed by use of method 1600 described below in reference to FIG. 16. Method 1500 ends by selecting at least a first neoantigen from a plurality of neoantigens for a respective clone a in the plurality of respective clones in step 1504 based upon the recognition potential of the first neoantigen to target as an immunotherapy for the cancer.

FIG. 16 illustrates a flowchart corresponding to a method performed by software components of a system for providing systems and methods for determining T-cell cross-reactivity between antigens according to embodiments of the present invention. Method 1600 begins by identifying a plurality of neoantigens in the respective clone a in step 1601. In step 1602, a recognition potential of each respective neoantigen in the plurality of neoantigens in the respective clone a is computing a T-cell cross-reactivity distance C between the respective neoantigen and the wildtype counterpart as a function of the half-maximal effective concentration for T-cell activation of the wildtype counterpart of the respective neoantigen relative to the half-maximal effective concentration for T-cell activation of the respective neoantigen. After the corresponding clone fitness score of the respective clone a is determined in step 1603 as an aggregate of the neoantigen recognition potentials across the plurality of neoantigens in the respective clone a, method 1600 ends.

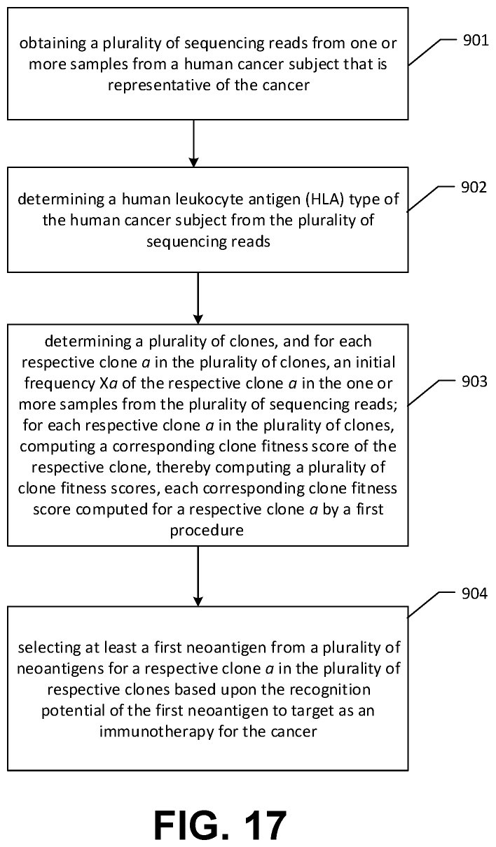

FIG. 17 illustrates a flowchart corresponding to a method performed by software components of a system for providing systems and methods for determining T-cell cross-reactivity between antigens according to embodiments of the present invention. Method 1700 obtains a plurality of sequencing reads from one or more samples in step 1701 from the human cancer subject that is representative of the cancer. In step 1702, an HLA type is determined of the human cancer subject from the plurality of sequencing reads. For each respective clone a in the plurality of clones in step 1703, an initial frequency Xa of the respective clone a is determined in the one or more samples from the plurality of sequencing reads.

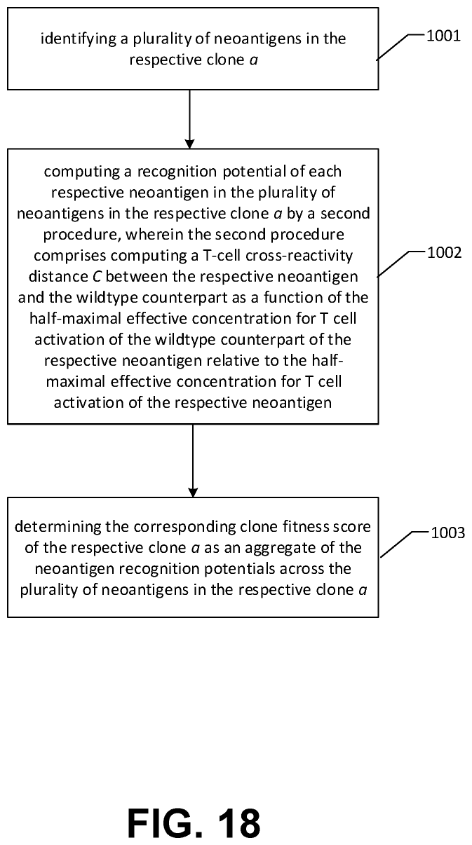

Method 1700 computes for each respective clone a in the plurality of clones, a corresponding clone fitness score of the respective clone in step 1704, thereby computing a plurality of clone fitness scores, each corresponding clone fitness score computed for a respective clone a by utilizing the method of FIG. 18. After selecting, in step 1705, at least a first neoantigen from a plurality of neoantigens for a respective clone a in the plurality of respective clones based upon the recognition potential of the first neoantigen as the immunotherapy for the cancer, method 1700 ends.

FIG. 18 illustrates a flowchart corresponding to a method performed by software components for computing each corresponding clone fitness score computed for a respective clone a according to embodiments of the present invention. For example, FIG. 18 illustrates a flowchart corresponding to a method performed by software components for computing each corresponding clone fitness score computed for a respective clone a, as described in Example 2. Method 1800 identifies a plurality of neoantigens in the respective clone a in step 1801. In step 1802, a recognition potential of each respective neoantigen in the plurality of neoantigens in the respective clone a is computed by computing a T-cell cross-reactivity distance C between the respective neoantigen and the wildtype counterpart as a function of the half-maximal effective concentration for T-cell activation of the wildtype counterpart of the respective neoantigen relative to the half-maximal effective concentration for T-cell activation of the respective neoantigen. Method 1800 ends when the corresponding clone fitness score of the respective clone a is determined in step 1803 as an aggregate of the neoantigen recognition potentials across the plurality of neoantigens in the respective clone a.

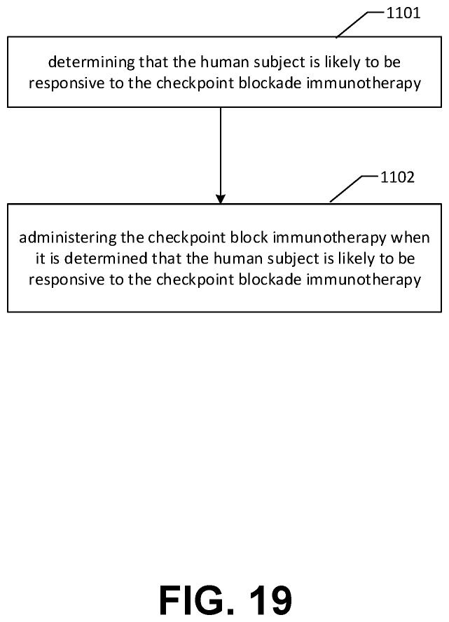

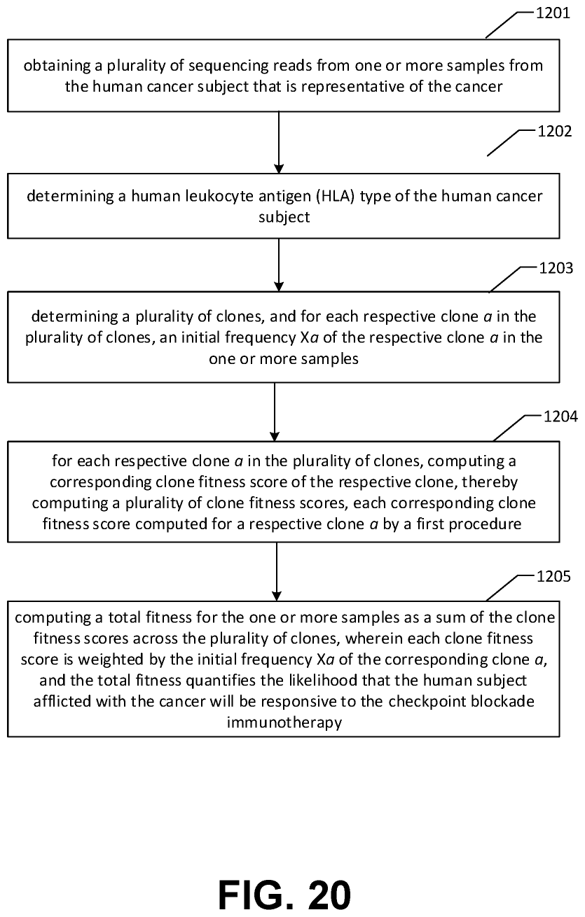

FIG. 19 illustrates a flowchart corresponding to a method performed by software components providing a method for treating a human subject afflicted with a cancer with a checkpoint blockade immunotherapy directed to the cancer according to embodiments of the present invention. Method 1900 begins by determining that the human subject is likely to be responsive to the checkpoint blockade immunotherapy in step 1901. In step 1902, the checkpoint block immunotherapy is administered when it is determined that the human subject is likely to be responsive to the checkpoint blockade immunotherapy. The determination that the human subject will be responsive to the checkpoint blockade immunotherapy is described in reference to FIG. 20 below.

FIG. 20 is a flowchart corresponding to a method performed by software components for the determination that the human subject will be responsive to the checkpoint blockade immunotherapy according to embodiments of the present invention. For example, FIG. 20 is a flowchart corresponding to a method performed by software components for the determination that the human subject will be responsive to the checkpoint blockade immunotherapy according to embodiments of the present invention, such as those described in Example 2. Method 2000 begins when step 2001 obtains a plurality of sequencing reads from one or more samples from the human cancer subject that is representative of the cancer. In step 2002, an HLA type is determined for the human cancer subject. For each respective clone a in the plurality of clones, an initial frequency Xa of the respective clone a is determined in the one or more samples in step 2003.

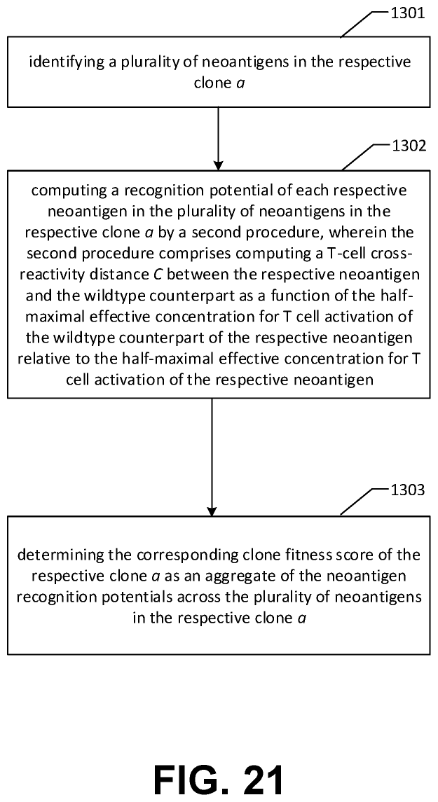

Method 2000 computes a corresponding clone fitness score for each respective clone a in the plurality of clones in step 2004, thereby computing a plurality of clone fitness scores, each corresponding clone fitness score computed for a respective clone a. Additional details of computing each corresponding clone fitness score are described in reference to FIG. 21 below. Method 2000 ends by determining the corresponding clone fitness score of the respective clone a in step 2005 as an aggregate of the neoantigen recognition potentials across the plurality of neoantigens in the respective clone a.

FIG. 21 illustrates a flowchart corresponding to a method performed by software components for providing a corresponding clone fitness score computed for a respective clone a according to embodiments of the present invention. Method 2100 begins by identifying a plurality of neoantigens in the respective clone a in step 2101. In step 2102, a recognition potential for each respective neoantigen in the plurality of neoantigens in the respective clone a is computed by computing a T-cell cross-reactivity distance C between the respective neoantigen and the wildtype counterpart as a function of the half-maximal effective concentration for T-cell activation of the wildtype counterpart of the respective neoantigen relative to the half-maximal effective concentration for T-cell activation of the respective neoantigen. Method 2100 ends by determining the corresponding clone fitness score of the respective clone a as an aggregate of the neoantigen recognition potentials across the plurality of neoantigens in the respective clone a in step 2103.

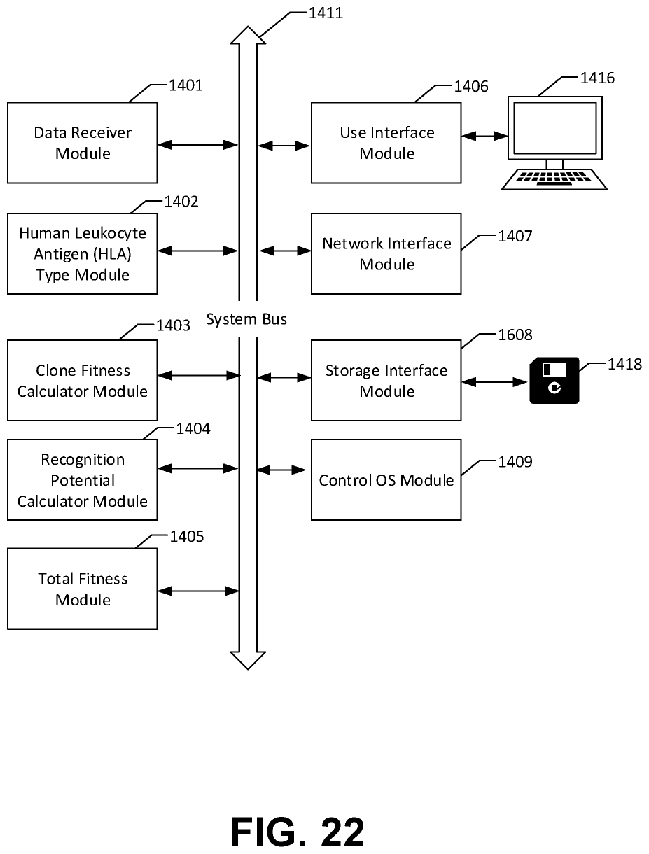

FIG. 22 illustrates a computing system of software components for providing a method for providing systems and methods for determining T-cell cross-reactivity between antigens according to embodiments of the present invention. The software components include a data receiver module 2201, an HLA type module 2202, a clone fitness calculator module 2203, a recognition potential calculator module 2204, a total fitness module 2205, a user interface module 2206, a network interface module 2207, a storage interface module 2208, and a control OS module 2209. All of these components 2201-2209 communicate with each other over a system bus 2211 that accesses memory in the computing system executing these components and mass storage devices 2218 as needed.

The data receiver module 2201 receives data from external sources data associated with and containing test sample data associated with a plurality of sequencing reads, HLA types of the human cancer, a plurality of clones, and a plurality of neoantigens data. The data receiver module 2201 performs data input operations, data formatting operations, and data storage operations needed to obtain the data and format the data into a form useful for processing by other software components 2202-2209. The data receiver module 2201 maintains the data within the memory and/or mass storage devices 2218 for later use

The method for determining a likelihood that a human subject afflicted with a cancer will be responsive to a treatment regimen that comprises administering a checkpoint blockade immunotherapy directed to the cancer to the subject utilizes the HLA type module 2202, clone fitness calculator module 2203, and the recognition potential calculator module 2204 within the computing system. The method for identifying a neoantigen to target as an immunotherapy for a cancer utilizes the data receiver module 2201, the HLA type module 2202, clone fitness calculator module 2203, and the recognition potential calculator module 2204 within the computing system. Similarly, the method of treating a human subject afflicted with a cancer with a checkpoint blockade immunotherapy directed to the cancer utilizes the data receiver module 2201, the HLA type module 2202, clone fitness calculator module 2203, and the recognition potential calculator module 2204 within the computing system. These software component modules are described herein.

The HLA type module 2202 performs the operations to determine an HLA type of the human cancer subject as described herein, such as in Example 2. The HLA type module 2202 receives data via the data receiver module 2201 and/or the storage interface module 2208 to generate the HLA type data disclosed herein. The HLA type module 2202 may pass the resultant HLA type data to other software component modules 2203-2205 for additional processes and/to the storage interface module 2208 for storage within mass storage devices 2218 for later retrieval and use.

The clone fitness calculator module 2203 performs the operations to determine clone fitness score values of the respective clone data as described herein in reference, such as in Example 2. The clone fitness calculator module 2203 receives data via the data receiver module 2201 and/or the storage interface module 2208 to generate clone fitness score values of the respective clone data disclosed herein. The clone fitness calculator module 2203 may pass the resultant clone fitness score of the respective clone data to other software component modules 2203-2205 for additional processes and/to the storage interface module 2208 for storage within mass storage devices 2218 for later retrieval and use.

The recognition potential calculator module 2204 the operations to determine a recognition potential value as described herein, such as in Example 2. The recognition potential calculator module 2204 receives data via the data receiver module 2201 and/or the storage interface module 2208 to generate recognition potential values of each respective neoantigen in the plurality of neoantigens in the respective clone a data disclosed herein. The recognition potential calculator module 2204 may pass the resultant recognition potential of each respective neoantigen in the plurality of neoantigens in the respective clone a data to other software component modules 2203-2205 for additional processes and/to the storage interface module 2208 for storage within mass storage devices 2218 for later retrieval and use.

The total fitness module 2205 performs the operations to determine a total fitness value as described herein, such as in Example 2. The total fitness module 2205 receives data via the data receiver module 2201 and/or the storage interface module 2208 to generate a total fitness value for the one or more samples as a sum of the clone fitness scores across the plurality of clone data disclosed herein. The total fitness module 2205 may pass the total fitness value data to other software component modules 2203-2205 for additional processes and/to the storage interface module 2208 for storage within mass storage devices 2218 for later retrieval and use.

The user interface module 2206 provides input and output processing to provide a user with messages and data needed for determining T-cell cross-reactivity between antigens, for determining a likelihood that a human subject afflicted with a cancer will be responsive to a treatment regimen that comprises administering a checkpoint blockade immunotherapy directed to the cancer, for identifying a neoantigen to target as an immunotherapy for a cancer, and for defining a treating a human subject afflicted with a cancer with a checkpoint blockade immunotherapy directed to the cancer. This user interface module 2206 also accepts commands from the user to instruct the application to perform these tasks.

The network interface module 2207 performs operations to communicate with remote computing devices over one or more communications networks 2217. The network interface module 2207 formats outgoing data into a form allowing a connection to a remote server to send and receive communication from users, laboratories, hospitals, doctors' offices, and the like. The network interface 2207 performs all necessary data formatting, data packet creation, data encryption for security, and data transmission and reception when the server communicates with other processing systems disclosed herein. The network interface 2207 is also responsible for ensuring reception of any communications to other computing systems and to log any errors or attempts to hack into the system.

The storage interface module 2208 performs operations to access mass storage devices 2218 to allow the storage and retrieval of data and software component modules while operating any of the software components disclosed herein. The storage interface module 2208 may communicate with remote mass storage devices via the network interface module 2207 in a similar manner. The storage interface module 2208 also may perform user access security functions, data encryption functions, and data backup and restore functions associated with the data stored within the mass storage devices 2218.

The control OS module 2209 receives user commands via the user interface module 2206 and network interface module 2207 to cause the software component modules 2201-2209 to perform supported operations. The control OS module 2209 controls the instantiation and engagement of the other computer component modules 2202-2209 with each other and various data sources to coordinate processing operations in response to the user commands. The control OS module 2209 may provide security functions related to user identification, authentication, and authorization allowing individual users to access the system and data. The control OS module 2209 also may perform periodic maintenance, system status and error reporting, and event logging in order to maintain efficient operation of the system.

FIG. 23 illustrates a computer system 2300 adapted according to certain embodiments of the server and/or the user interface device utilized to execute the software component modules disclosed herein. The central processing unit (“CPU”) 2302 is coupled to the system bus 2304. The CPU 2302 may be a general-purpose CPU or microprocessor, graphics processing unit (“GPU”), and/or microcontroller. The present embodiments are not restricted by the architecture of the CPU 2302 so long as the CPU 2302, whether directly or indirectly, supports the operations as described herein. The CPU 2302 may execute the various logical instructions according to the present embodiments.

The computer system 2300 also may include random access memory (RAM) 2308, which may be synchronous RAM (SRAM), dynamic RAM (DRAM), synchronous dynamic RAM (SDRAM), or the like. The computer system 2300 may utilize RAM 2308 to store the various data structures used by a software application. The computer system 2300 may also include read only memory (ROM) 2306 which may be PROM, EPROM, EEPROM, optical storage, or the like. The ROM may store configuration information for booting the computer system 2300. The RAM 2308 and the ROM 2306 hold user and system data, and both the RAM 2308 and the ROM 2306 may be randomly accessed.

The computer system 2300 also may include an input/output (I/O) adapter 2310, a communications adapter 2314, a user interface adapter 2316, and a display adapter 2322. The I/O adapter 2310 and/or the user interface adapter 2316 may, in certain embodiments, allow a user to interact with the computer system 2300. In a further embodiment, the display adapter 2322 may display a graphical user interface (GUI) associated with a software or web-based application on a display device 2324, such as a monitor or touch screen.

The I/O adapter 2310 may couple one or more storage devices 2312, such as one or more of a hard drive, a solid-state storage device, a flash drive, a compact disc (CD) drive, flash drive, solid state device (SSD), a floppy disk drive, and a tape drive, to the computer system 2300. According to one embodiment, the data storage 2312 may be a separate server coupled to the computer system 2300 through a network connection to the I/O adapter 2310. The communications adapter 2314 may be adapted to couple the computer system 2300 to the network 2312, which may be one or more of a LAN, WAN, and/or the Internet. The communications adapter 2314 also may be adapted to couple the computer system 2300 to other networks such as a global positioning system (GPS) or a Bluetooth network. The user interface adapter 2316 couples user input devices, such as a keyboard 2320, a pointing device 2318, and/or a touch screen (not shown) to the computer system 2300. The keyboard 2320 may be an on-screen keyboard displayed on a touch panel. Additional devices (not shown) such as a camera, microphone, video camera, accelerometer, compass, and or gyroscope may be coupled to the user interface adapter 2316. The display adapter 2322 may be driven by the CPU 2302 to control the display on the display device 2324. Any of the devices 2302-2322 may be physical and/or logical.

The applications of the present disclosure are not limited to the architecture of the computer system 2300. Rather the computer system 2300 is provided as an example of one type of computing device that may be adapted to perform the functions of a processing modules 2200 and/or the user interface device 2310. For example, any suitable processor-based device may be utilized including, without limitation, personal data assistants (PDAs), tablet computers, smartphones, computer game consoles, and multi-processor servers. Moreover, the systems and methods of the present disclosure may be implemented on application specific integrated circuits (ASIC), very large scale integrated (VLSI) circuits, state machine digital logic-based circuitry, or other circuitry.

The embodiments described herein are implemented as logical operations performed by a computer. The logical operations of these various embodiments of the present invention are implemented (1) as a sequence of computer implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules. As such, persons of ordinary skill in the art may utilize any number of suitable electronic devices and similar structures capable of executing a sequence of logical operations according to the described embodiments. For example, the computer system 800 may be virtualized for access by multiple users and/or applications.

Additionally, the embodiments described herein are implemented as logical operations performed by a computer. The logical operations of these various embodiments of the present invention are implemented (1) as a sequence of computer implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps, or modules.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention. This written description provides an illustrative explanation and/or account of the present invention. It may be possible to deliver equivalent benefits using variations of the specific embodiments, without departing from the inventive concept. This description and these drawings, therefore, are to be regarded as illustrative and not restrictive.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the testing measurements.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

### EXAMPLES

Embodiments of the present disclosure can be further defined by reference to the following non-limiting examples. It will be apparent to those skilled in the art that many modifications, both to materials and methods, can be practiced without departing from the scope of the present disclosure.

### Example 1 Neoantigen Quality Predicts Immunoediting in Survivors of Pancreatic Cancer

Cancer immunoediting 1 is a hallmark of cancer 2 that predicts that lymphocytes kill more immunogenic cancer cells to cause less immunogenic clones to dominate a population. Although proven in mice 1,3, whether immunoediting occurs naturally in human cancers remains unclear. Here, to address this, we validated how 70 human pancreatic cancers evolved over 10 years. We find that, despite having more time to accumulate mutations, rare long-term survivors of pancreatic cancer who have stronger T cell activity in primary tumors develop genetically less heterogeneous recurrent tumors with fewer immunogenic mutations (neoantigens). To quantify whether immunoediting underlies these observations, we infer that a neoantigen can be immunogenic (high-quality) by two features—‘non-selfness’ based on neoantigen similarity to known antigens 4,5, and ‘selfness’ based on the antigenic distance required for a neoantigen to differentially bind to the MHC or activate a T cell compared with its wild-type peptide. Using these features, we estimate cancer clone fitness as the aggregate cost of T cells recognizing high-quality neoantigens offset by gains from oncogenic mutations. Without wishing to be bound by theory, this model indicates the clonal evolution of tumors to reveal that long-term survivors of pancreatic cancer develop recurrent tumors with fewer high-quality neoantigens. Thus, evidence indicates that that the human immune system naturally edits neoantigens. Furthermore, we present a model to predict how immune pressure induces cancer cell populations to evolve over time. More broadly, our results indicate that the immune system fundamentally surveils host genetic changes to suppress cancer.

In 1957, Burnet and Thomas proposed that the immune system in multicellular organisms must eliminate transformed cells as an evolutionary necessity to maintain tissue homeostasis. This theory of ‘cancer immunosurveillance’ was later redefined more broadly as ‘cancer immunoediting’ 6—as a consequence of the immune system protecting the host from cancer, the immune system must also sculpt developing cancers 1,7. When cancers develop, they accumulate mutations, some of which generate new protein sequences (neoantigens) 8. As neoantigens are mostly absent from the human proteome, they can escape T cell central tolerance in the thymus to become antigens in cancers 8. However, neoantigens typically arise in passenger mutations, and therefore distribute heterogeneously in cancer cell clones with variable immunogenicity. Thus, T cells selectively ‘edit’ clones 1 with more immunogenic neoantigens 3, inducing less immunogenic clones to outgrow in cancers.

Although cancer immunoediting has been demonstrated through longitudinal studies in immune-proficient and immune-deficient mice 1,3,8, whether it is a general principle of how human cancers evolve remains uncertain. 9-11 Definitive evidence requires longitudinal tracking of large numbers of patients and cancers over time. As this is logistically challenging, whether the human immune system naturally edits cancers and whether edited clones can be predicted a priori remain unclear.

### Quantifying Selection Pressures on Neoantigens

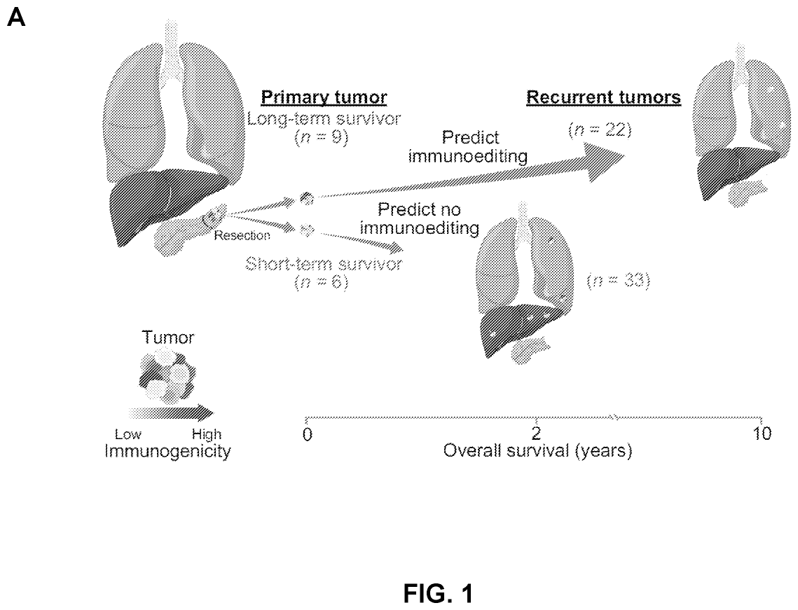

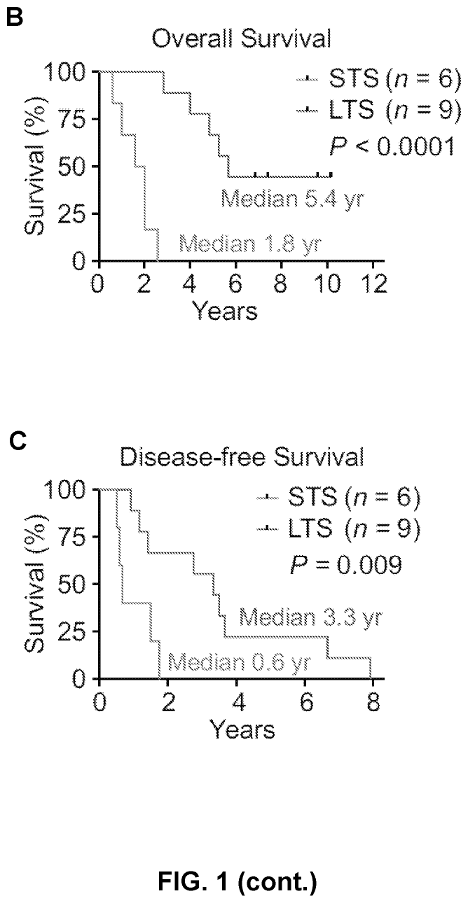

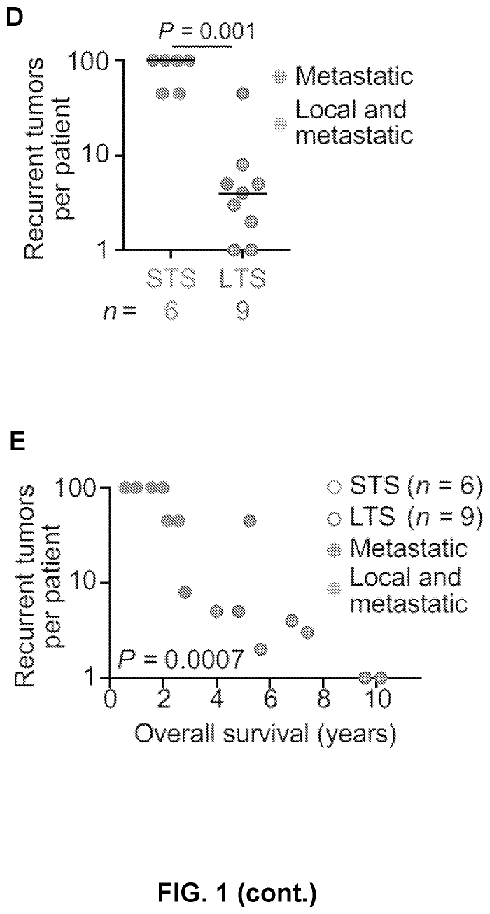

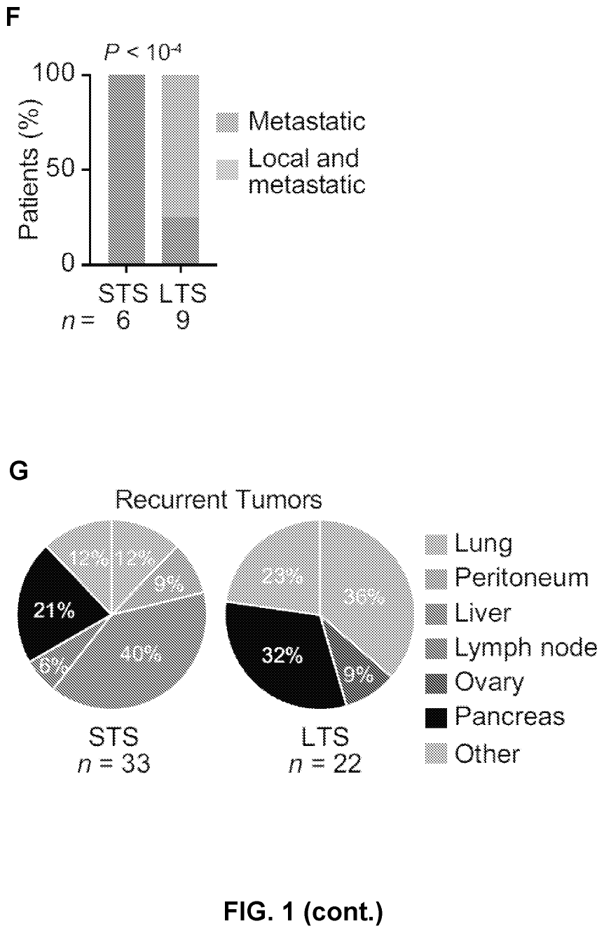

To address this, we examined how 70 pancreatic ductal adenocarcinomas (PDACs) from 15 patients evolved longitudinally over 10 years (FIG. 1, panel A). We reasoned that PDAC is an ideal cancer to validate the concept of immunoediting. First, human PDACs have fewer neoantigens (35 on average) 5,12 compared with more immunogenic cancers (112 in non-small-cell lung cancer 13, 370 in melanoma 14 on average). This theoretically maximizes our ability to both distinguish true neoantigen selection from neutral genomic changes over time and isolate effects of individual neoantigens on clonal selection. Second, T cell infiltrates in PDACs range from nearly zero to 1,000-fold higher 5. Thus, PDACs have subsets that approximate immune-deficient and immune-proficient cancers, allowing us to observe how differential immune selection pressures modulate cancer cell clones. Finally, mutations in oncogenes occur early in PDAC carcinogenesis and are clonal 15—this largely equalizes the cell-intrinsic oncogenic pressures among clones, maximizing our ability to detect how cell-extrinsic immune pressures affect clonal evolution.

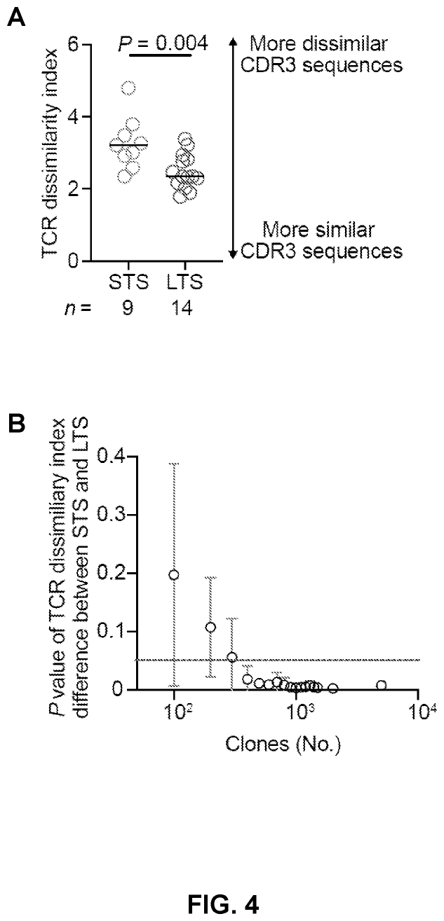

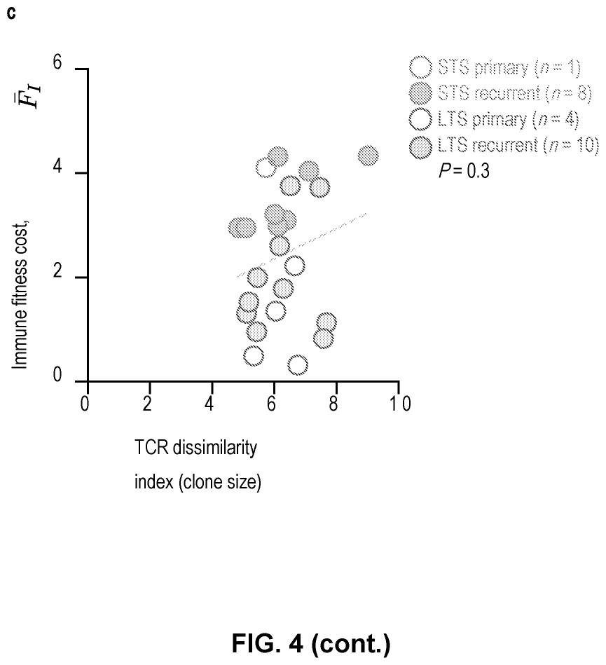

To model how immune-proficient and immune-deficient human cancers evolve, we compared how primary PDACs evolve to recurrence in a cohort of long-term survivors (LTSs) and short-term survivors (STSs) (FIG. 1, panels A and B; FIG. 24). We previously demonstrated that, compared with STSs, LTSs have primary tumors with around a 12-fold greater number of activated CD8+ T cells 5,16,17 that are predicted to target immunogenic neoantigens 5, therefore phenocopying relative greater immune pressure. Furthermore, in the current cohort we find that the largest T cell clones of LTS tumors have more similar CDR3β sequences 18 compared with the largest T cell clones in STS tumors (FIG. 4, panels A and B), indicating T cell clonal expansion and therefore greater immune activity in LTSs. Without wishing to be bound by theory, this higher immune pressure in LTSs would induce tumors to preferentially lose tumour clones with immunogenic neoantigens over time (FIG. 1, panel A). To validate this, we compared how tumors evolved from primary to recurrent tumors. We found that compared with STSs, LTSs had later (FIG. 1, panel C) and fewer recurrent tumors (FIG. 1, panel D) that inversely correlated with longer survival times (FIG. 1, panel E). Moreover, 75% of LTSs versus 0% of STSs had recurrent tumors that were only metastatic (FIG. 1, panel F), with distinct tissue-tropic recurrence patterns (FIG. 1, panel G). Thus, LTS tumors recur with distinct latency, multiplicity and tissue-dependent evolutionary trajectories.

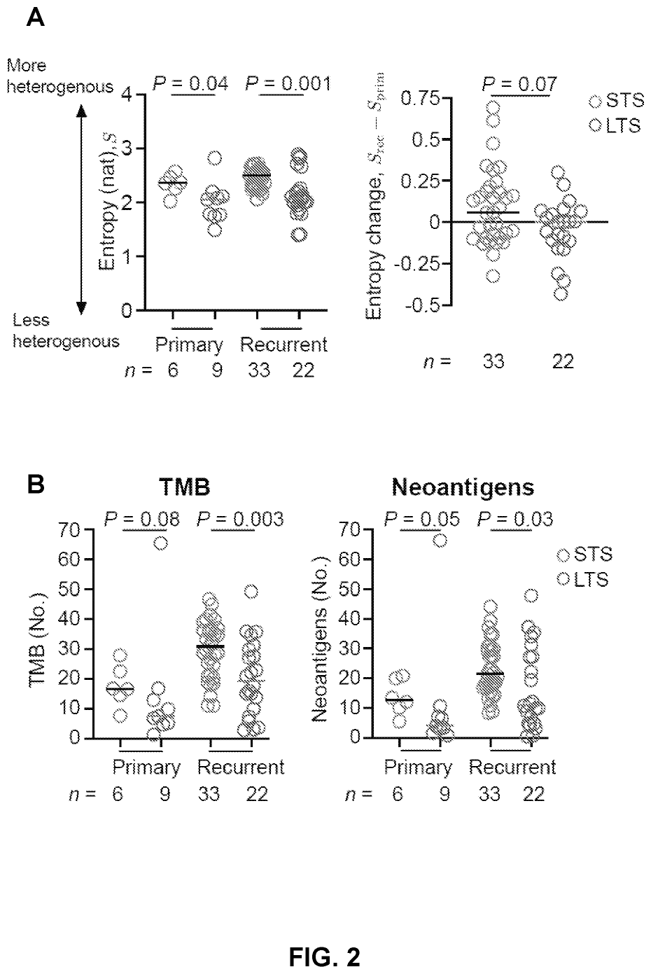

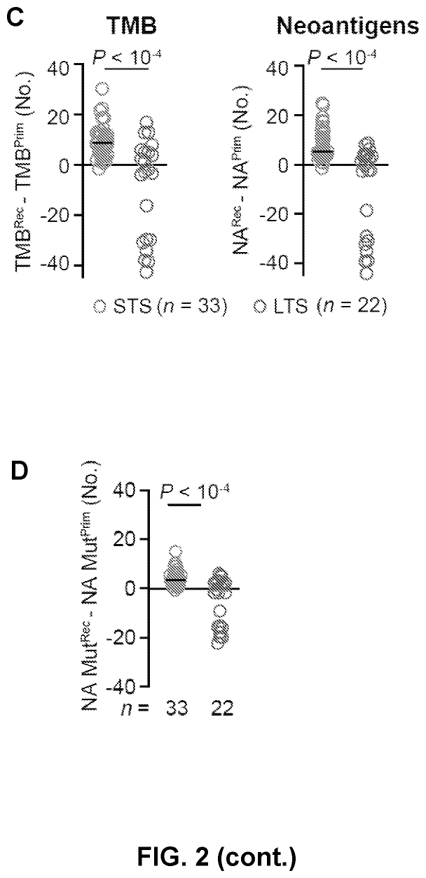

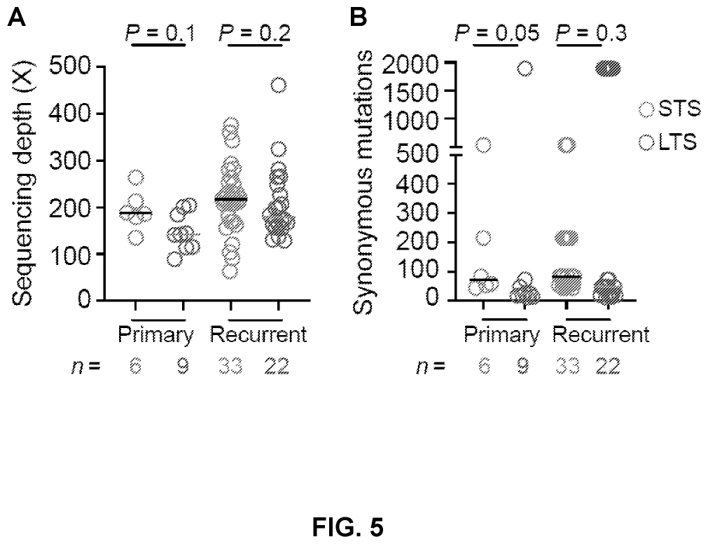

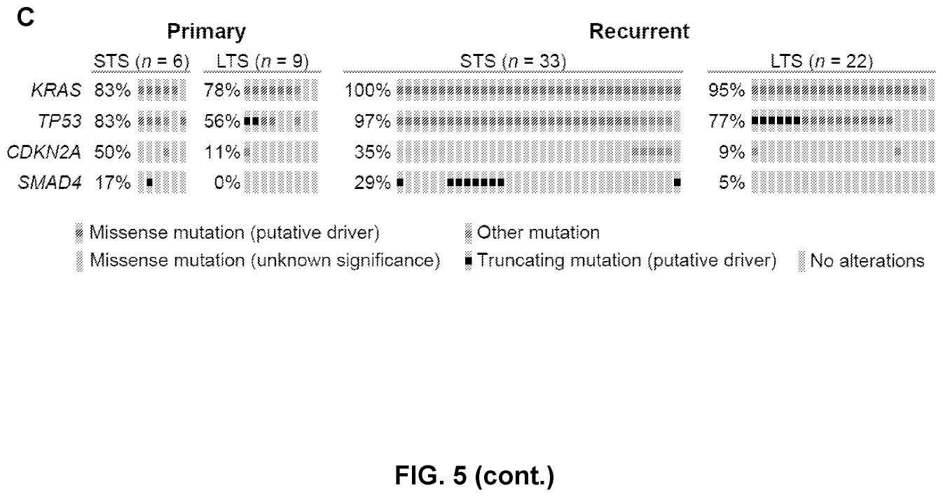

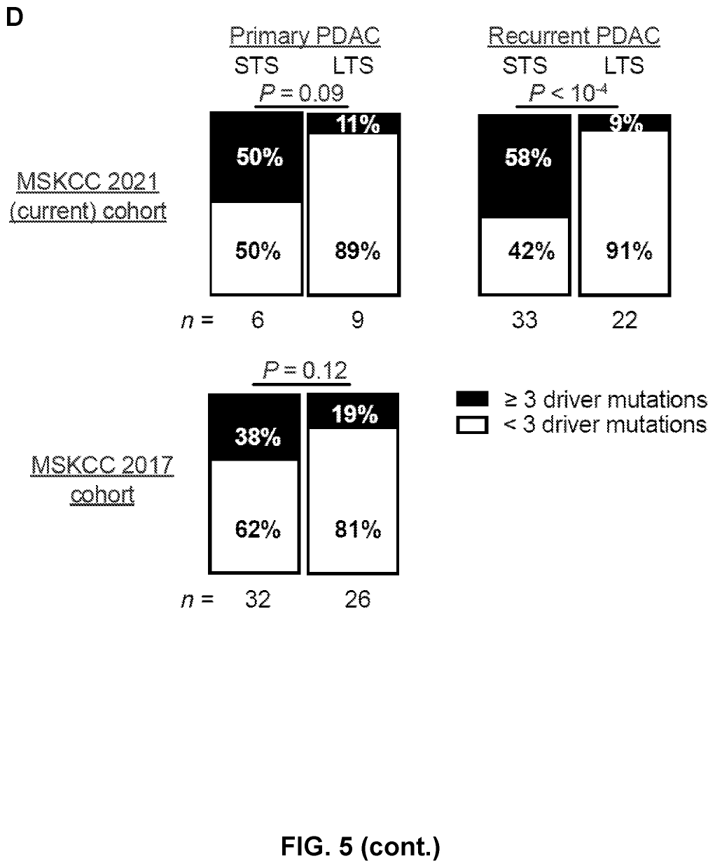

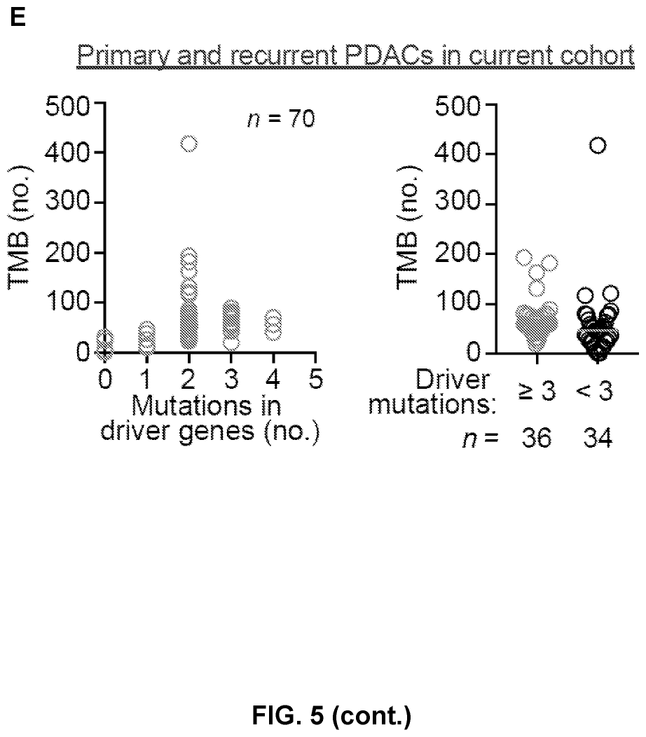

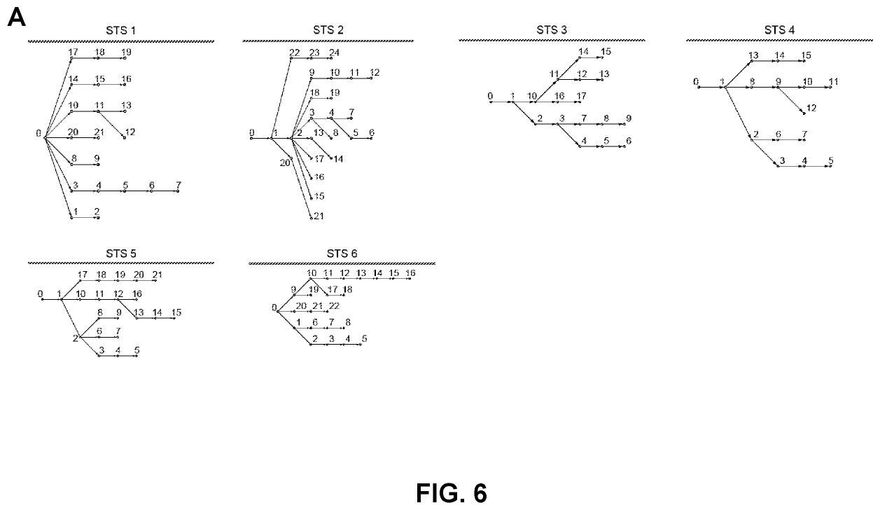

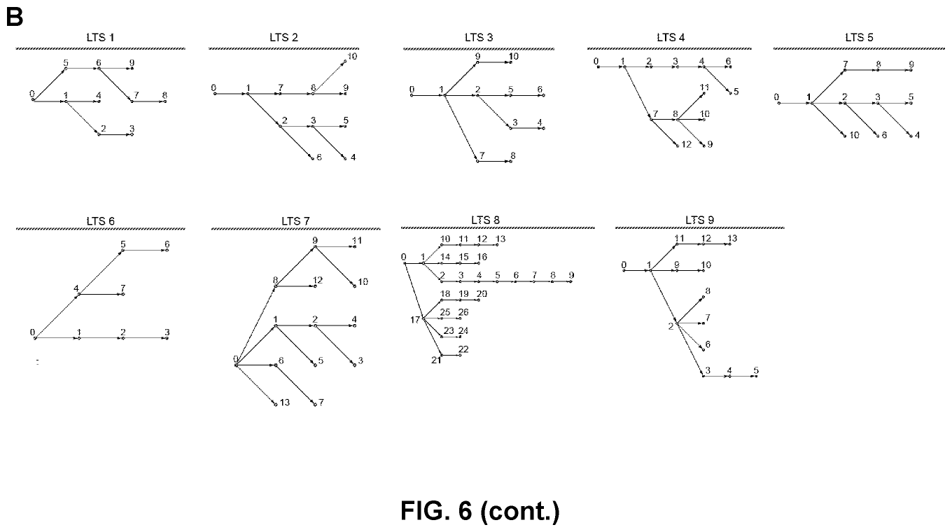

To examine whether differential selection pressure could explain these unique recurrence patterns, we performed whole-exome sequencing (FIG. 5, panel A) and inferred the clonal structures of matched primary and recurrent tumors. Without wishing to be bound by theory, greater immune selection pressure in LTS tumors should limit the diversity of tumour clones over time, due to immunoediting of neoantigens. Consistently, we found that, although primary tumors in LTSs were only slightly more homogeneous than in STSs, recurrent tumors in LTSs were much more homogeneous (FIG. 2, panel A, left), indicating that LTSs probably evolved fewer clones (FIG. 2, panel A, right; FIG. 6, panels A and B). To examine whether this could be explained by greater selection pressure on neoantigens, we compared the total number of non-synonymous mutations (tumour mutational burden (TMB)) and computationally predicted MIC-I restricted neoantigens 4,5. Consistently, although primary LTS tumors had a similar TMB with a comparable number of neoantigens as STS tumors (FIG. 2, panel B), recurrent LTS tumors had a lower TMB with fewer neoantigens (FIG. 2, panel B). Despite these differences, LTS and STS tumors had comparable numbers of synonymous mutations and mutations in driver oncogenes (FIG. 5, panels B and C). Although recurrent tumors of LTSs had fewer co-occurring mutations in oncogenes compared with recurrent tumors of STSs (FIG. 5, panel D), the number of mutations in oncogenes did not correlate with TMB (FIG. 5, panel E). Furthermore, LTS recurrent tumors gained significantly fewer mutations and neoantigens compared with STS recurrent tumors (FIG. 2, panel C), remaining largely neutral over time 19. LTS tumors also gained fewer mutations that generate neoantigens than STS tumors (FIG. 2, panel D), indicating that LTS tumors preferentially depleted neoantigenic mutations. These data indicate that greater immune selection in LTS tumors edited tumour clones and neoantigens.

### The Neoantigen Quality Model

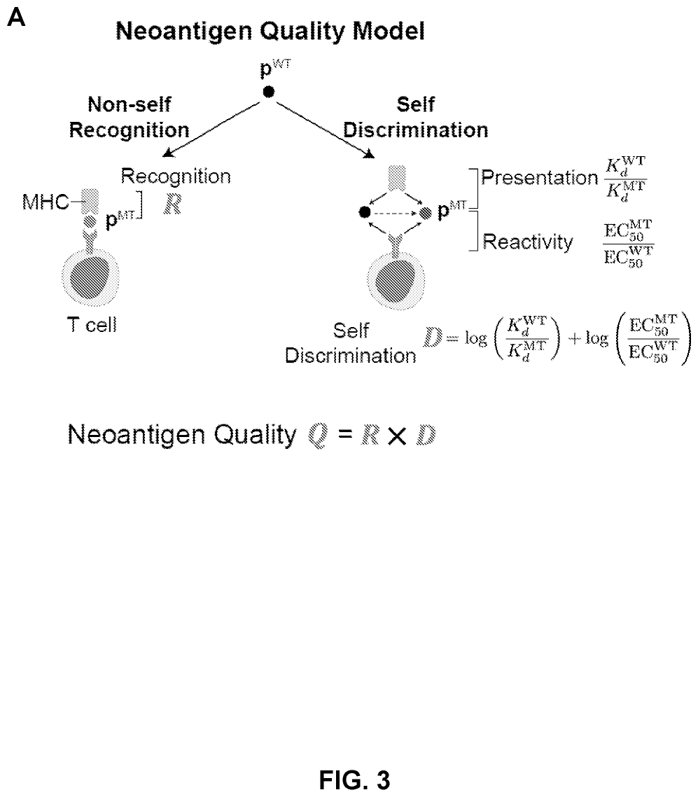

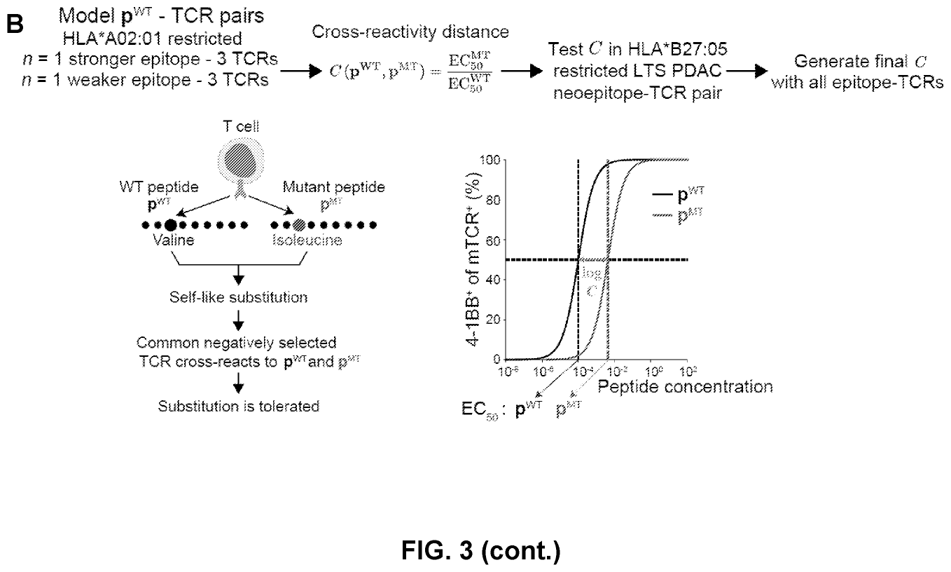

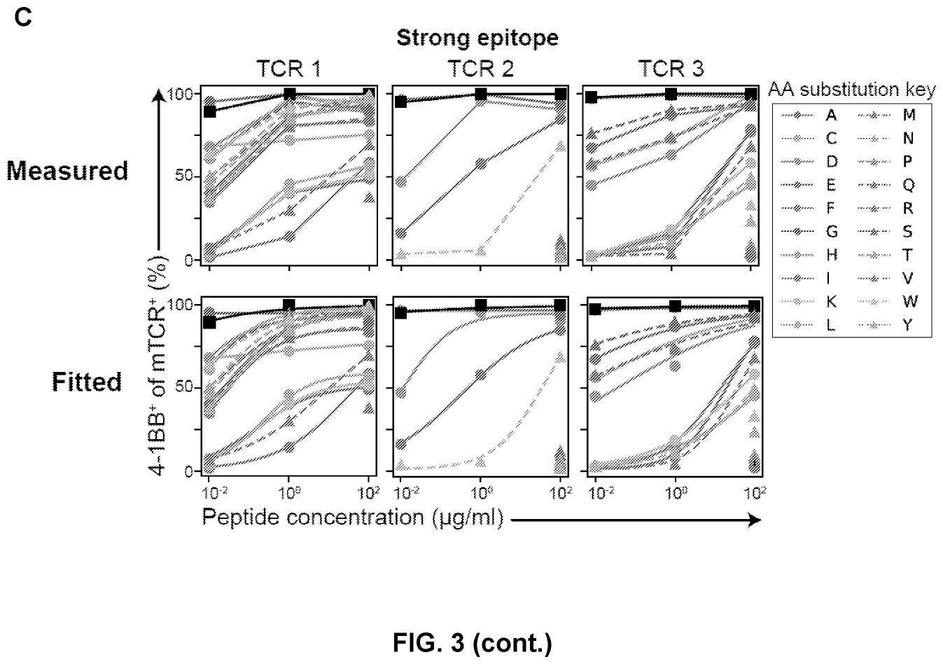

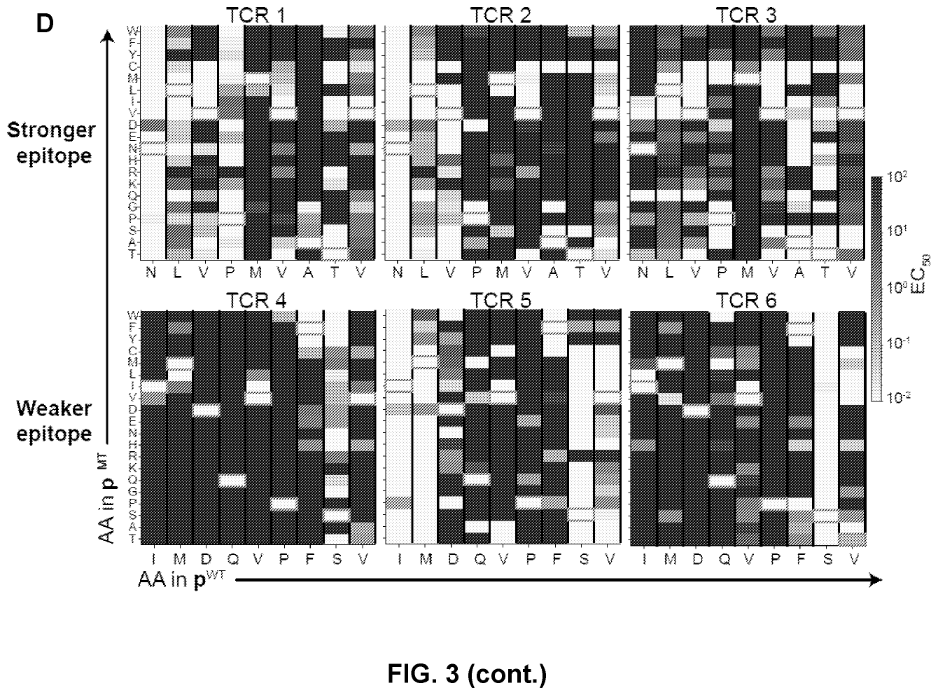

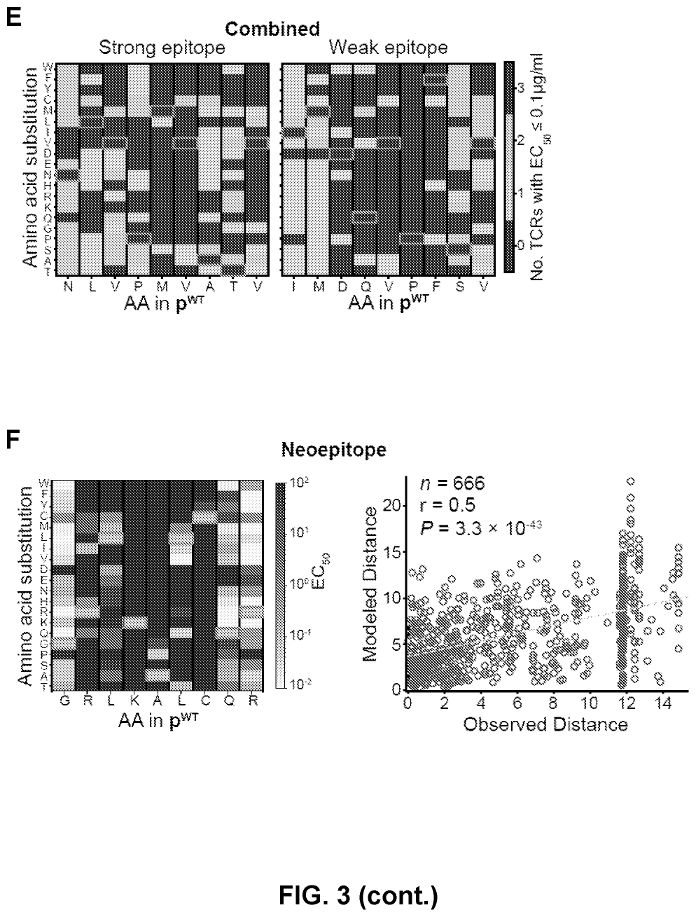

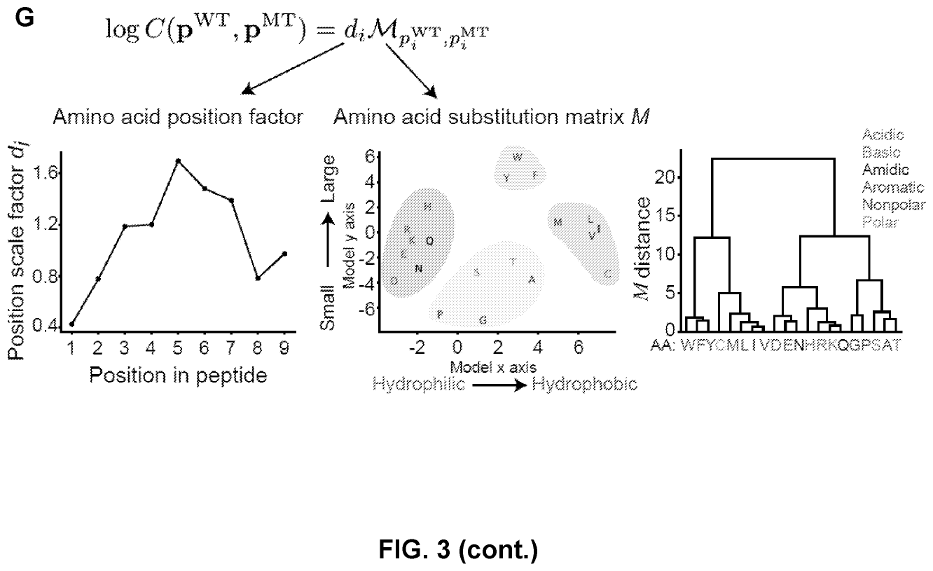

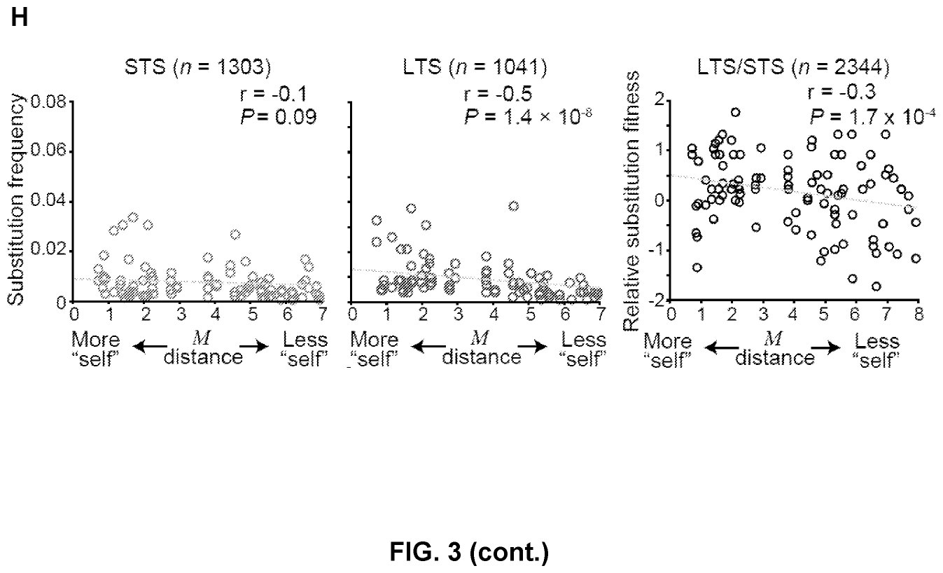

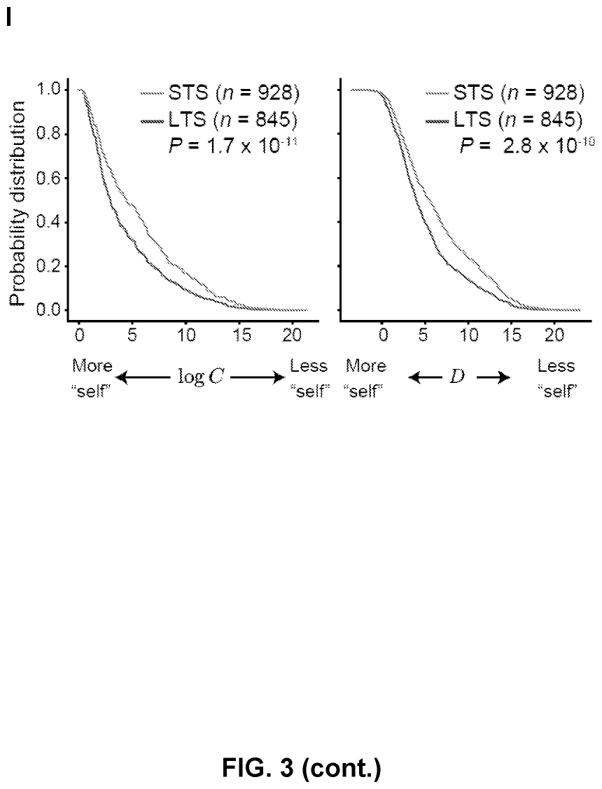

To identify the edited neoantigens, we extended our previous neoantigen quality model 4,5 that quantifies the immunogenic features of a neoantigen to provide that two competing outcomes determine whether a neoantigen is high-quality—whether the immune system recognizes or tolerates a neoantigenic mutation (FIG. 3, panel A). To estimate the likelihood the immune system recognizes a neoantigen, we measure the sequence similarity of the mutant neopeptide (p^{MT}) to known immunogenic antigens. This infers the ‘non-self’ recognition potential R of p^{MT}, a proxy for peptides within the recognition space of the T cell receptor (TCR) repertoire.

By contrast, the immune system can also fail to discriminate p^{MT} from its wild-type (WT) peptide (p^{WT}), and therefore tolerate it as ‘self’. The immune system must therefore exert greater self-discrimination D (FIG. 3, panel A) in tumors to overcome the principles of negative T cell selection, the adaptation that limits autoreactivity to host tissues. We approximate the D between p^{WT} and p^{MT} by two features—differential MHC presentation and differential T cell reactivity. Differential MHC presentation of p^{WT} and p^{MT} (K_{d}^{WT}/K_{d}^{MT}), previously introduced as the MHC amplitude A (refs. 4,5), estimates the availability of T cells to recognize p^{MT}. If p^{WT} is not presented to T cells in the thymus or the periphery (as with a high K_{d}^{WT}, which implies poor p^{WT}-MHC binding), p^{WT}-specific T cells escape negative selection to expand the peripheral T cell precursor pool available to recognize a p^{MT} presented on MHC (low K_{d}^{MT}) 20. Here we extend this concept and introduce cross-reactivity distance C, a new model term that estimates the antigenic distance required for T cells to discriminate between p^{MT} and p^{WT}. Thus, self-discrimination D=log(A)+log (C) is a proxy for peptides outside the toleration space of the TCR repertoire. In summary, we define neoantigen quality as Q=R×D (FIG. 3, panel A), now with components that estimate whether a neoantigen can be recognized as non-self and discriminated from self.

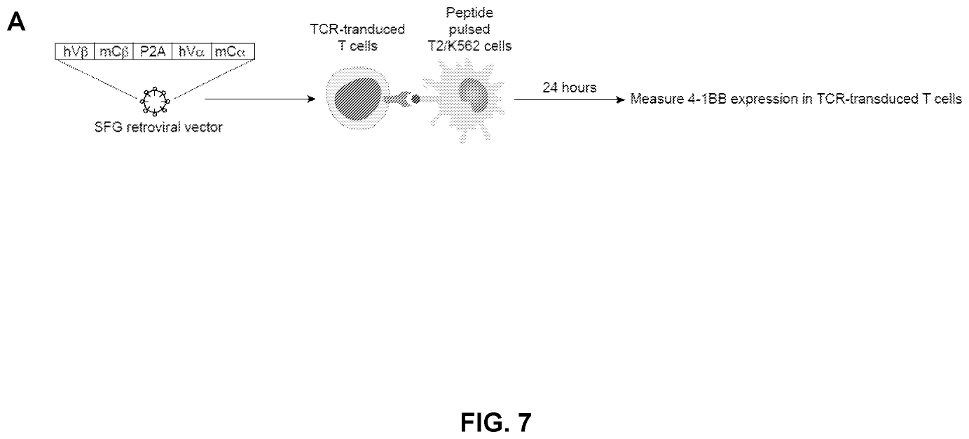

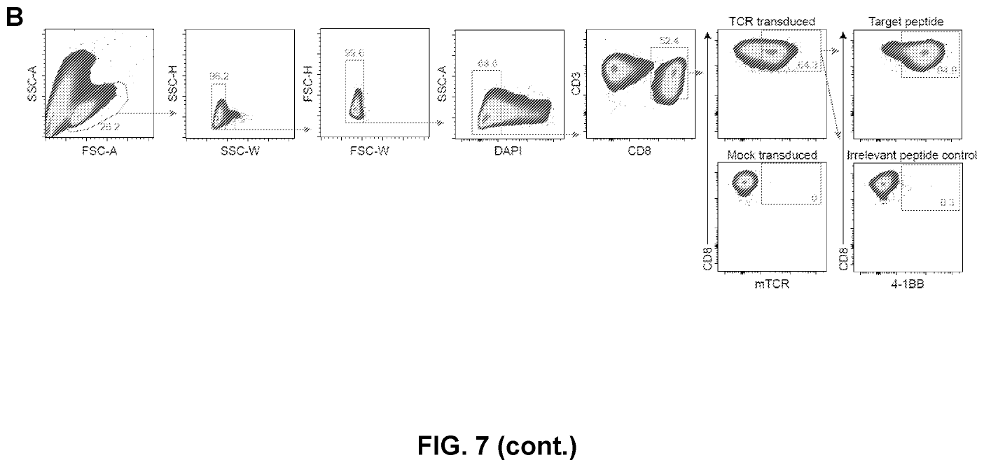

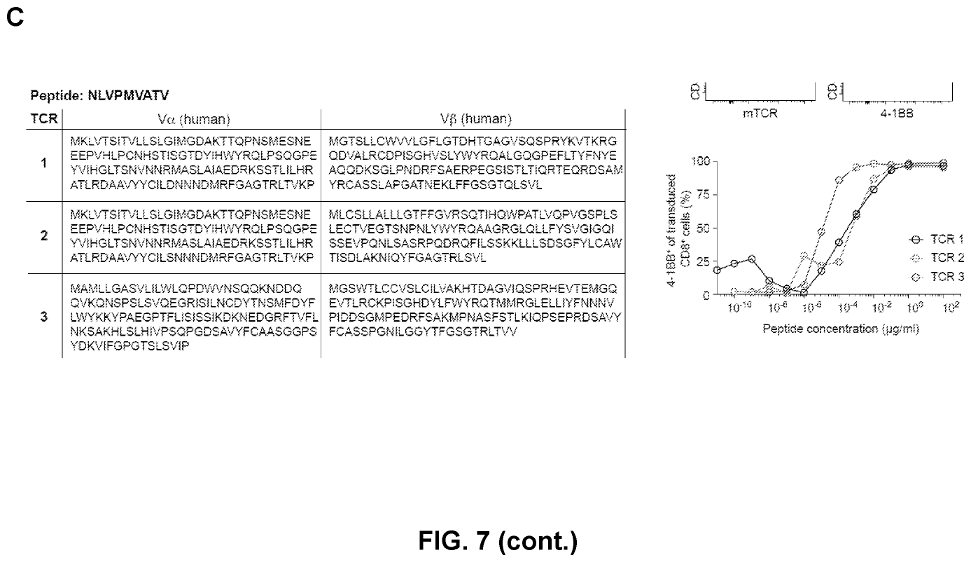

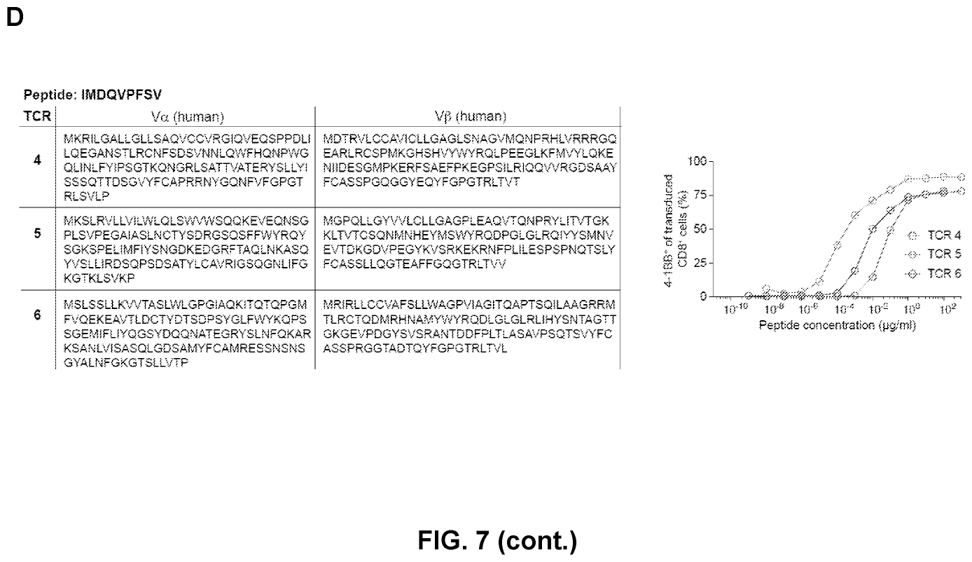

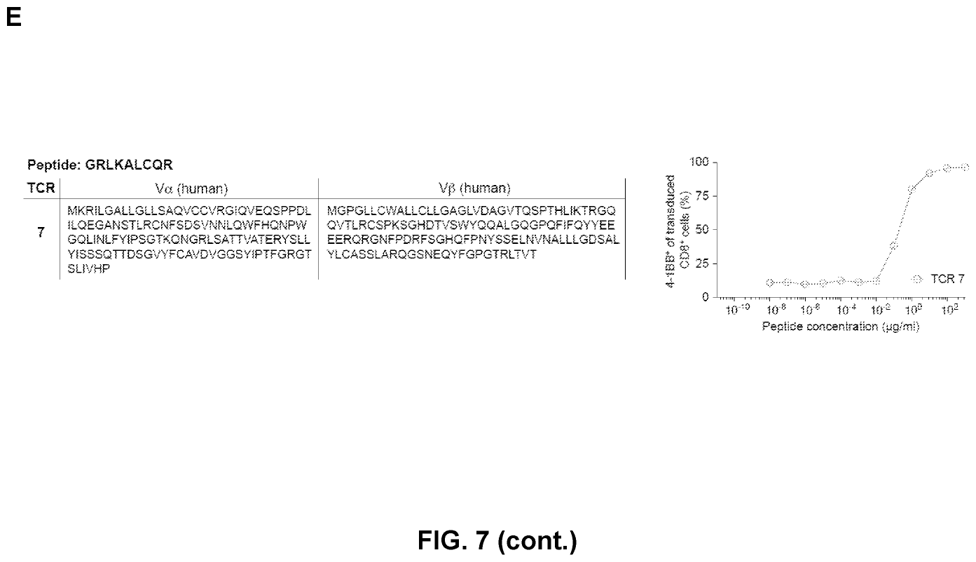

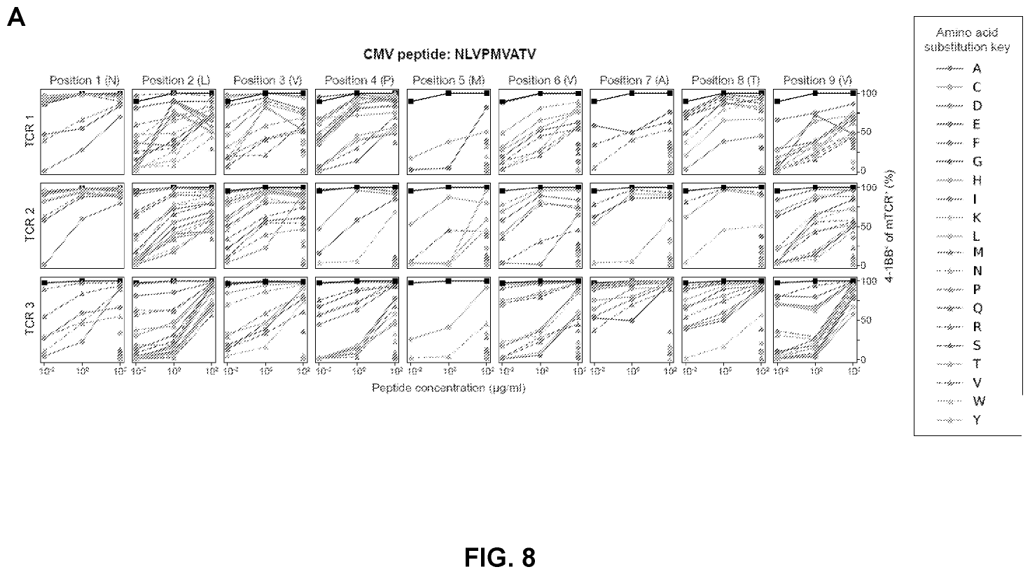

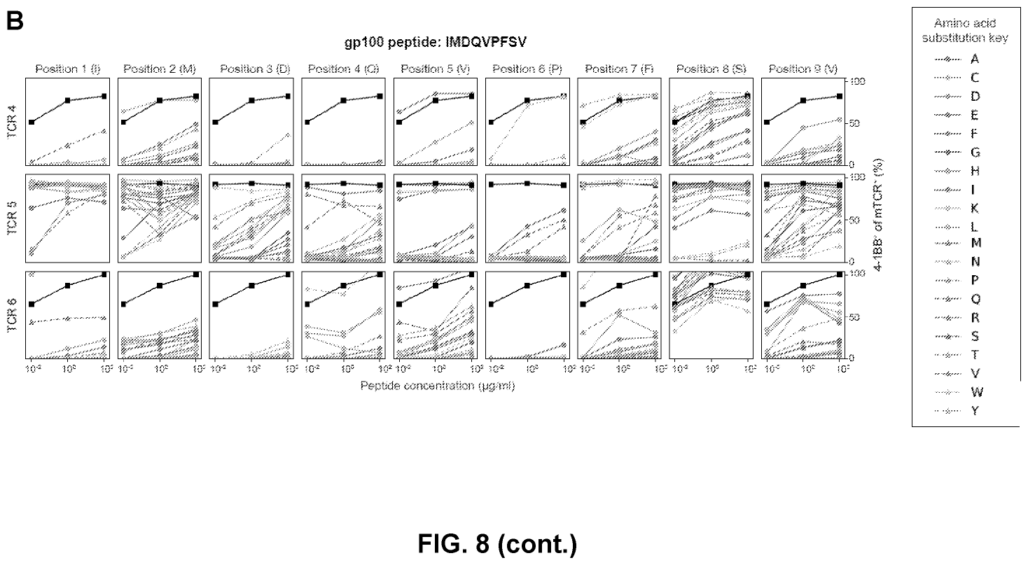

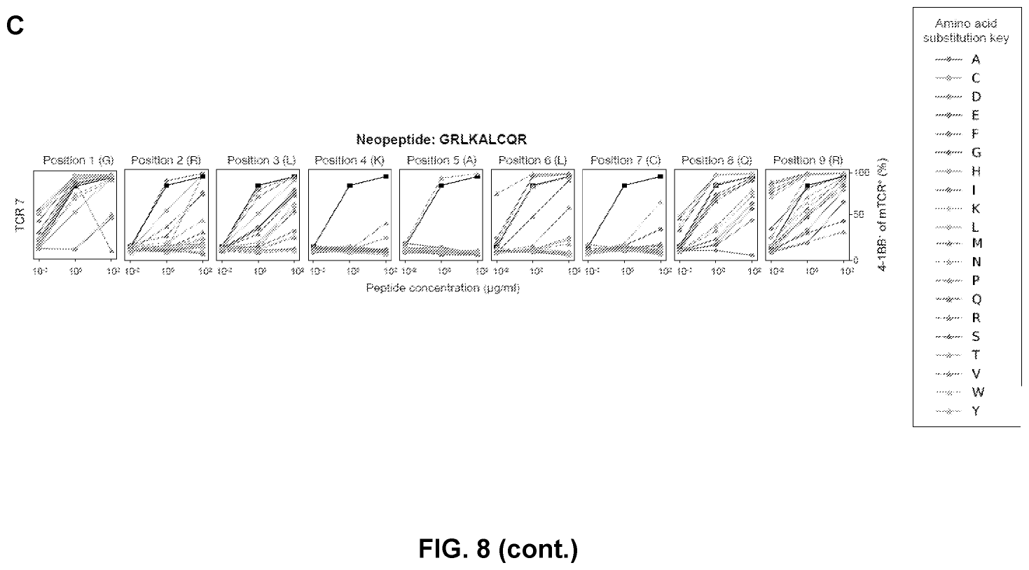

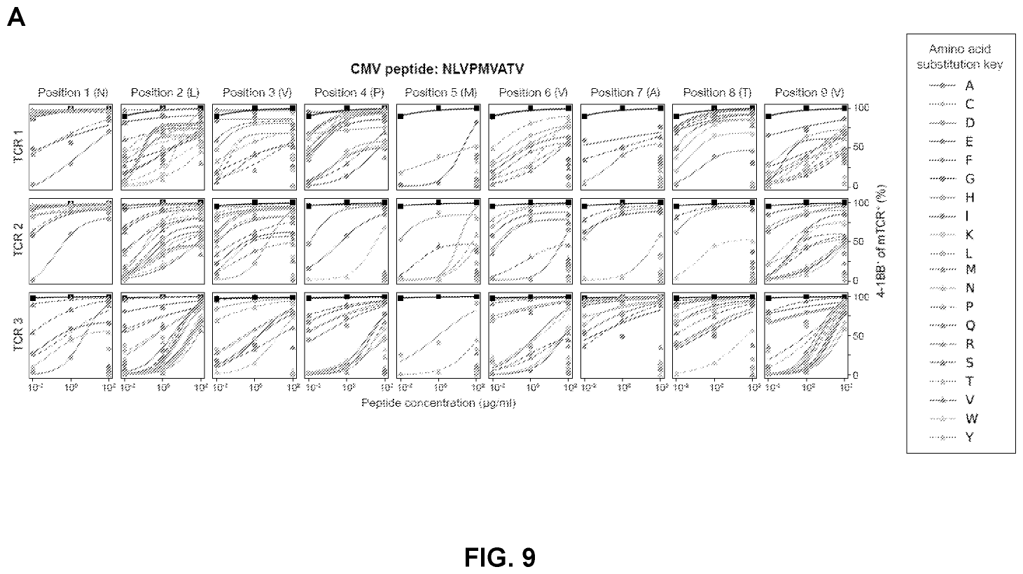

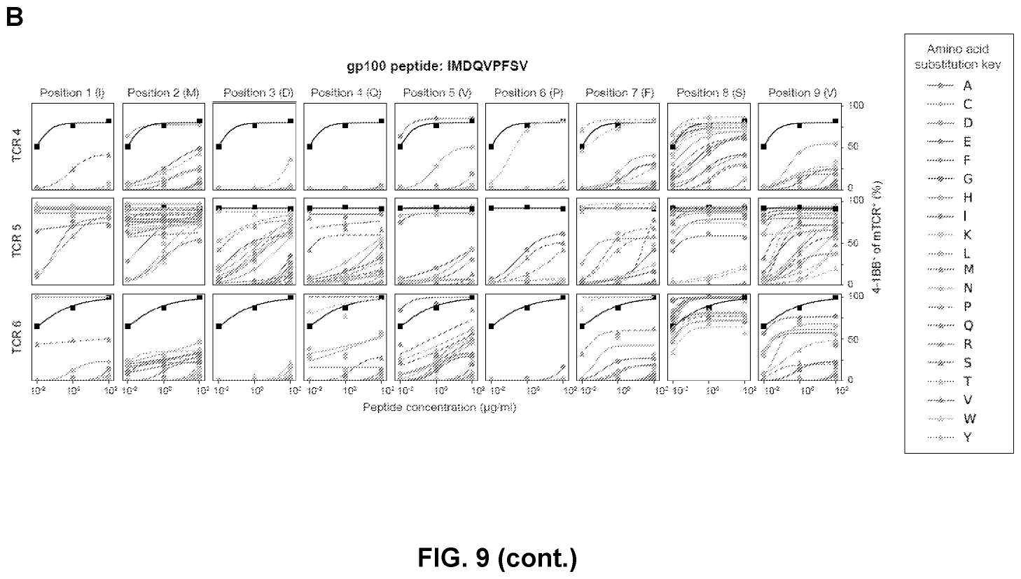

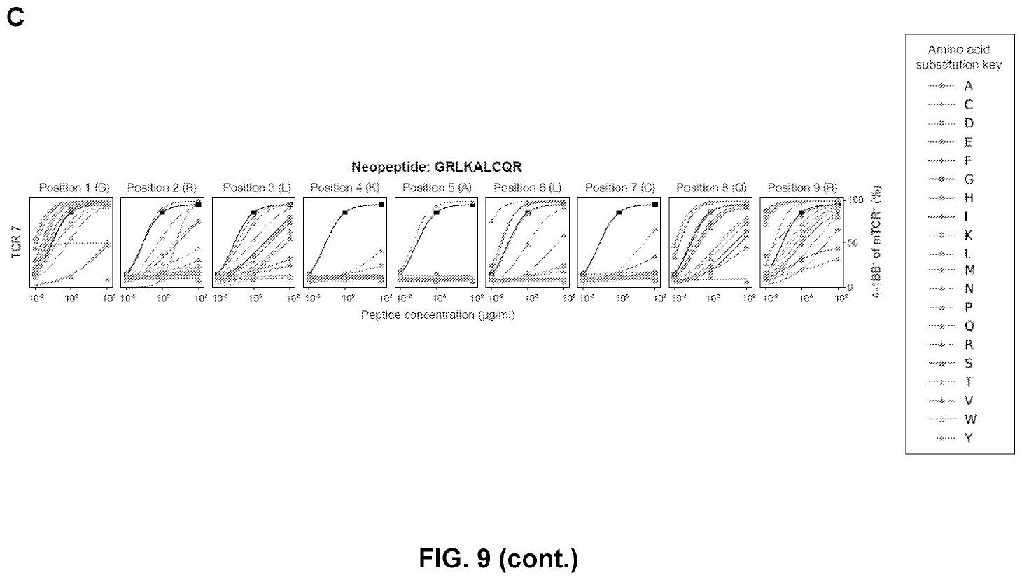

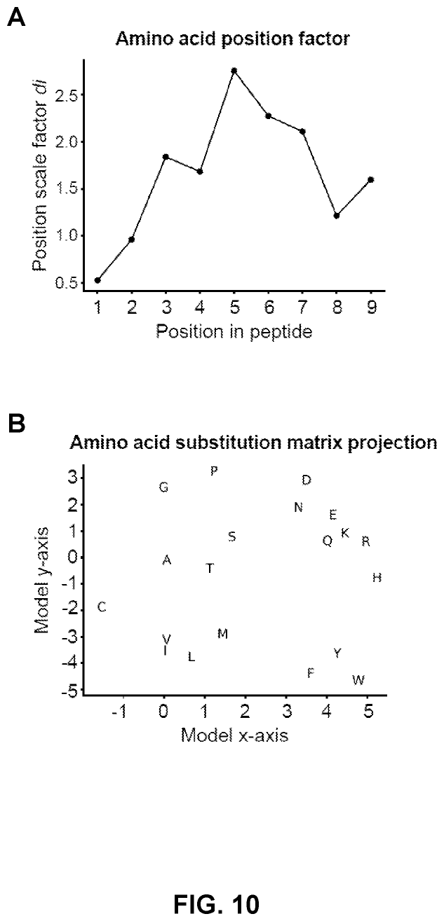

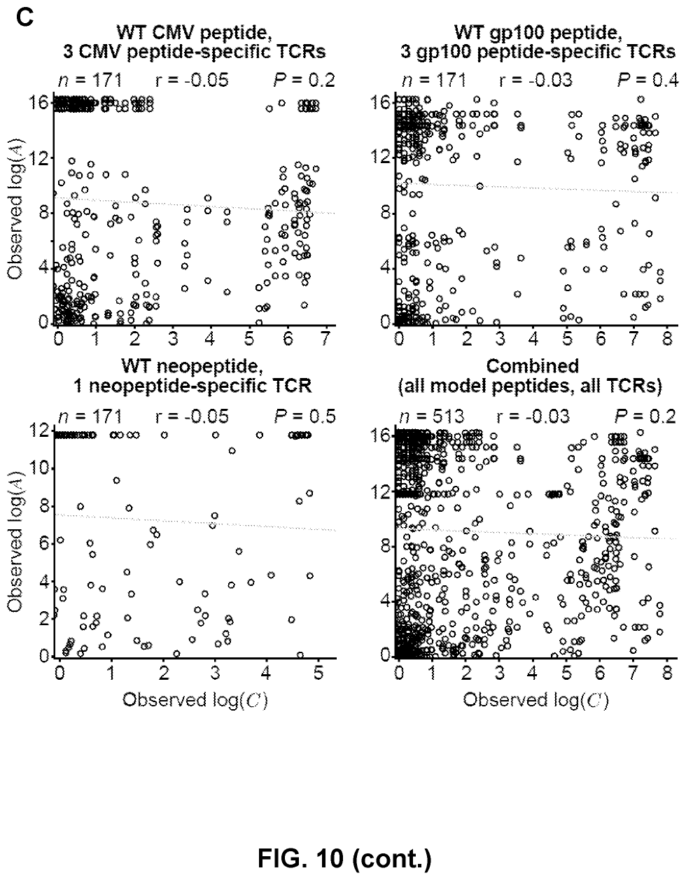

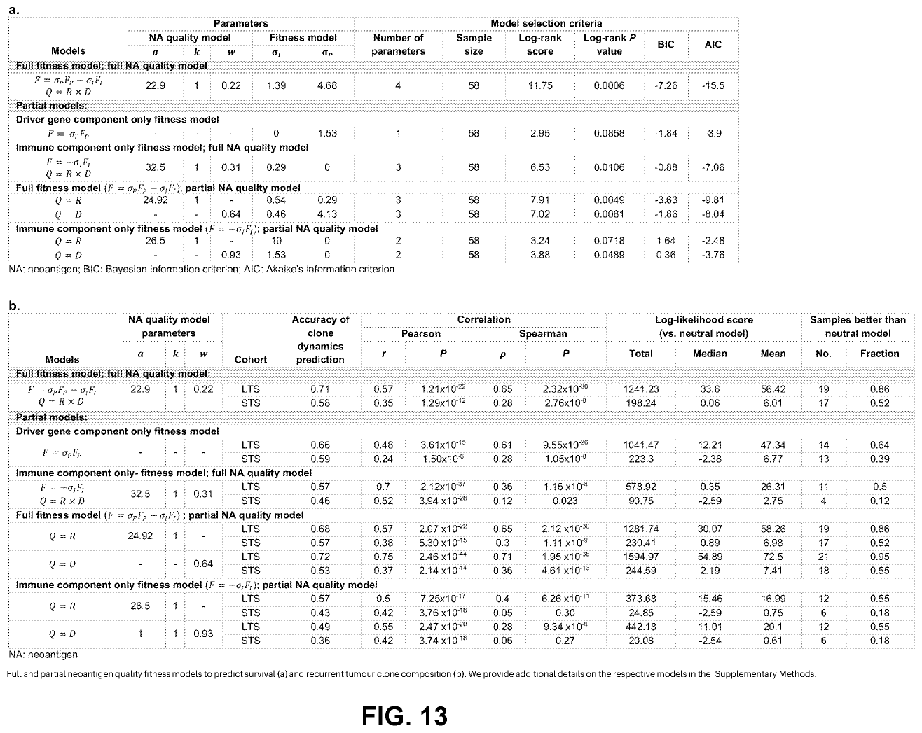

To model C, we leveraged findings that conserved structural features underlie TCR-peptide recognition. Specifically, the binding domains of peptide-degenerate TCRs 21,22 and TCR-degenerate peptides 23 share common amino acid motifs, indicating that T cell cross-reactivity between p^{MT} and p^{WT} could estimate the relative C of different neoantigenic substitutions (FIG. 3, panel B). We selected an HLA-A*02:01-restricted strong epitope (NLVPMVATV (NLV)) from human cytomegalovirus 24 that was previously used to model TCR-peptide degeneracy 21,22 as a model p^{WT}, and three NLV-specific TCRs (FIG. 7, panels A-C). We then varied the NLV peptide by every amino acid at each position to model p^{MT} substitutions, and compared how TCRs cross-react between each p^{MT} and its p^{WT} across a 10,000-fold concentration range where p^{WT} changes maximally altered T cell activation (FIG. 3, panel B). We observed that substitutions were either highly, moderately or poorly cross-reactive (FIG. 3, panels C and D), and the cross-reactivity pattern depended on the substituted position and residue (FIG. 8, panel A). We found similar patterns of cross-reactivity between a model HLA-A*02:01-restricted weaker p^{WT} epitope in the melanoma self-antigen gp100 25,26 (FIG. 7, panel D; FIG. 8, panel B), three p^{WT}-specific TCRs and single-amino-acid-substituted p^{MT}S, indicating that conserved substitution patterns define C (FIG. 3, panel E; FIG. 8, panel B). Thus, we quantified the cross-reactivity distance C between a p^{WT} and its corresponding p^{MT} as C(P^{WT}, p^{MT})=EC₅₀^{MT}/EC₅₀^{WT}. We chose the half maximal effective concentration (EC₅₀) to model C, as T cell activation to p^{WT} was consistently a sigmoidal function (FIG. 7, panels C and D; FIG. 9, panels A and B) described by a Hill equation, where EC₅₀ determines how a ligand activates a receptor. We next estimated the EC₅₀ of all 1,026 TCR-p^{MT} pairs to infer a model for C that estimates whether a neoantigenic substitution is cross-reactive (and therefore tolerated) based on the substituted amino acid position and residue (FIG. 9, panels A and B; FIG. 10, panels A and B). We then validated whether C predicted cross-reactive substitutions in an HLA-B*27:05-restricted neopeptide-TCR pair from an LTS (FIG. 7, panel E). Notably, C predicted cross-reactive p^{WT} P^{MT} and p^{MT} p^{MT} substitutions in this neopeptide-TCR pair (FIG. 3, panel F; FIG. 8, panel C; FIG. 9, panel C). Thus, we combined all 1,197 TCR-p^{MT} pairs to derive a composite C—the antigenic distance for a TCR to cross-react between amino-acid-substitution pairs (FIG. 3, panel G; FIG. 10, panel C). Two factors promote cross-reactivity: substitutions at peptide termini 27 and within amino acid biochemical families (driven by amino acids of similar size and hydrophobicity; FIG. 3, panel G. With this composite C, we now define self-discrimination D between a p^{WT} and its corresponding p^{MT} (FIG. 3, panel A) as

$$D ( {p}^{WT} \to {p}^{MT} ) = ( 1 - w ) log ( \frac{{K}_{d}^{WT}}{{K}_{d}^{MT}} ) + w log ( \frac{{EC}_{50}^{MT}}{{EC}_{50}^{WT}} ) , \qquad ( 1 )$$

where w sets the relative weight between the two terms. We chose the parameters of the neoantigen quality model to maximize the log-rank test score of survival analysis on an independent cohort of 58 patients with PDAC5 (FIG. 13, panel a).

### Immunoediting of Neoantigens

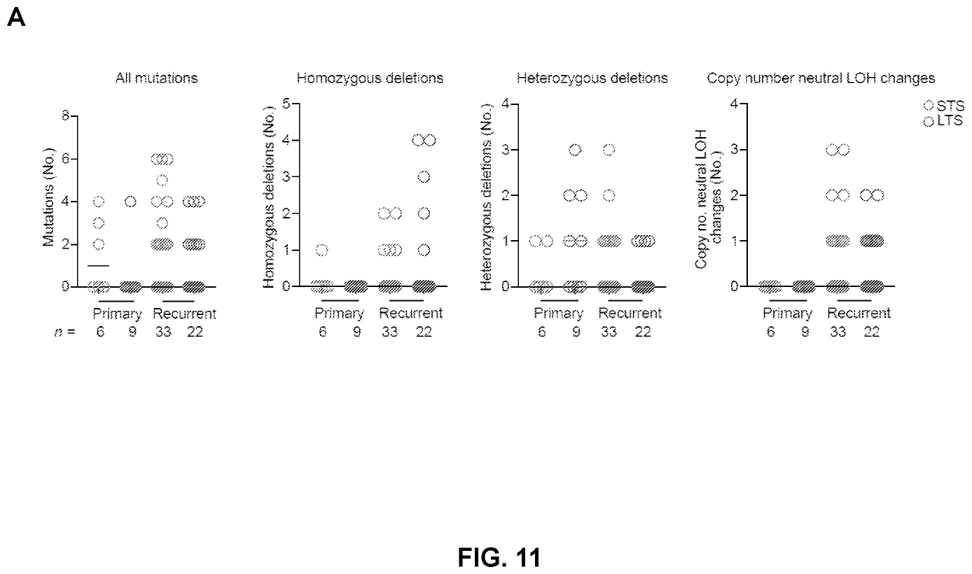

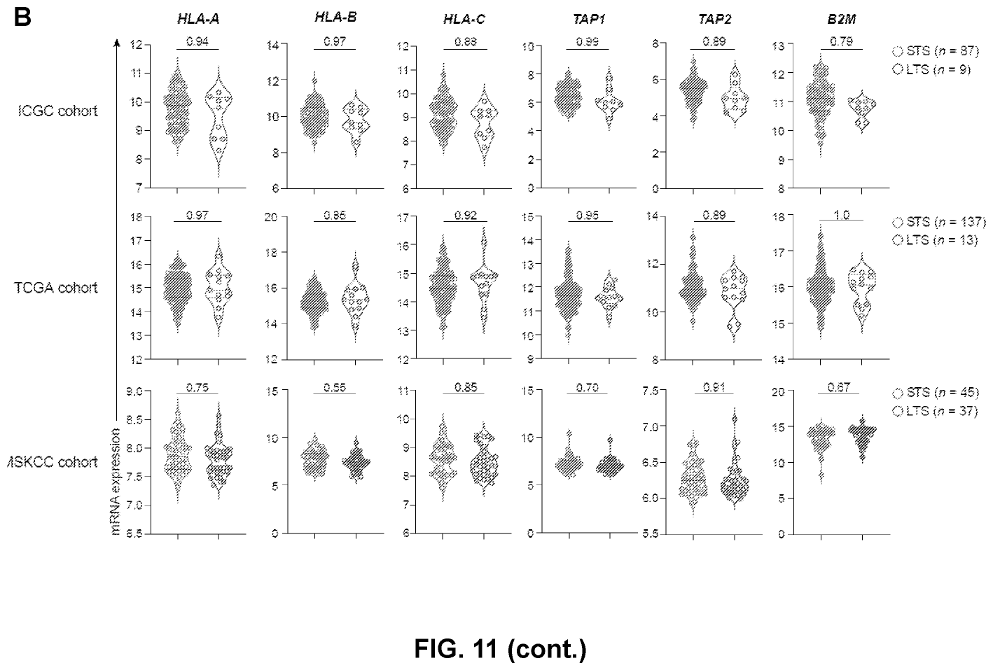

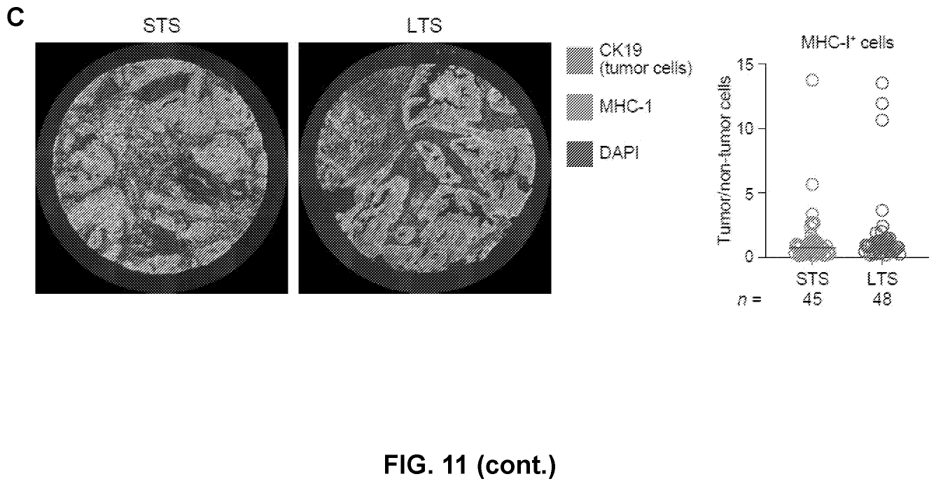

We applied our model to PDAC to demonstrate that immunoediting will differentially deplete neoantigens with higher D (less self) in LTS versus STS PDACs. First, we stratified the frequency of mutations by the antigenic distance as defined by C (FIG. 3, panel G). Compared with mutations with a lower antigenic distance, mutations with a greater antigenic distance from self were more significantly depleted in both LTS and STS PDACs (FIG. 3, panel H, left and middle) and more depleted in LTS compared with STS PDACs (FIG. 3, panel H, right). To further examine these observations, we applied the full D model to find that neoantigens with both a higher C and D were strikingly more depleted in LTS versus STS PDACs (FIG. 3, panel I). Genes in the HLA class-I pathway were not differentially mutated, deleted, expressed or localized in STS versus LTS PDACs, indicating that neoantigen depletion was not accompanied by acquired resistance in the HLA class-I pathway in LTSs (FIG. 11, panels A-C). Thus, tumors in LTSs selectively lose high-quality neoantigens.

### Predicting Recurrent Tumour Composition

We next incorporated neoantigen quality parameters into a fitness model 4,5 to validate whether our model that predicts clonal tumour evolution can identify immunoedited clones. We reconstructed joint multisample phylogenies 28 for all tumors from each patient to provide a common clonal structure and track clone frequencies between the tumors of the same patient. To describe selective pressures acting on tumour clones, we accounted for positive selection due to cumulative mutations in driver oncogenes. We quantify this effect in a minimal model F_{P}^{∝}, which counts the number of missense mutations in canonical PDAC driver genes (KRAS, TP53, CDKN2A and SMAD4) in each clone α. The composite fitness model (FIG. 4, panel A) defines fitness function, F^{α}, of clone α as the sum of a negative fitness cost due to immune recognition of high-quality neoantigens and positive fitness gain due to the accumulation of mutations in driver oncogenes,

$${F}^{∝} = - {σ}_{{l}_{{p}^{{MT}_{∈ clone ∝}^{max}}}} Q ( {p}^{MT} ) + {σ}_{P} {F}_{P}^{∝} \qquad ( 2 )$$

with the free parameters σₗ and σ_{P} setting the amplitude of the fitness components. We use the model to predict the frequencies of clones propagated to recurrent tumors as

$${\hat{X}}_{rec}^{∝} = \frac{1}{Z} {X}_{prim}^{∝} exp ( {F}^{∝} ) \qquad ( 3 )$$

where Xₚᵣᵢₘ^{∝} is the frequency of clone α in the primary tumour, \hat{X}_{rec}^{∝} is its predicted frequency in the recurrent tumour and constant Z ensures correct normalization. We evaluated how closely the fitness model predicted clonal evolution in the recurrent tumors. To do this, for each recurrent tumor in the LTS and STS cohorts, we performed maximum-likelihood fitting of the model parameters σₗ and σ_{P} in equation (3).

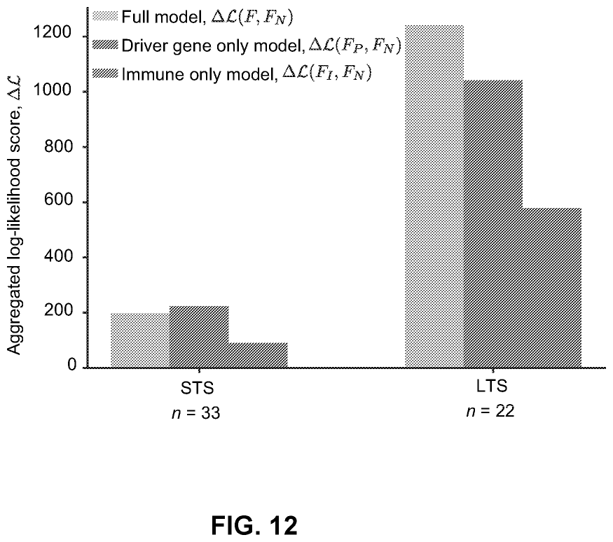

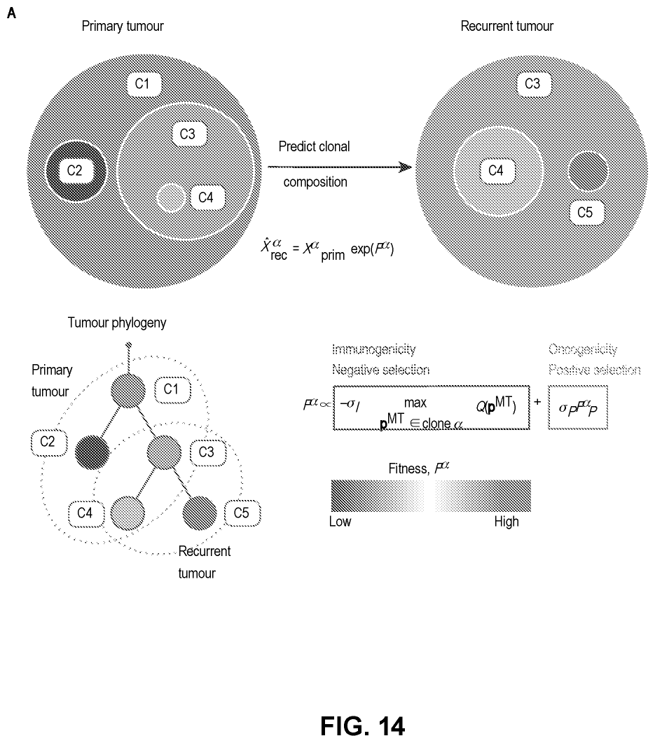

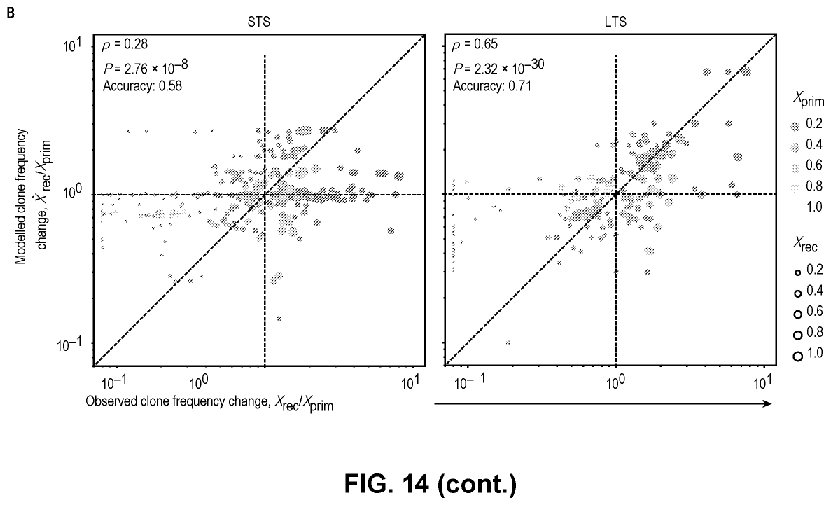

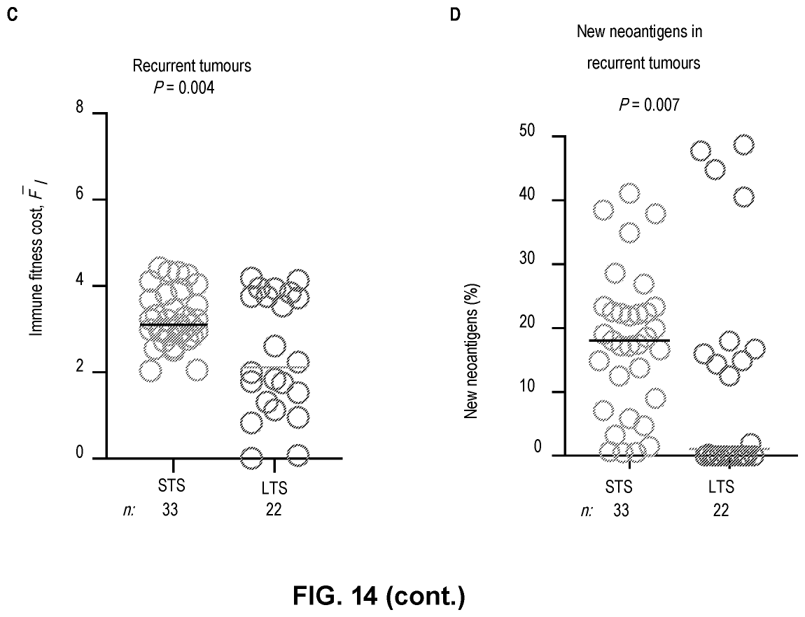

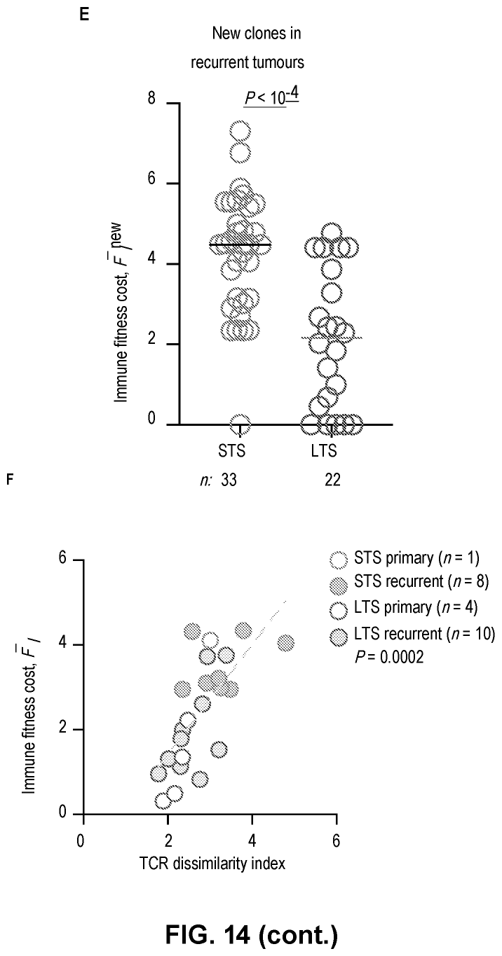

We found that our model provided a better fit of the observed evolution of LTS compared to STS tumour clones, predicting observed evolution in 86% of LTS tumors versus 52% of STS tumors (FIG. 13, panel b) when compared with a neutral model (no selection pressure on clones; differences were quantified with a Bayesian information criterion). Notably, a partial fitness model that incorporates only the oncogenicity component, F^{∝}=σ_{P}F_{P}^{∝} showed reduced performance for the LTS tumors but not STS tumors FIG. 13, panel b and FIG. 12. To illustrate this further, we compared observed and model-fitted clone frequency changes between the primary and recurrent tumors, X_{rec}^{∝}/Xₚᵣᵢₘ^{∝} and \hat{X}_{rec}^{∝}/Xₚᵣᵢₘ^{∝} (FIG. 4b), for all reliably predictable clones in the primary tumor (above 3% frequency). The direction of frequency changes was correctly predicted for 71% of LTS and 58% of STS tumour clones (rank correlation ρ of 0.65 and 0.28, respectively; FIG. 14, panel B and FIG. 13, panel b). We attribute the model's better predictions in LTS tumors to the presence of immune selection in these tumors.

Next, we computed the overall tumour immune cost (averaging the immune component,

$${F}_{l}^{∝} = {p}^{MT} \begin{matrix}max \\ ∈ clone ∝\end{matrix} Q ( {p}^{MT} )$$

over all tumour clones). Consistently, the immune fitness cost was lower in recurrent LTS tumors compared with in STS tumors (FIG. 14, panel C). Furthermore, we considered the immune cost only of clones that are new in recurrent tumors, but not present in primary tumors. Recurrent LTS tumors contained both fewer new neoantigens (1% versus 18%; FIG. 14, panel C) and new clones with markedly lower immune fitness cost (FIG. 14, panel E) compared with recurrent STS tumors. These observations again indicate that the LTS recurrent tumors had been subject to immunoediting.

Finally, we confirmed these results by analyzing TCR sequencing data in the available recurrent tumour samples. We quantified the specificity of T cell clonal expansion using the TCR dissimilarity index 18 (FIG. 4, panels A and B) and correlated this index to immune fitness cost. We found greater T cell clonal expansion in tumors (lower TCR dissimilarity index) correlated with more highly edited tumors (lower immune fitness cost) (FIG. 14, panel F and FIG. 4, panel c). In summary, these results strongly indicate that neoantigens are immunoedited in PDAC, and that our fitness model captures the selective pressures by T cells acting on tumour clones.

### DISCUSSION

Here we clarify several questions on how the immune system interacts with cancer. First, does cancer immunoediting occur in humans? As the theory of cancer immunoediting was developed by studying carcinogen-induced highly mutated murine sarcomas 1,3, it has remained uncertain whether these principles apply to human cancers 29-31. We postulated that spontaneous immunoediting of a human cancer should manifest when the immune system recognizes an immunogenic antigen in a primary tumour, as this should induce the antigen to be subsequently eliminated in the recurrent tumour. Indeed, this is what we found—tumors that evolve under stronger immune pressure lose more immunogenic neoantigens. Although we did not assess the changes in non-mutated antigens or address how different cellular compositions and tissue environments may modulate editing, it is notable that the proof for immunoediting is revealed in PDAC, a low-mutated cancer that is considered to be resistant to endogenous immunity. This strengthens the claim that immunoediting is a broadly conserved principle of carcinogenesis.

Second, does immunoediting manifest as loss of immunogenic antigens, or do cancers also acquire genetic resistance?Interestingly, we observed the former but not the latter. We postulate that such phenotypes are governed by the magnitude of the selective pressure. Although LTSs exhibit higher immune pressures in tumors than STSs, this is ostensibly still lower than pharmacologically boosted immune pressure in a tumour 32. Thus, in LTSs, as pressure is moderate, tumors lose immunogenic antigens; by contrast, where pressure is maximal, such as perhaps when under therapy, tumors acquire resistance 32. This distils cancer evolution under immune selection to a simpler concept-selection determines clonal composition, and pressure determines adaptive change. Further studies will test these concepts.

Third, can we quantify how the immune system recognizes mutations?We combined experimental techniques and machine learning to present a new metric that captures how T cells cross-react between peptides. We use C to quantify the antigenic distance of mutated peptides in the TCR-recognition space and the qualities that render individual mutations immunogenic, building on our previous efforts 4,5 to formalize antigen quality. Although we used our quality model to identify immunogenic neoantigens, we propose that it captures common immunogenic features in antigens. Thus, without wishing to be bound by theory, our model can further illuminate the biology of antigens beyond cancer, including T cell cross-reactivity between antigens, pathologies of cross-reactivity (such as autoimmunity) and therapies that require rational antigen selection (such as vaccines). Finally, quantifying the ability of the immune system to discriminate changes in mere single amino acids can predict how cancers evolve. This reflects that a fundamental function of the immune system is to maintain integrity of the host genome. Thus, without wishing to be bound by theory, our model in essence captures the mechanisms through which the immune system preserves genomic integrity.

### REFERENCES CITED IN THIS EXAMPLE

1. Shankaran, V. et al. IFNγ and lymphocytes prevent primary tumour development and shape tumour immunogenicity. Nature 410, 1107-1111 (2001).
2. Hanahan, D. & Weinberg, R. A. Hallmarks of cancer: the next generation. Cell 144, 646-674 (2011).
3. Matsushita, H. et al. Cancer exome analysis reveals a T-cell-dependent mechanism of cancer immunoediting. Nature 482, 400-404 (2012).
4. Luksza, M. et al. A neoantigen fitness model predicts tumour response to checkpoint blockade immunotherapy. Nature 551, 517-520 (2017).
5. Balachandran, V. P. et al. Identification of unique neoantigen qualities in long-term survivors of pancreatic cancer. Nature 551, 512-516 (2017).
6. Burnet, F. M. The concept of immunological surveillance. Prog. Exp. Tumor Res. 13, 1-27 (1970).
7. Dunn, G. P., Bruce, A. T., Ikeda, H., Old, L. J. & Schreiber, R. D. Cancer immunoediting: from immunosurveillance to tumor escape. Nat. Immunol. 3, 991-998 (2002).
8. Schumacher, T. N. & Schreiber, R. D. Neoantigens in cancer immunotherapy. Science 348, 69-74 (2015).
9. Rosenthal, R. et al. Neoantigen-directed immune escape in lung cancer evolution. Nature 567, 479-485 (2019).
10. Zhang, A. W. et al. Interfaces of malignant and immunologic clonal dynamics in ovarian cancer. Cell 173, 1755-1769 (2018).
11. Jiménez-Sánchez, A. et al. Heterogeneous tumor-immune microenvironments among differentially growing metastases in an ovarian cancer patient. Cell 170, 927-938 (2017).
12. Balli, D., Rech, A. J., Stanger, B. Z. & Vonderheide, R. H. Immune cytolytic activity stratifies molecular subsets of human pancreatic cancer. Clin. Cancer Res. 23, 3129-3138 (2017).
13. Rizvi, N. A. et al. Mutational landscape determines sensitivity to PD-1 blockade in non-small cell lung cancer. Science 348, 124-128 (2015).
14. Allen, E. M. V. et al. Genomic correlates of response to CTLA-4 blockade in metastatic melanoma. Science 350, 207-211 (2015).
15. Yachida, S. et al. Distant metastasis occurs late during the genetic evolution of pancreatic cancer. Nature 467, 1114-1117 (2010).
16. Ino, Y. et al. Immune cell infiltration as an indicator of the immune microenvironment of pancreatic cancer. Brit. J. Cancer 108, 914-923 (2013).
17. Riquelme, E. et al. Tumor microbiome diversity and composition influence pancreatic cancer outcomes. Cell 178, 795-806 (2019).
18. Bravi, B. et al. Probing T-cell response by sequence-based probabilistic modeling. PLoS Comput. Biol. 17, e1009297 (2021).
19. Sakamoto, H. et al. The evolutionary origins of recurrent pancreatic cancer. Cancer Discov. 10, 792-805 (2020).
20. Dyall, R. et al. Heteroclitic immunization induces tumor immunity. J. Exp. Med. 188, 1553-1561 (1998).
21. Dash, P. et al. Quantifiable predictive features define epitope-specific T cell receptor repertoires. Nature 547, 89-93 (2017).
22. Glanville, J. et al. Identifying specificity groups in the T cell receptor repertoire. Nature 547, 94-98 (2017).
23. Birnbaum, M. E. et al. Deconstructing the peptide-MHC specificity of T cell recognition. Cell 157, 1073-1087 (2014).
24. Solache, A. et al. Identification of three HLA-A*0201-restricted cytotoxic T cell epitopes in the cytomegalovirus protein pp65 that are conserved between eight strains of the virus. J. Immunol. 163, 5512-5518 (1999).
25. Kawakami, Y. et al. Recognition of multiple epitopes in the human melanoma antigen gp100 by tumor-infiltrating T lymphocytes associated with in vivo tumor regression. J. Immunol. 154, 3961-3968 (1995).
26. Parkhurst, M. R. et al. Improved induction of melanoma-reactive CTL with peptides from the melanoma antigen gp100 modified at HLA-A*0201-binding residues. J. Immunol. 157, 2539-2548 (1996).
27. Capietto, A.-H. et al. Mutation position is an important determinant for predicting cancer neoantigens. J. Exp. Med. 217, e20190179 (2020).
28. Deshwar, A. G. et al. PhyloWGS: reconstructing subclonal composition and evolution from whole-genome sequencing of tumors. Genome Biol. 16, 35 (2015).
29. Evans, R. A. et al. Lack of immunoediting in murine pancreatic cancer reversed with neoantigen. JCI Insight 1, e88328 (2016).
30. Barthel, F. P. et al. Longitudinal molecular trajectories of diffuse glioma in adults. Nature 576, 112-120 (2019).
31. Freed-Pastor, W. A. et al. The CD155/TIGIT axis promotes and maintains immune evasion in neoantigen-expressing pancreatic cancer. Cancer Cell 39, 1342-1360 (2021).
32. Zaretsky, J. M. et al. Mutations associated with acquired resistance to PD-1 blockade in melanoma. N. Engl. J. Med. 375, 819-829 (2016).

### Example 2

### Methods

### Patient Samples

We collected matched primary and recurrent PDACs through surgical resection at Memorial Sloan Kettering Cancer Center (MSK) (n=5/9 LTS), and the Garvan Institute of Medical Research (n=1/9 LTS) (FIG. 24). Additional matched primary and recurrent PDACs were previously obtained through the Gastrointestinal Cancer Rapid Medical Donation Program at The Johns Hopkins Hospital (JHH) (n=3/9 LTS, 6/6 STS) and have been previously described¹⁹ (FIG. 24) under Institutional Review Board-approved study protocols. Cohorts of primary only PDAC were collected at MSK (MSK primary PDAC cohort), the International Cancer Genome Consortium (ICGC primary PDAC cohort), and The Cancer Genome Atlas (TCGA) through surgical resection as previously described.^{5,33} We obtained informed consent from all patients. We performed the study in strict compliance with all institutional ethical regulations and institutional review boards. All tumor samples were PDACs. We excluded adenocarcinomas in cystic pancreatic neoplasms and neuroendocrine tumors. We defined LTS and STS consistent with our previous work.⁵ We identified all tumors through histopathologic evaluation following surgery or at autopsy. We conservatively estimated that patients had 100 recurrent tumors when the number of tumors were too numerous to count.

### Nucleic Acid Extraction, Whole Exome Sequencing and Mutation Identification

We previously described methods to extract DNA and sequence samples collected at MSK, Garvan Medical Center,⁵ and JHH.¹⁹ All samples from MSK, Garvan, and JHH were examined by an expert GI pathologist and confirmed to have at least 20% neoplastic cellularity and preserved tissue quality. We macrodissected samples meeting these criteria from serial unstained sections, and extracted genomic DNA as previously described.^{5,19} 500 ng of genomic DNA was then fragmented to a target size of 150-200 bp on a LE220 ultrasonicator (Covaris). Barcoded libraries (Kapa Biosystems) were subjected to exon capture by hybridization using the SureSelect Human All Exon 51 MB V3 (JHH samples) or V4 (all other samples) kits (Agilent). DNAlibraries were subsequently sequenced on a HiSeq 2500 (JHH samples) or 4000 (all other samples) (Illumina) in paired end 100/100 reads, using the TruSeq SBS Kit v3 (Illumina) with target coverage of 150-250× for tumor samples and 70× for matched normal. Sequence data were demultiplexed using Illumina CASAVA software. After removal of adapter sequences using BIC,³⁴ reads were aligned to the reference human genome (hg19) using the Burrows-Wheeler Alignment tool (bwa mem v0.7.17) and samtools (v1.6). Duplicates were marked with picard-2.11.0 MarkDuplicates (http://broadi-nstitute.github.io/picard). Indel realignments were done with the Genome Analysis toolkit (GenomeAnalysisTK-3.8-1-0-gf15c1c3ef) RealignerTargetCreator and IndelRealigner³⁵ using 1000 genome phase1 indel (1000G_phase1.indels.b37.vcf) and Mills indel calls (Mills_and_1000G gold_standard.indels.b37.vcf) as references. Base calls were recalibrated with BaseRecalibrator³⁵ and dbSNP version 138. Average unique sequence coverages of 207×, 152×, 212×, and 221× were achieved for STS primary, LTS primary, STS recurrent, and LTS recurrent tumor samples respectively, and 88× and 84× were achieved for STS normal and LTS normal samples, respectively.

MuTect 1.1.7³⁵ and Strelka 1.0.15³⁶ were used to call SNVs and indels on pre-processed sequencing data. For the MuTect calls, dbSNP 138 and CosmicCodingMuts.vcf version 8637 were used as reference files. For the Strelka calls, we set “isSkipDepthFilters=1” to prevent filtering-out of mutation calls from exome sequencing due to exome-sequencing mapping breadth. Unbiased normal/tumor read counts for each SNV and indel call were then assigned with the bam-readcount software 0.8.0 (https://github.com/genome/bam-readcount). A minimum base quality filter was set with the “−b 15” flag. The reads were counted in an insertion-centric way with the “−i” flag, so that reads overlapping with insertions were not included in the per-base read counts. We then use the normal/tumor read counts to filter mutations. Filtering criteria are 1) total coverage for tumor ≥10, 2) variant allele frequency (VAF) for tumor ≥4%, 3) number of reads with alternative allele ≥9 for tumor, 4) total coverage for normal ≥7, and 5) VAF for normal ≤1% at a given mutation. These filters exist for all mutations except for mutations in the KRAS gene.

In order to avoid missing possible KRAS driver mutations, common mutations in KRAS known to be pathogenic were manually curated if the sample did not already contain a KRAS mutation. Using IGV Viewer 2.4.19,³⁸ we inspected the top ten coding mutations in KRAS denoted in the Genomic Data Commons Database³⁹ at the following positions on chromosome 12; bases 25378562(C>T), 25380275(T>G), 25398281(C>T), 25398282(C>A), 25398284(C>A, T, or G), and 25398285(C>A, T, or G). One mutation was selected at most based on the number of reads containing the alternative allele at the position. In total, six additional KRAS mutations were added for the STS samples, and six were added to the LTS samples.

### HLA Typing

HLA-I typing for PDAC patients was performed in silico with the OptiType version 1.3.3 tool⁴⁰ using non-tumor sequencing reads.

### Neoantigen Prediction

Putative neoantigens were identified in silico. In brief, all wild-type (WT) and mutant genomic sequences corresponding to coding mutations were translated to an amino acid sequence consistent with the GRCh37 reference genome (GRCh37.75) using the snpEff.v4.3t software⁴¹ with options set as “-noStats -strict -hgvs1LetterAa -hgvs -canon -fastaProt [fasta file name]”. Only annotations without “WARNING” or “ERROR” were kept and the most deleterious missense mutation was prioritized in mapping a genomic mutation to a gene. Missense mutations were centrally located in a peptide of up to 17 amino acids long, which depended on the location of the missense mutation within a protein. This corresponded to nine 9-mers in a left-to-right sliding fashion, each containing the mutant amino acid in a different position. Predictions of MHC class-I binding for both the WT peptide (p^{WT}) and mutant peptide (p^{MT}) were estimated using the NetMHC 3.4 software^{42,43} with patient-specific HLA-I types. All p^{MT}s with predicted IC₅₀ affinities below 500 nM to a patient-specific HLA-I type were defined as neoantigens.

### Cell Lines and Cell Culture

We purified peripheral blood mononuclear cells (PBMCs) from healthy donor buffy coats (New York Blood Center, New York, USA) and isolated T cells using a Pan-T cell isolation kit (Miltenyi). We activated T cells with CD3/CD28 beads (Thermo Fisher, MA, USA) with IL-7 (3000 IU/mL) and IL-15 (100 IU/mL) (Miltenyi Biotec, Germany), and transduced T cells on day 2 post activation. Virus-producing cell lines (H29 and RD114-envelope producers) were previously described.^{44,45} We cultured T cells or HLA-transduced K562 cells and T2 cells in RPMI media supplemented with 10% fetal bovine serum (FBS, Nucleus Biologics), 100 U/ml Penicillin/Streptomycin (Gibco), and 2 mM Glutamine (Gibco). We cultured virus-producing cell lines in DMEM media supplemented with 10% FBS (Nucleus Biologics), 100 U/ml Penicillin/Streptomycin (Gibco), and 2 mM Glutamine (Gibco).

### TCR Cloning, Transduction, and Peptide Stimulation

We constructed TCR fragments as previously described.⁴⁶ Briefly, we fused epitope specific TRB V-D-J and TRA V-J sequences to mouse constant TRB and TRA chain sequences respectively to prevent mispairing of transduced TCRs with the endogenous TCRs.⁴⁷ We used modified mouse constant regions to further improve pairing and increase cell surface TCR expression as previously described (mTCR).⁴⁶ We joined the TRB and TRA chains with a furin SGSG P2A linker, cloned the TCR constructs into an SFG γ-retroviral vector,⁴⁸ and sequence-verified all plasmids (Genewiz). We transfected retrovirus vectors into H29 cells (gpg29 fibroblasts) using calcium phosphate to produce VSV-G pseudo-typed retroviruses.⁴⁴ We next used Polybrene (Sigma) and viral-containing supernatants to generate stable RD 114-enveloped producer cell lines.⁴⁵ We collected and concentrated virus-containing supernatants using Retro-X™ Concentrator (Takara). For T cell transductions, we coated non-tissue culture treated 6-well plates with Retronectin (Takara) as per the manufacturer's protocol. We plated a titrated viral quantity to 3×10⁶ activated T cells per well, and centrifuged cells for 1 hour at room temperature at 300 g, and used transduced T cells either between day 7-14 post transduction or cryopreserved them for future use. We used mock-transduced T cells as controls.

To stimulate transduced T cells with peptide, we used T2 cells as antigen presenting cells (APCs). All peptide panels were commercially custom synthesized by Genscript (Piscataway, NJ), and were >95% pure. Briefly, we pulsed 5×10⁴ T2 cells per well in a 96-well U-bottom plate for 1 hour at 37° C. with the indicated peptide at the indicated concentrations. After 1 hour, we washed out the peptide by centrifugation, and added 5×10⁴ TCR-transduced T cells per well. We used an equivalent number of mock transduced total T cells, and irrelevant peptides as controls. We measured CD137 (4-1BB) expression on CD8⁺mTCR⁺ T cells 24 hours later.

### Flow Cytometry

We defined TCR transduced CD8⁺ T cells as live, CD3⁺, CD8⁺, mTCR⁺ cells (FIG. 7, panel B). We purchased antibodies from Biolegend (CD3—clone SK-7, PE-Cy7; CD8—clone SK1, Alexa Fluor 700; mTRB—clone H57-597, PE-Cy5; and CD137—clone 4B4-1, PE), and viability dye (DAPI solution) from BD Biosciences. We stained cells using antibody cocktails in the dark at 4° C. according to manufacturer's instructions, washed, and analyzed on a FACS LSR Fortessa (BD Biosciences). Flow cytometric data were collected using FACSDiva (BD Biosciences, version 8.0.1). We diluted reagents according to manufacturer's instructions. We used Flowjo (version 10, Tree Star) to perform our analyses.

### Neoantigen Quality Model: Q

We consider 9-amino-acid long peptides containing a single point mutation as potential neoantigens if they are a predicted binder to a patient specific HLA allele. For a given neoantigen with a peptide sequence p^{MT} (with corresponding wildtype peptide sequence denoted as p^{WT}), and an HLA allele h, we define the neoantigen quality Q as:

$$Q ( {p}^{MT} ; h ) = R ( pMT ) \times D ( {p}^{MT} , {p}^{WT} ; h ) . \qquad ( 1 )$$

The non-self recognition component R quantifies the recognition probability of peptide p^{MT} by comparison to validated non-self epitopes from infectious diseases and it has been introduced previously.^{4,5} The self discrimination component, D, expands on the previously proposed measure based only on MHC-presentation^{4,5} by including a new measure of cross-reactivity distance, C, between p^{MT} and p^{WT}. Below we briefly describe R and derive D.

### Non-Selfness: R

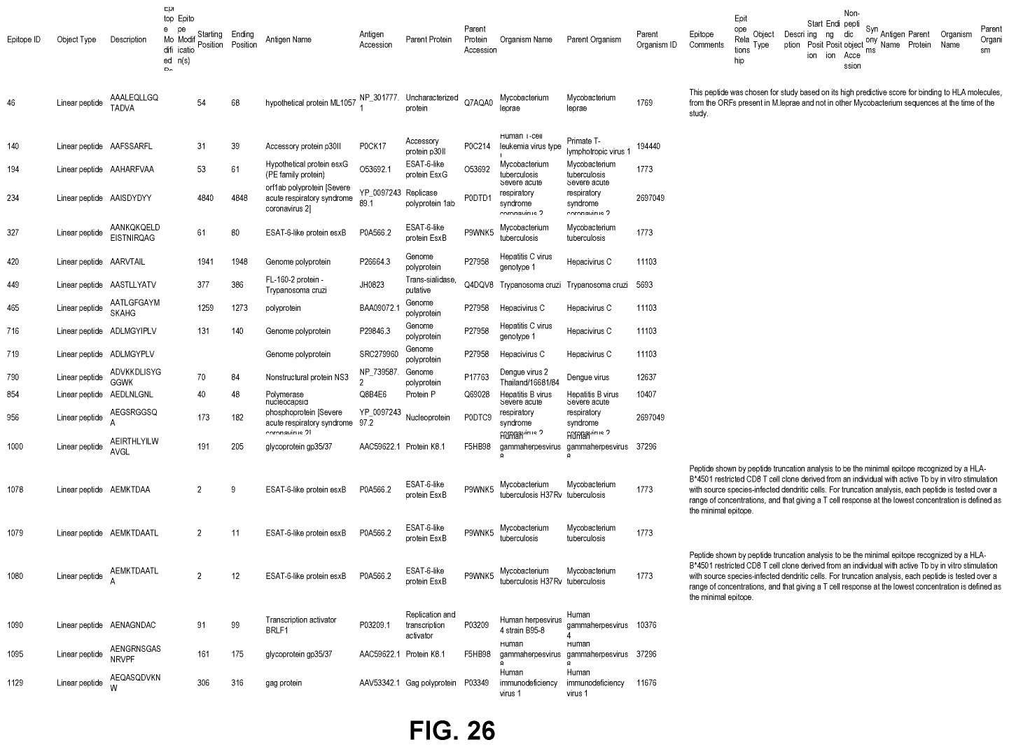

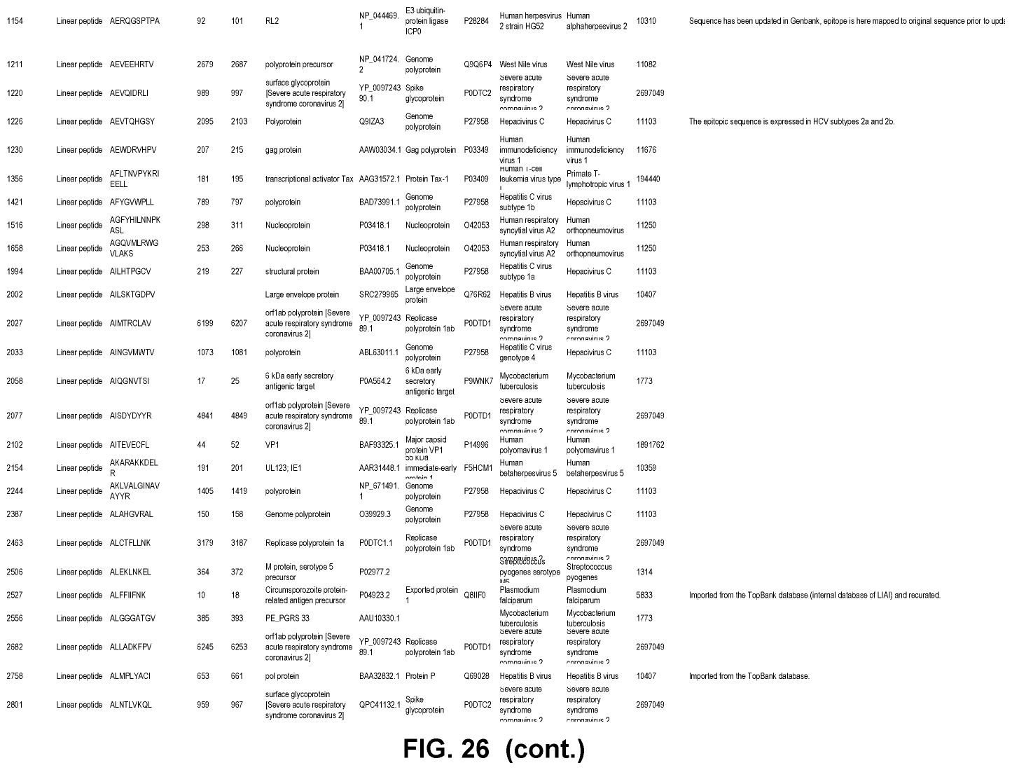

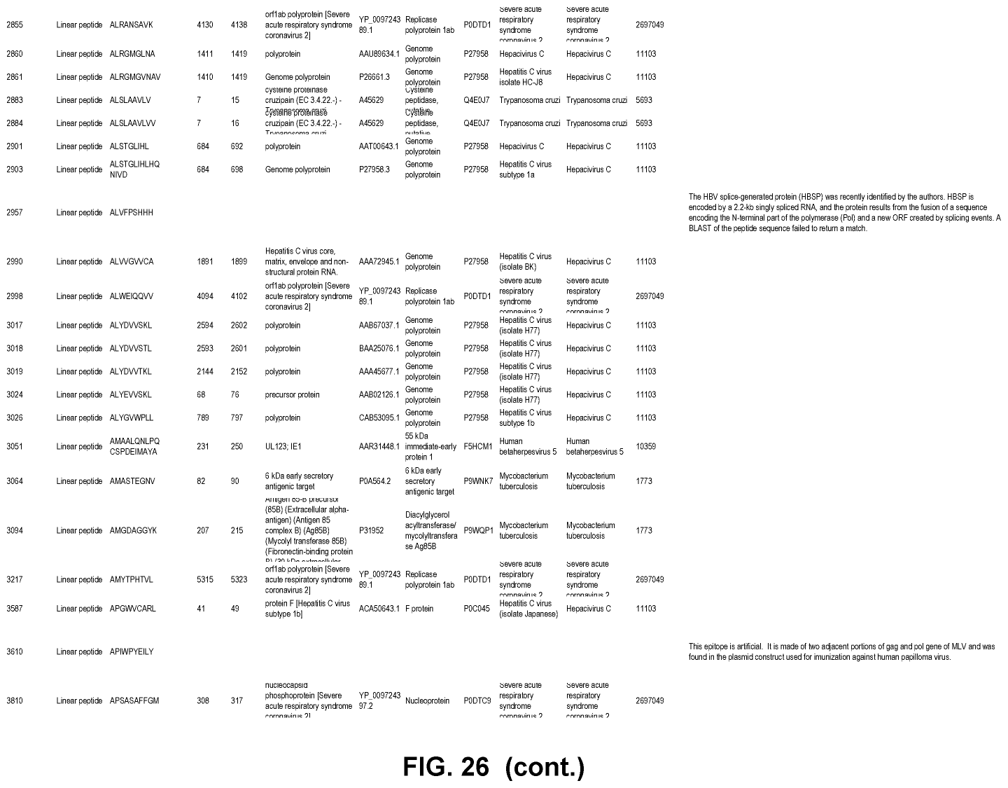

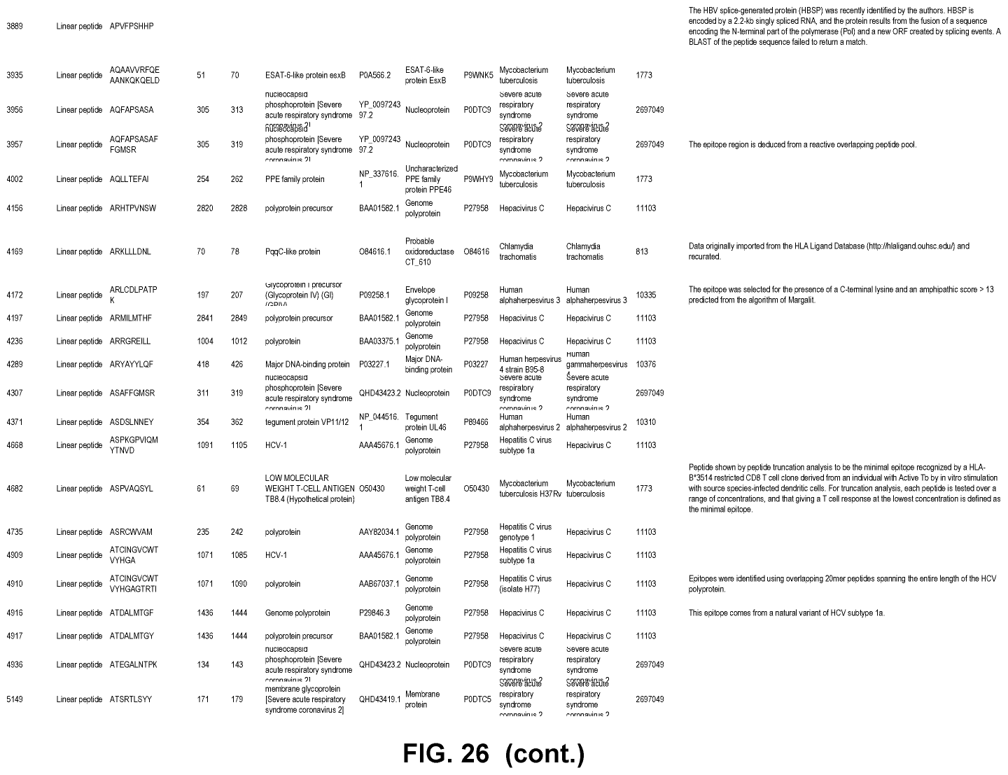

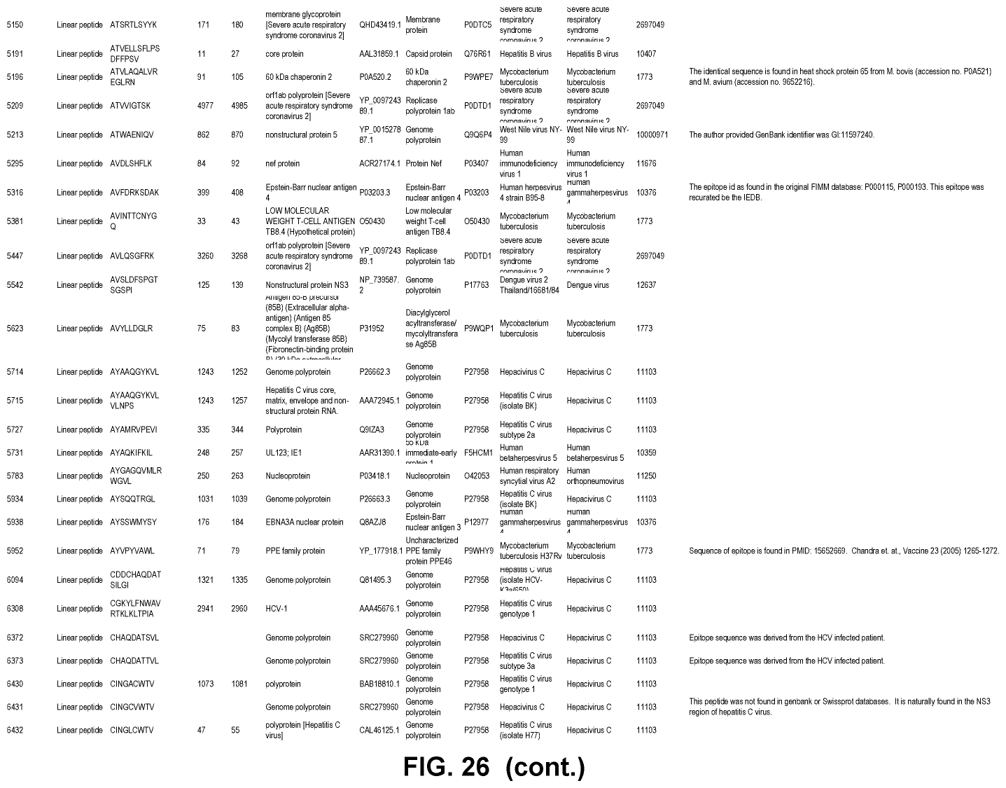

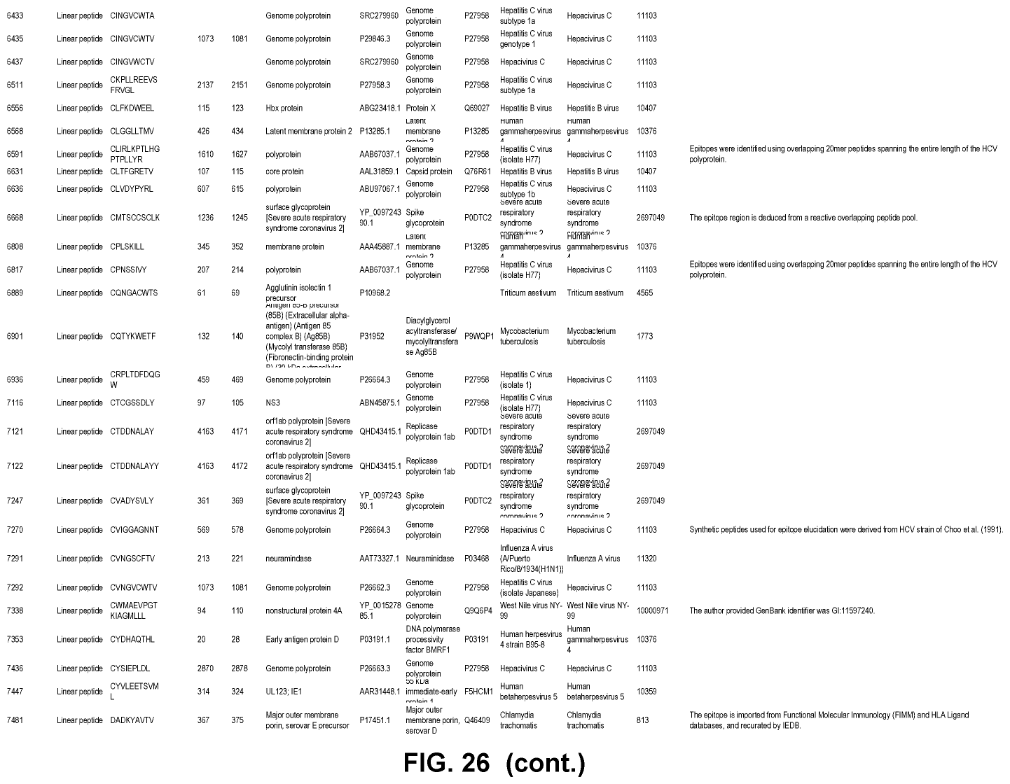

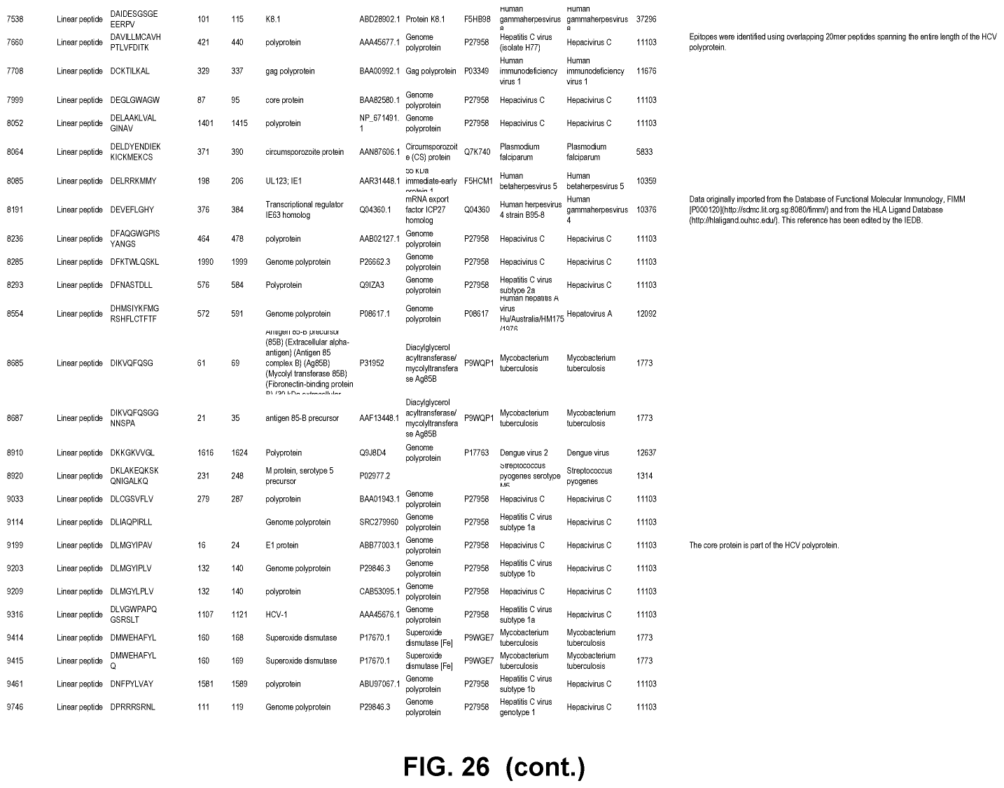

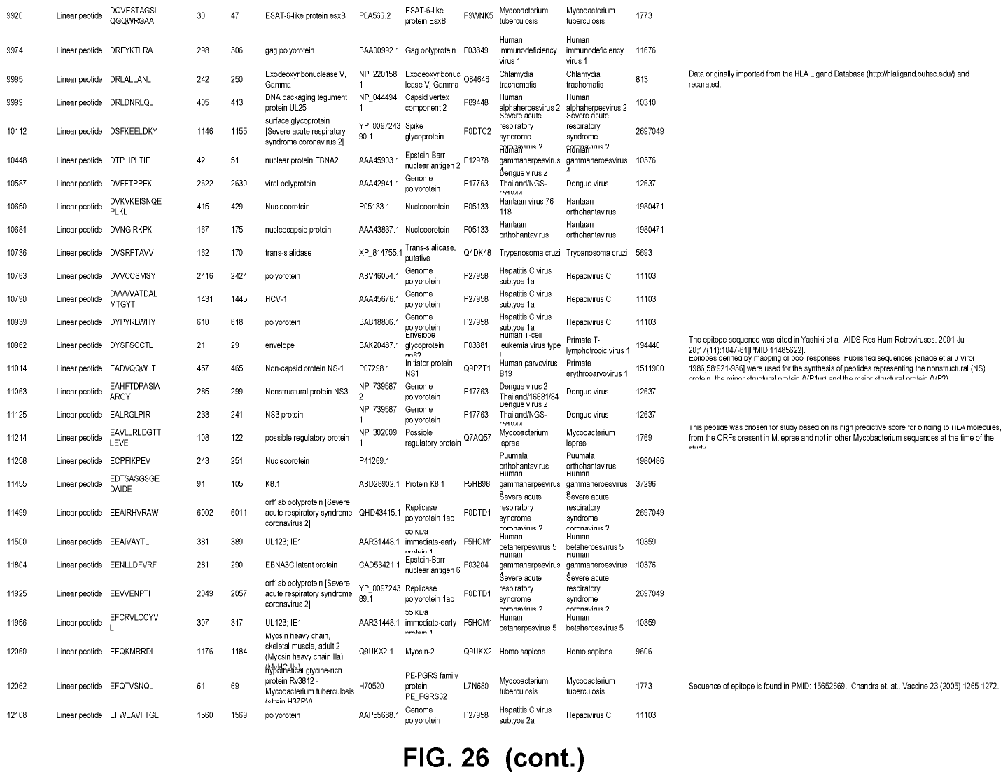

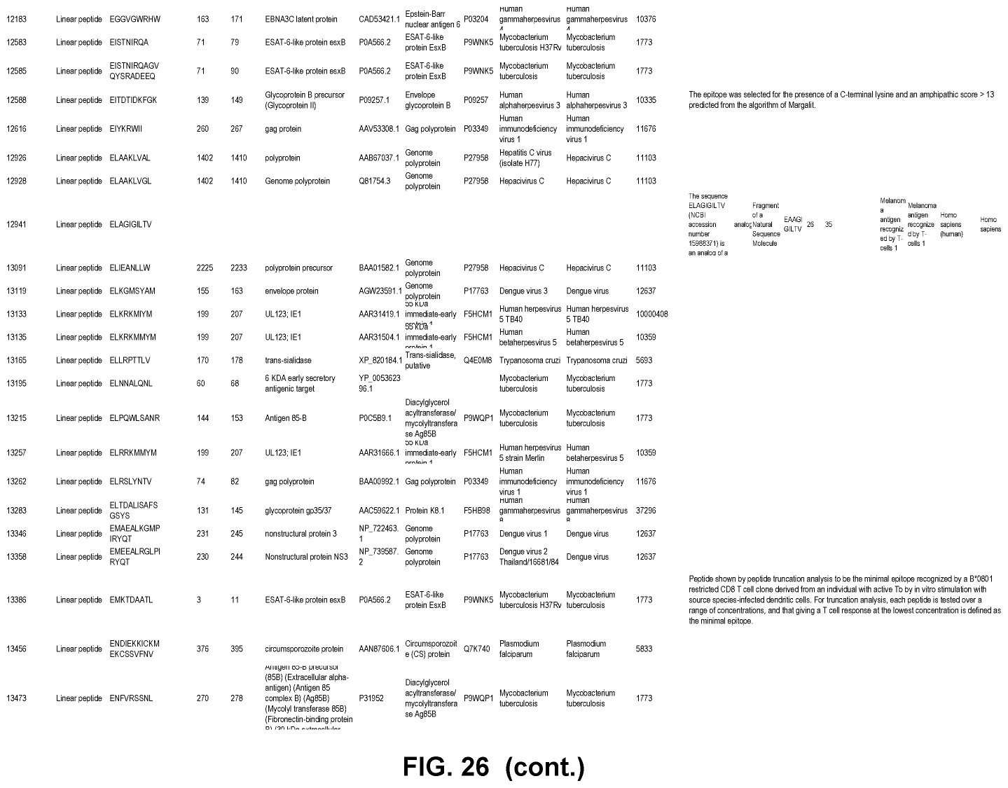

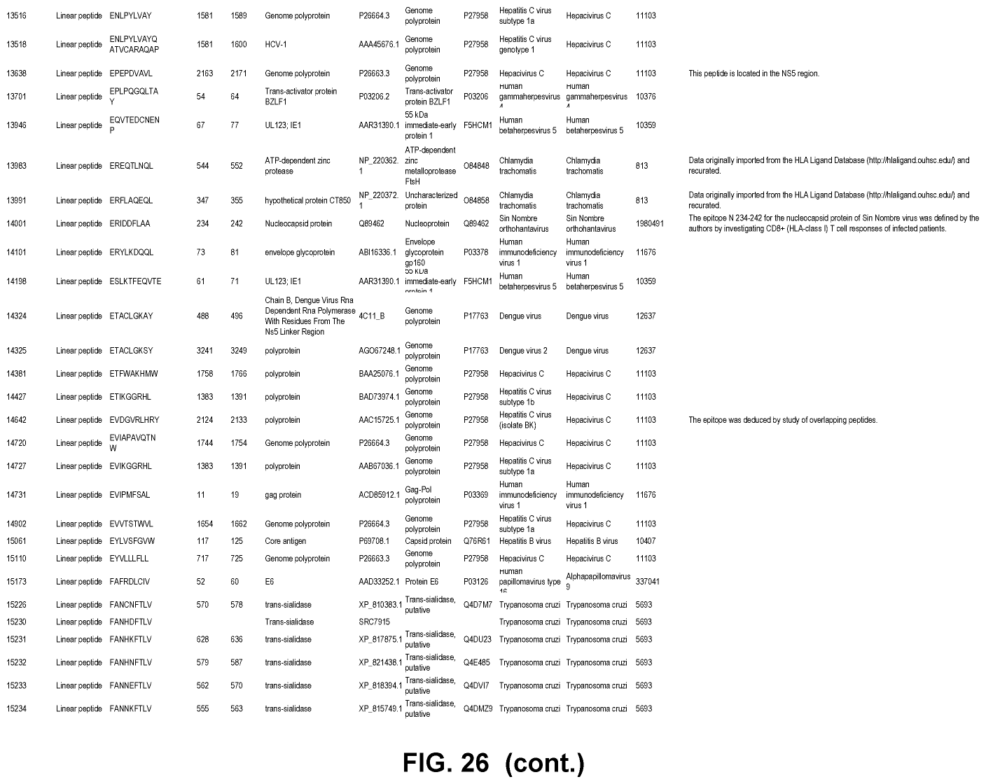

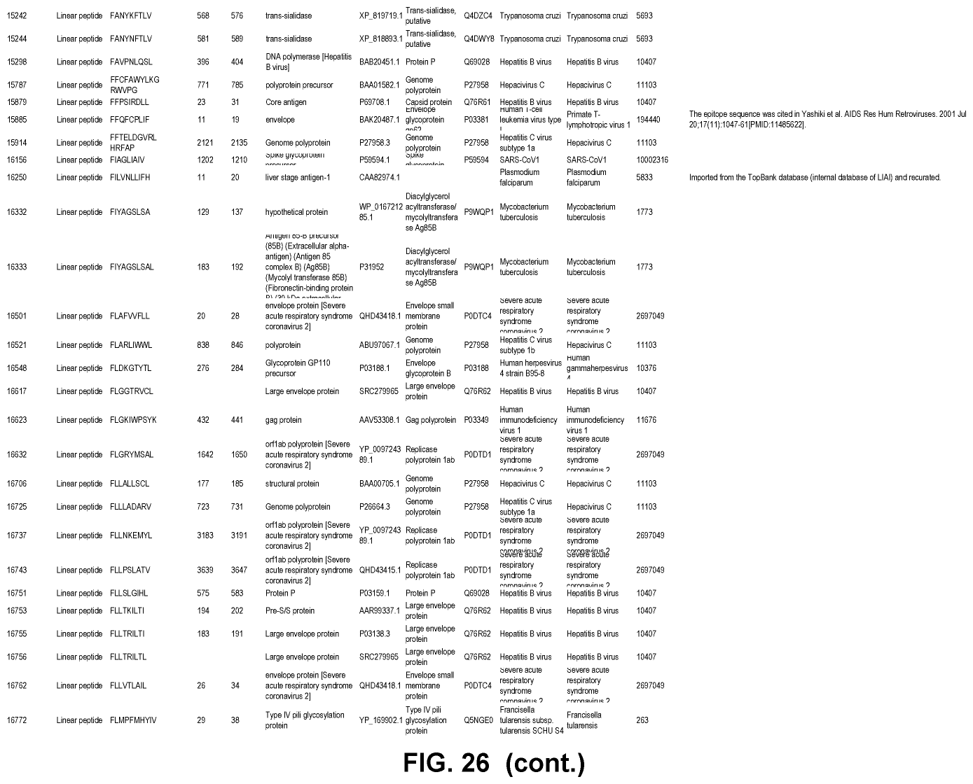

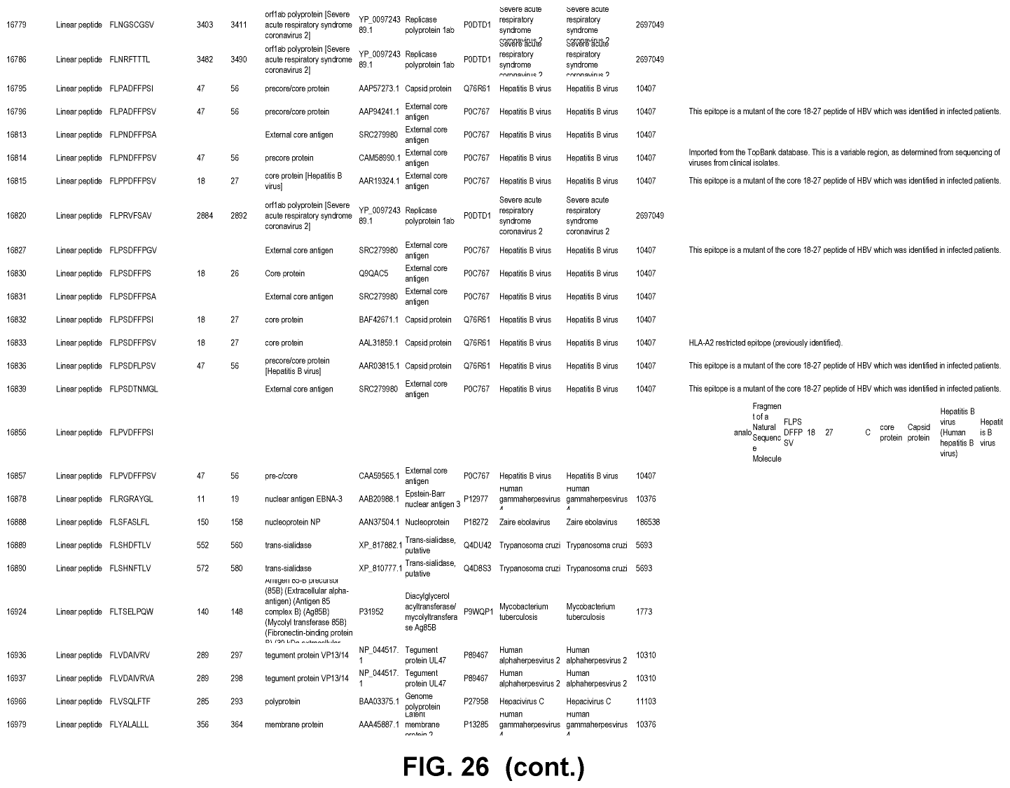

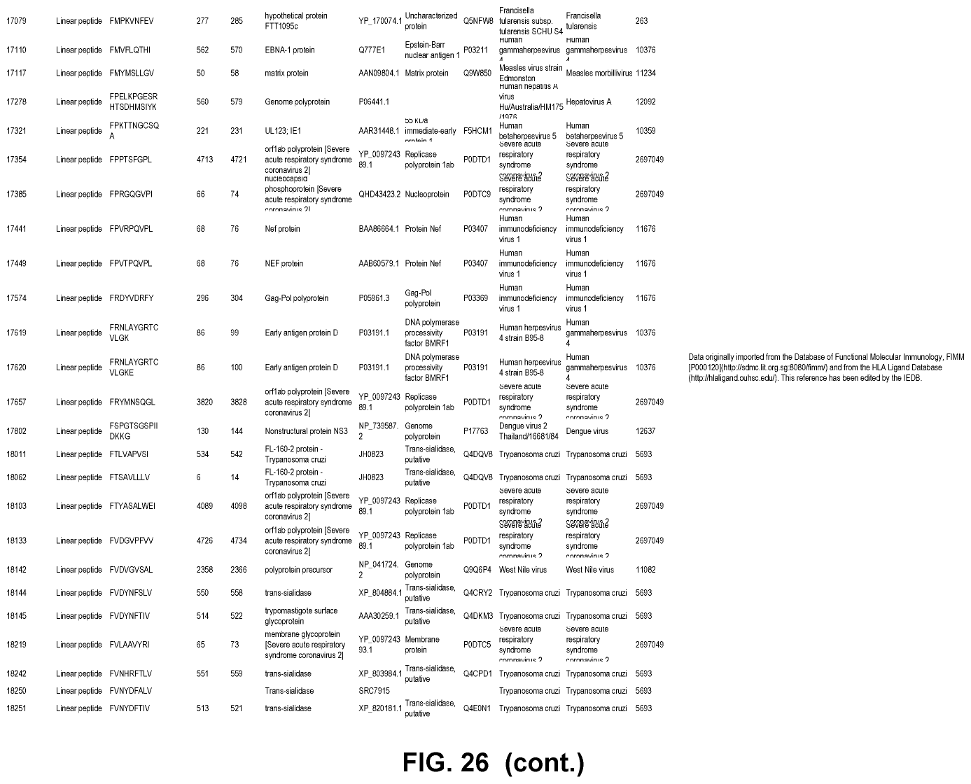

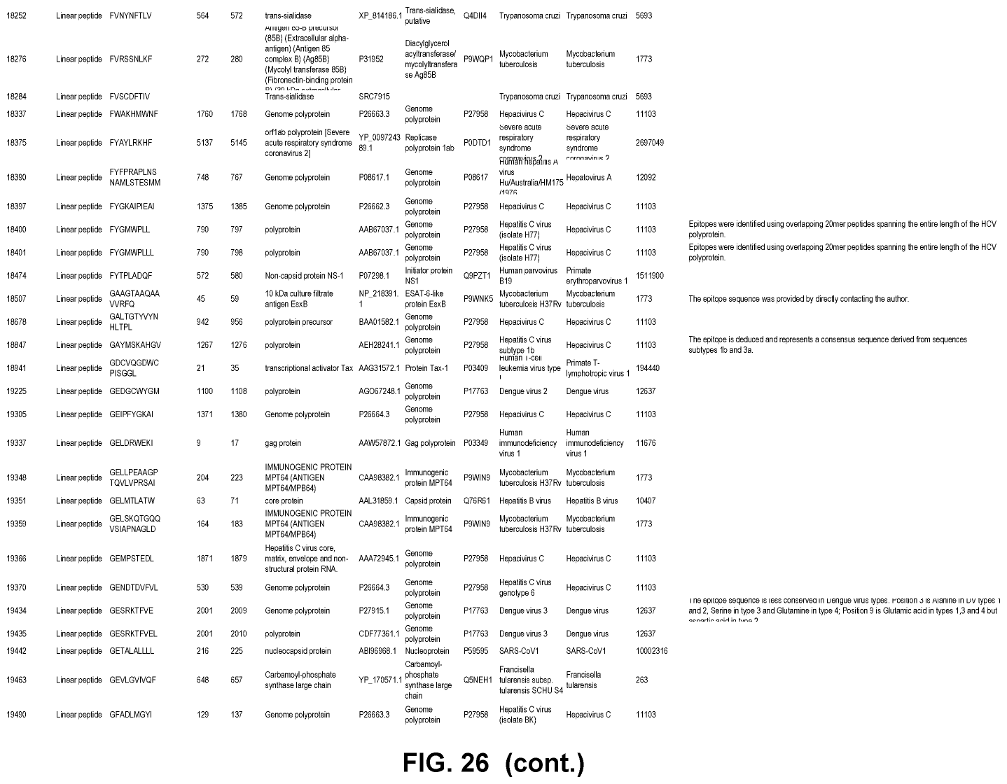

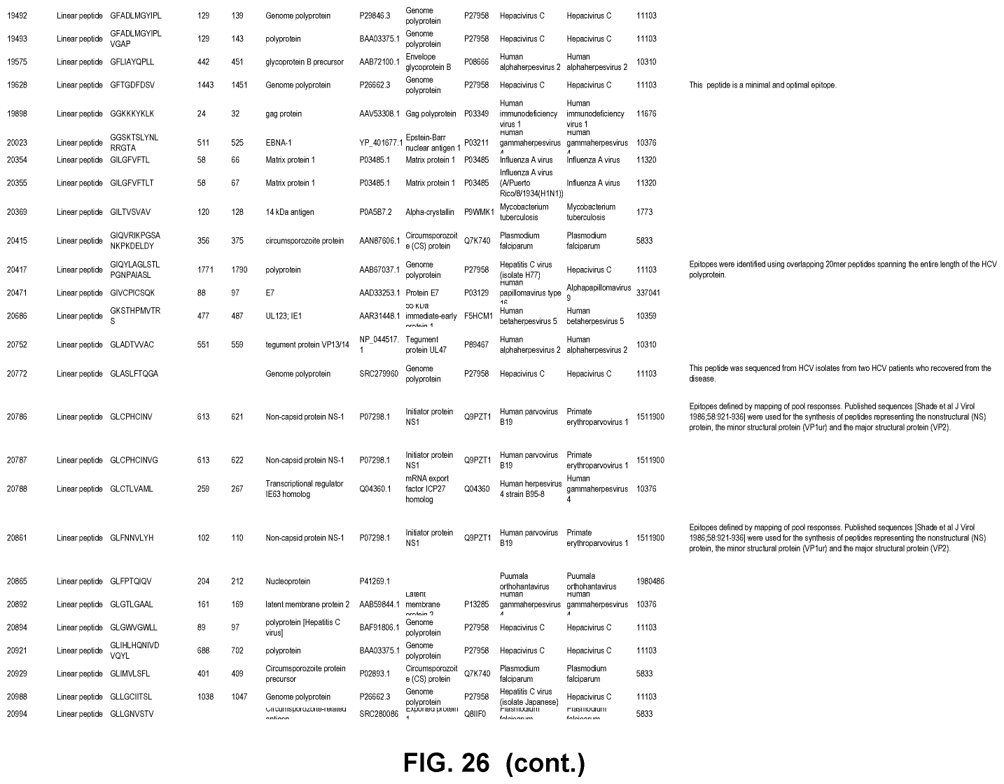

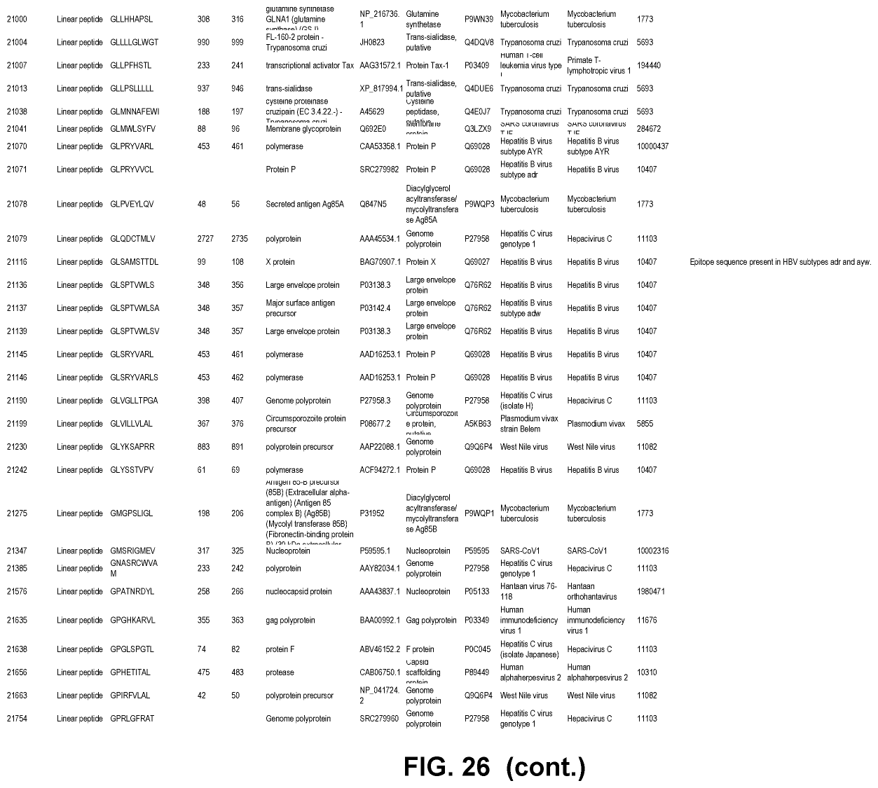

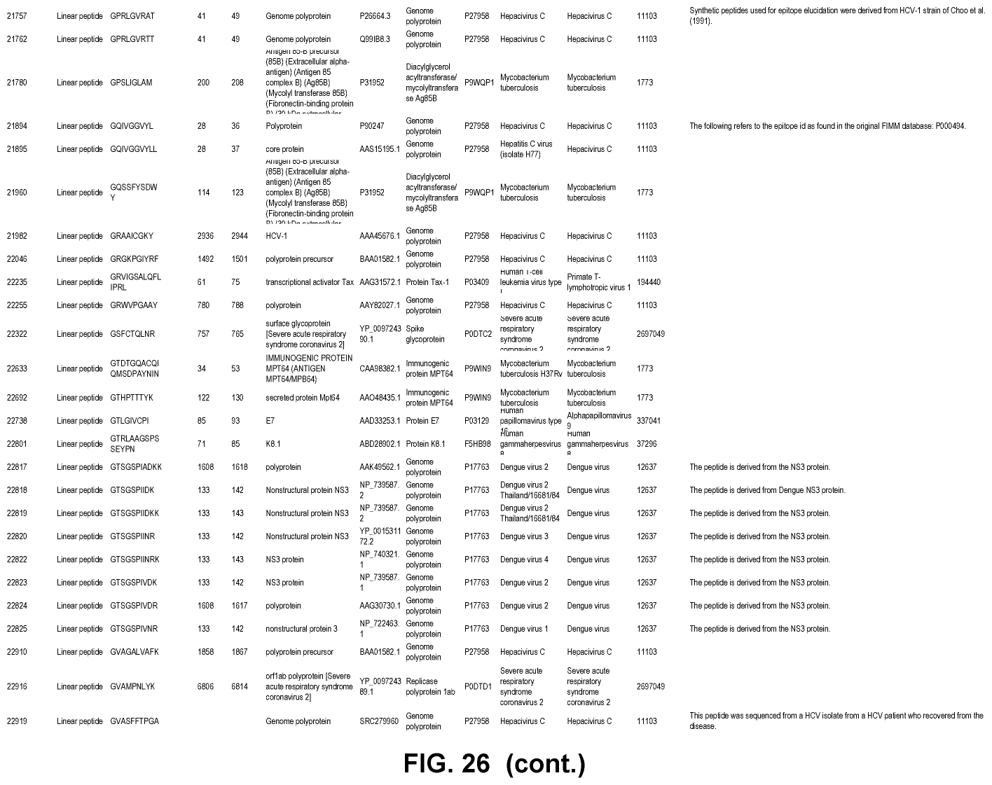

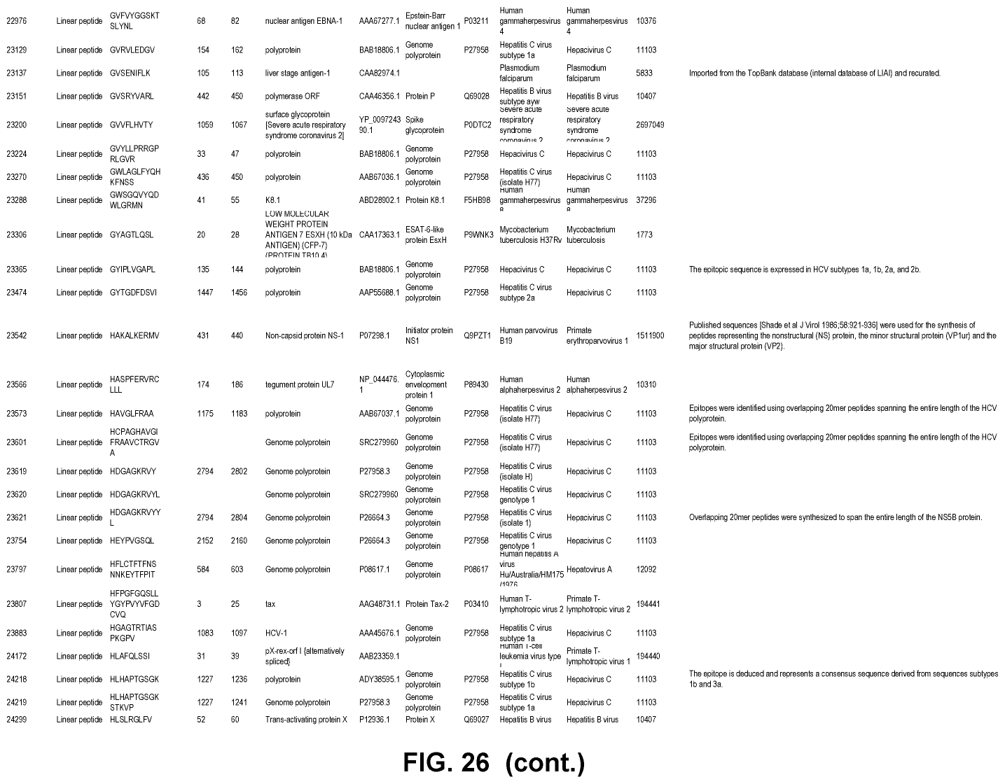

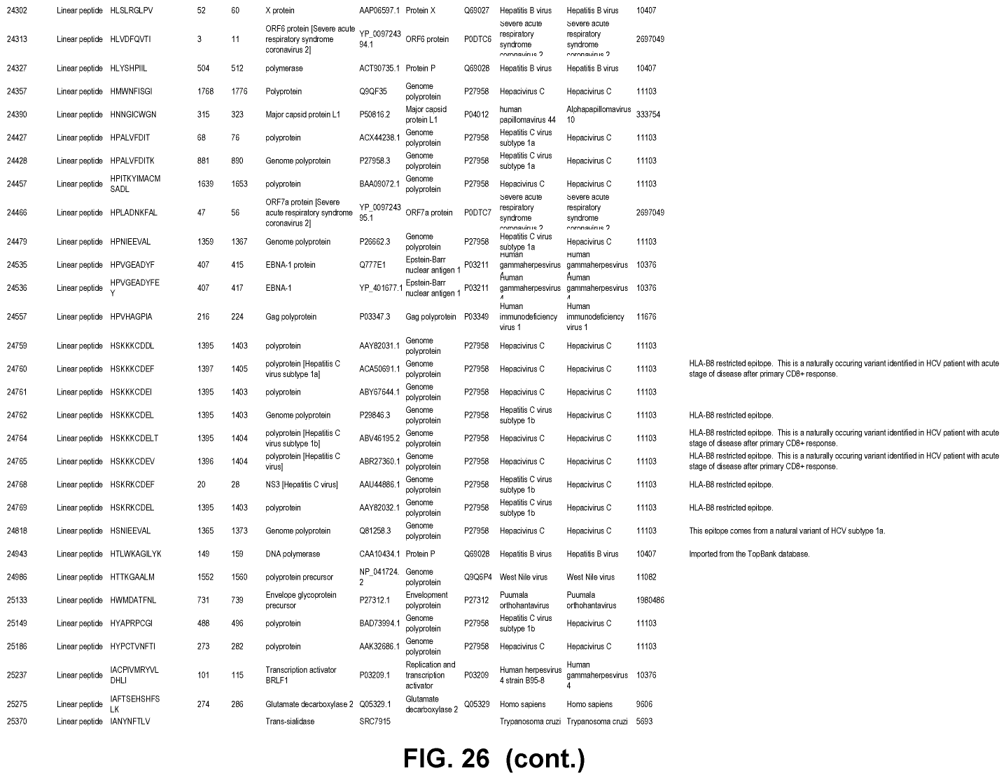

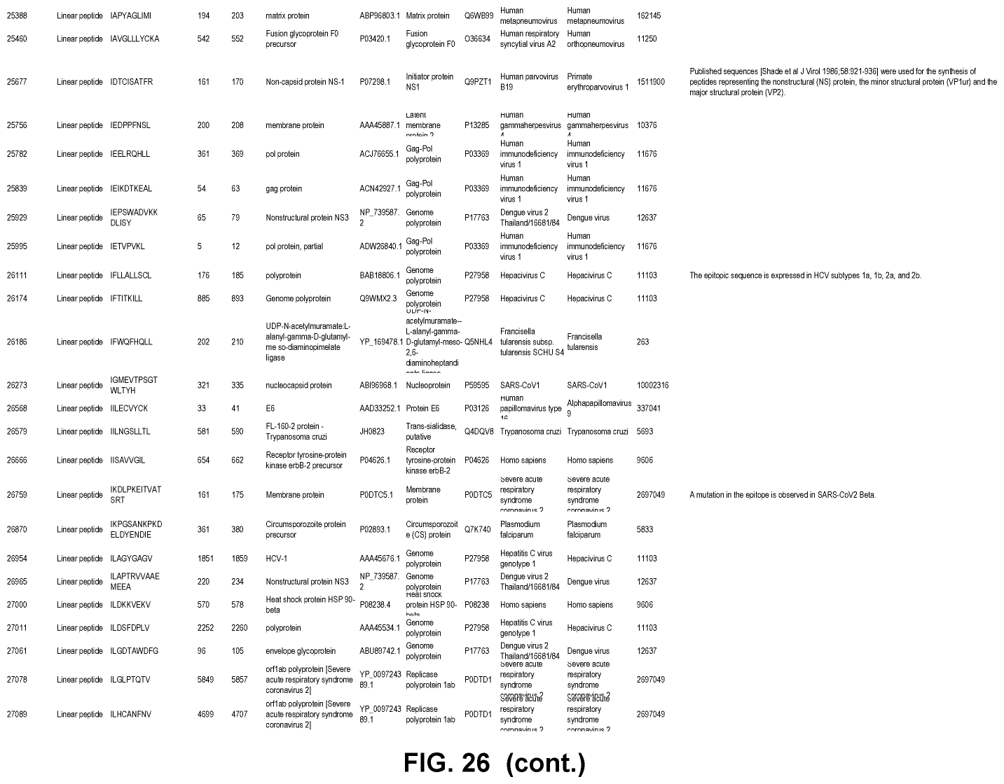

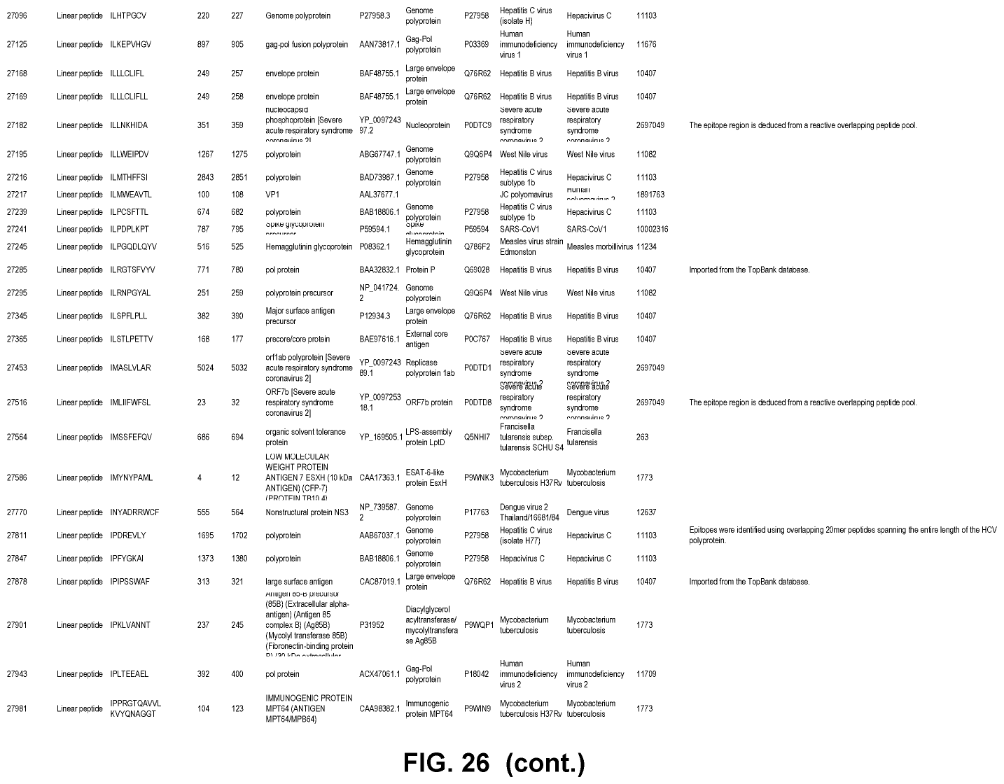

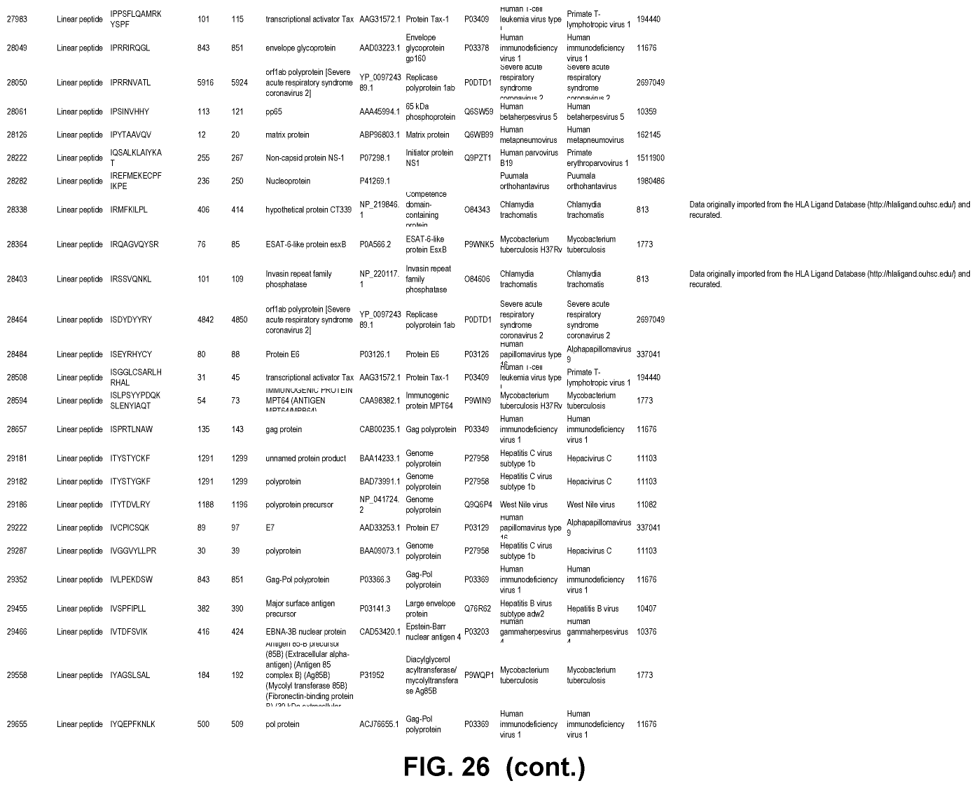

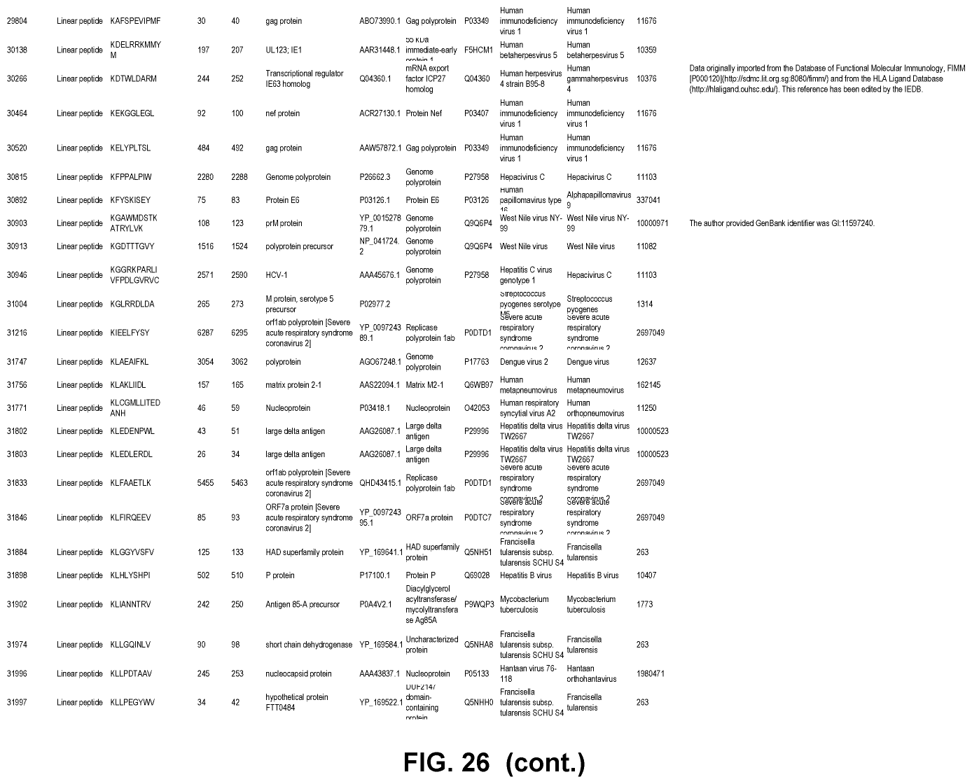

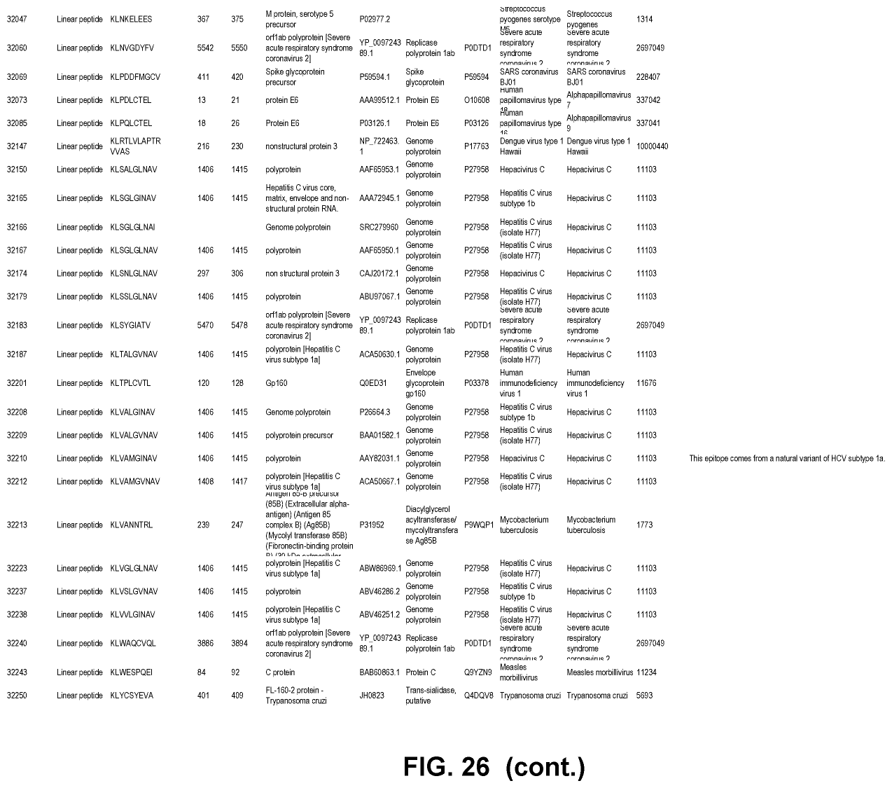

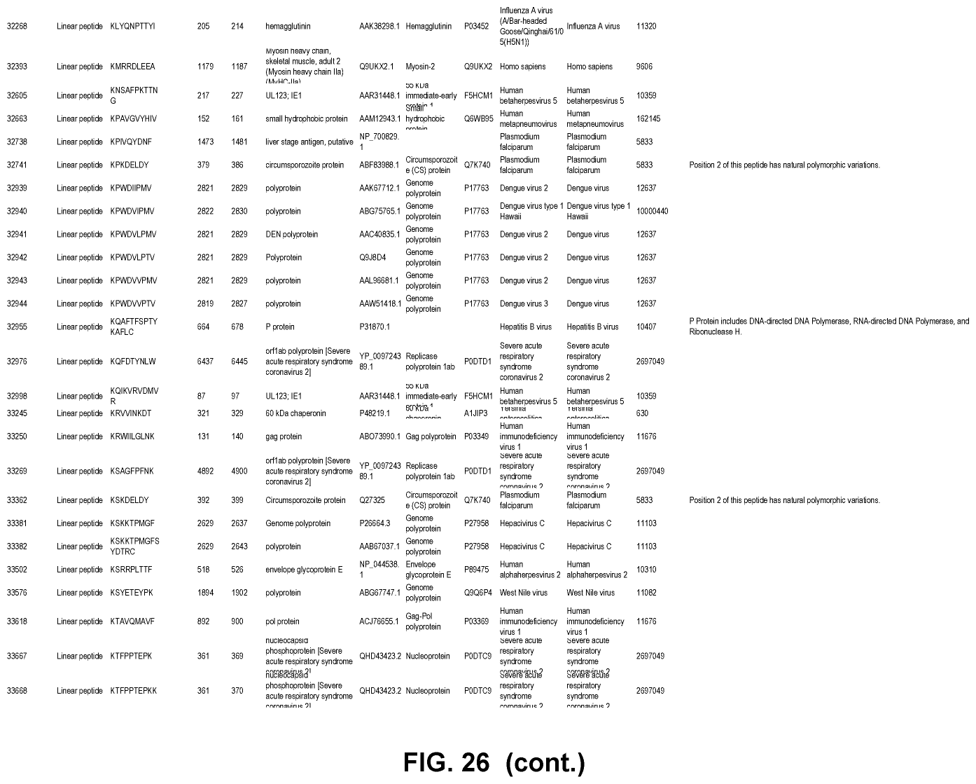

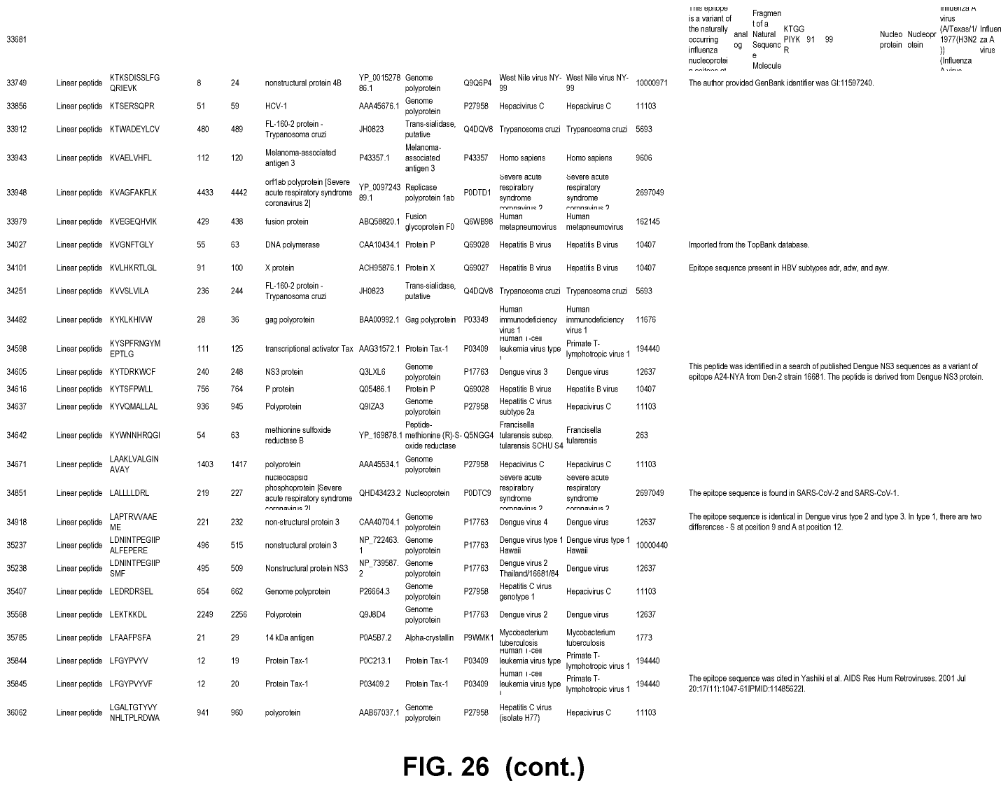

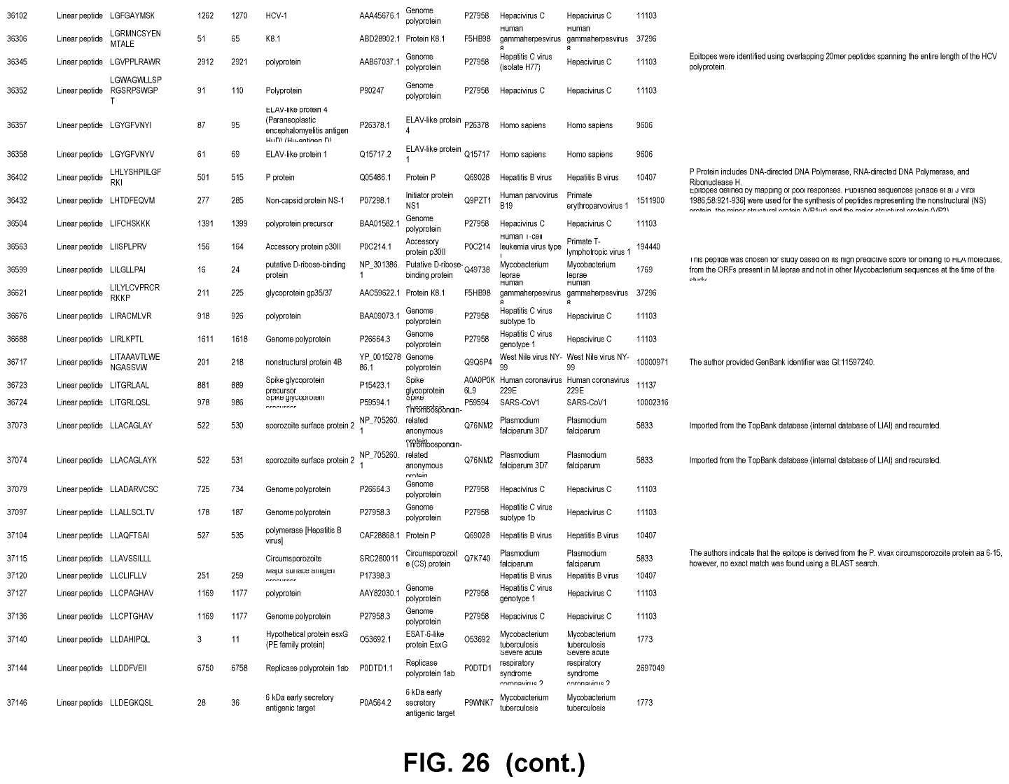

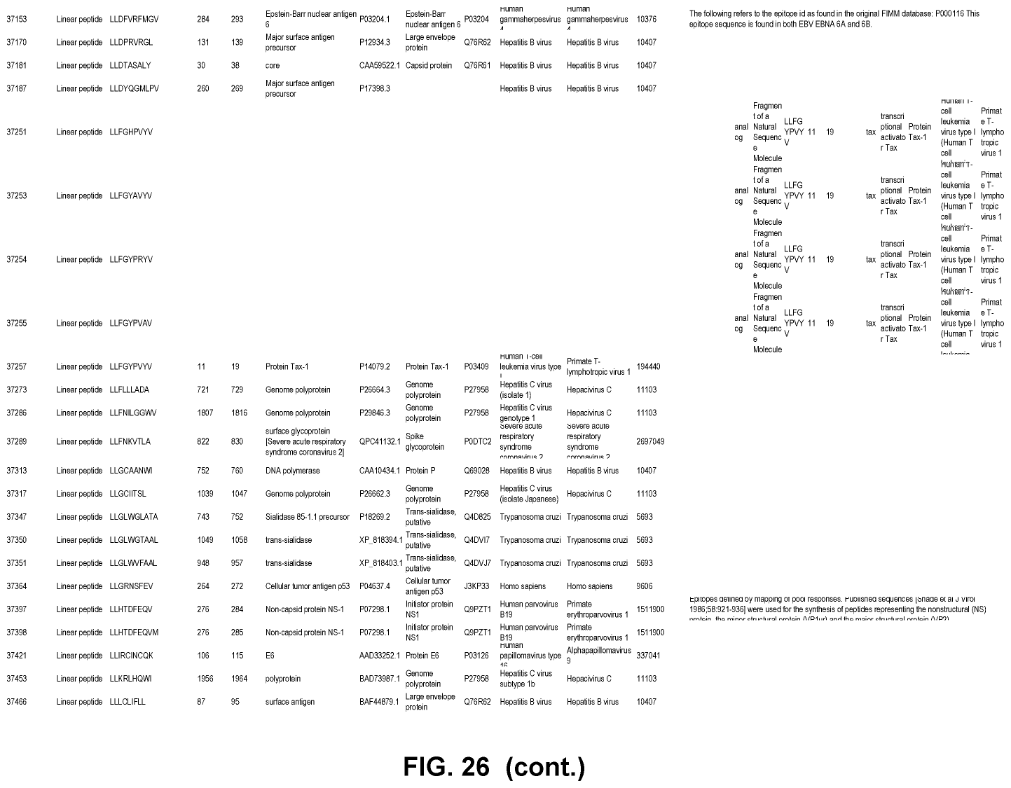

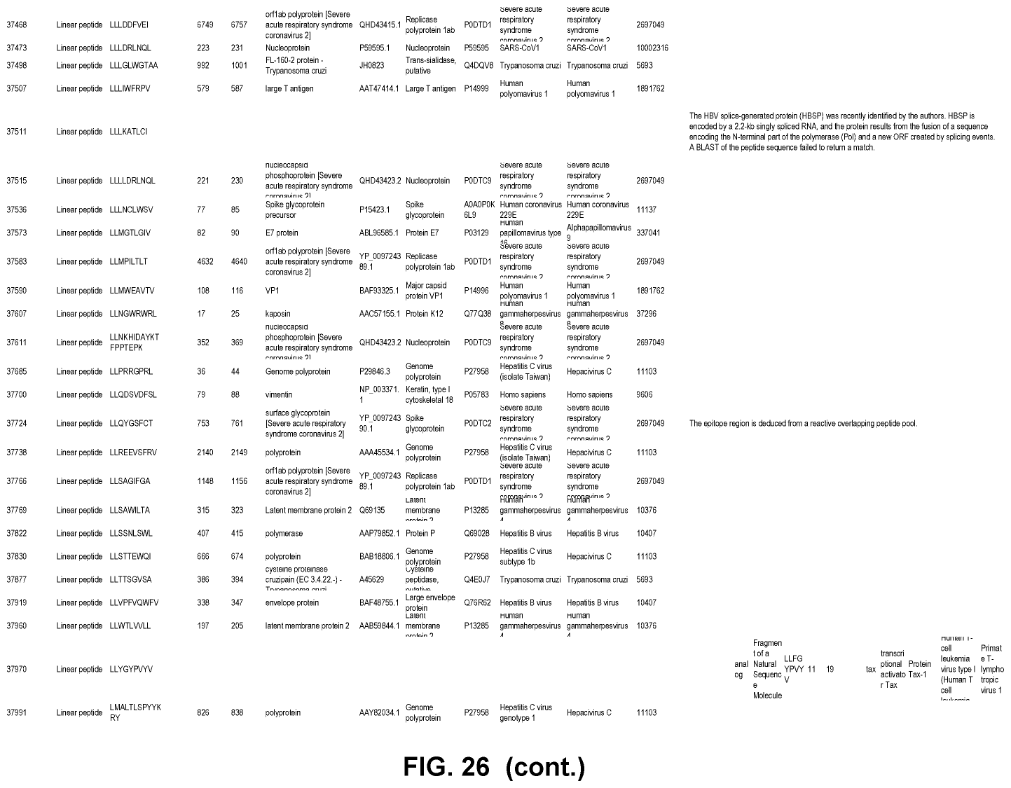

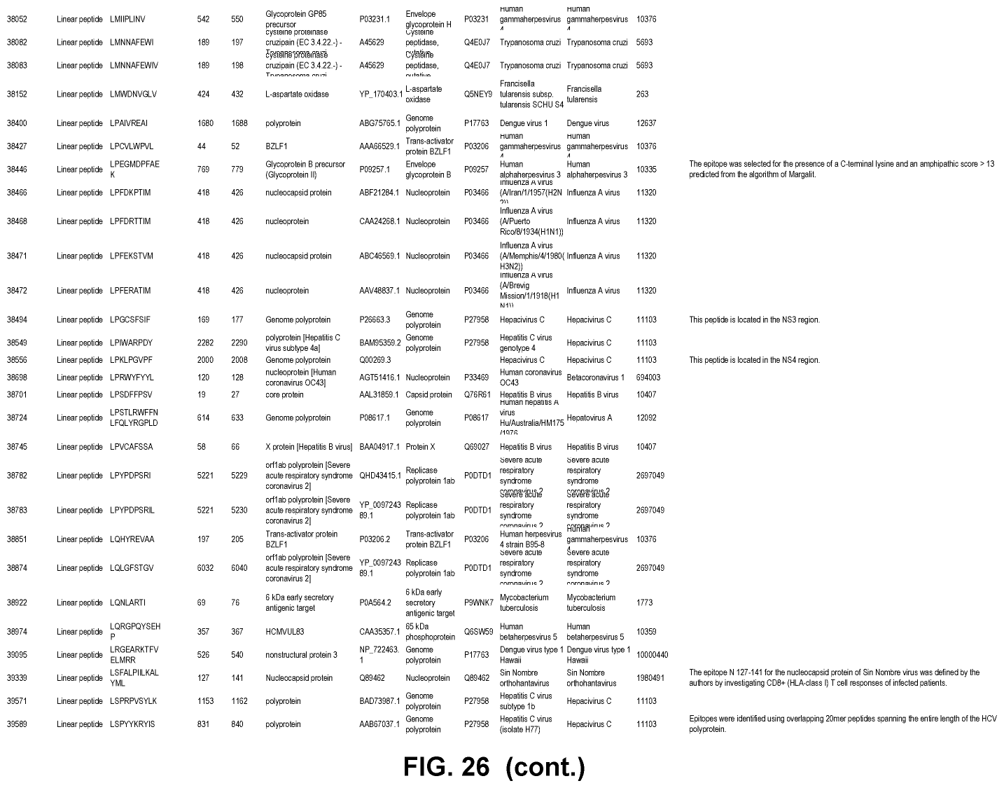

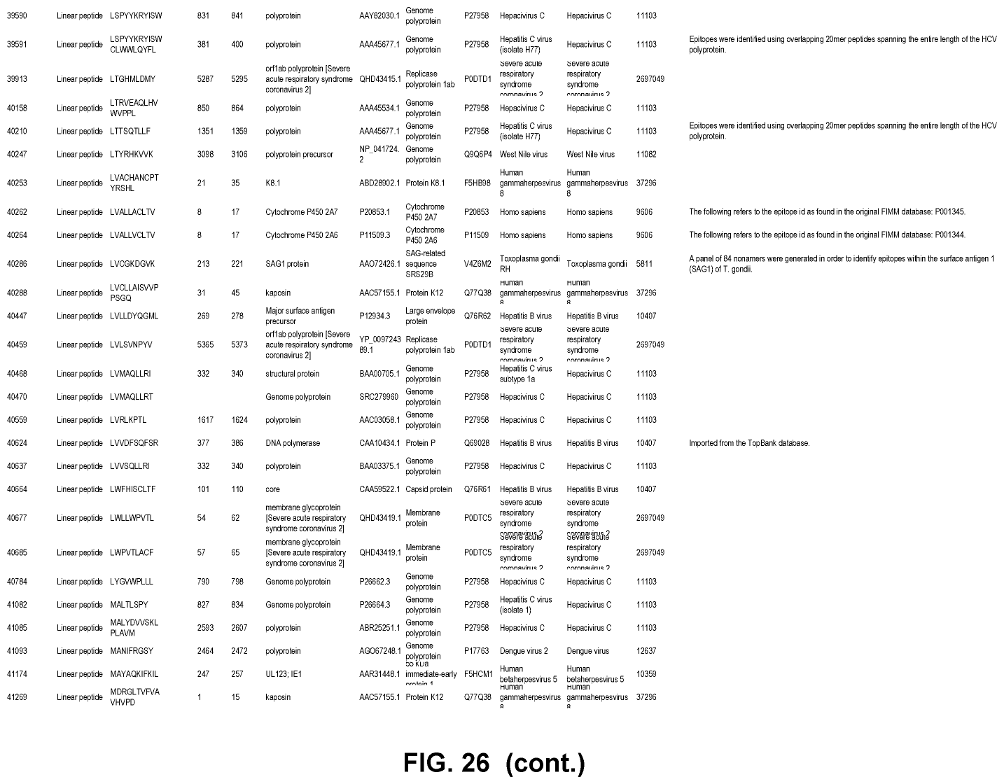

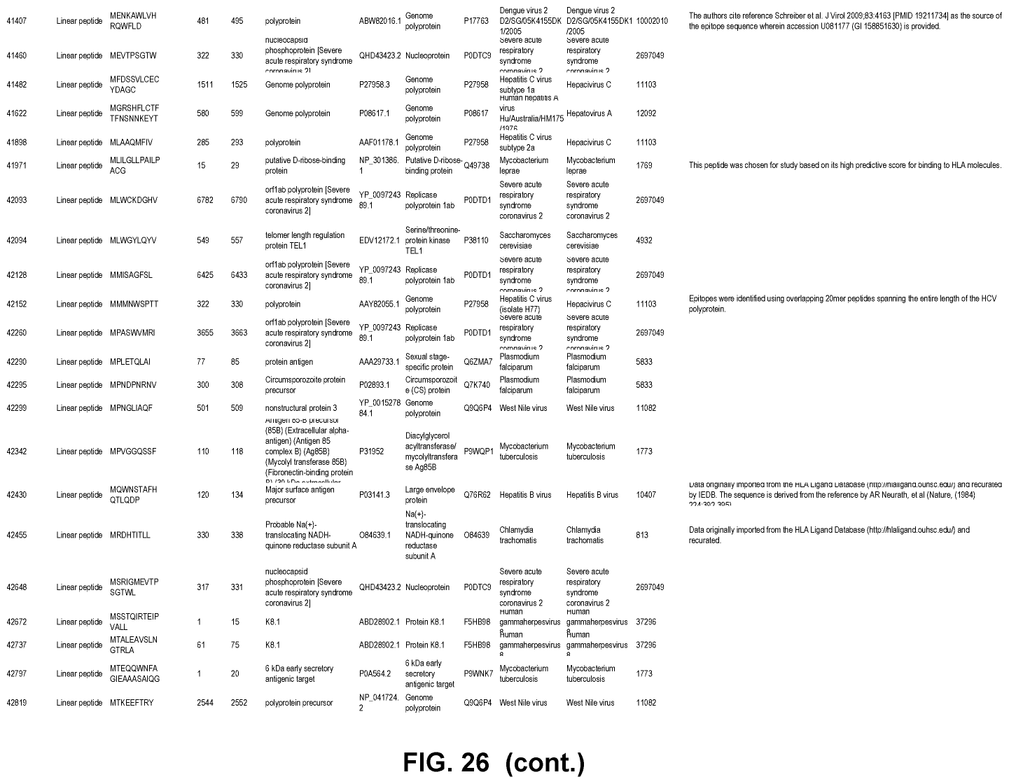

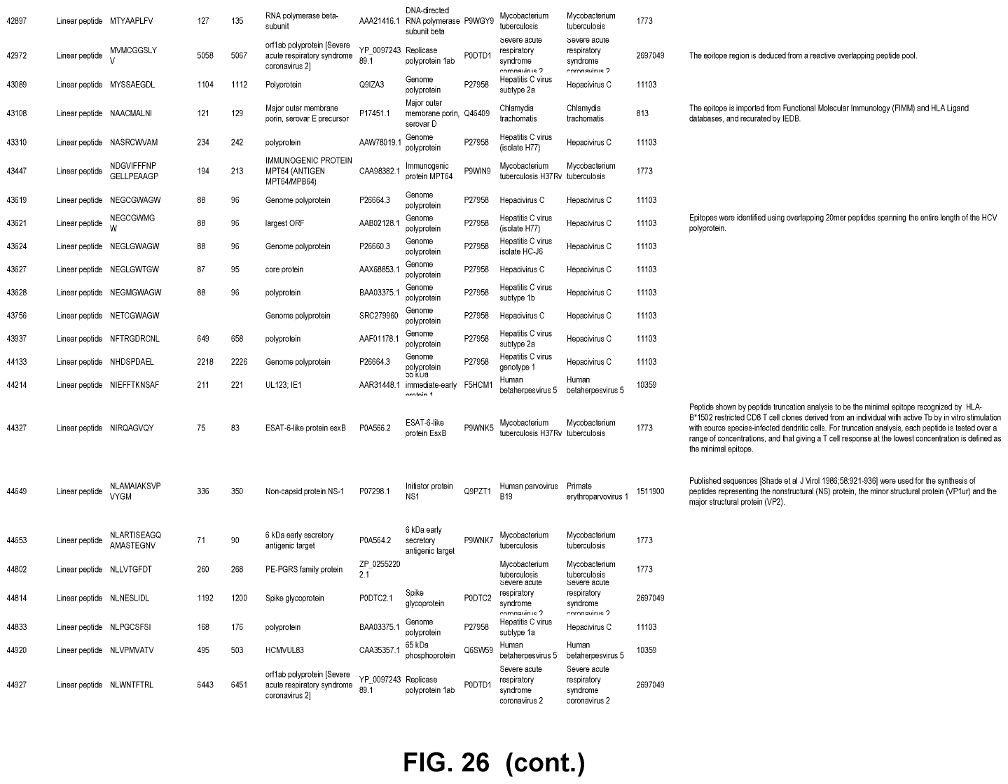

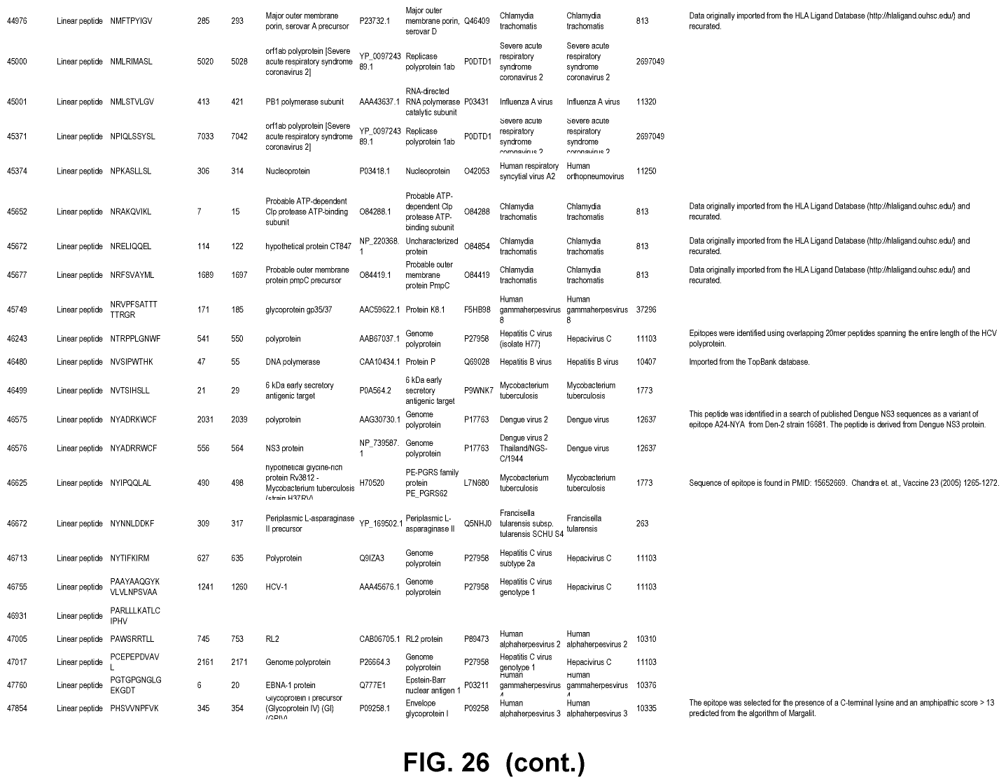

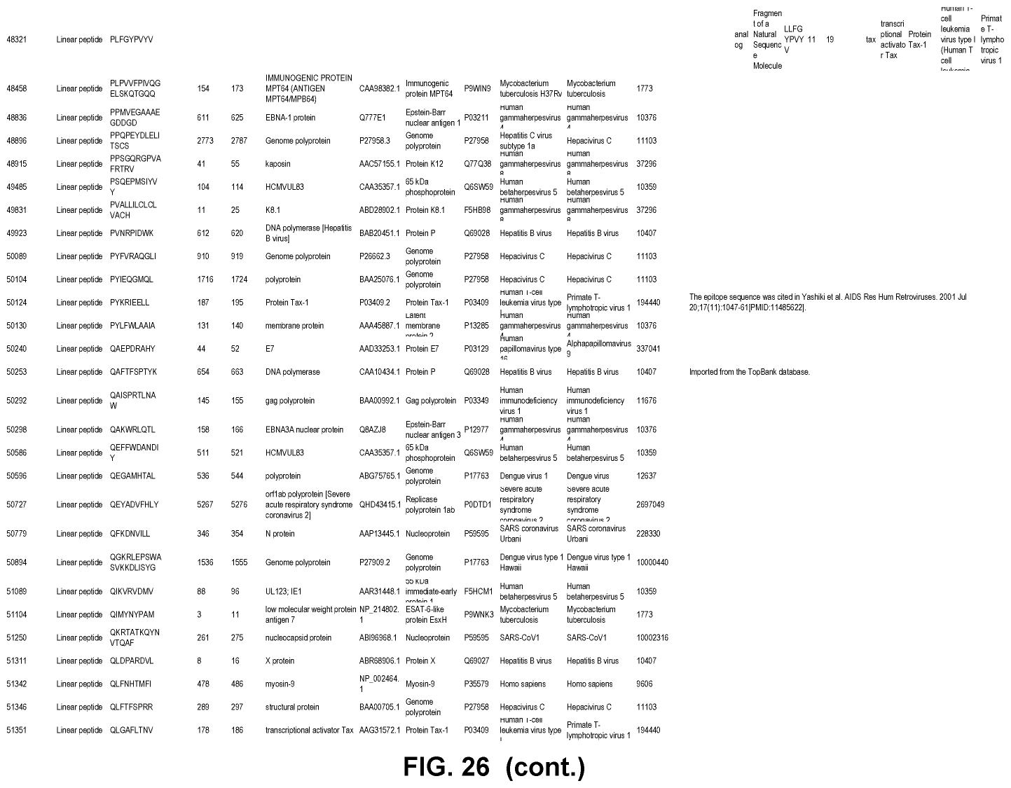

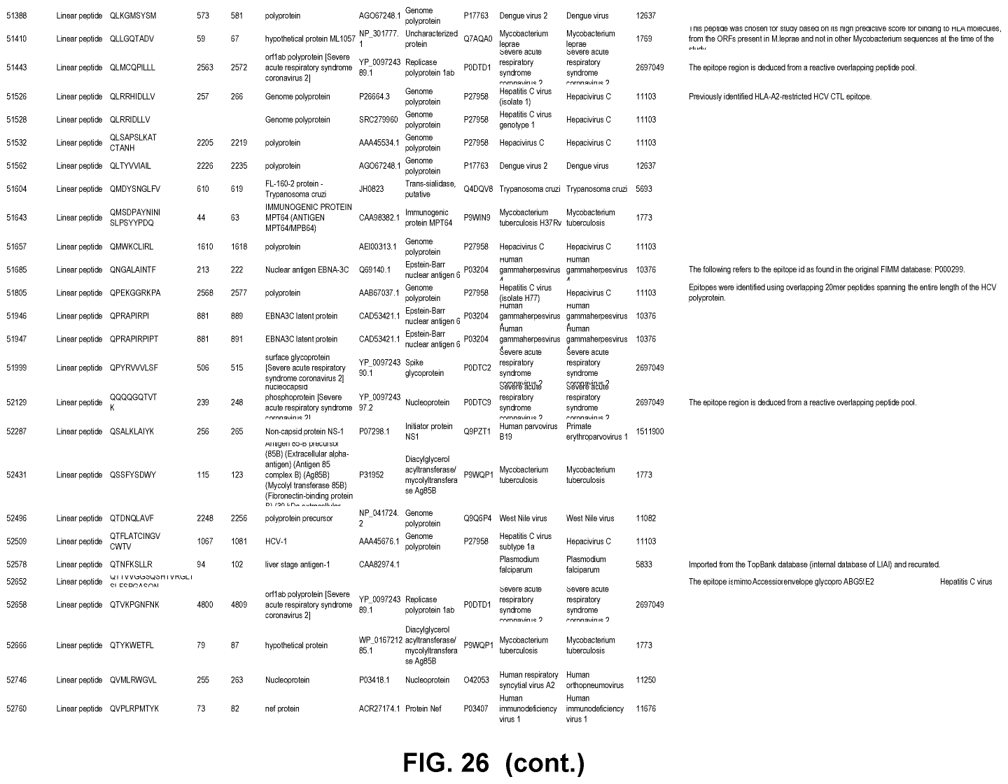

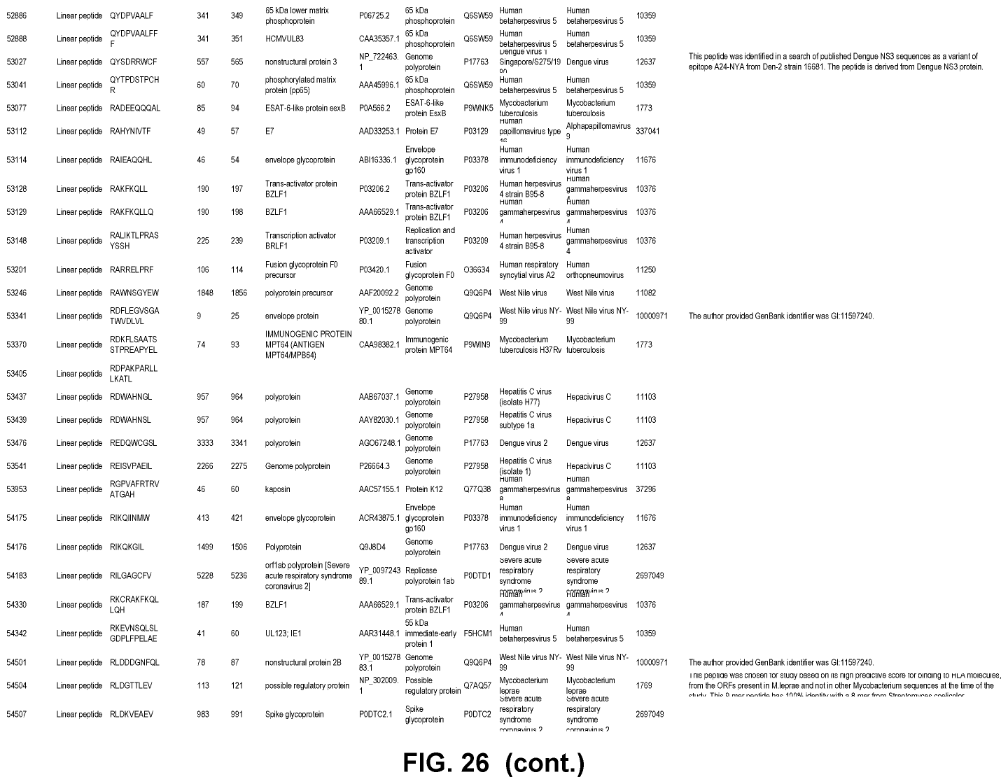

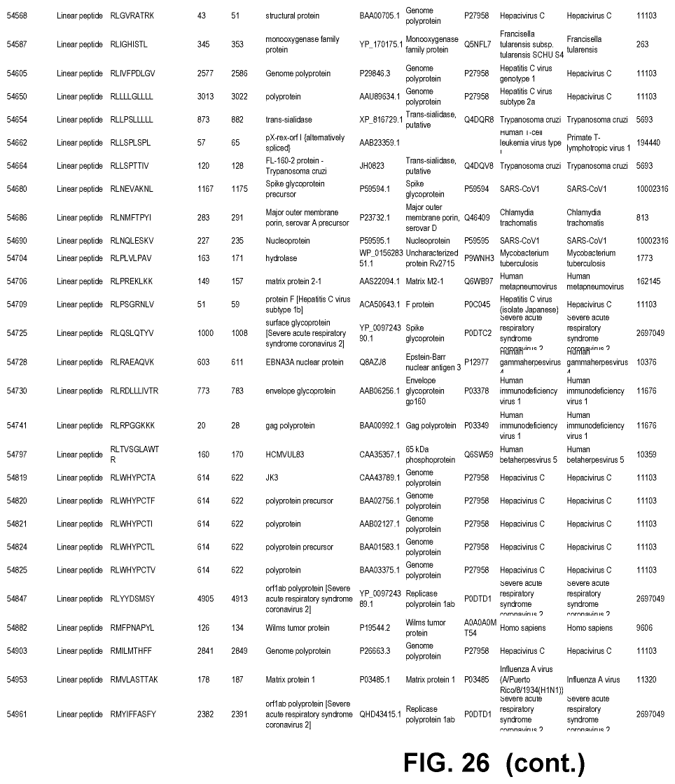

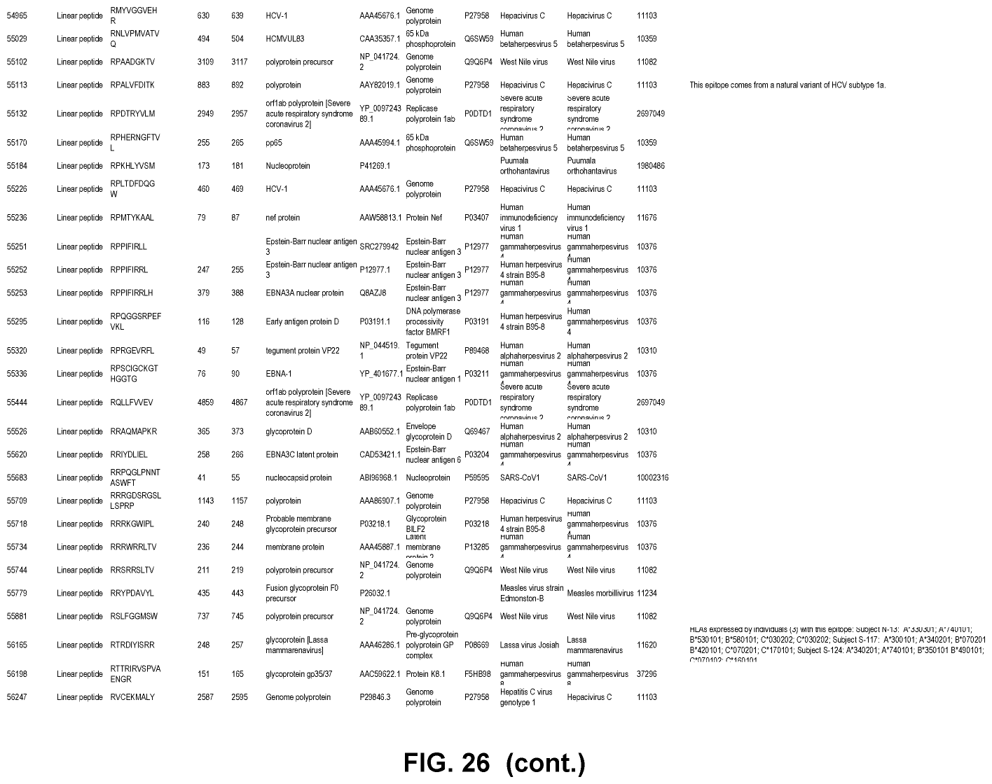

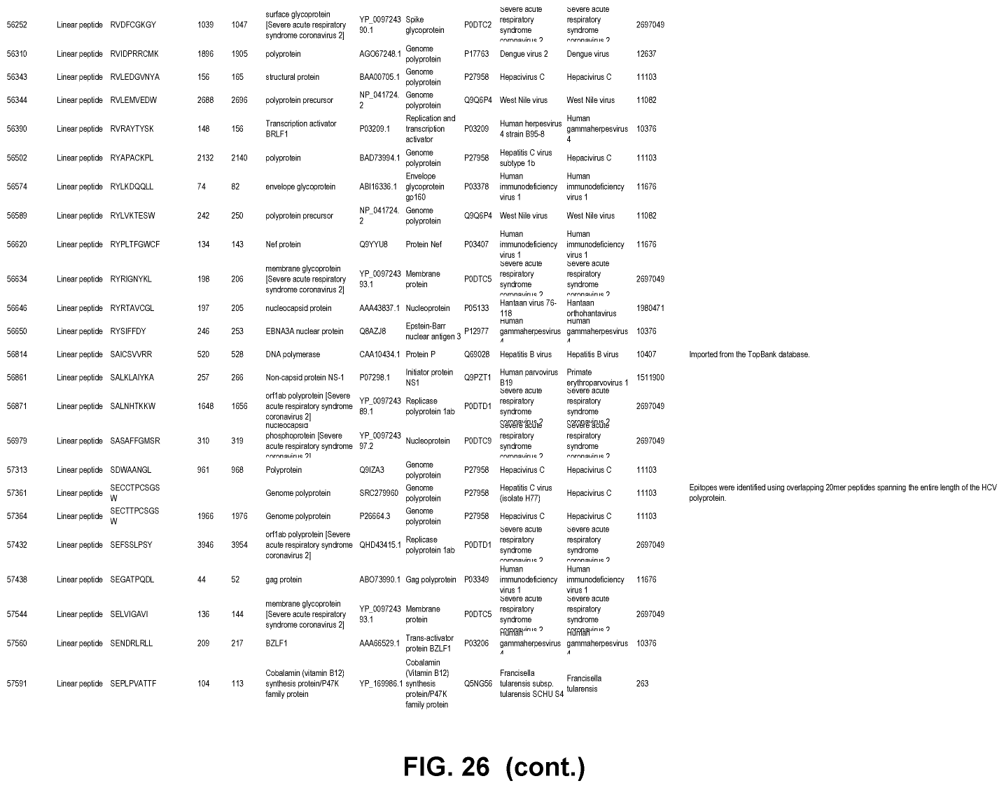

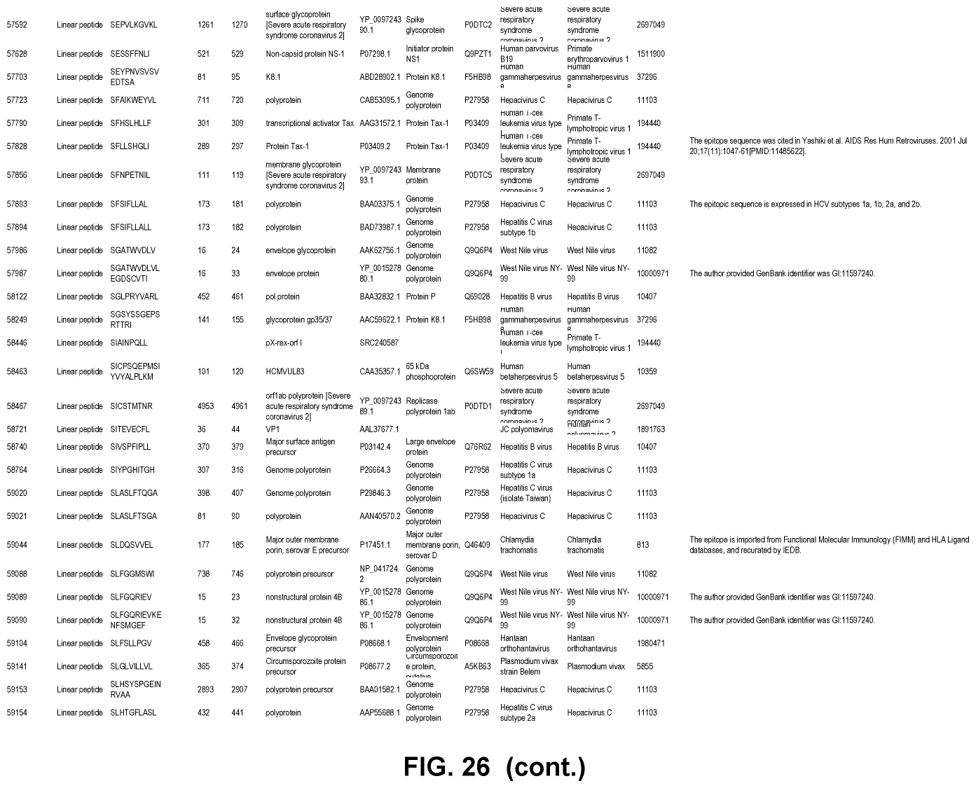

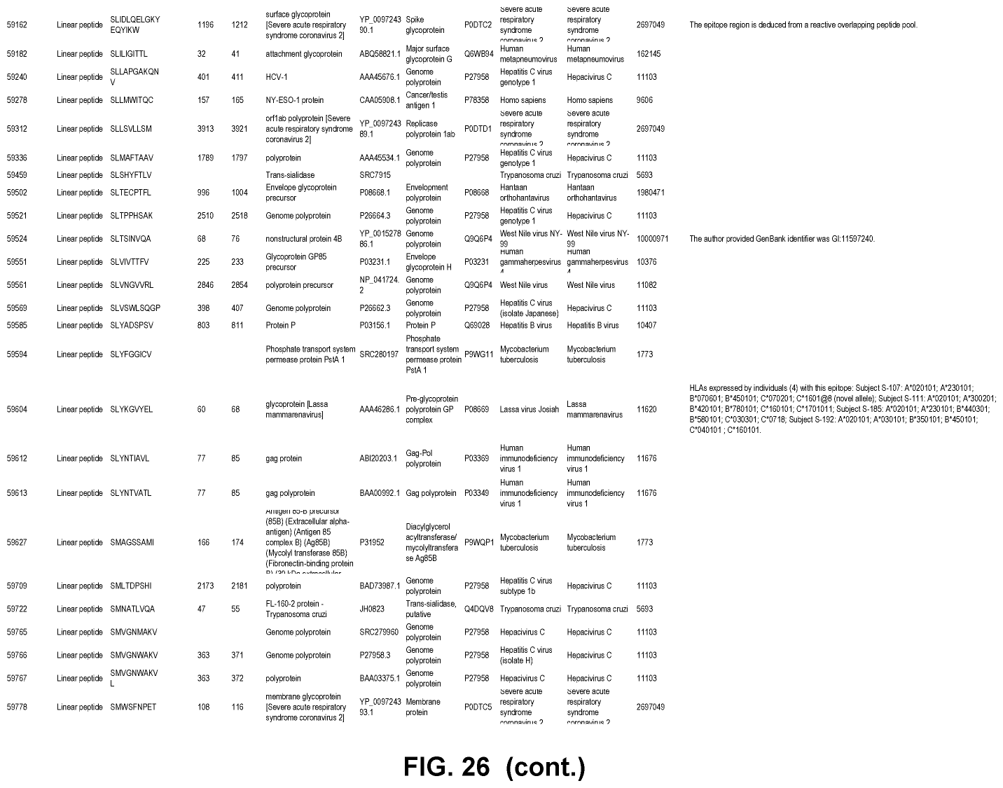

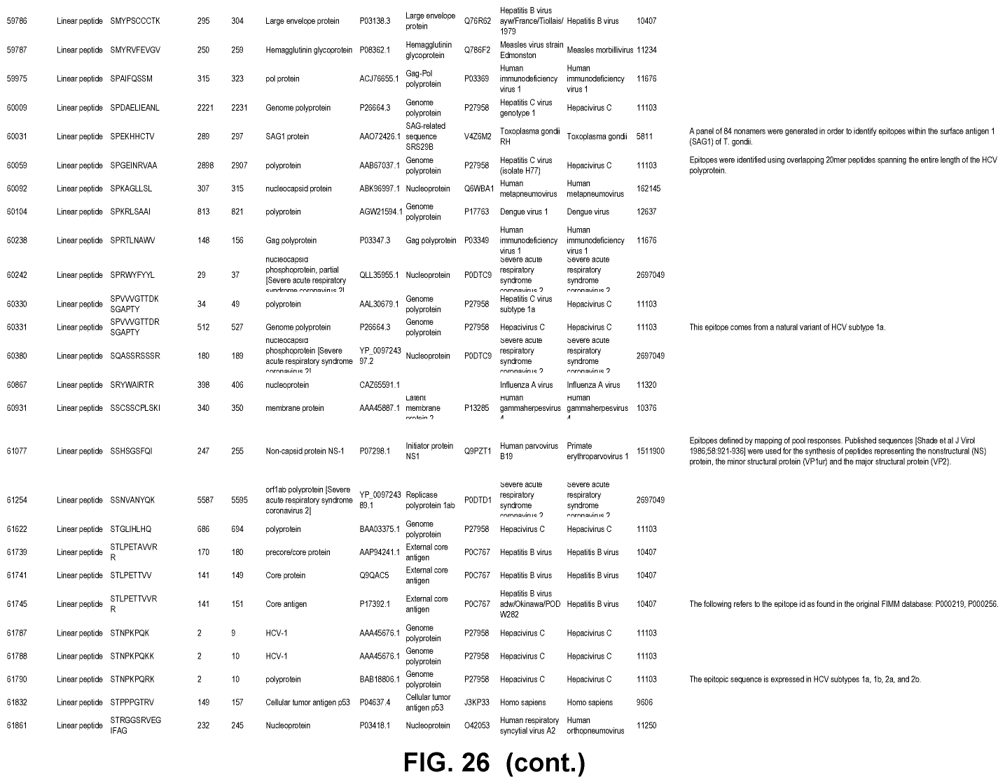

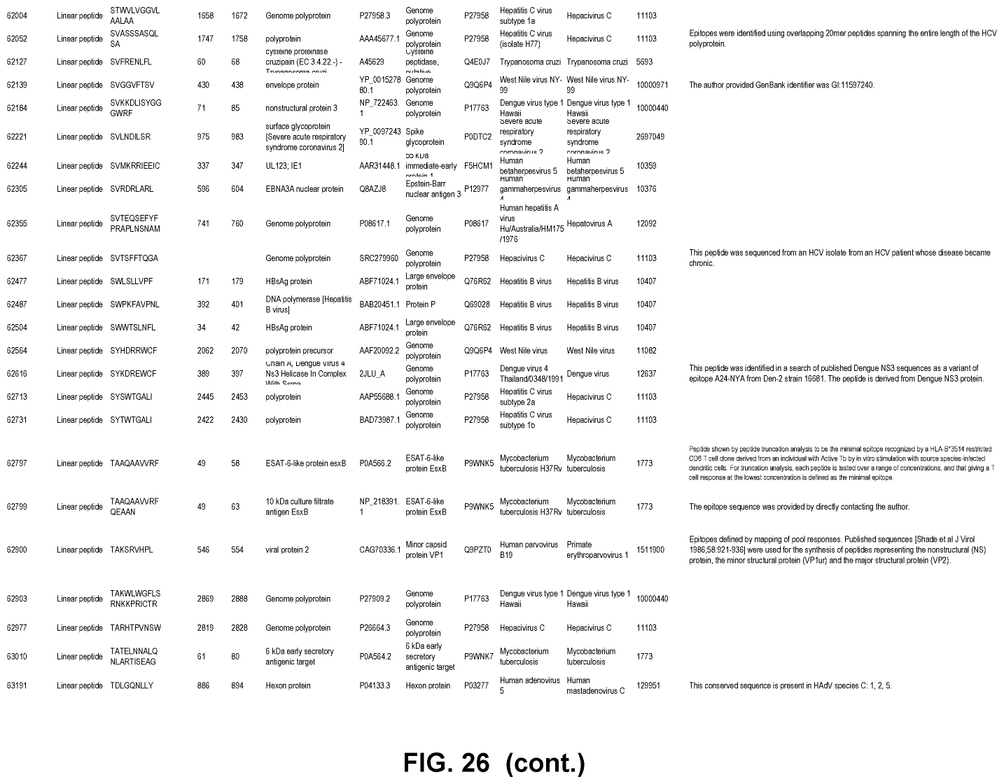

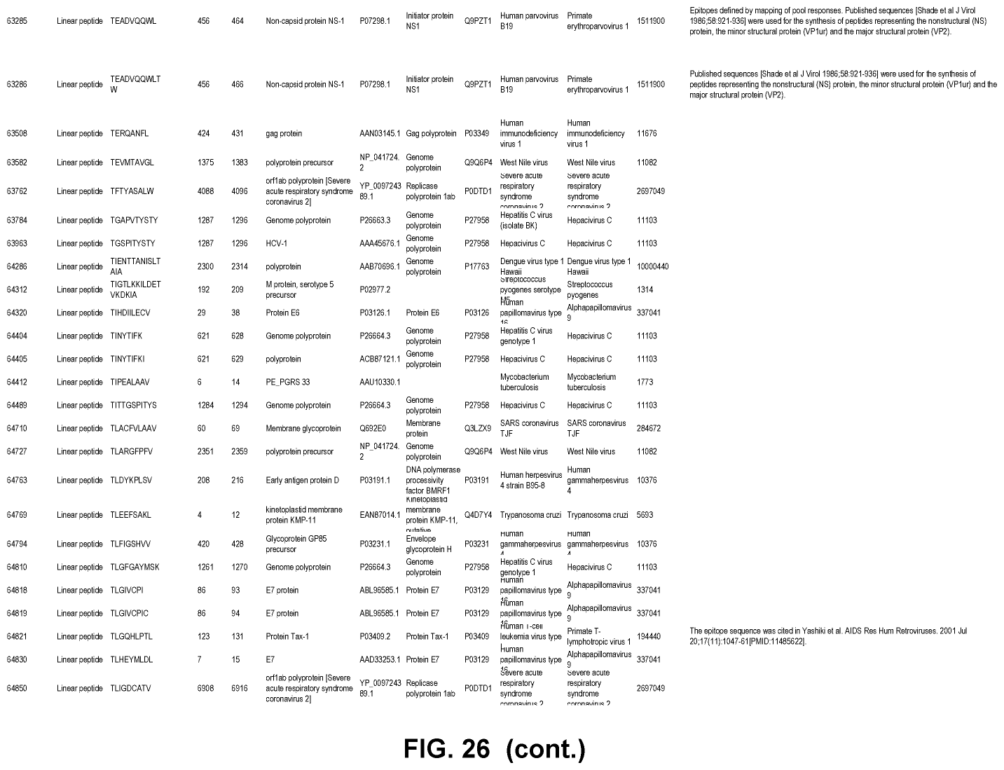

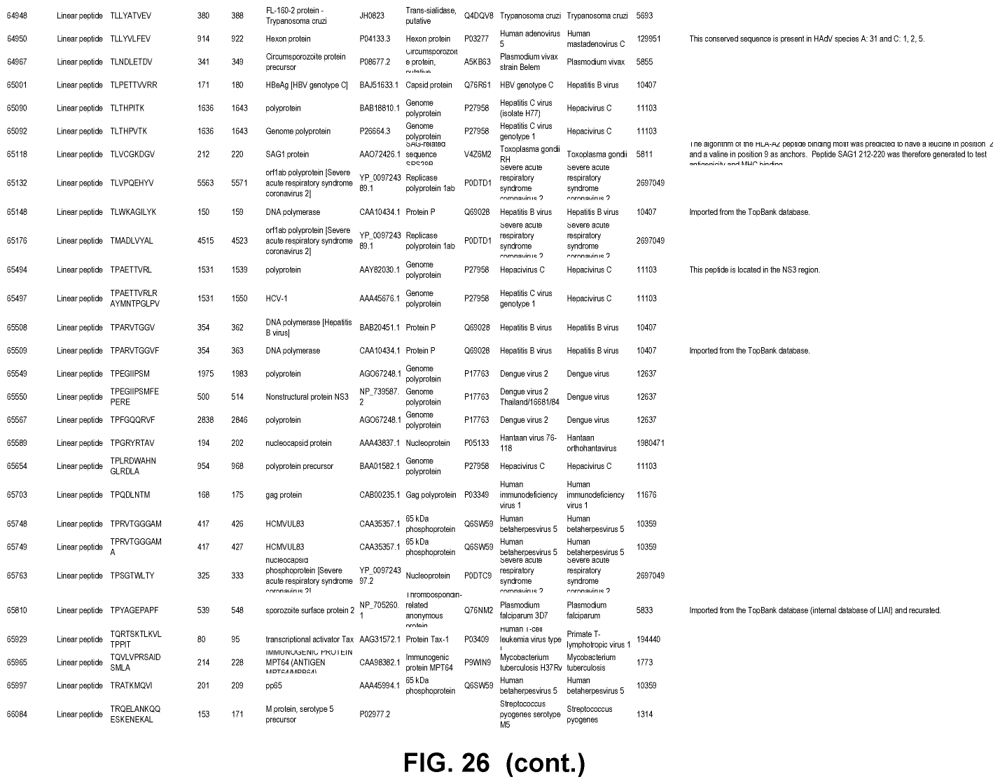

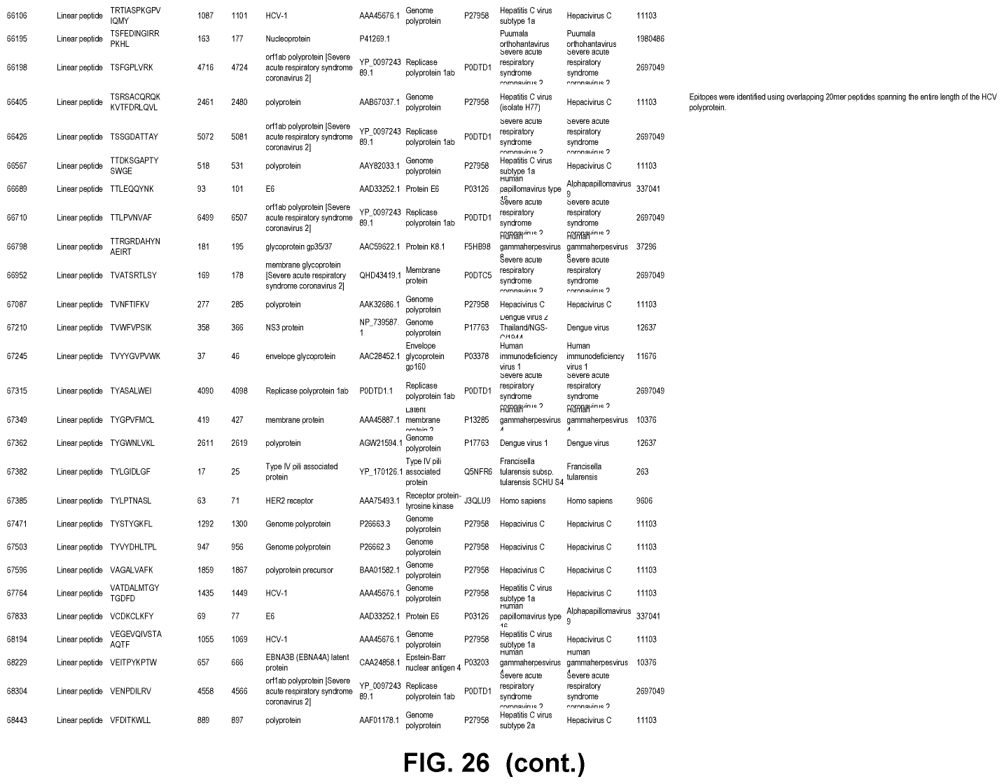

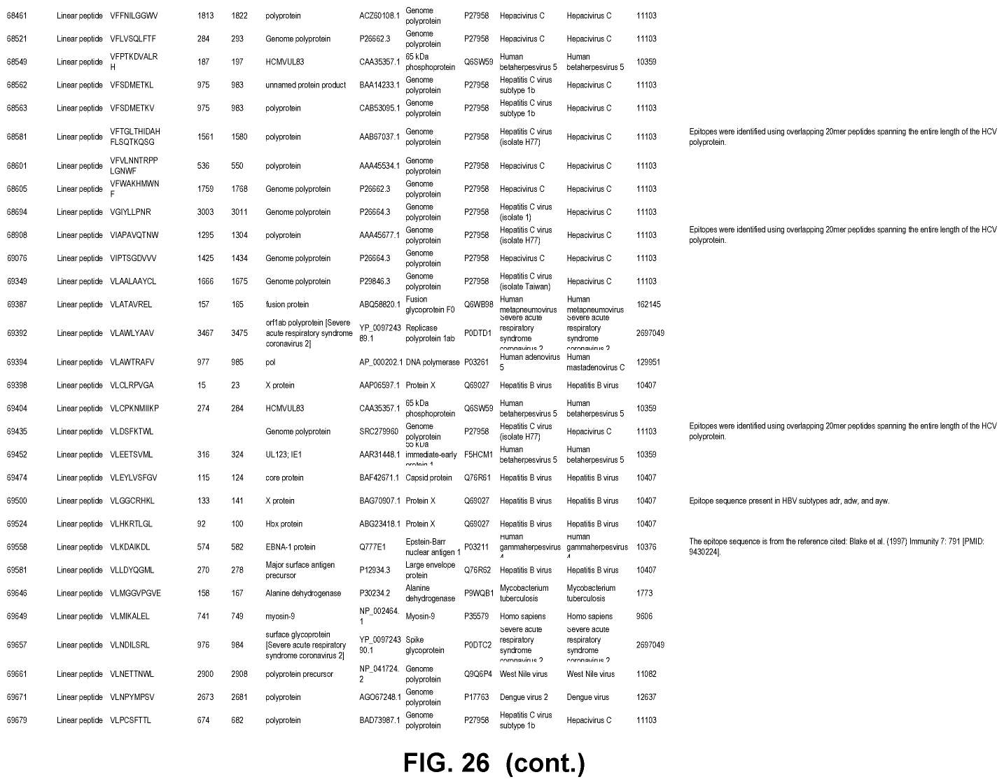

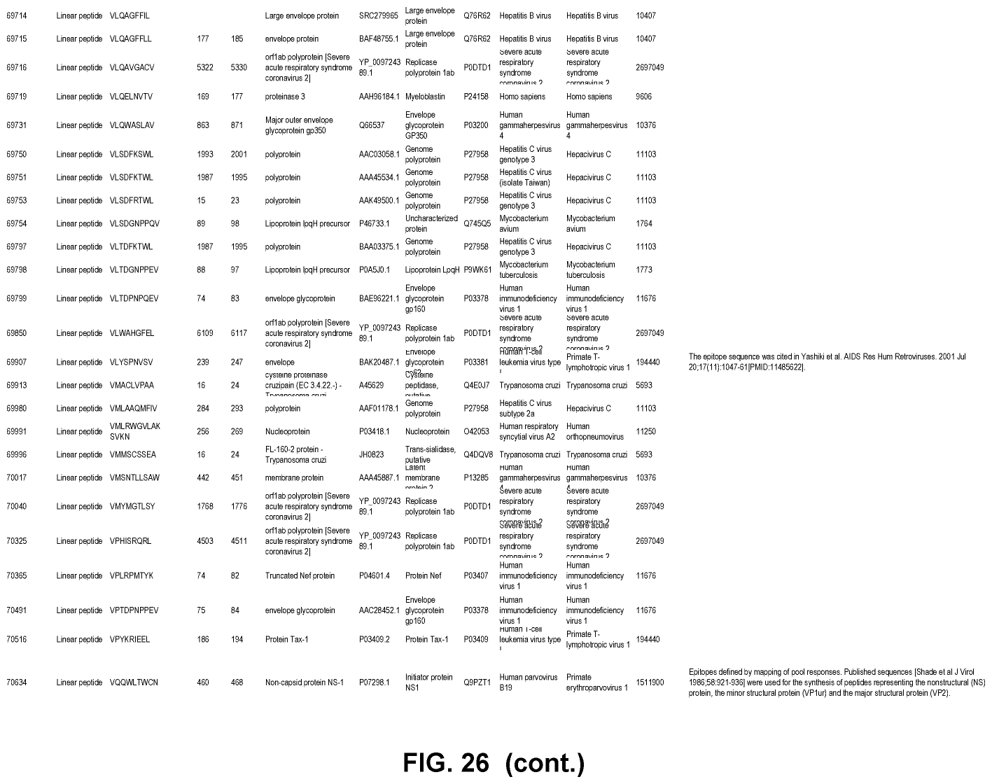

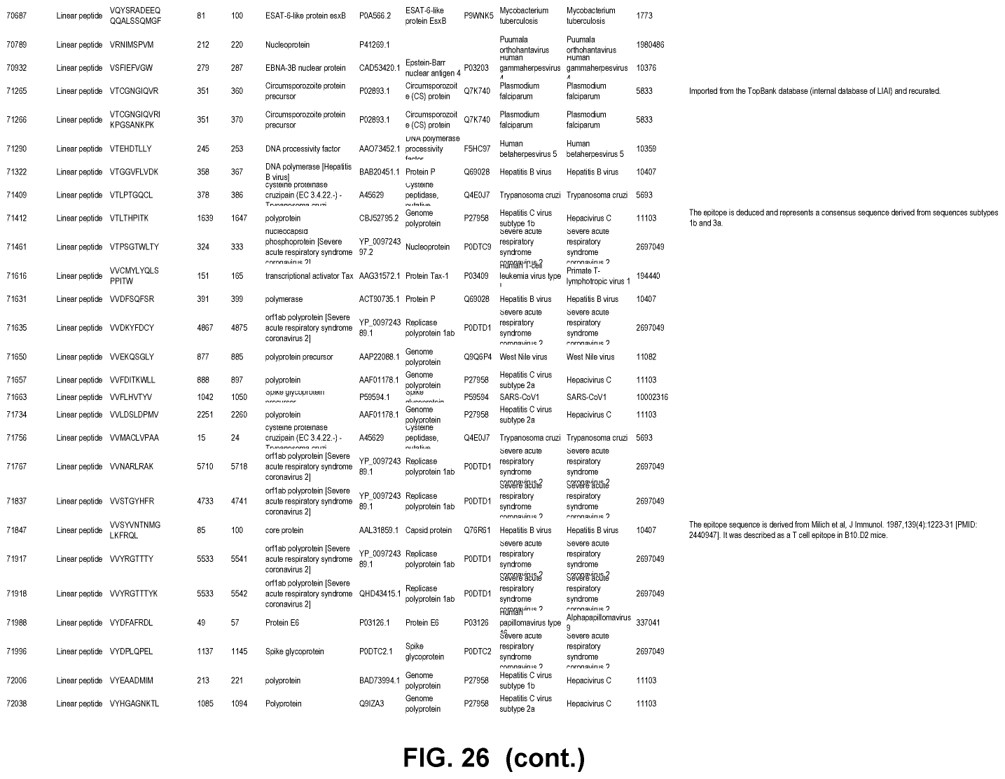

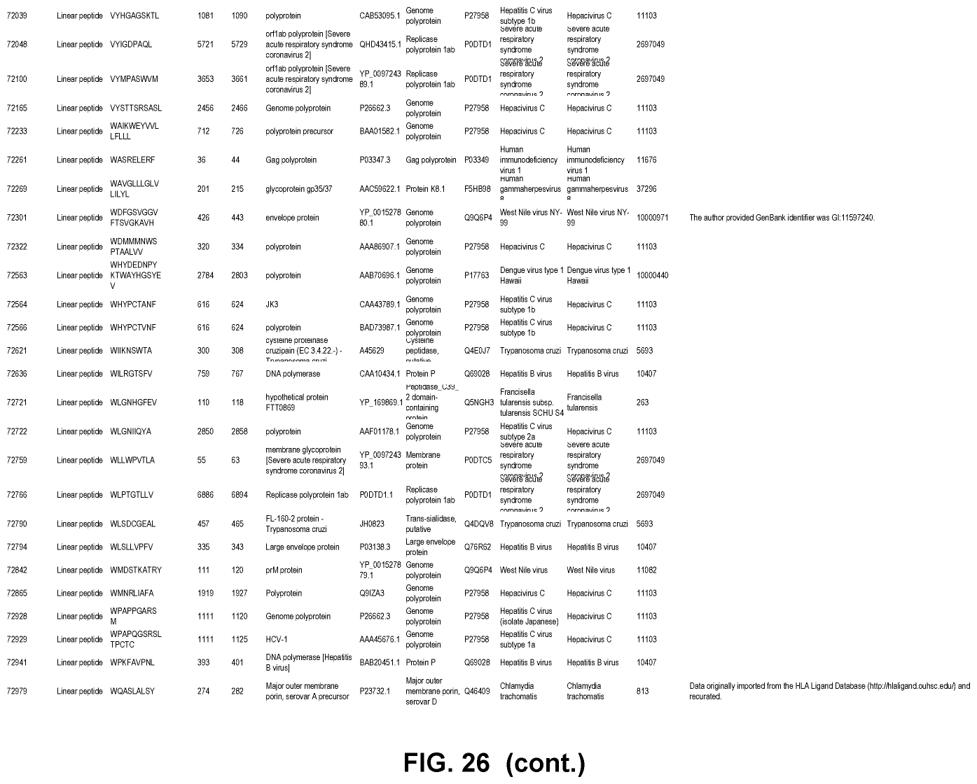

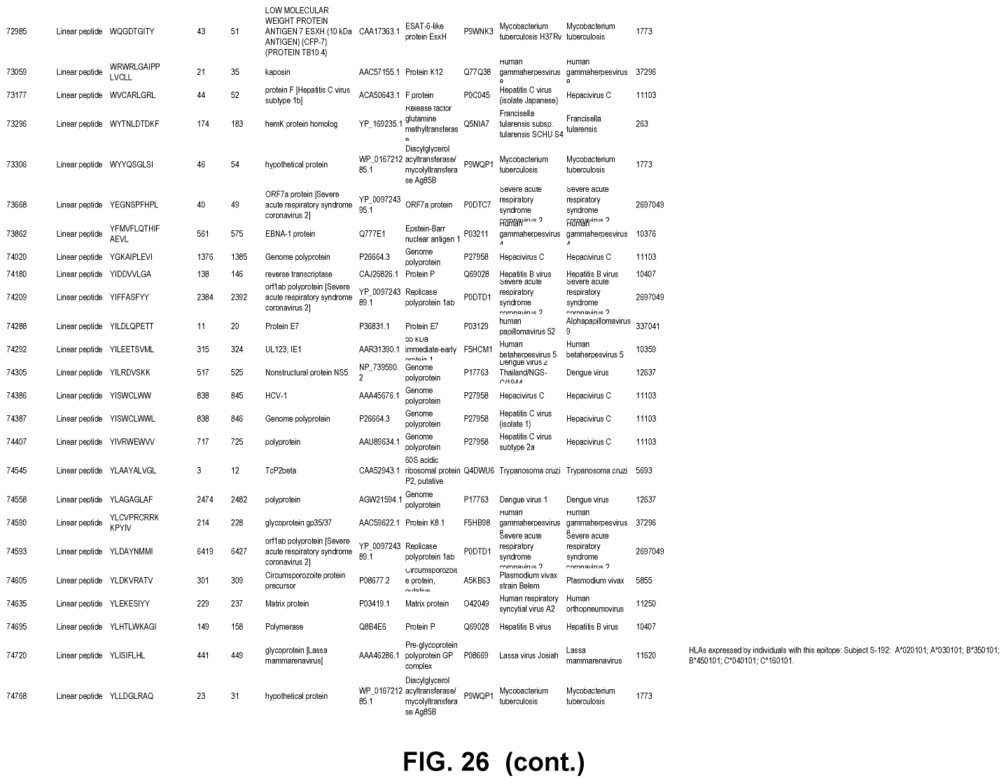

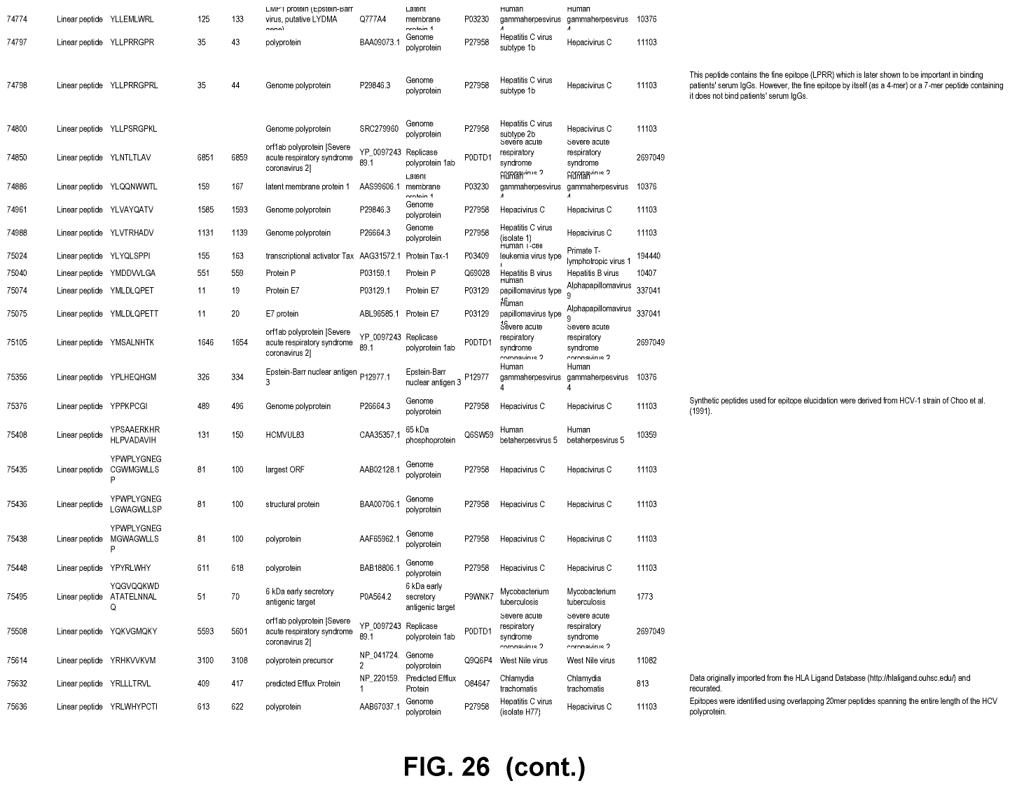

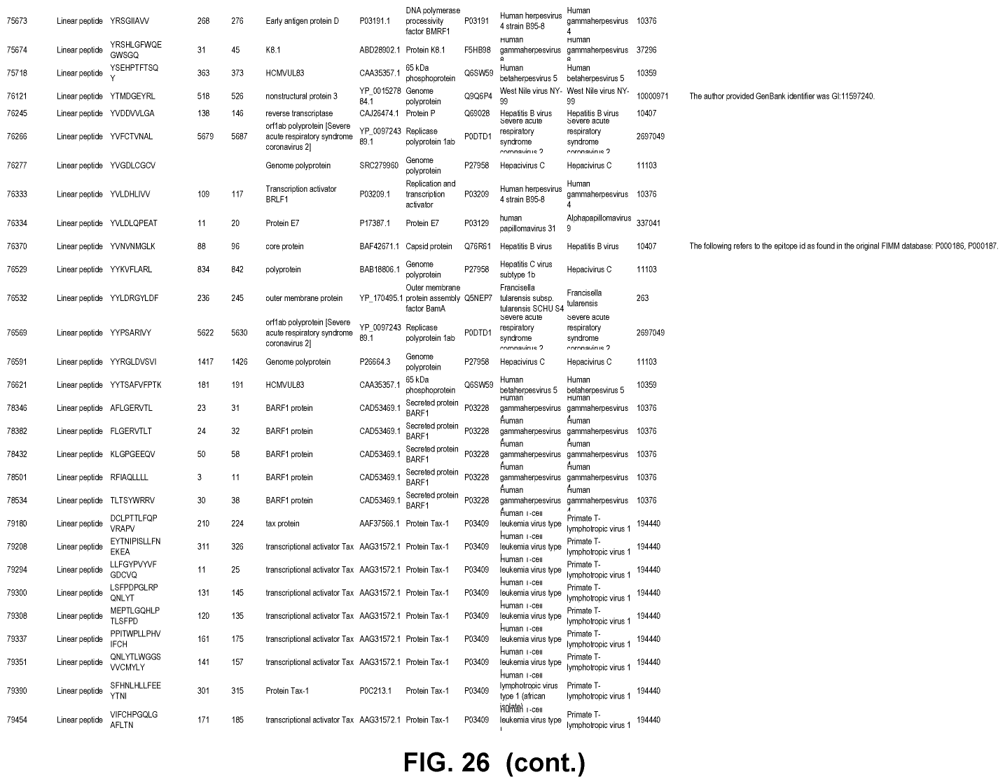

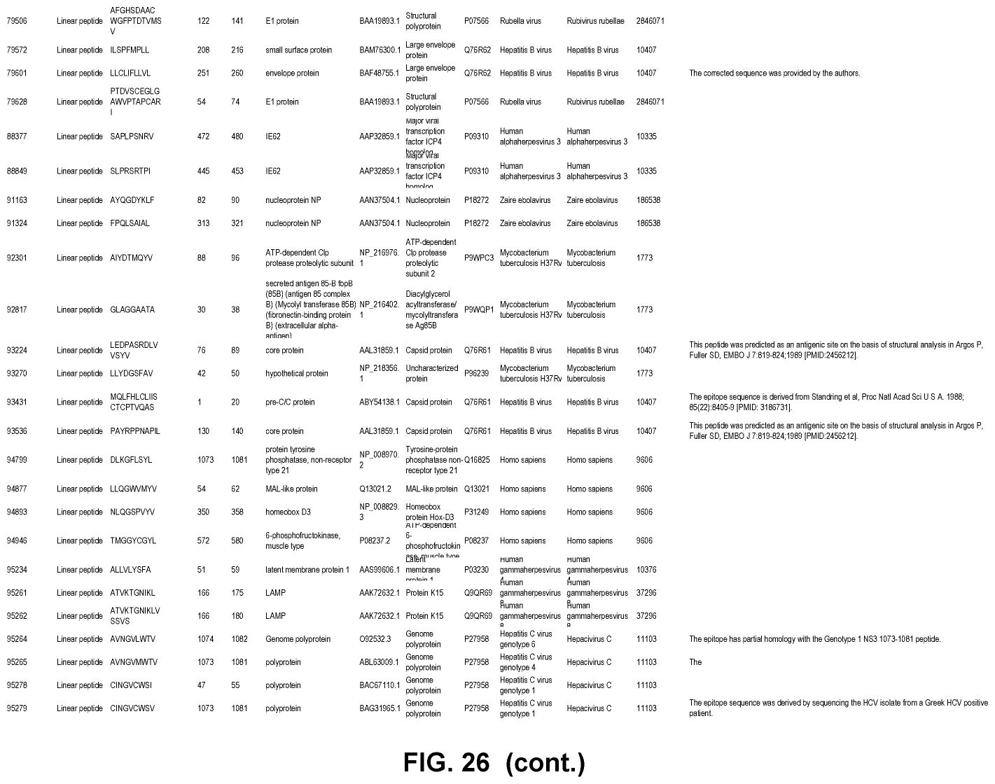

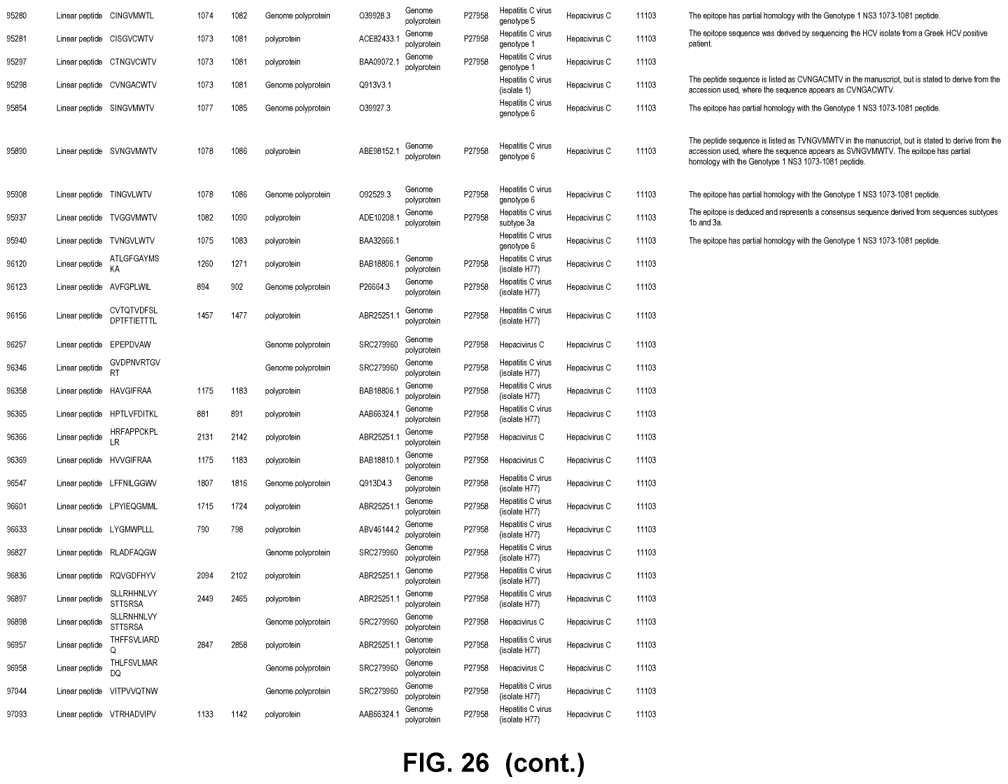

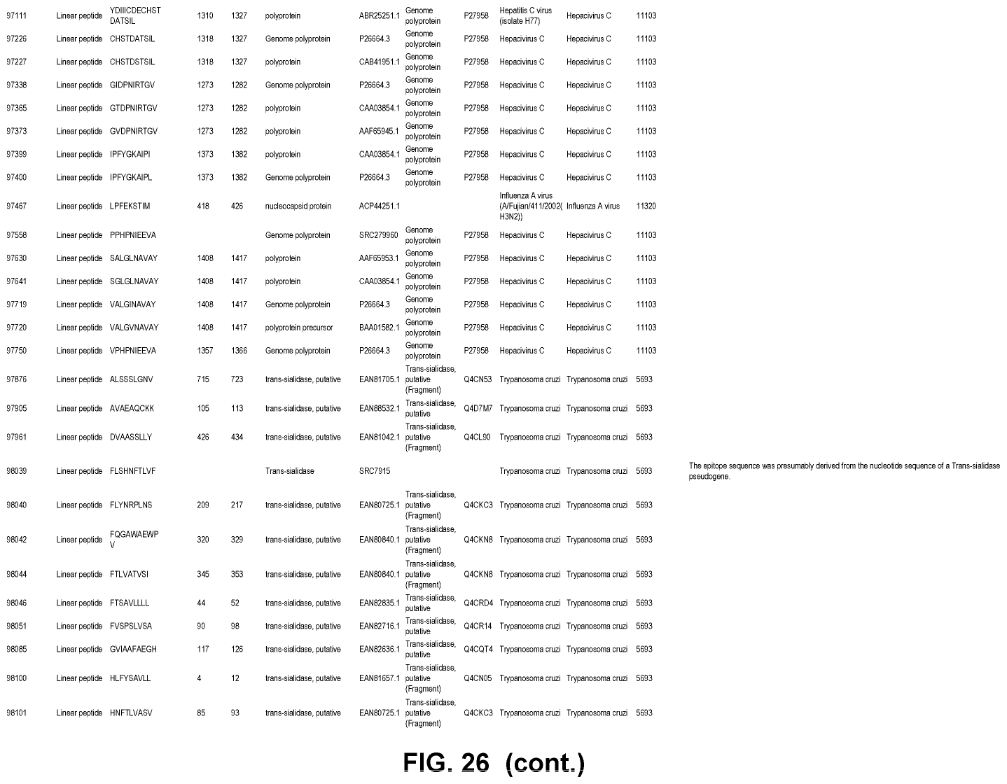

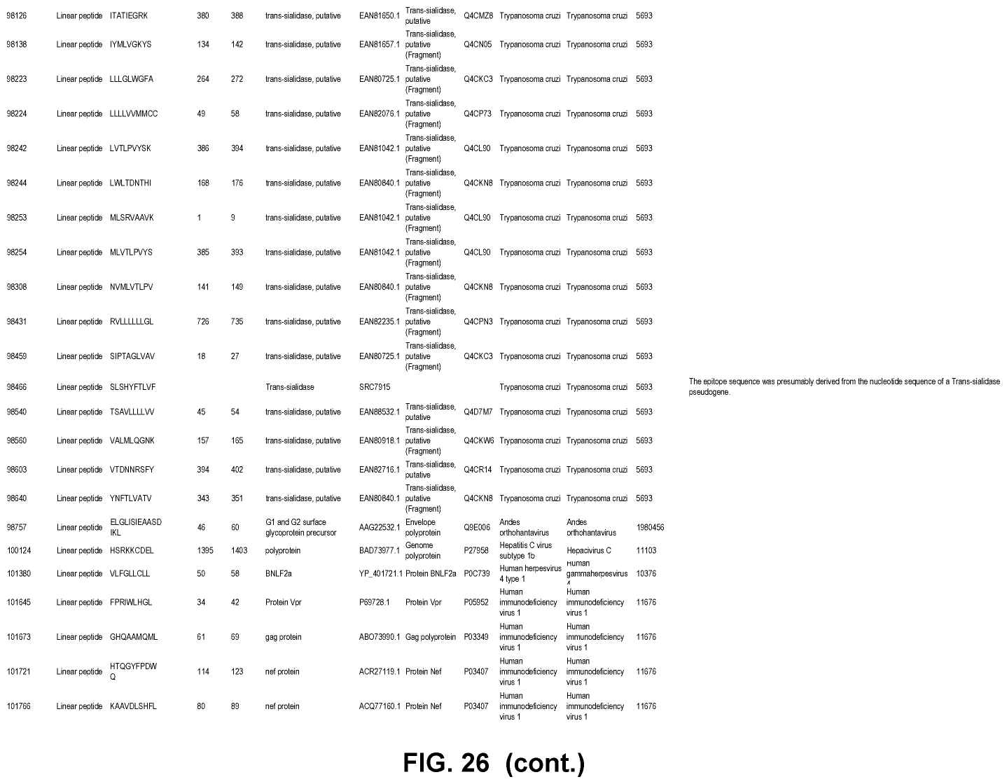

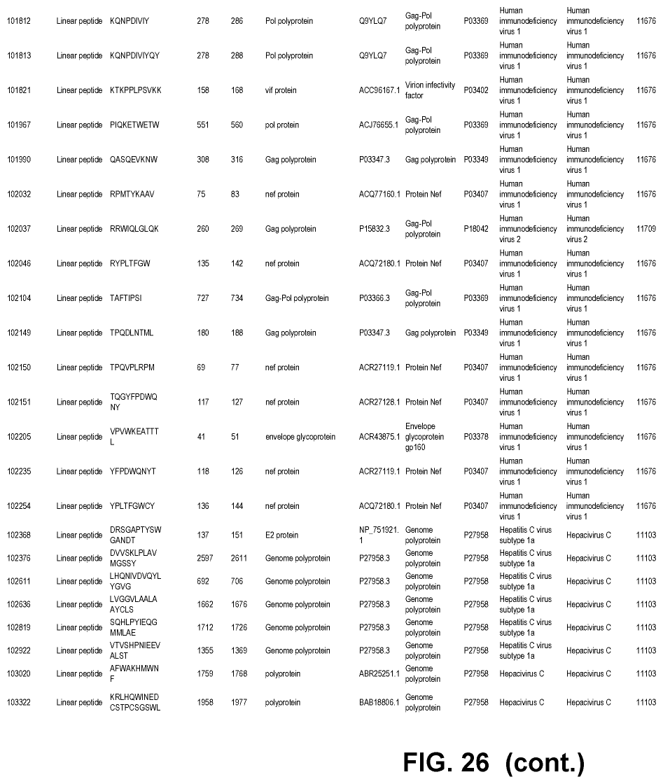

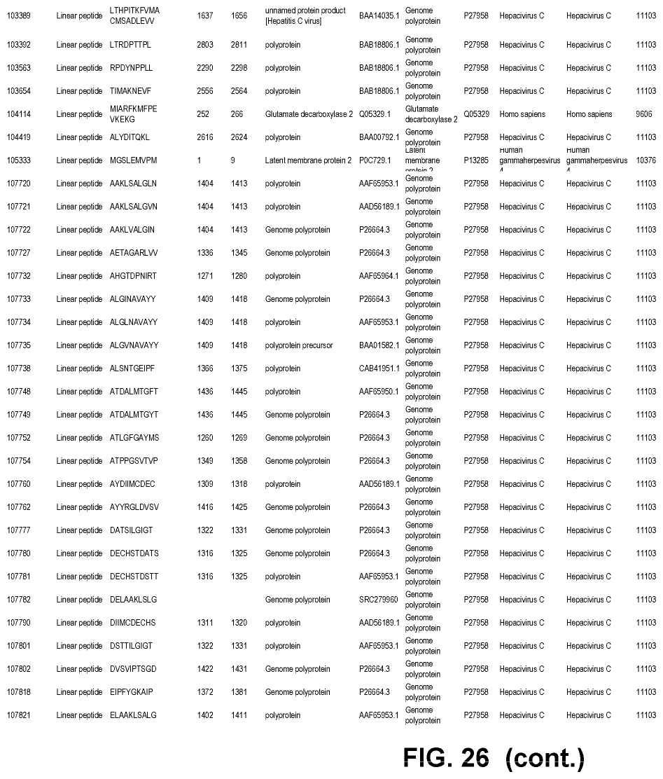

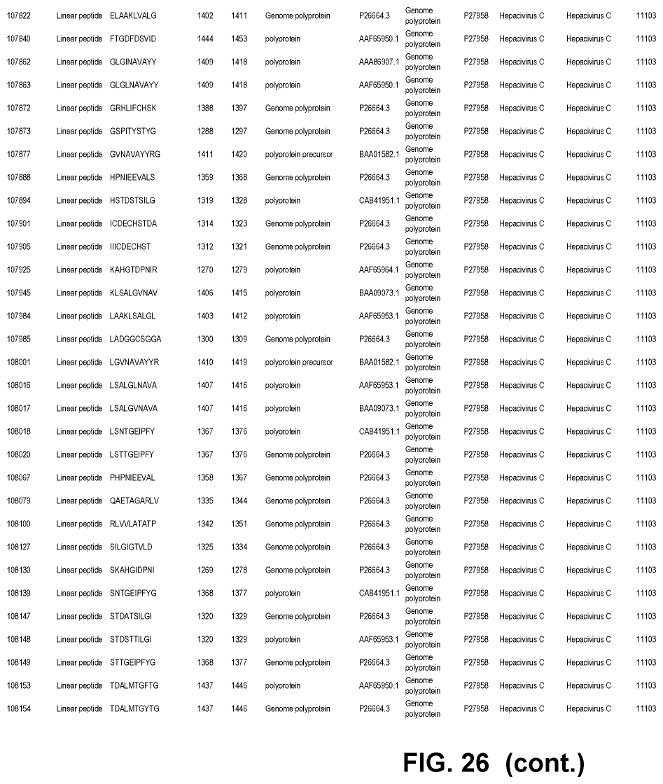

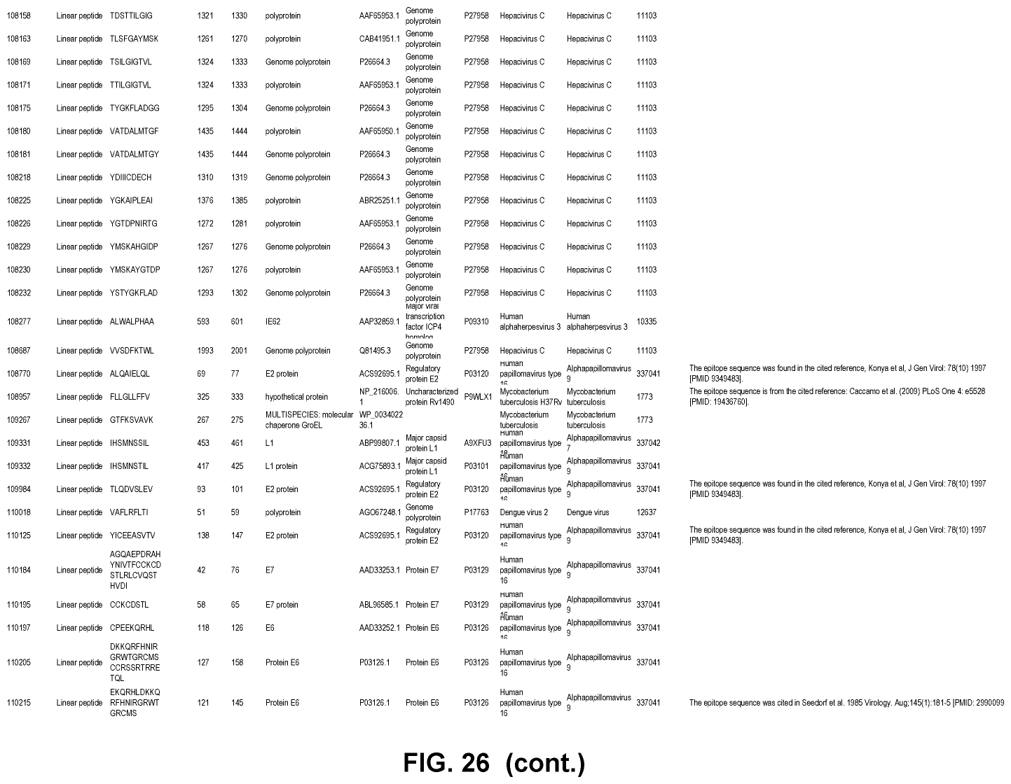

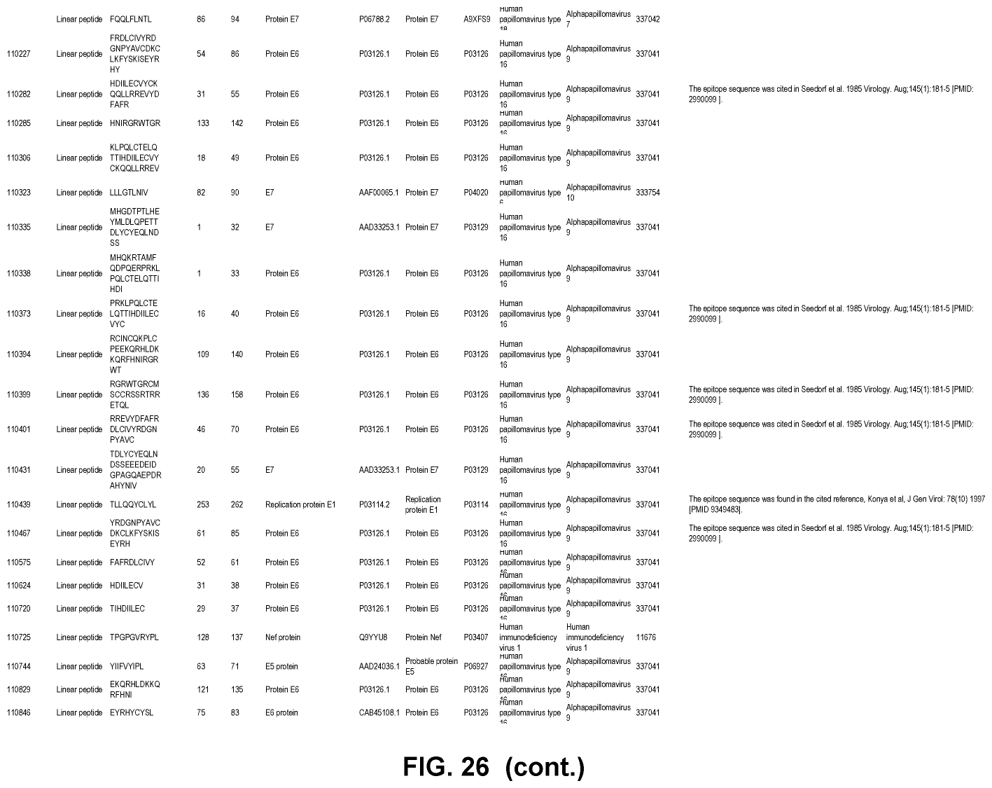

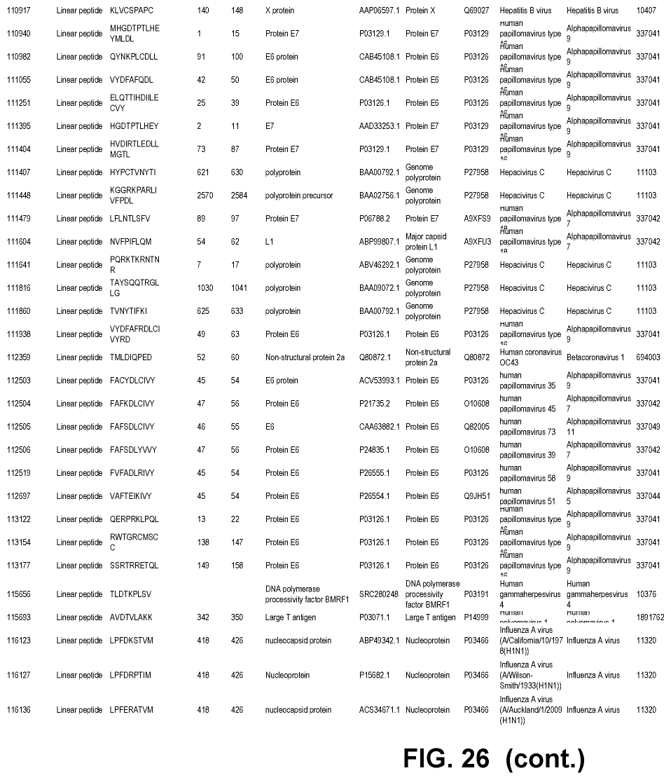

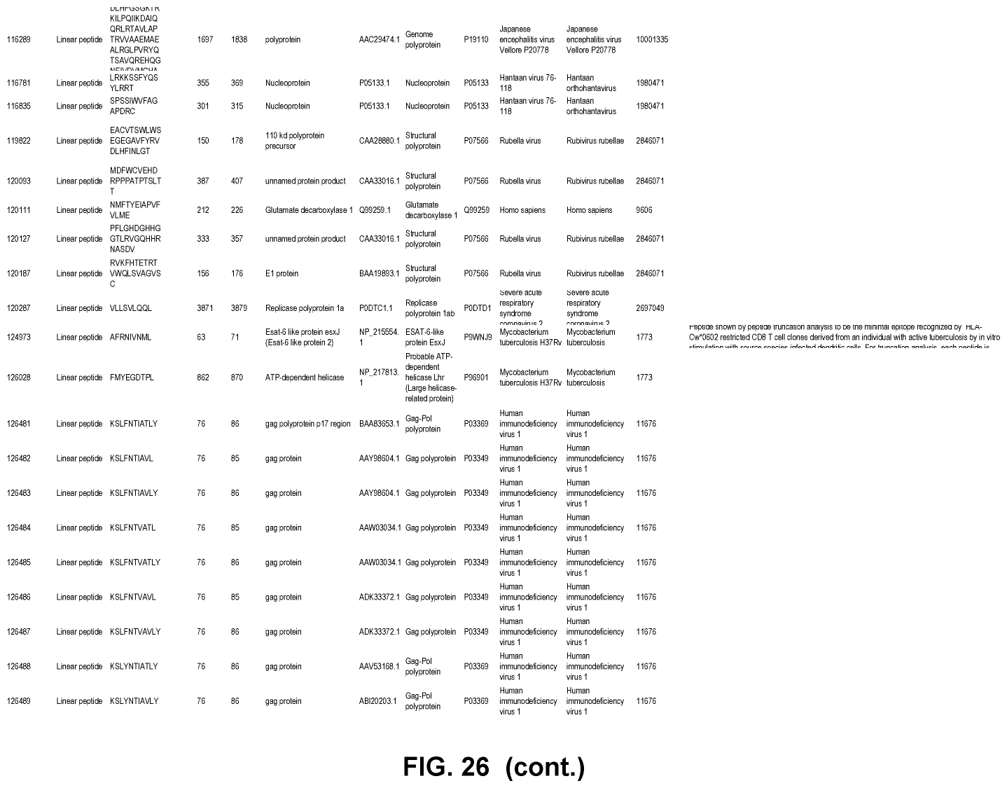

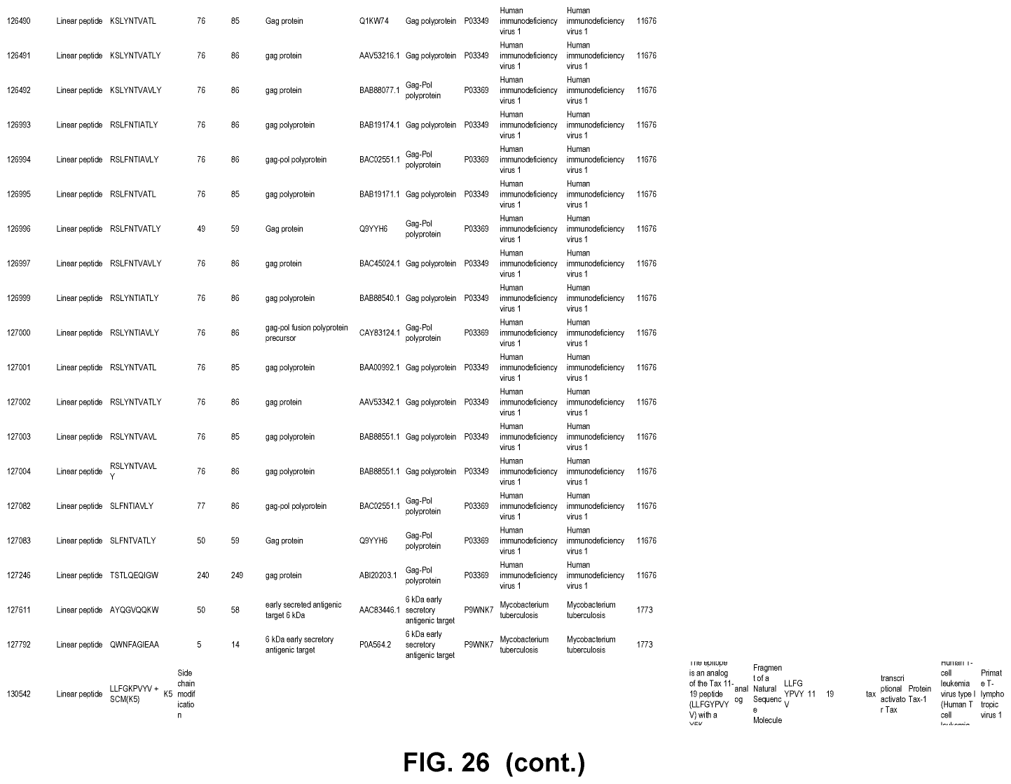

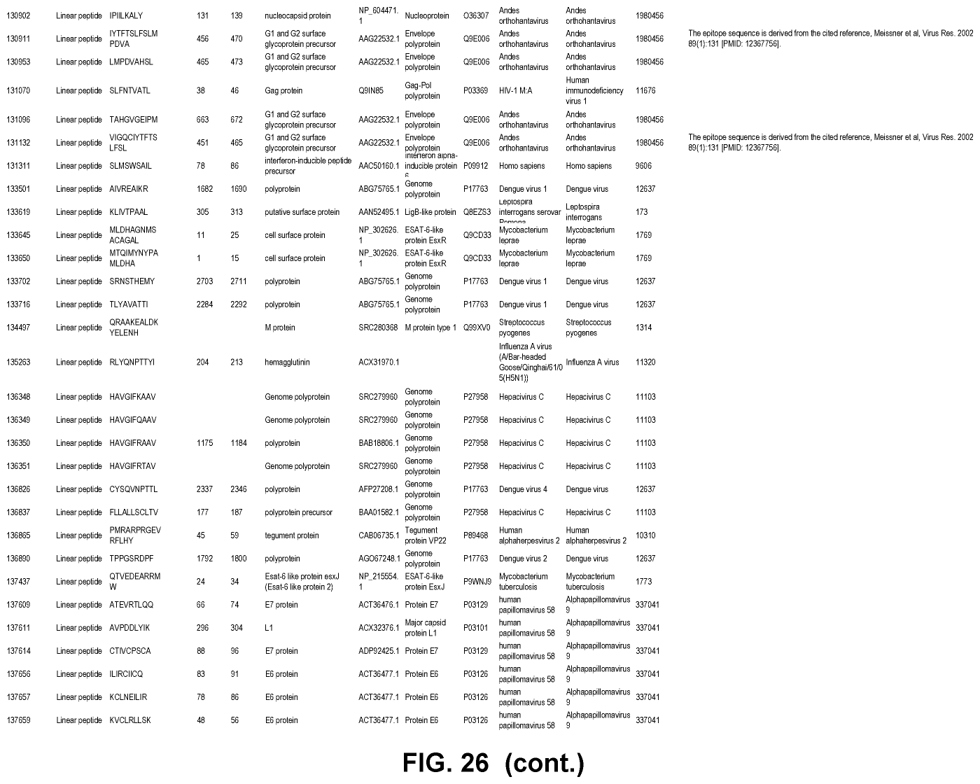

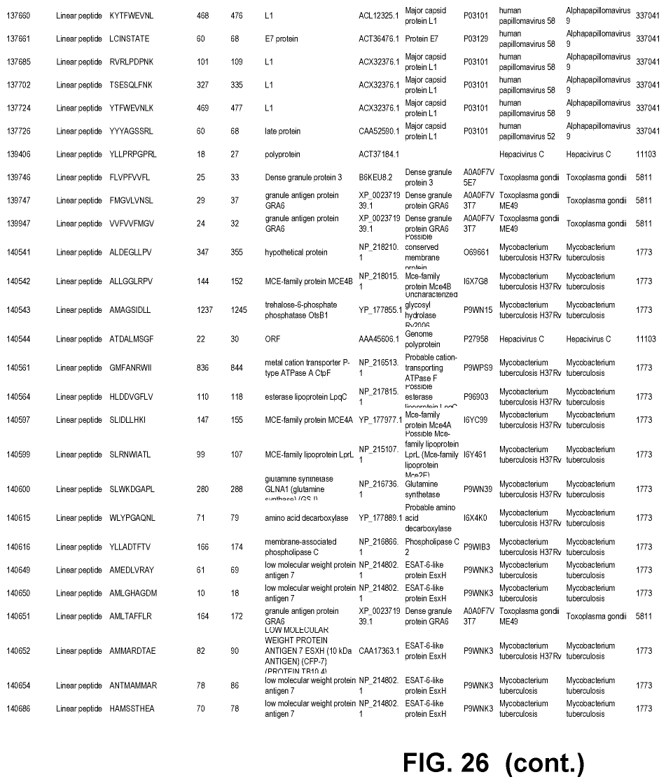

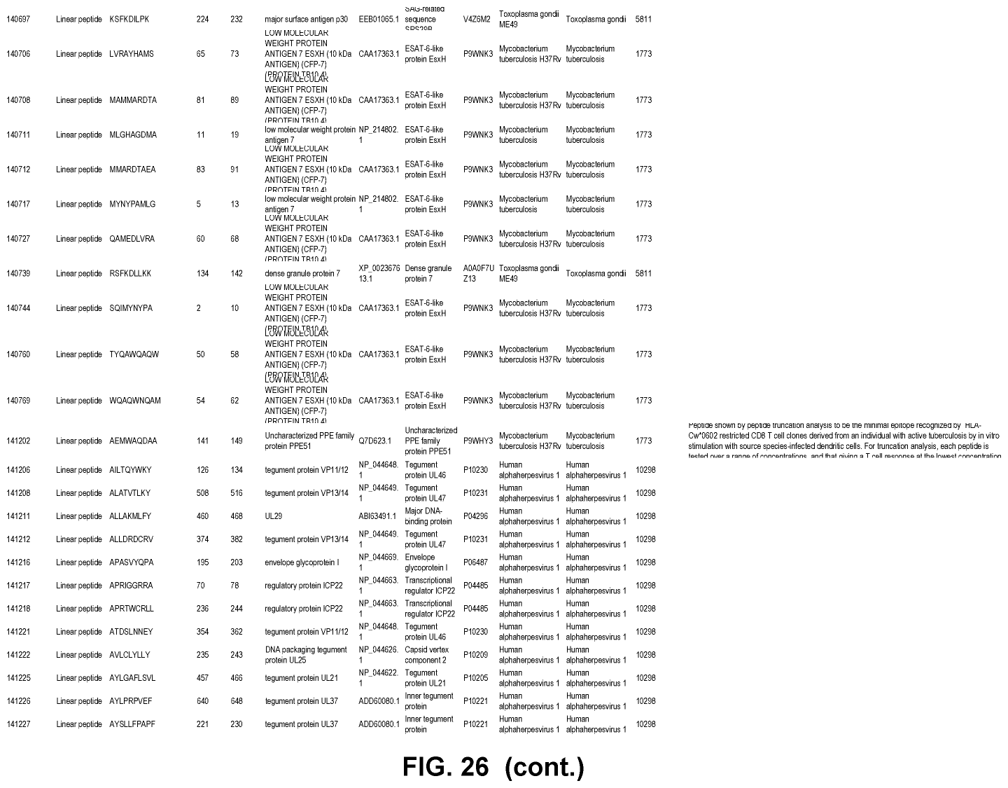

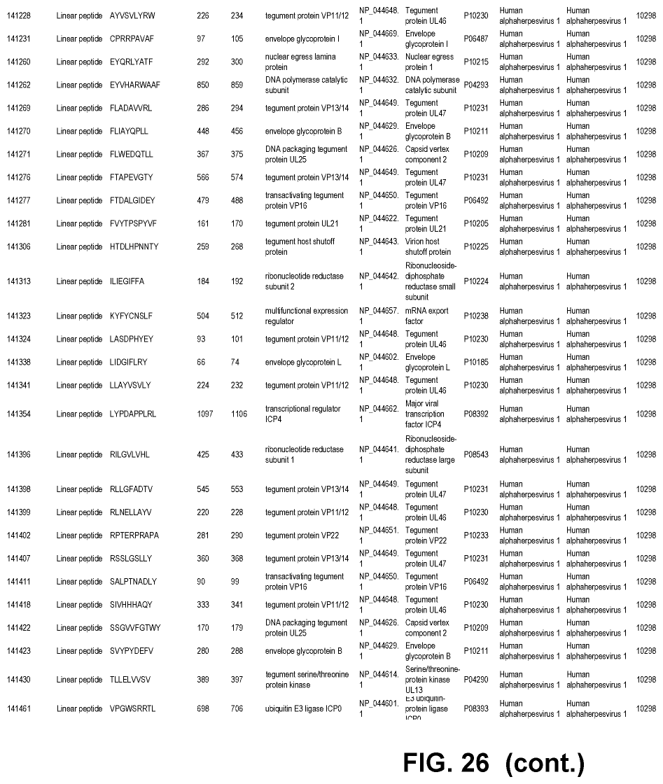

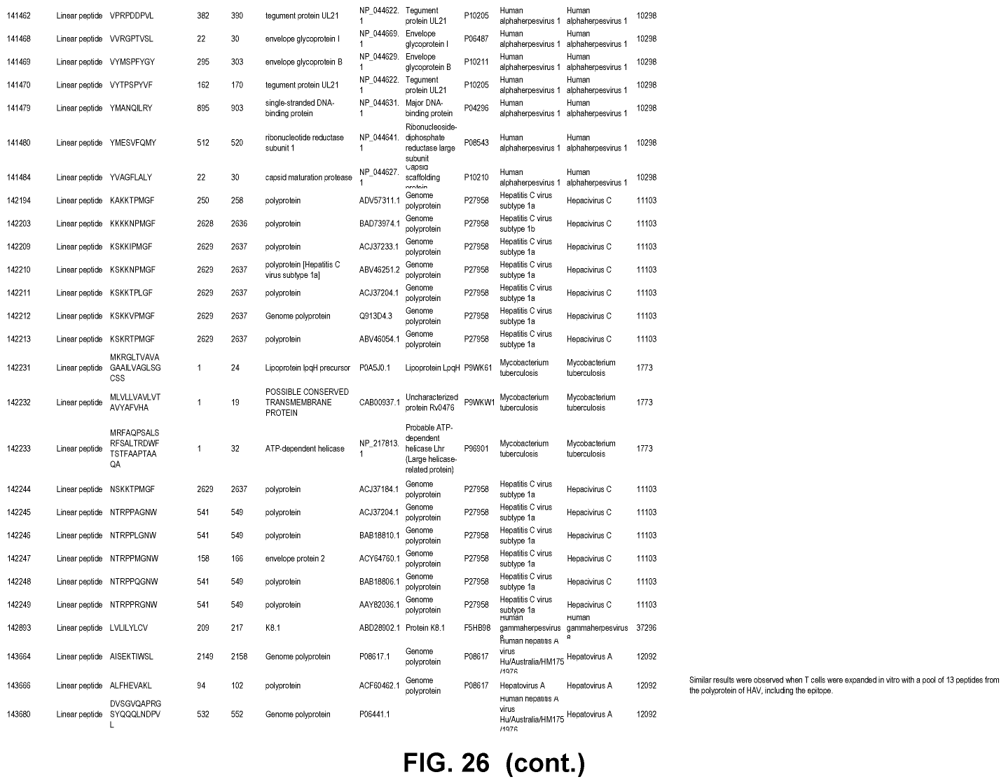

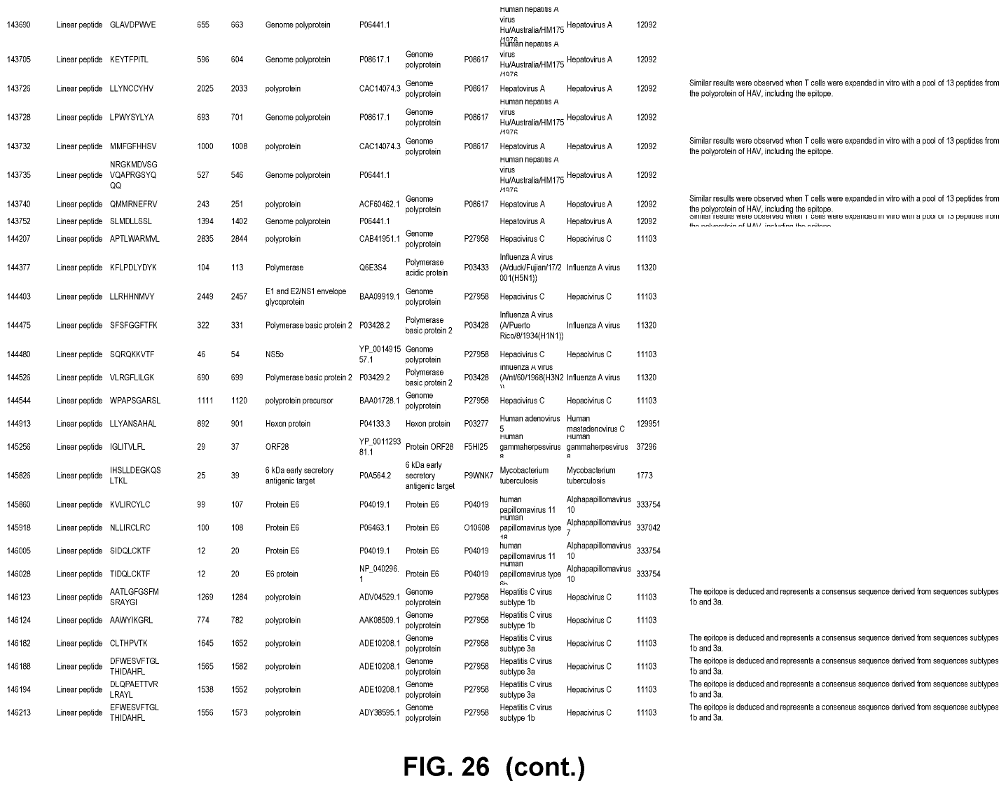

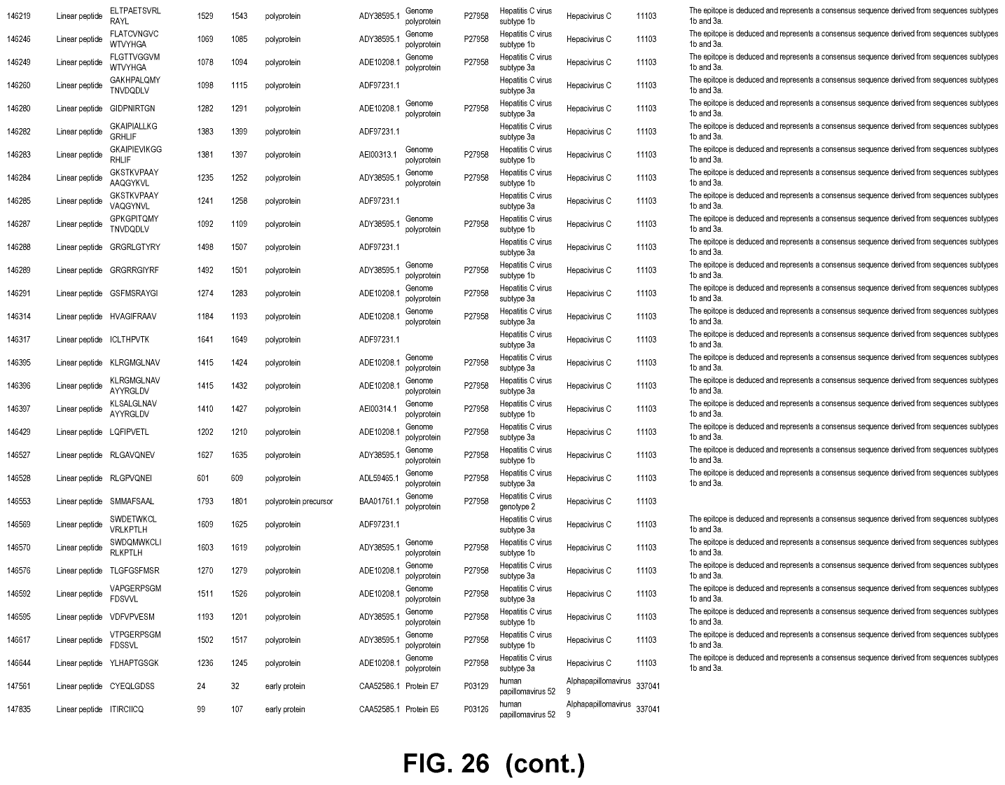

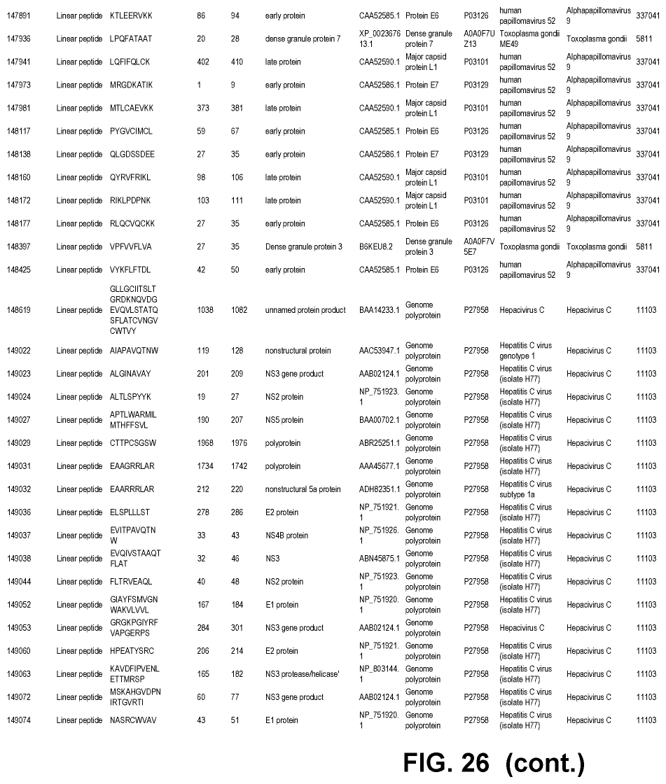

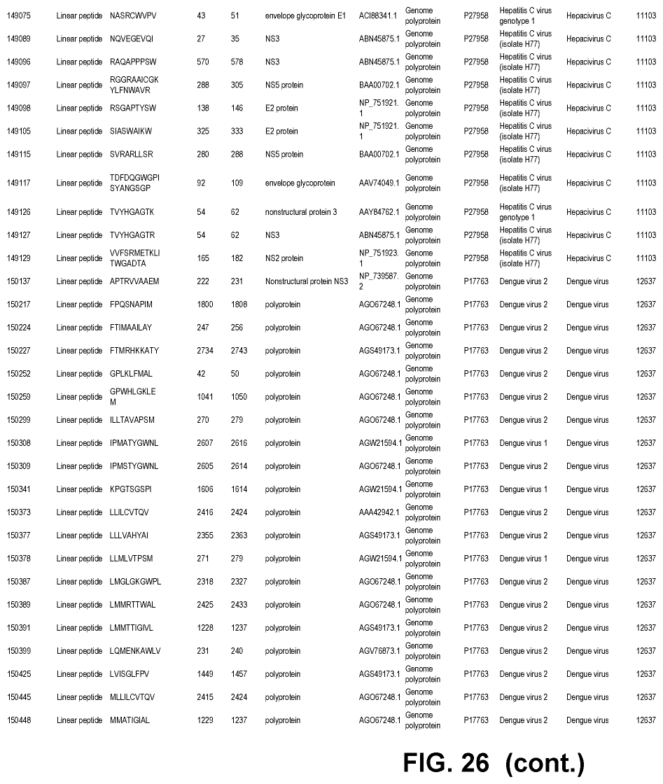

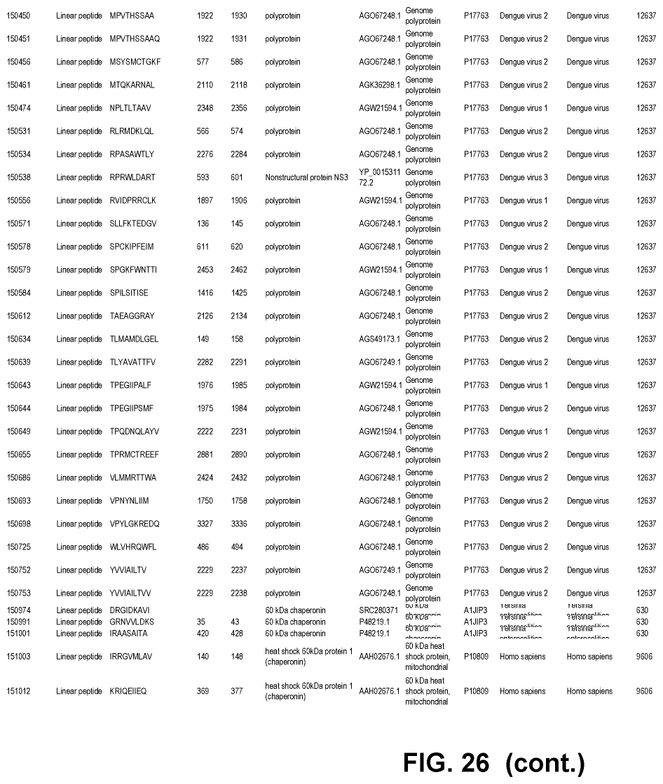

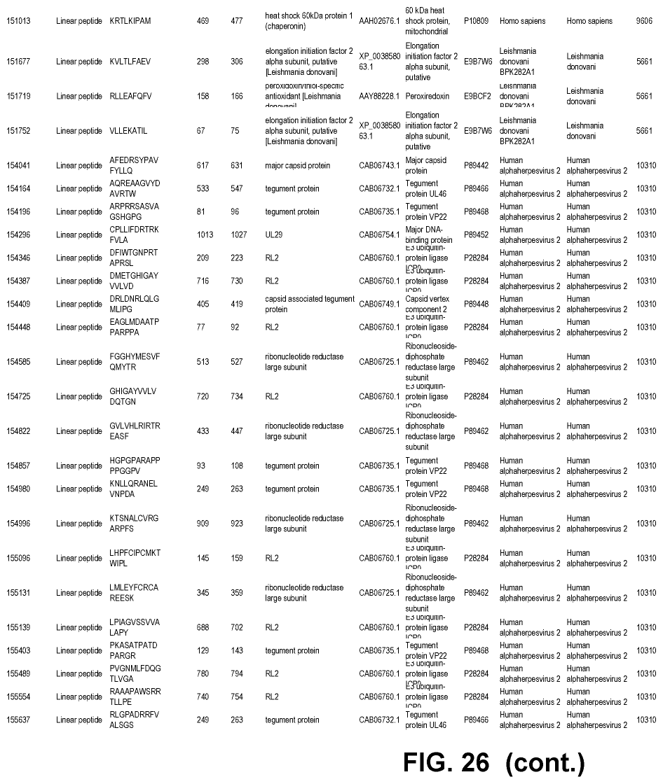

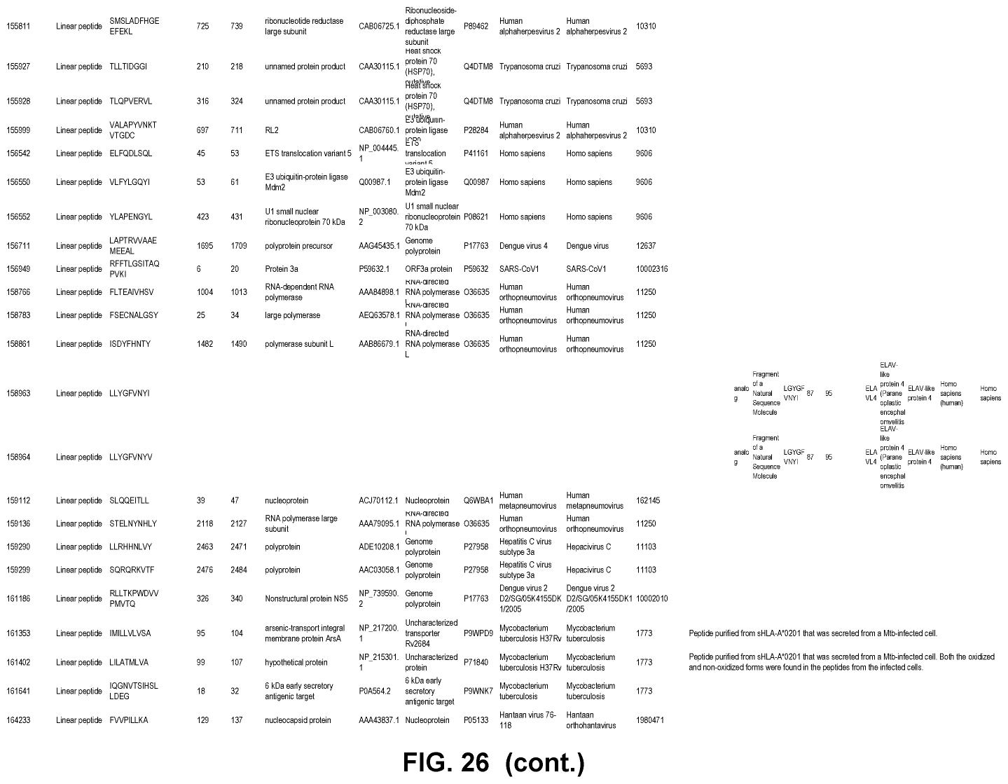

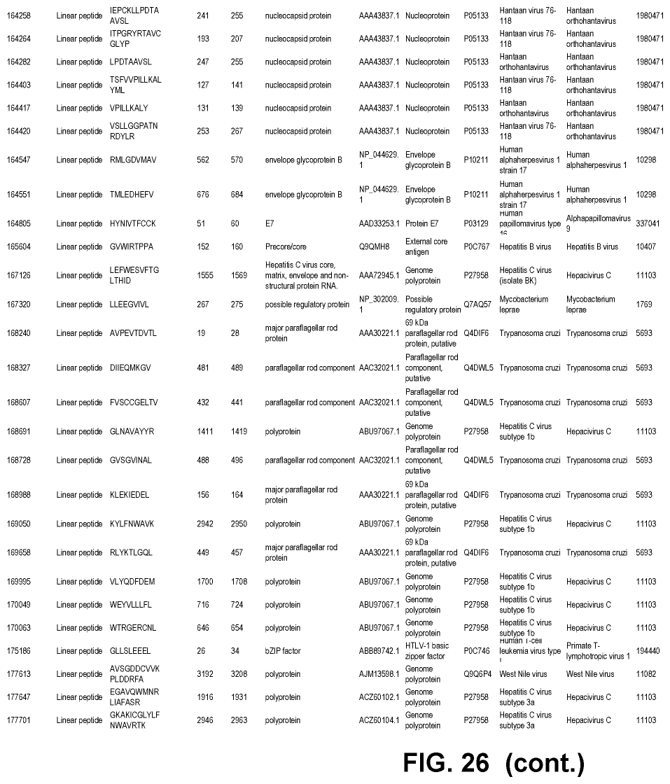

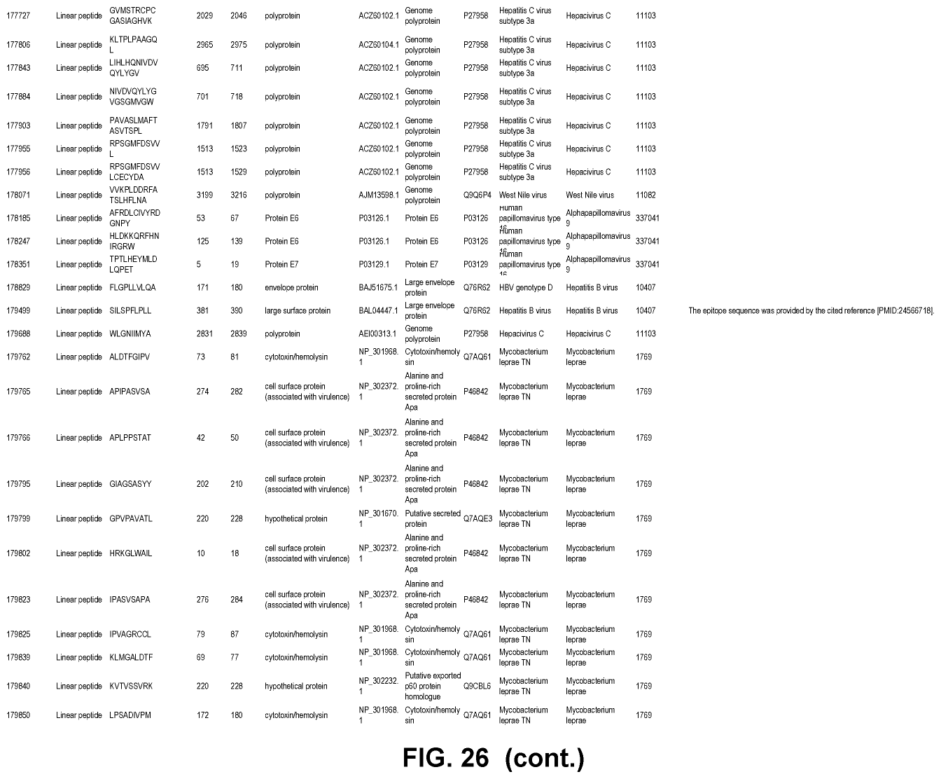

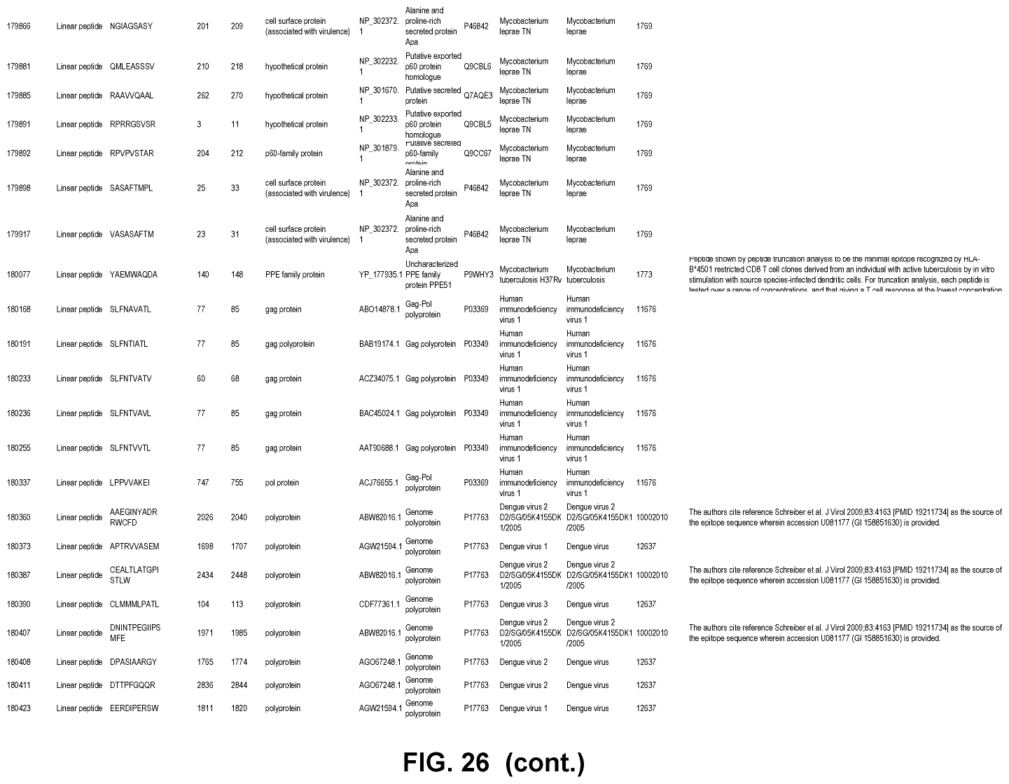

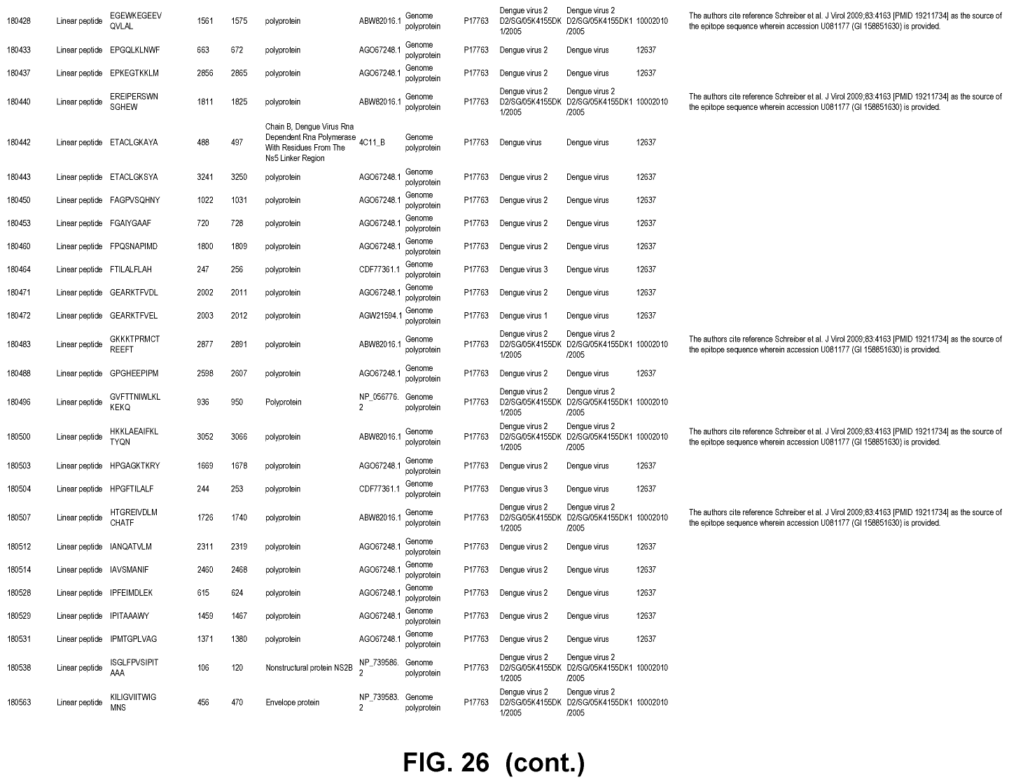

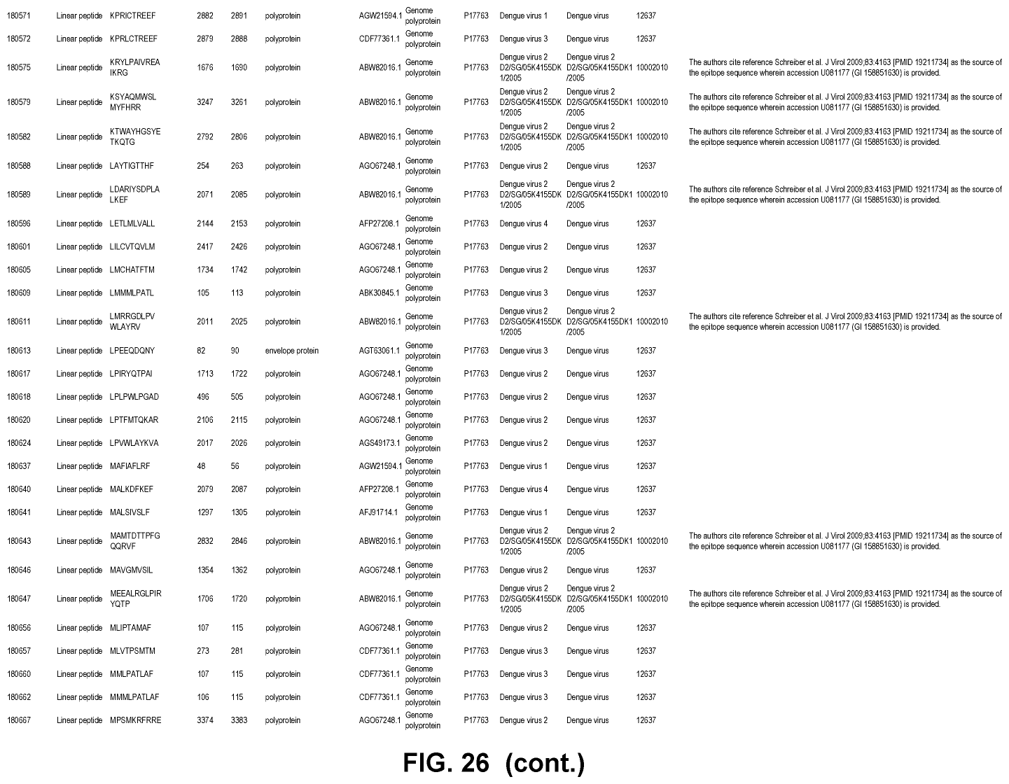

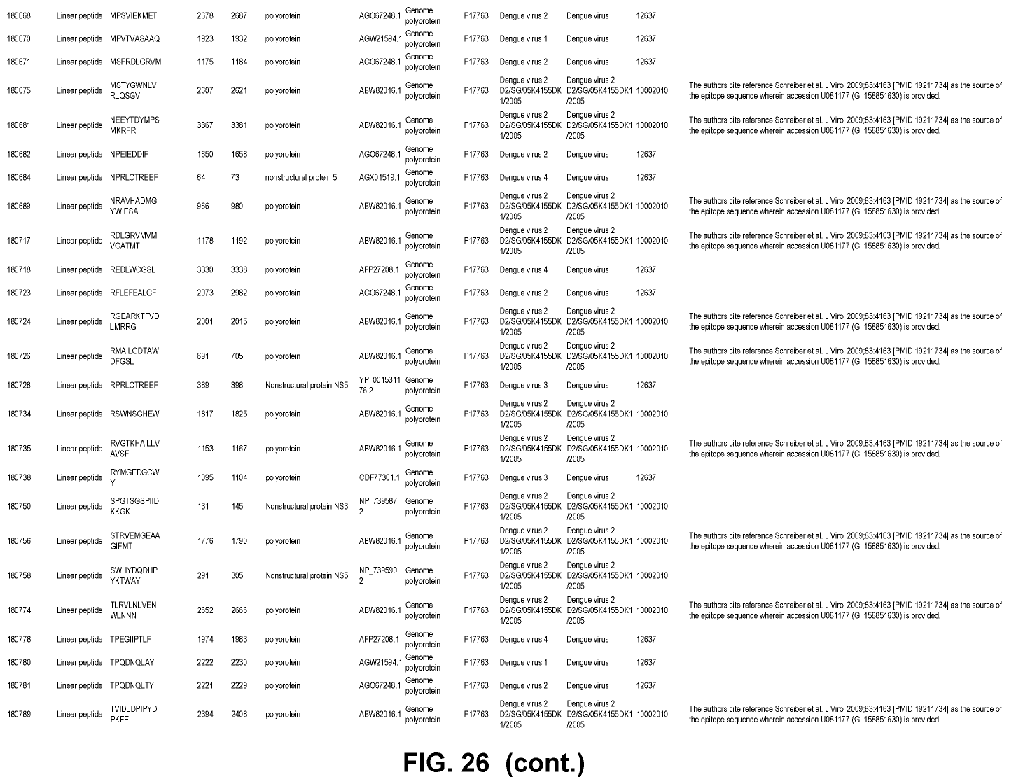

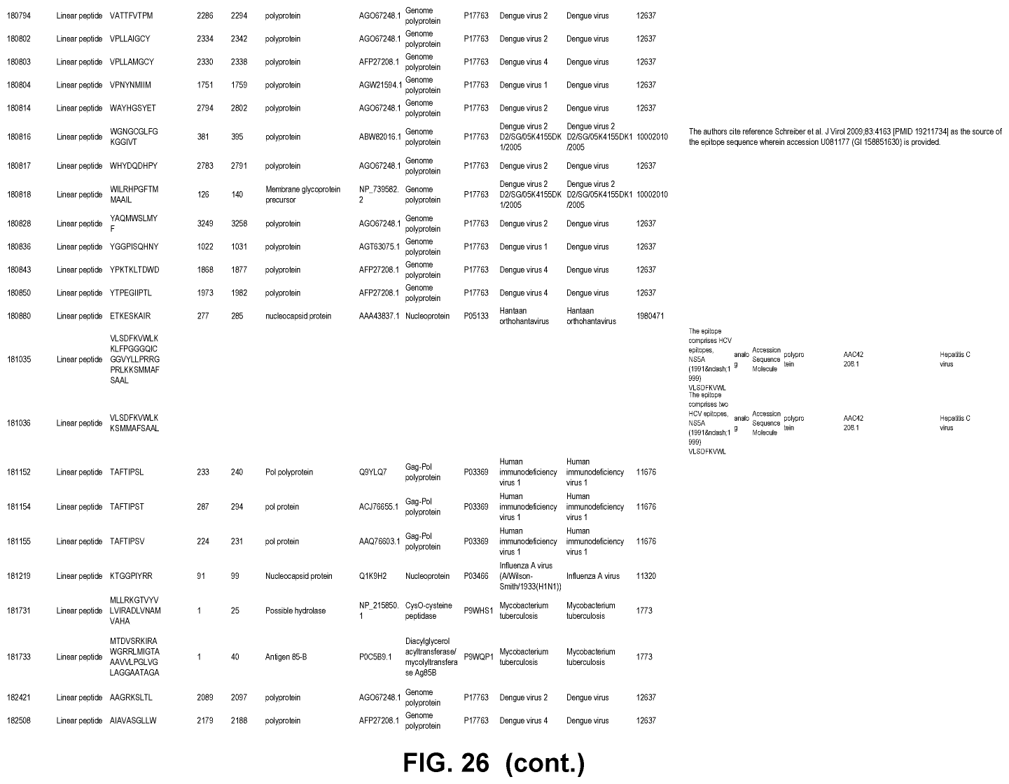

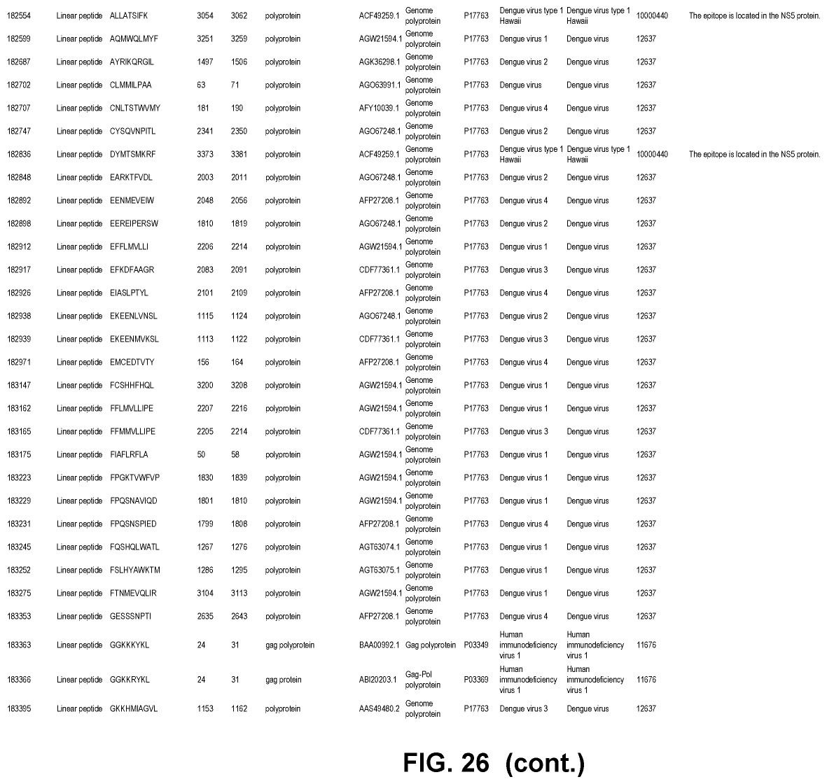

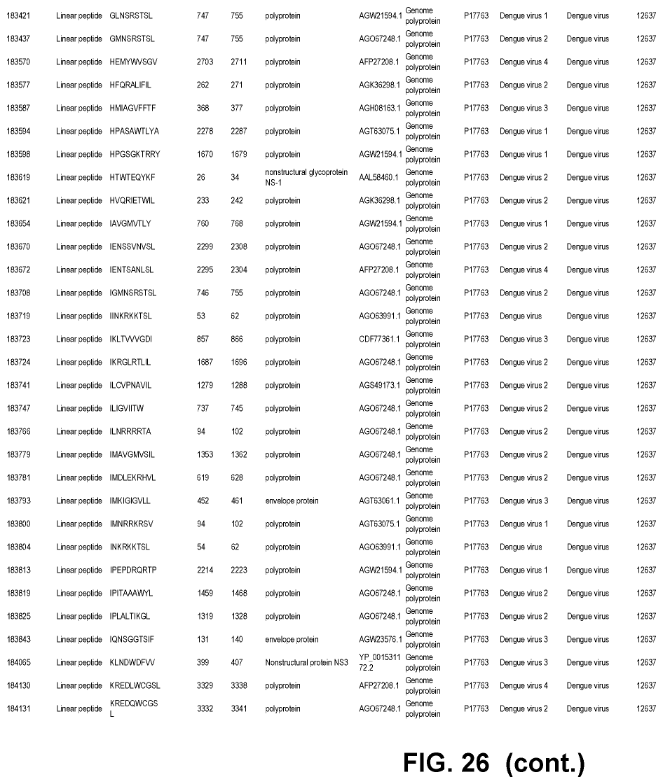

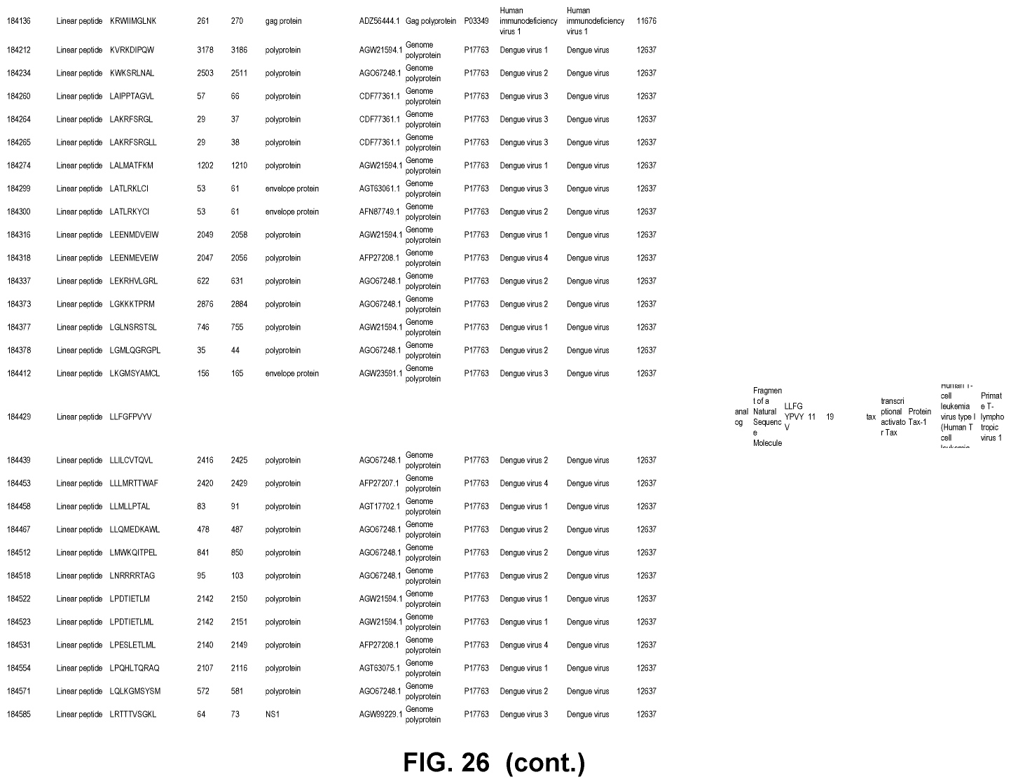

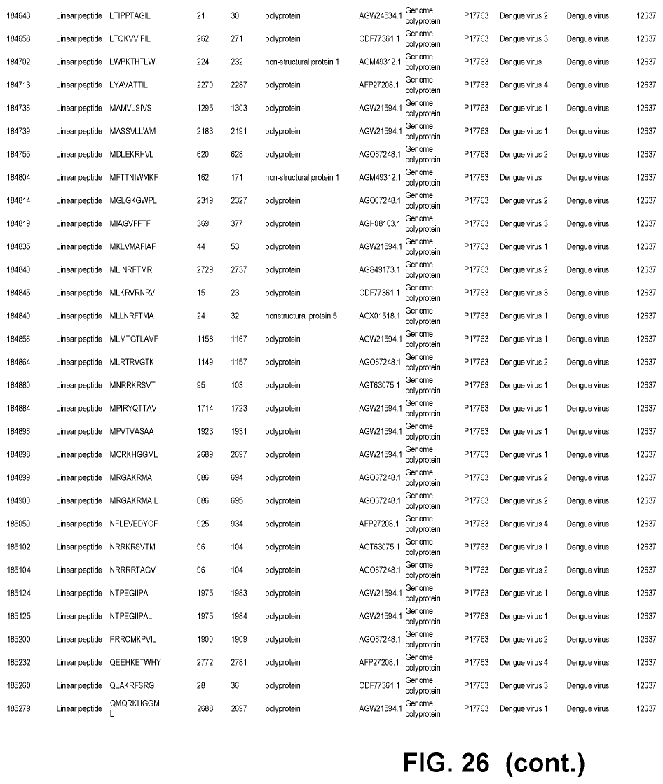

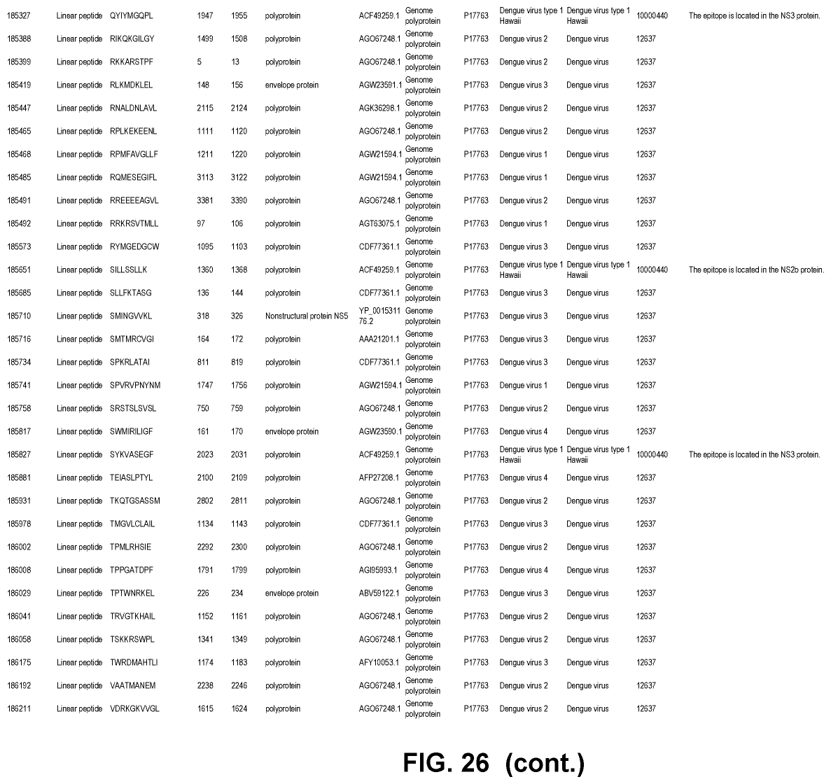

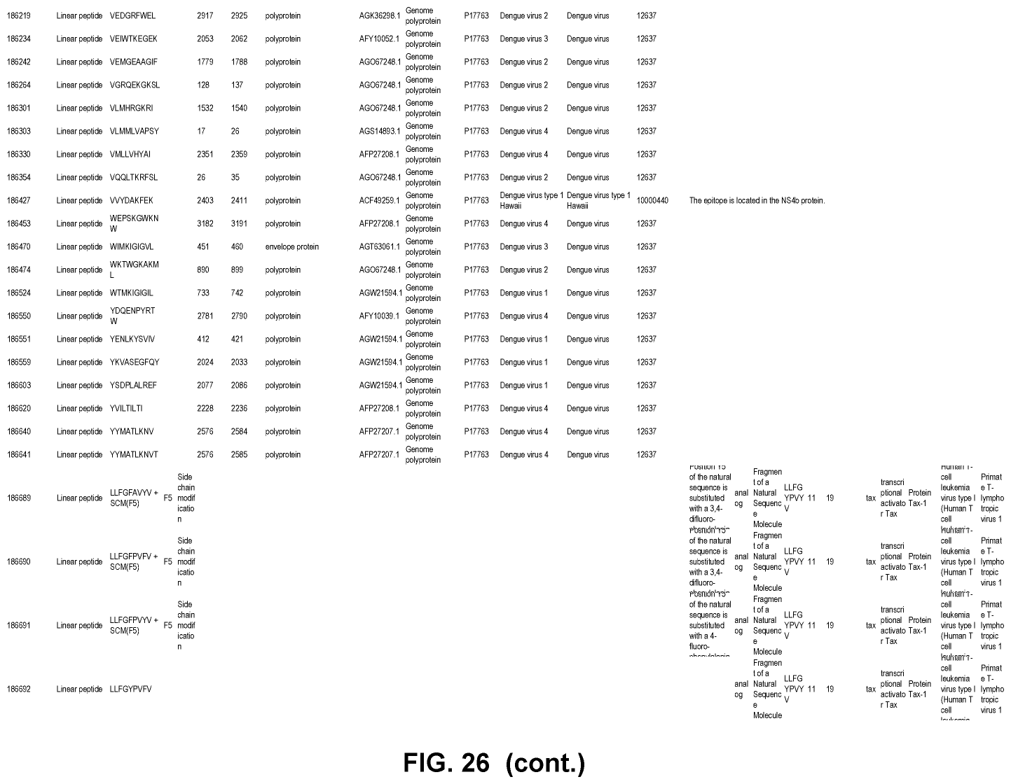

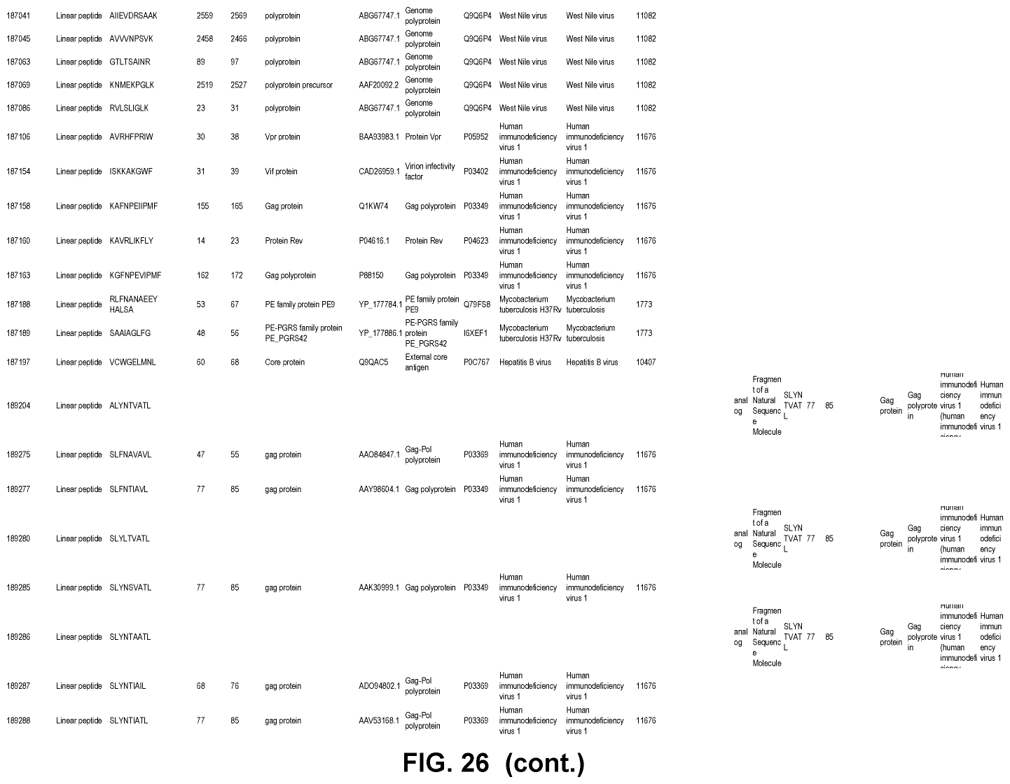

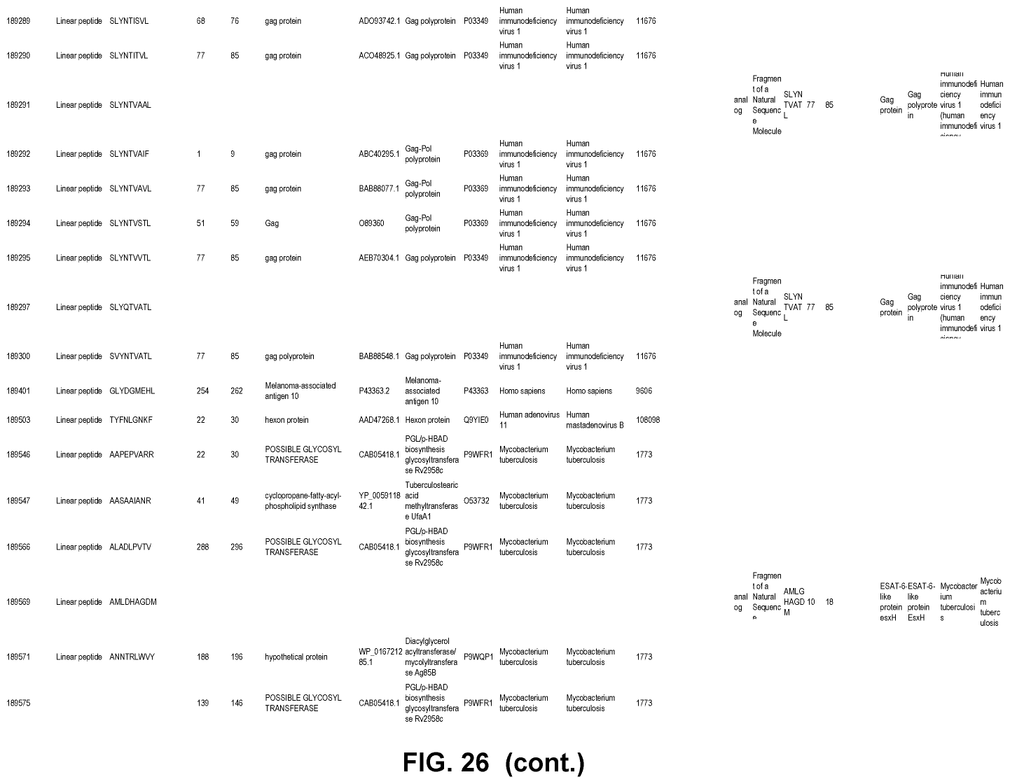

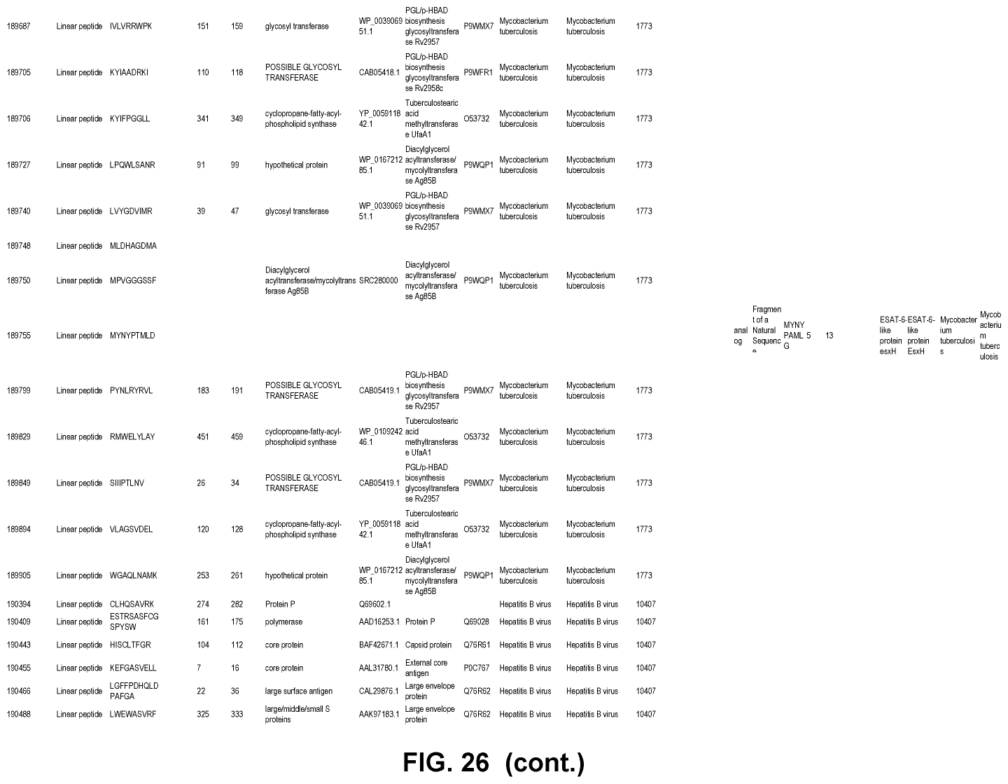

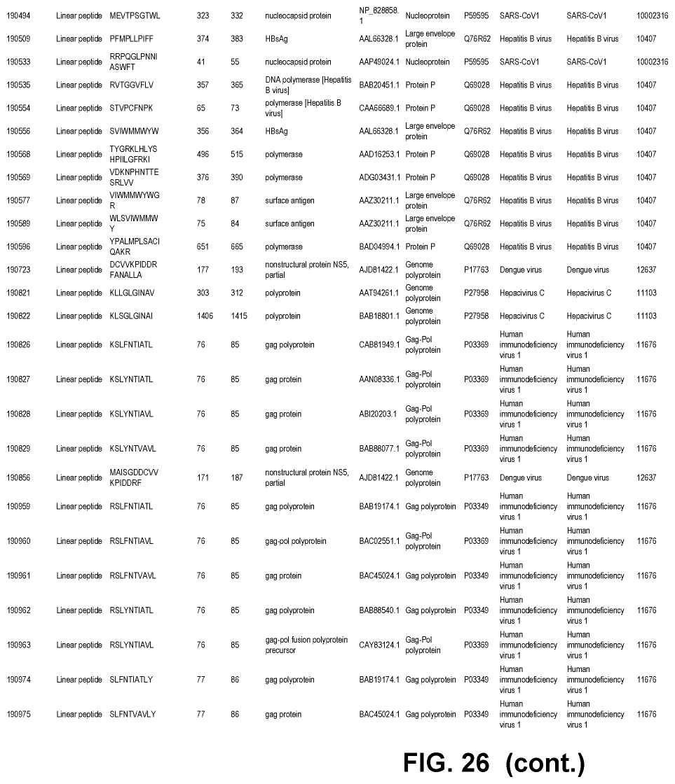

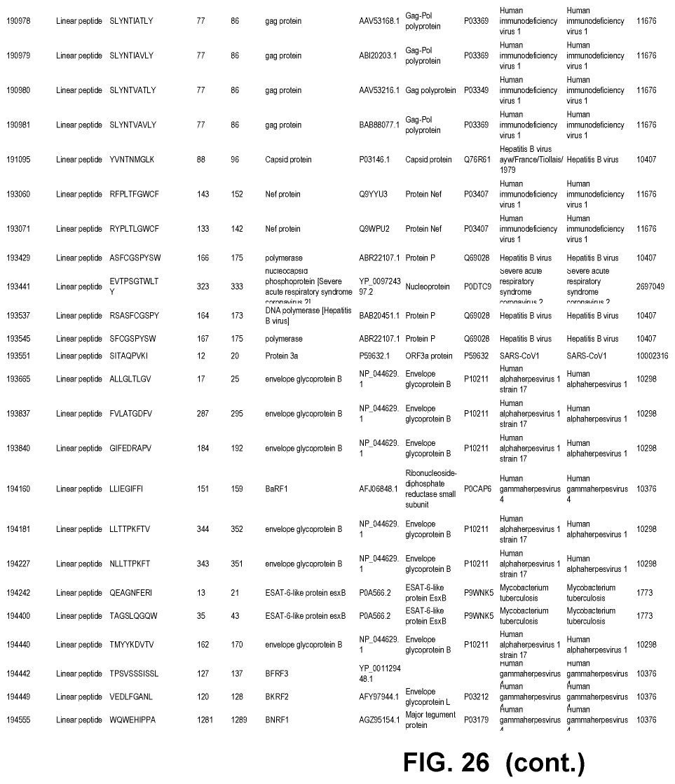

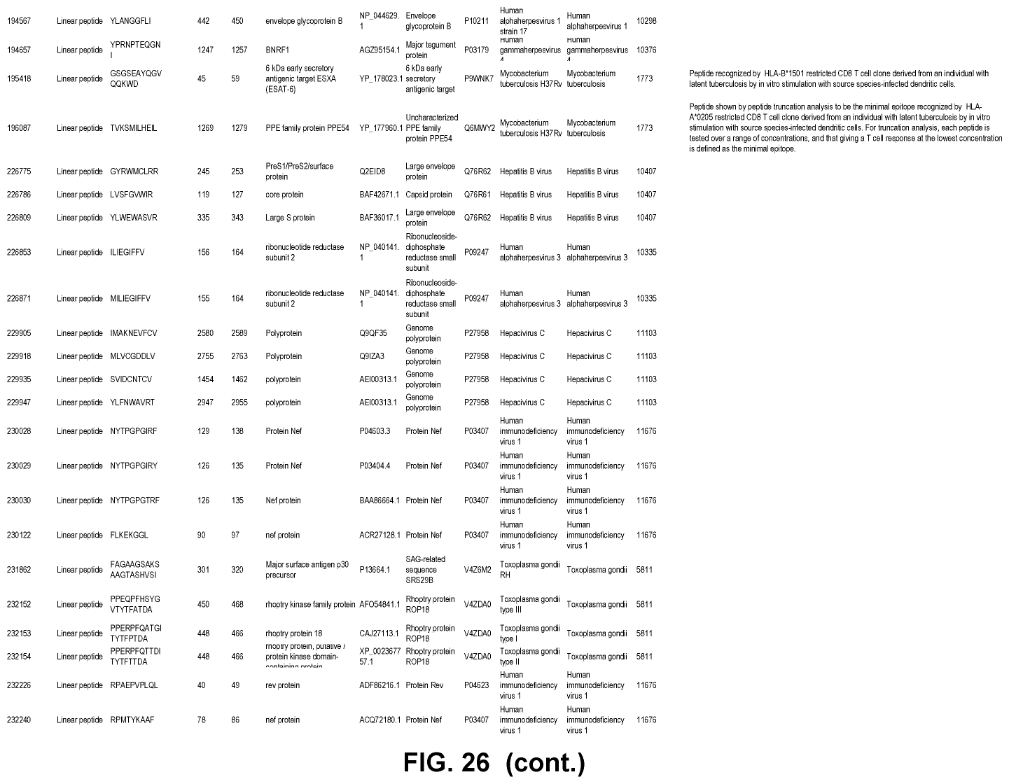

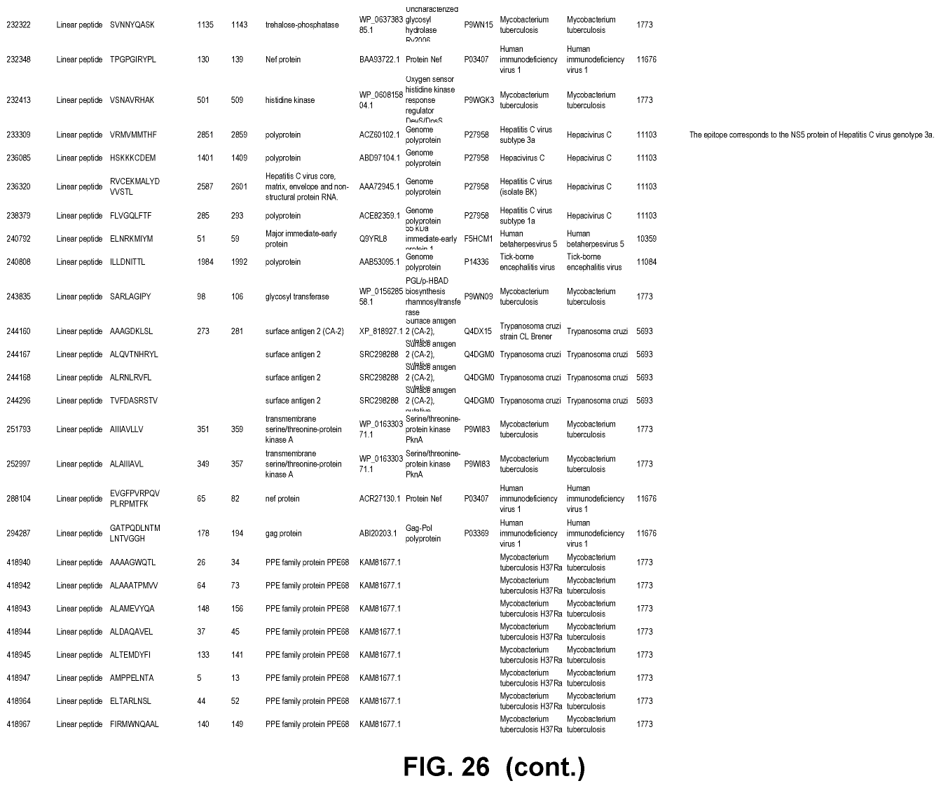

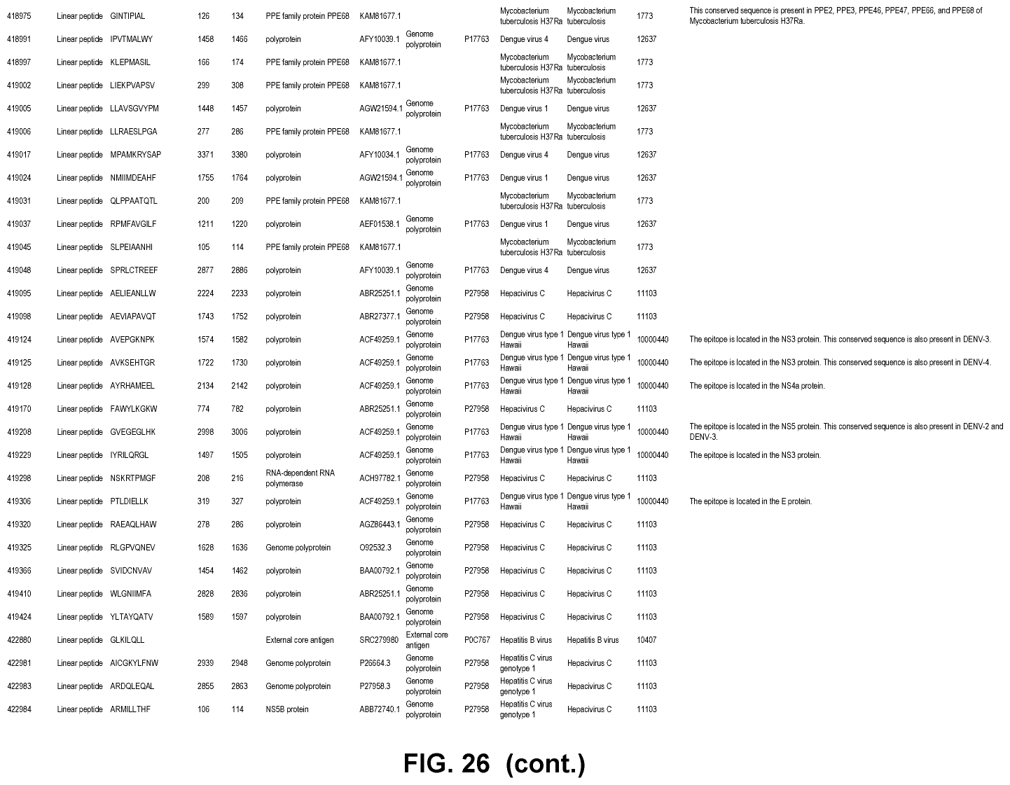

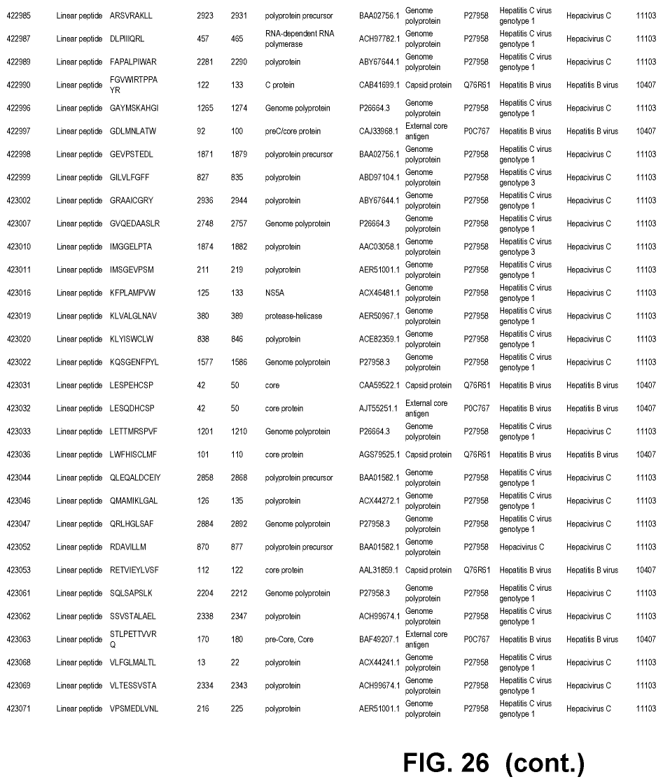

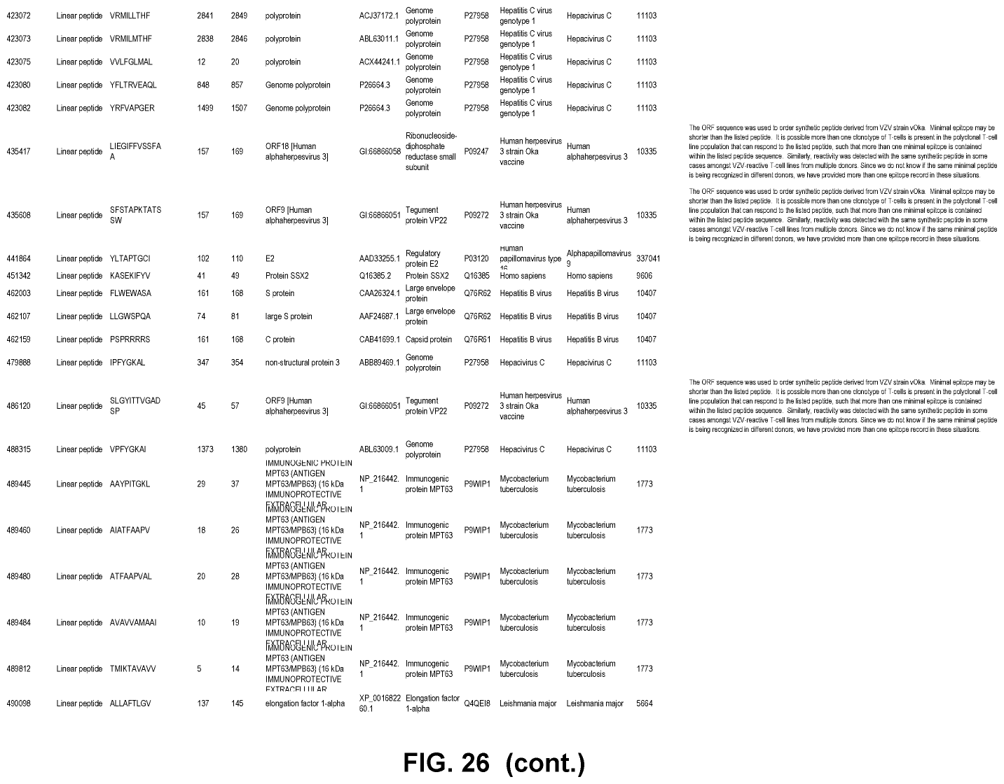

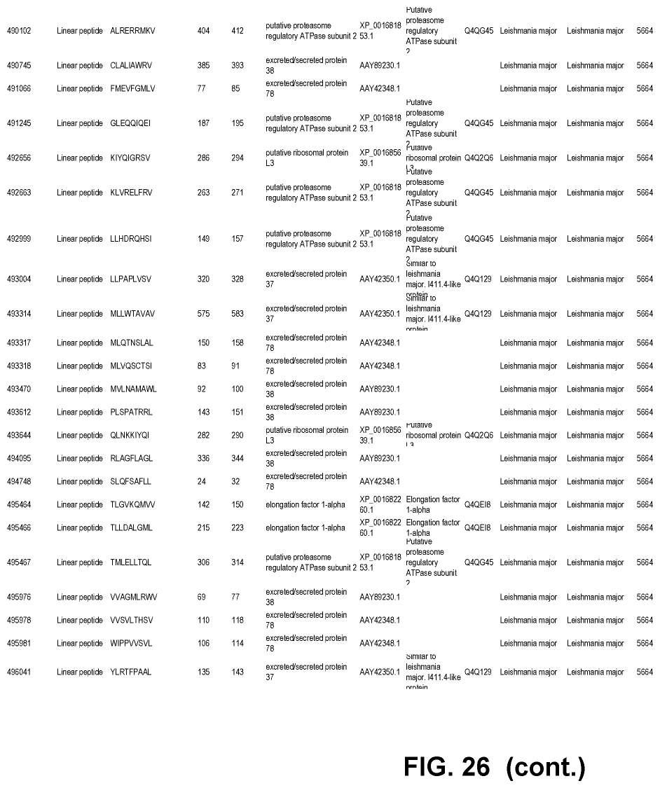

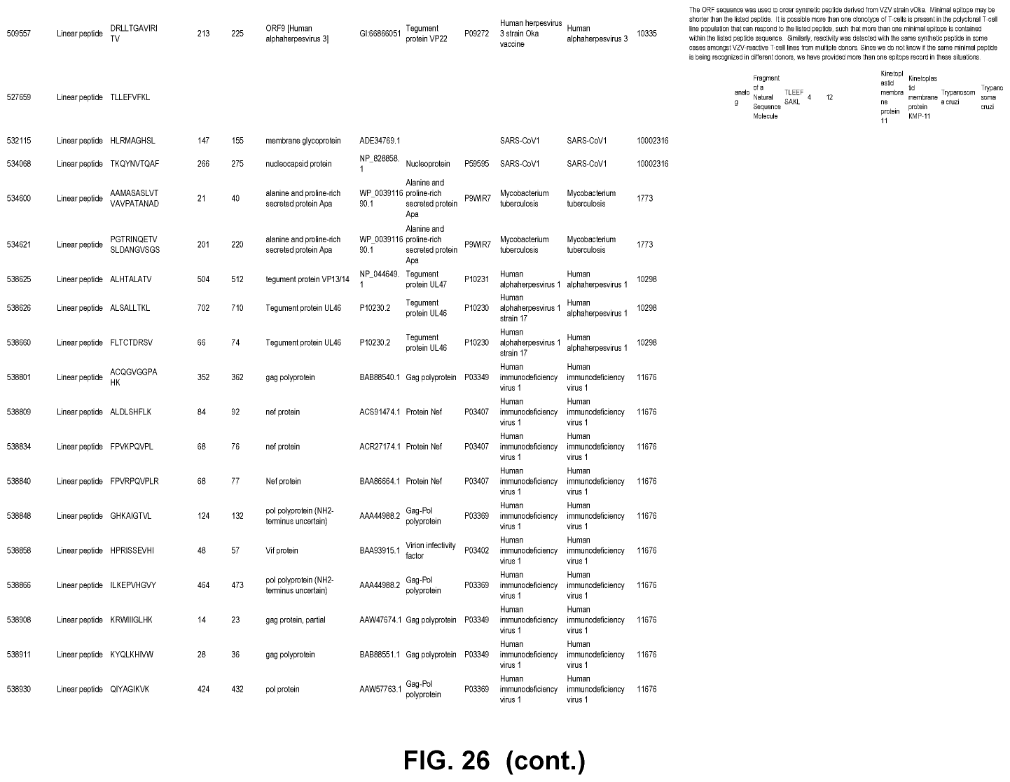

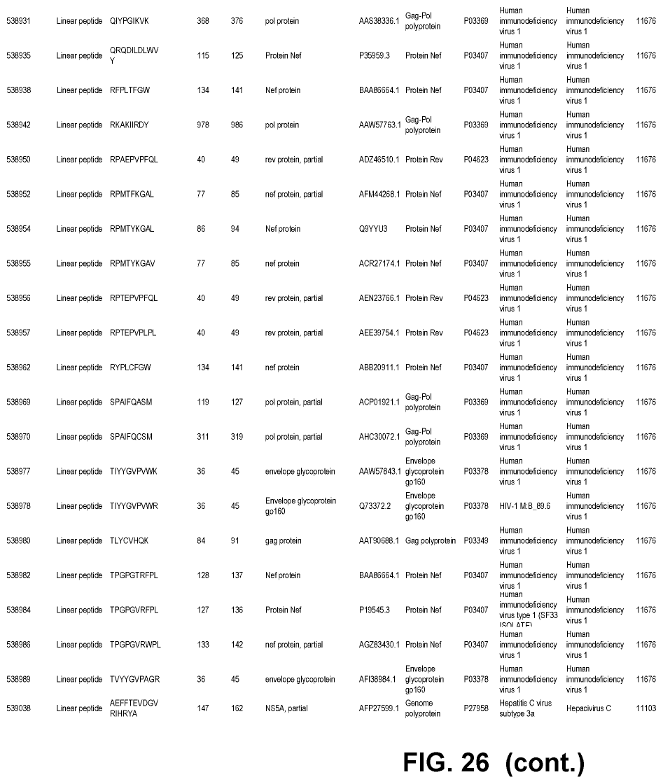

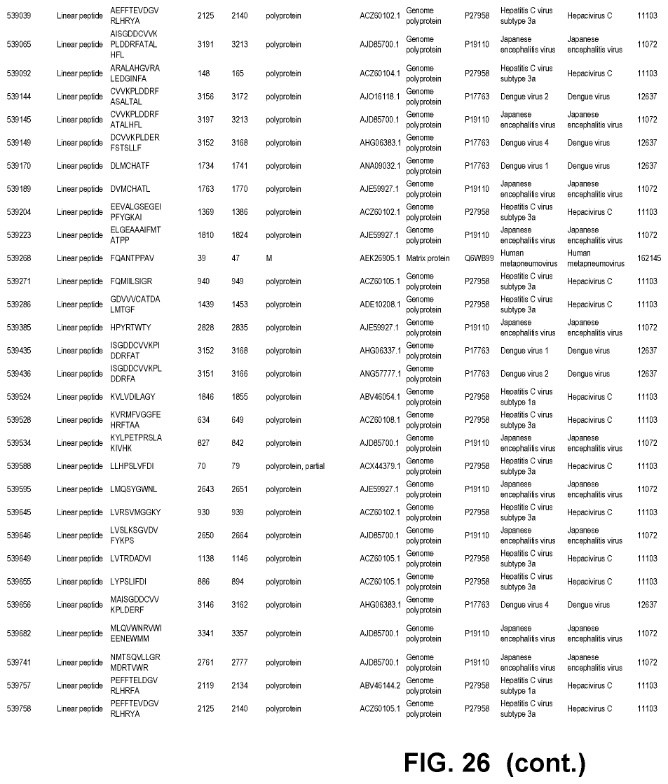

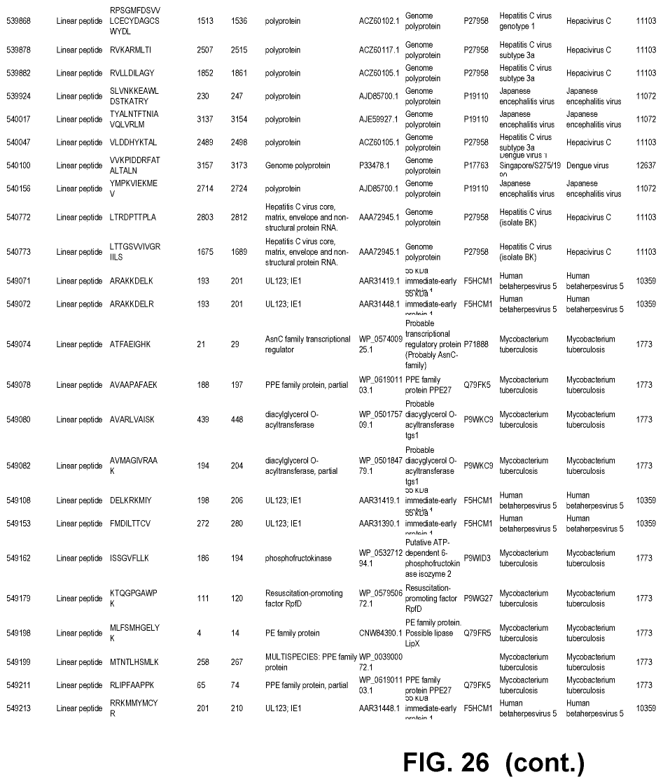

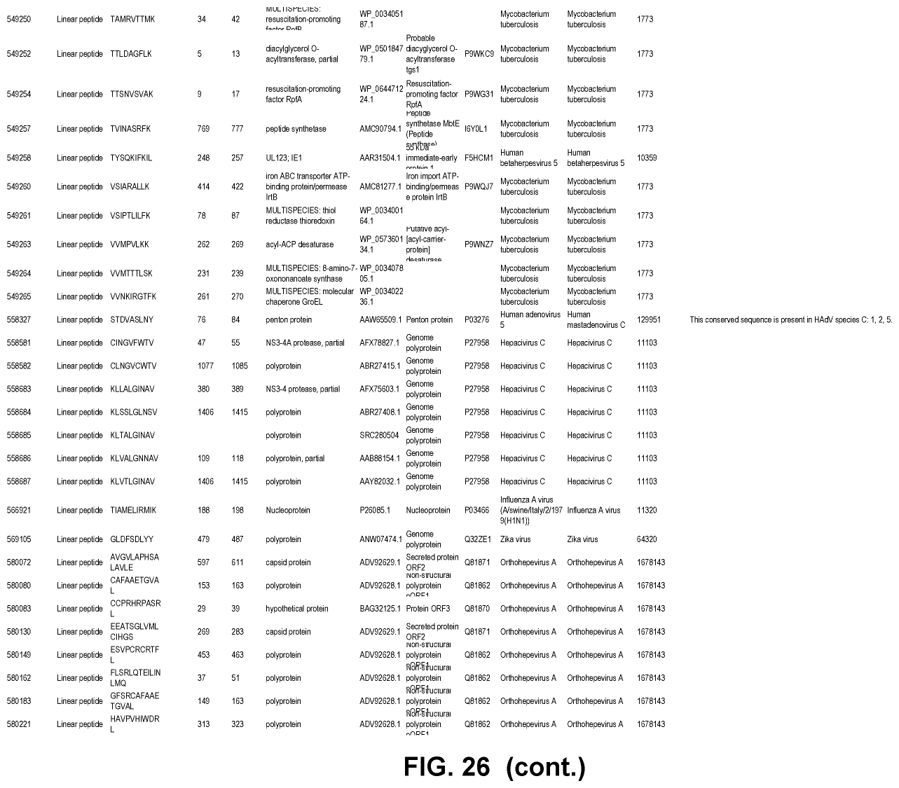

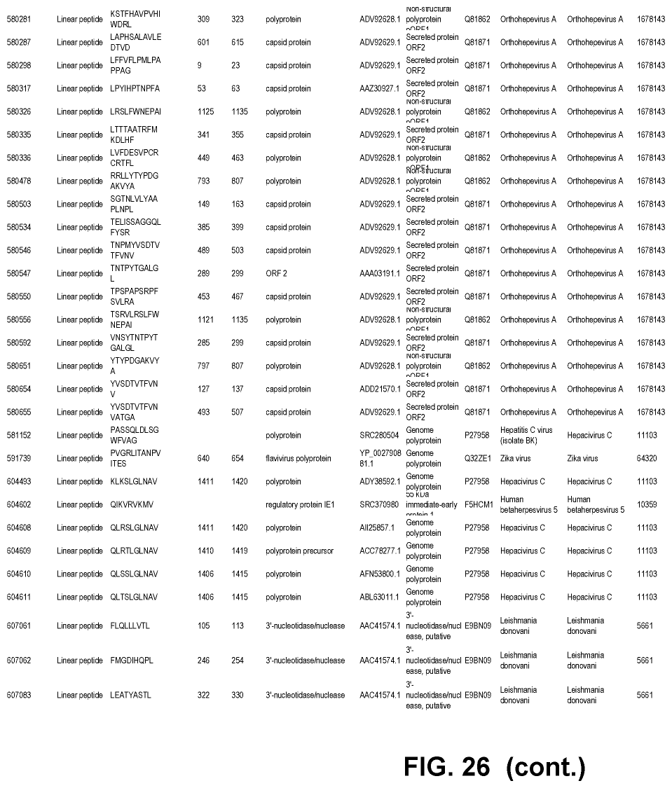

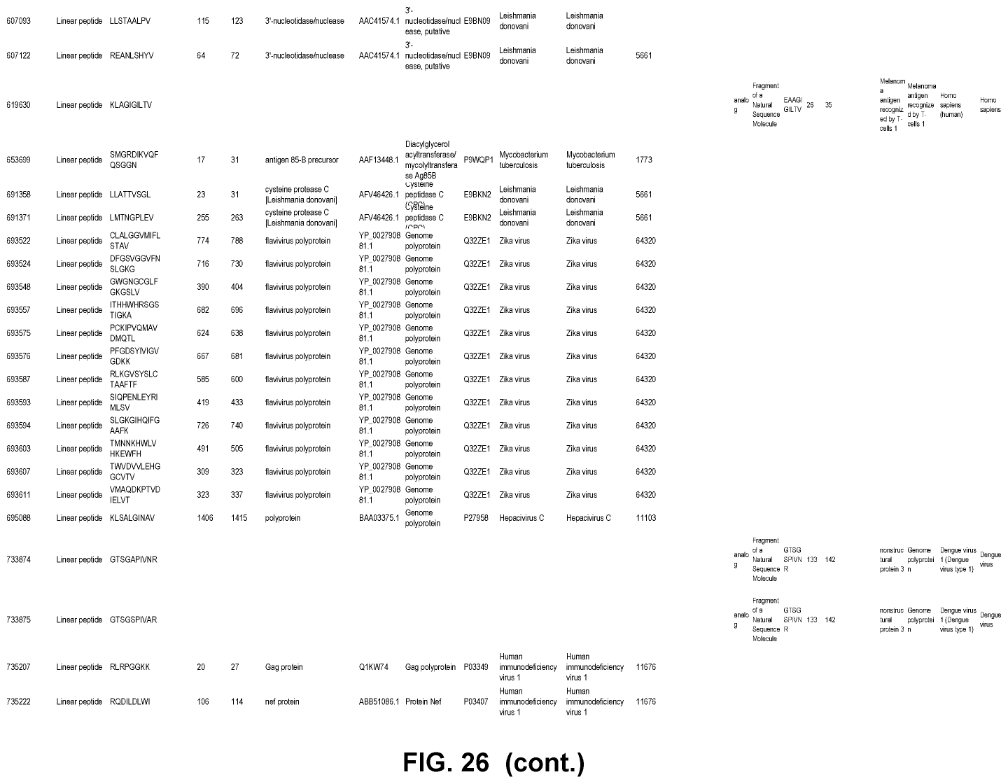

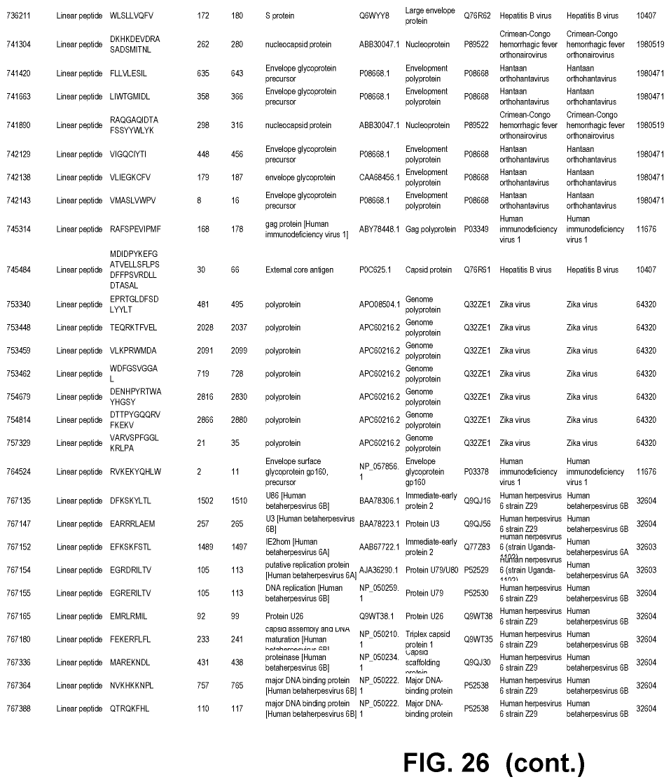

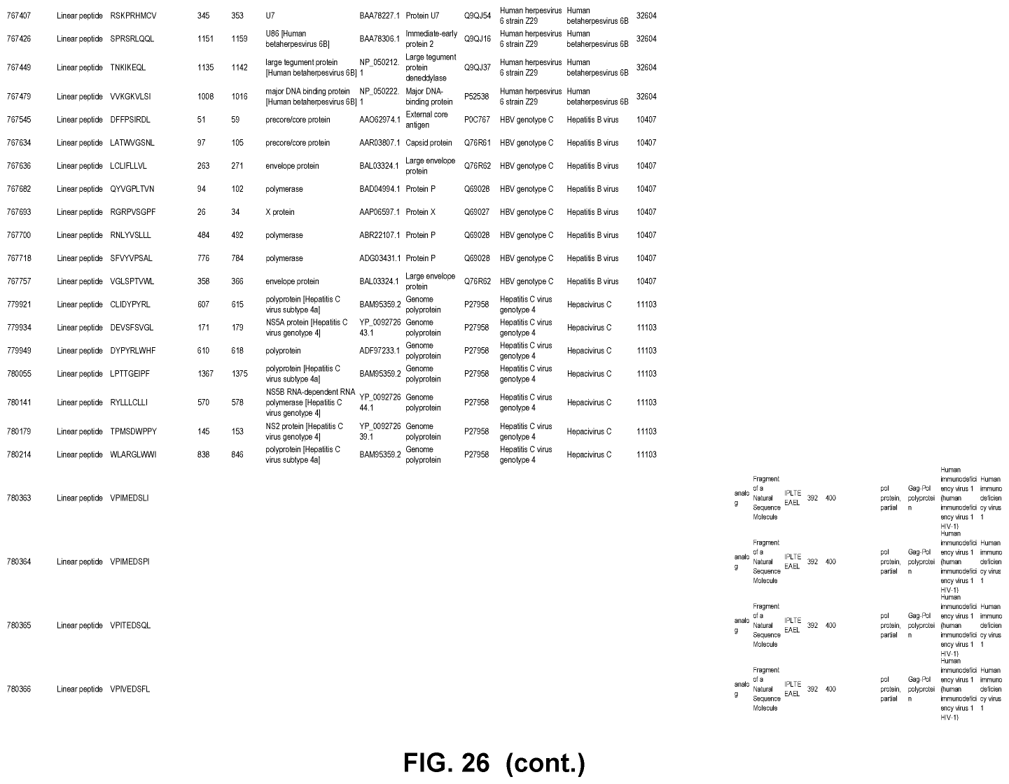

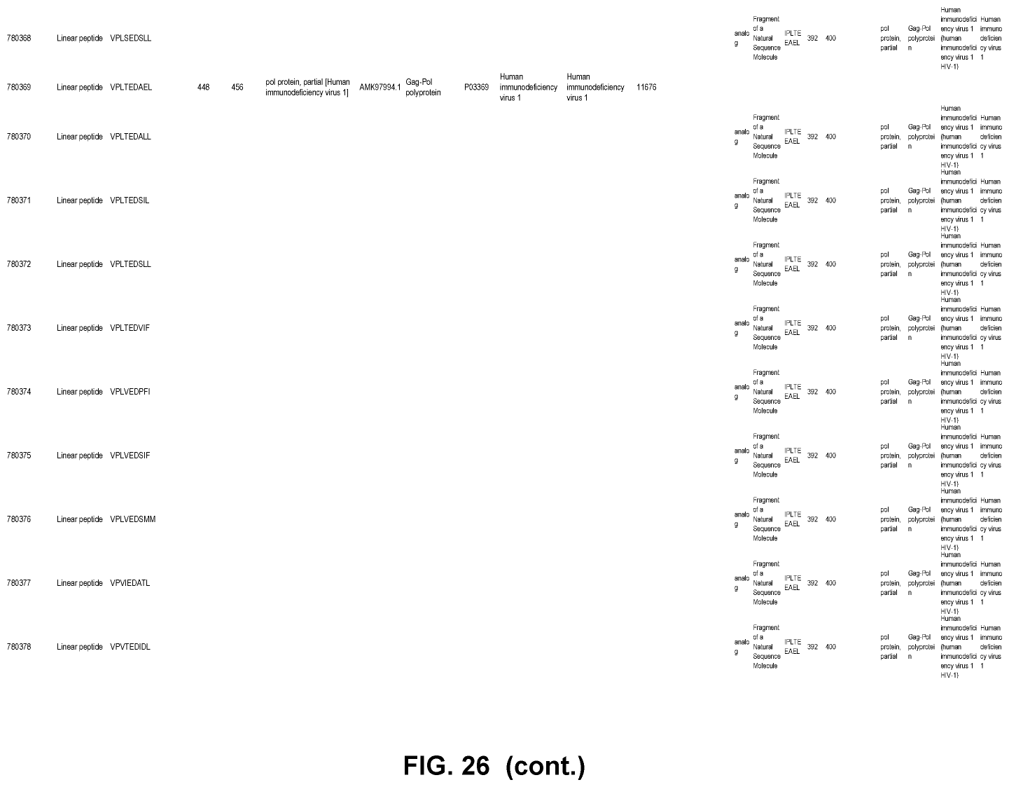

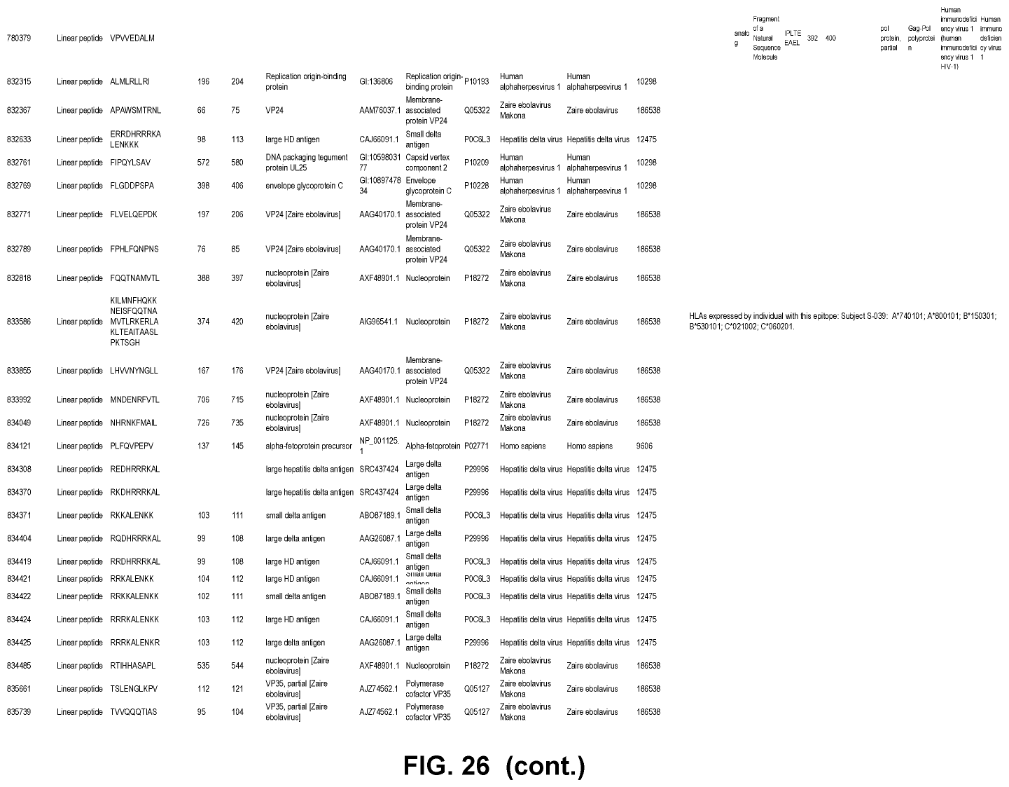

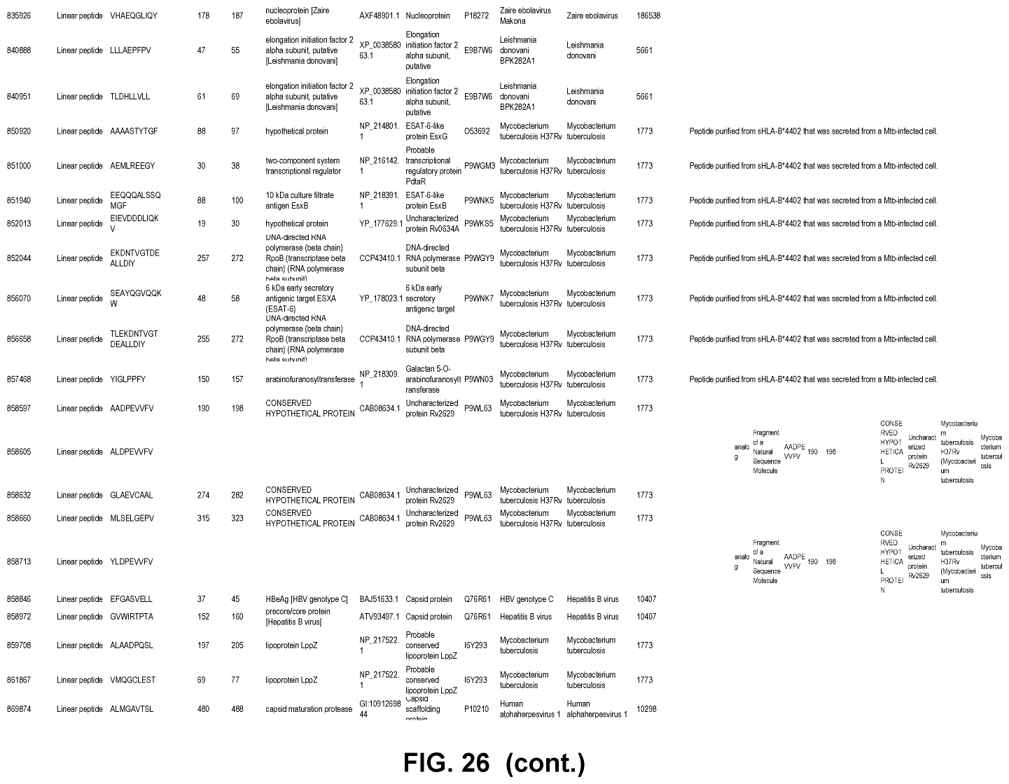

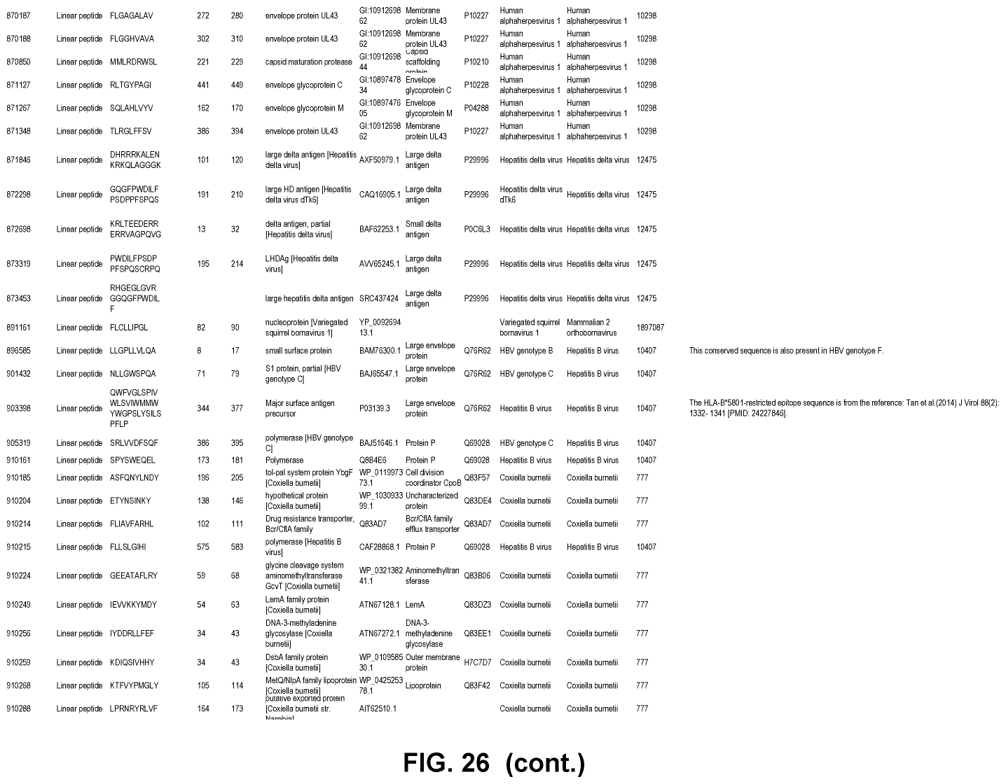

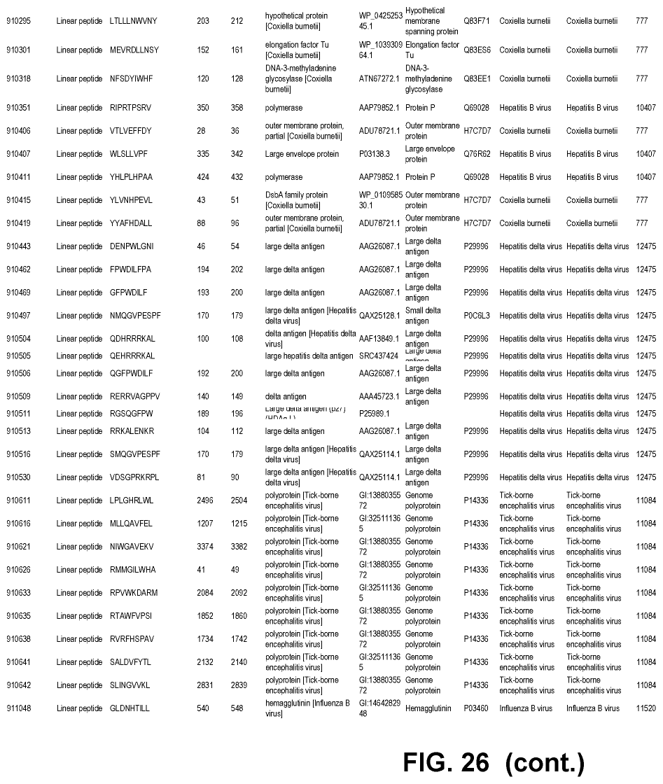

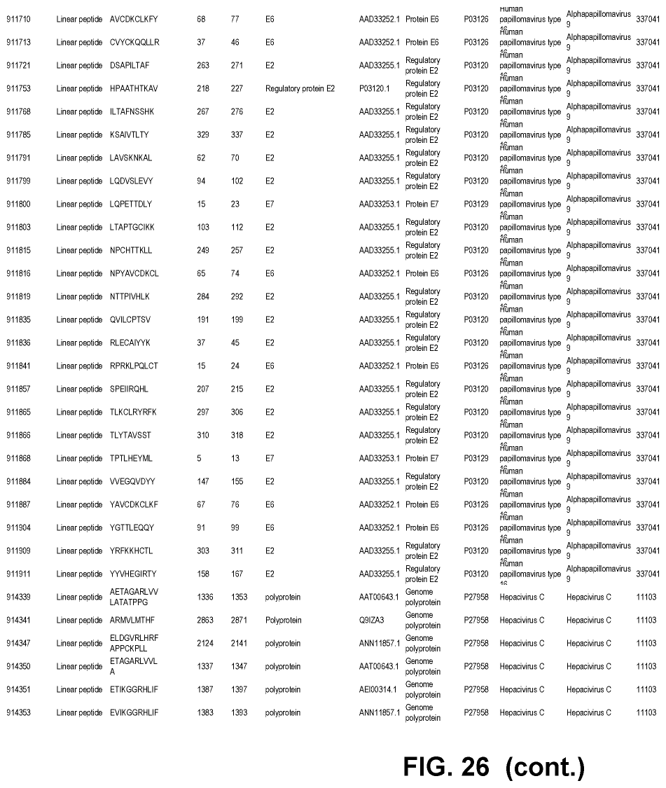

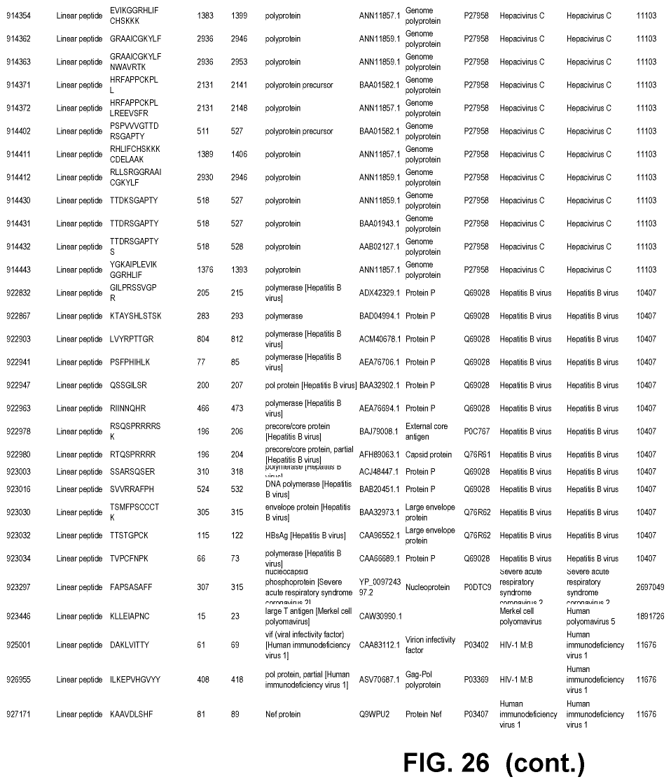

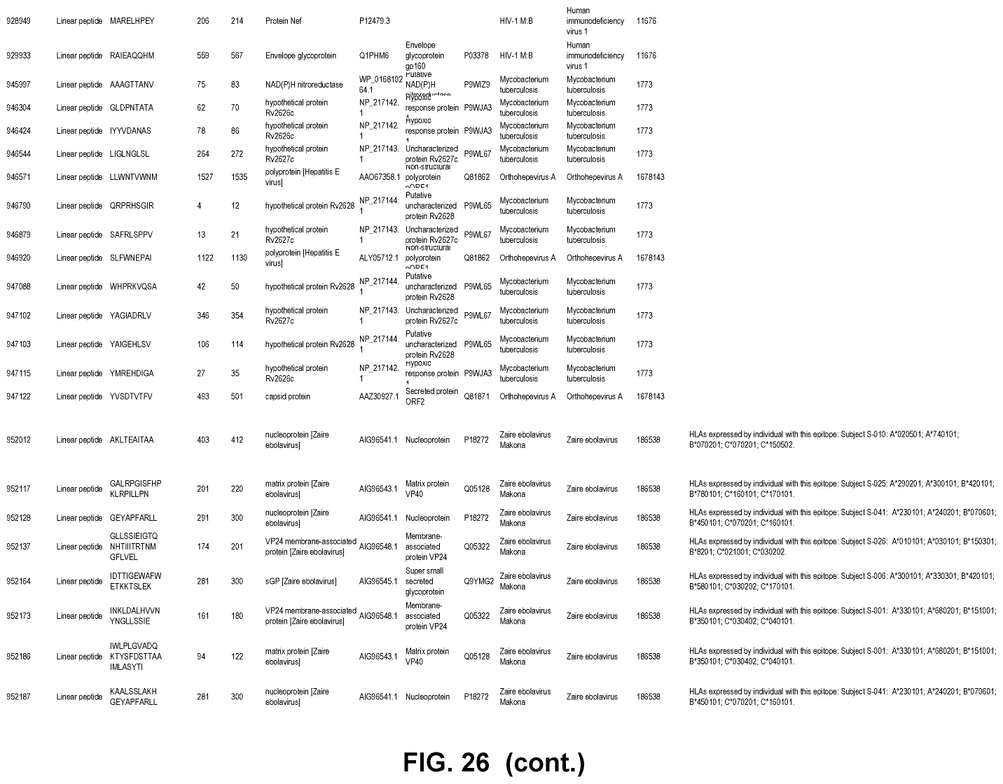

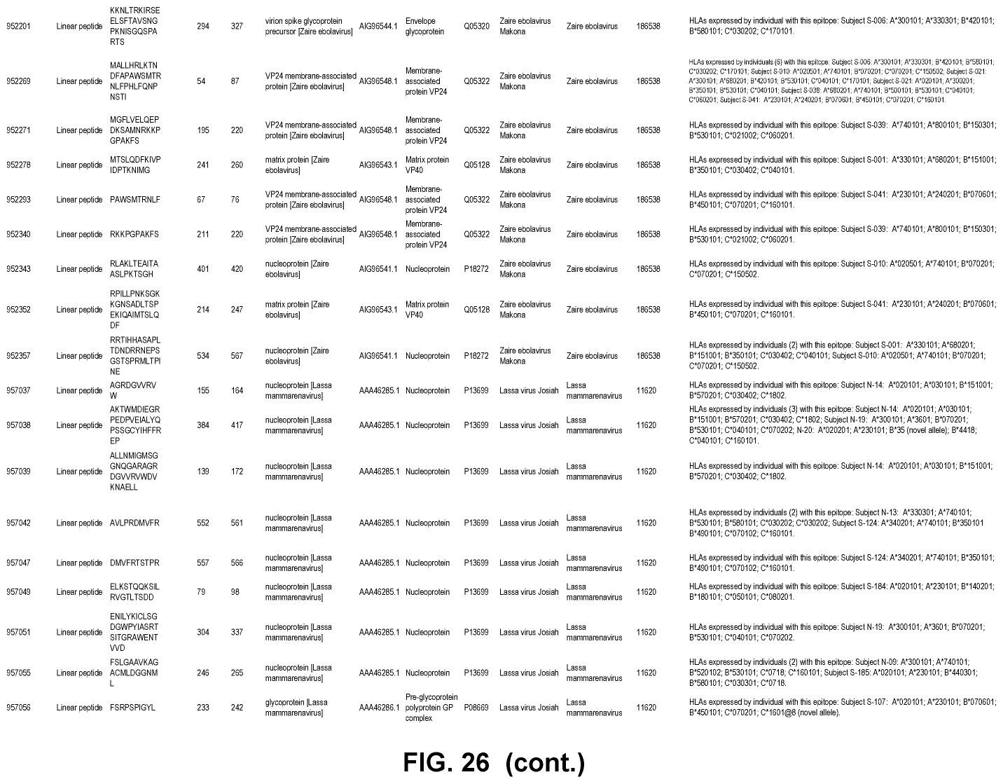

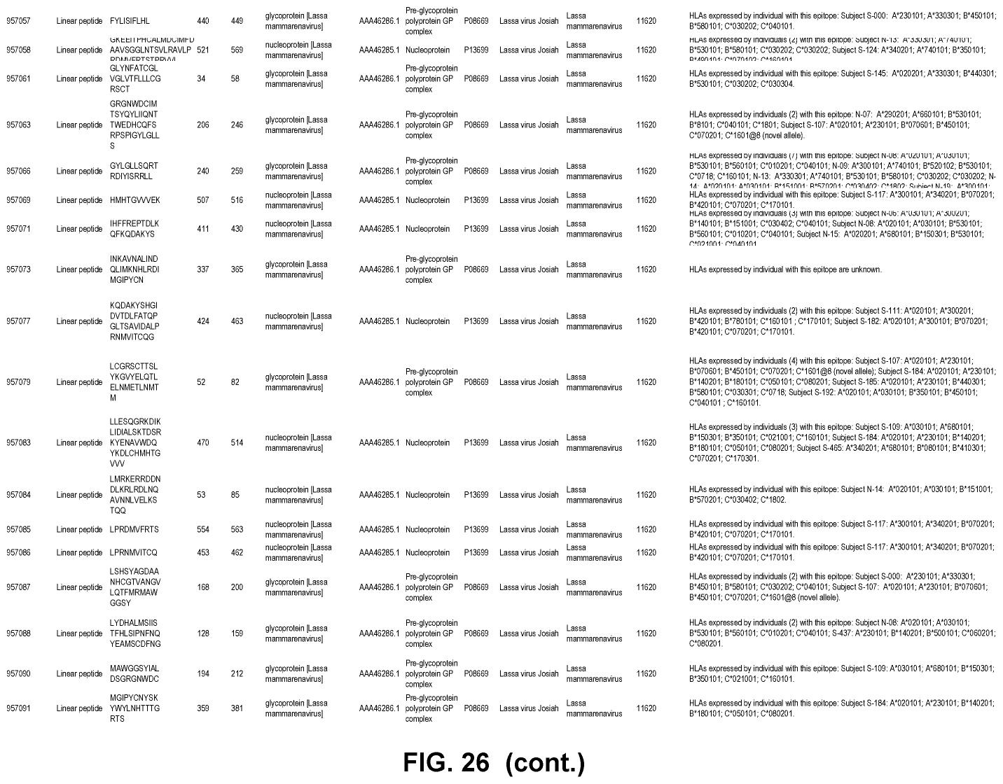

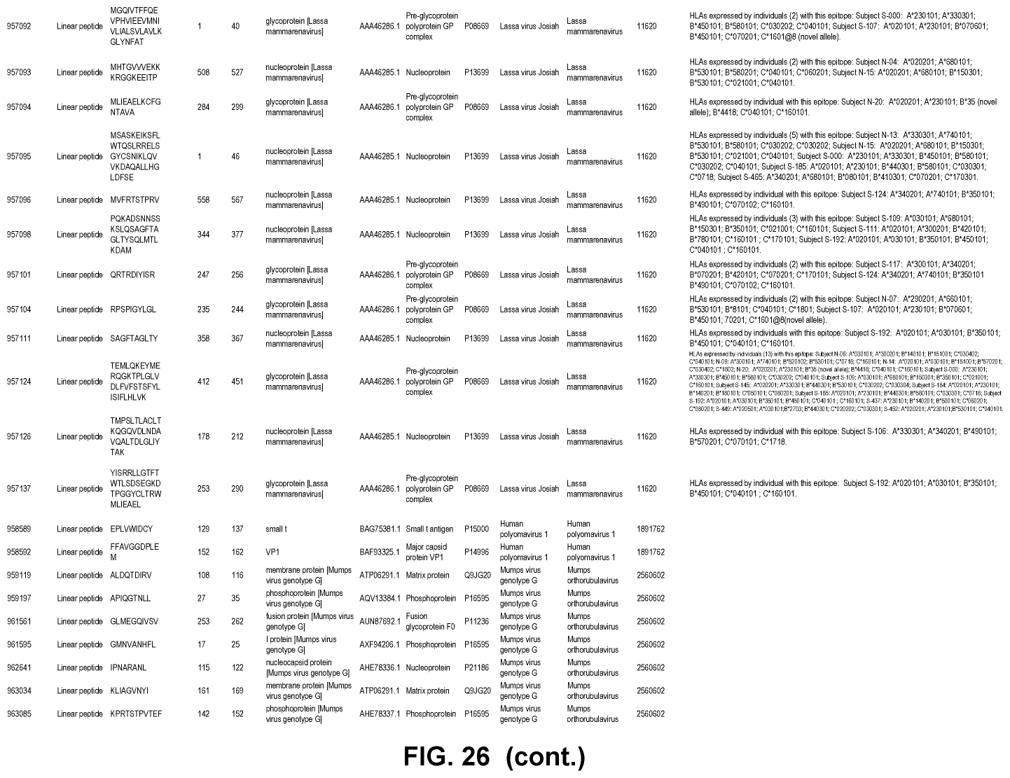

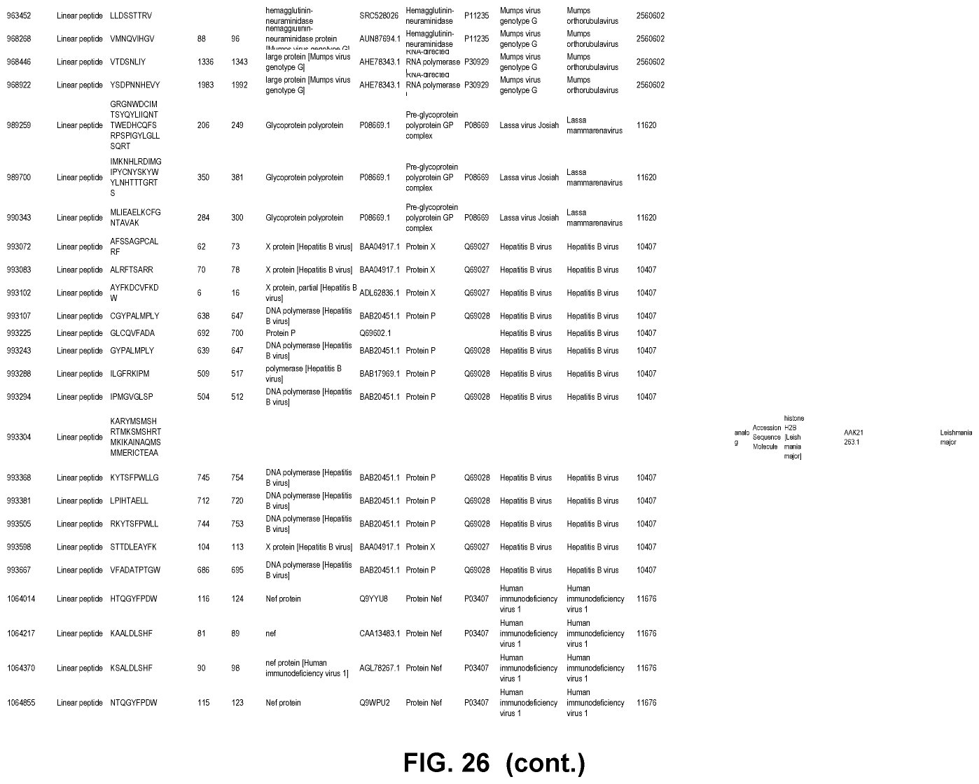

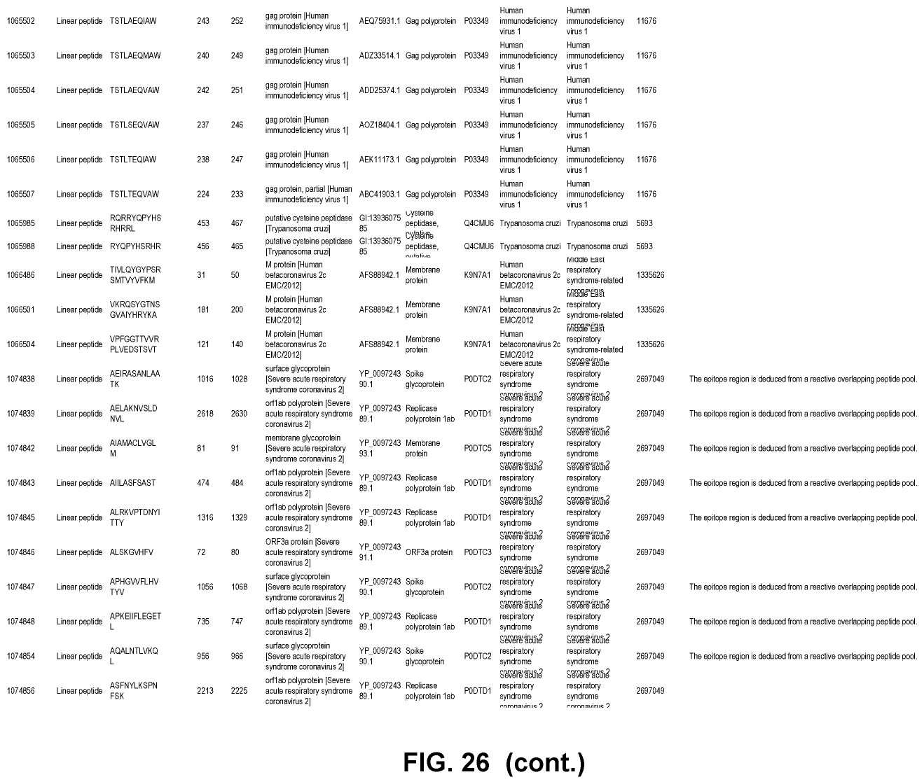

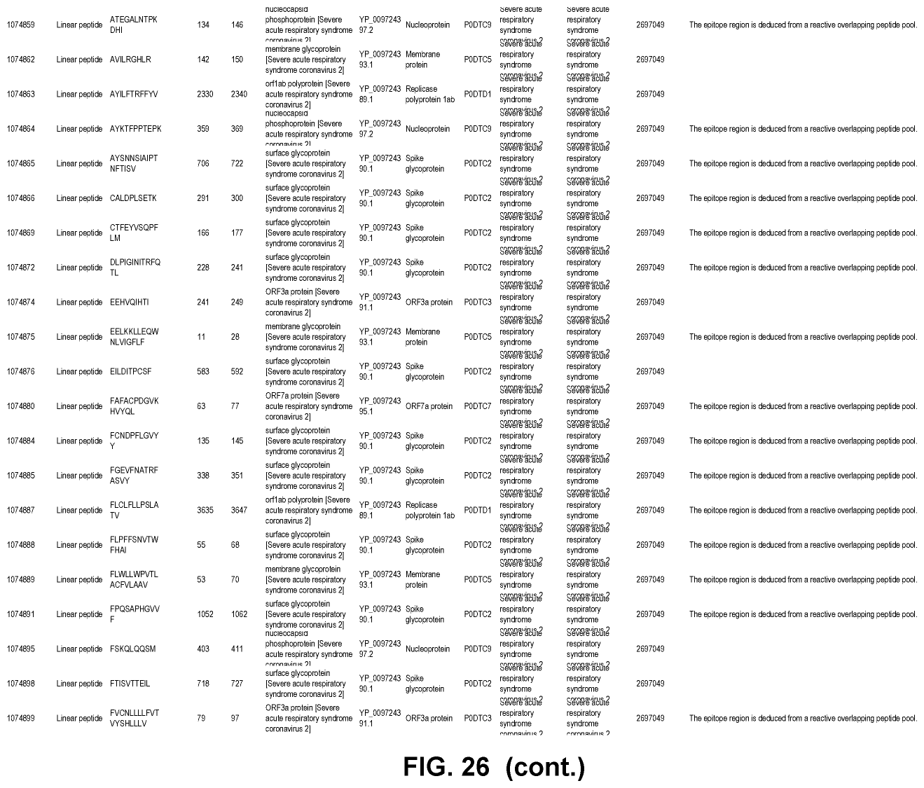

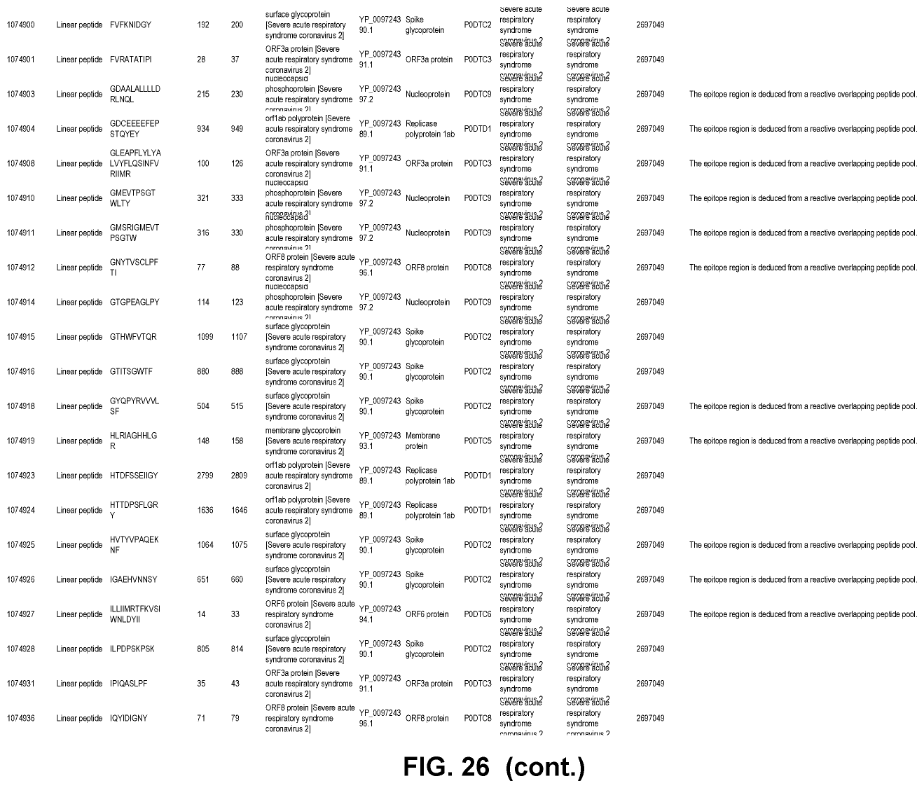

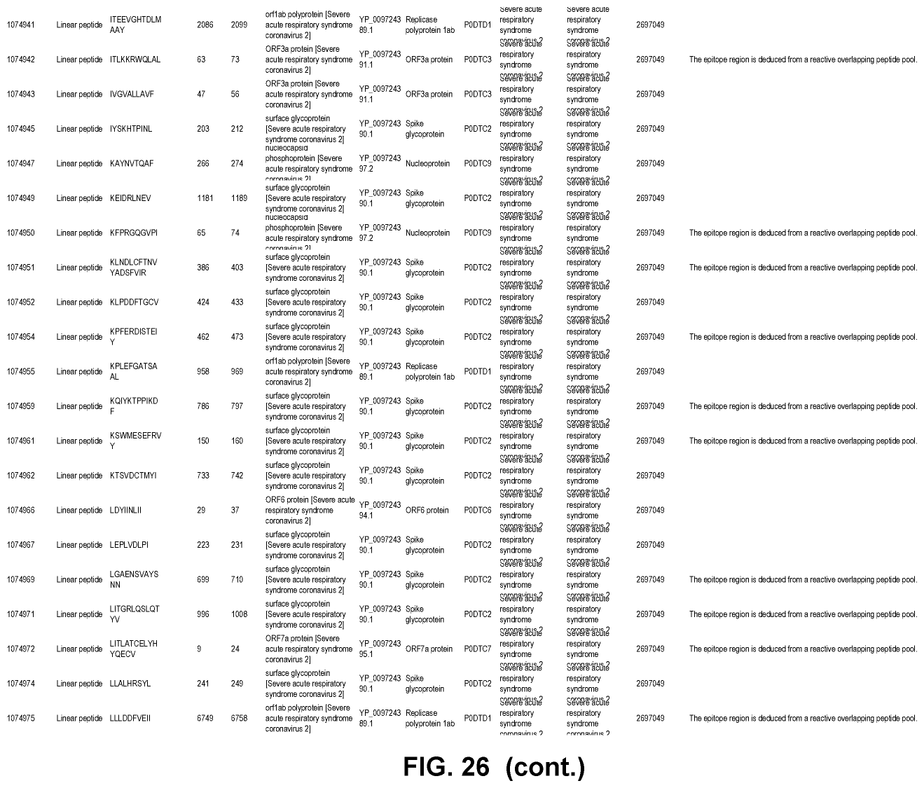

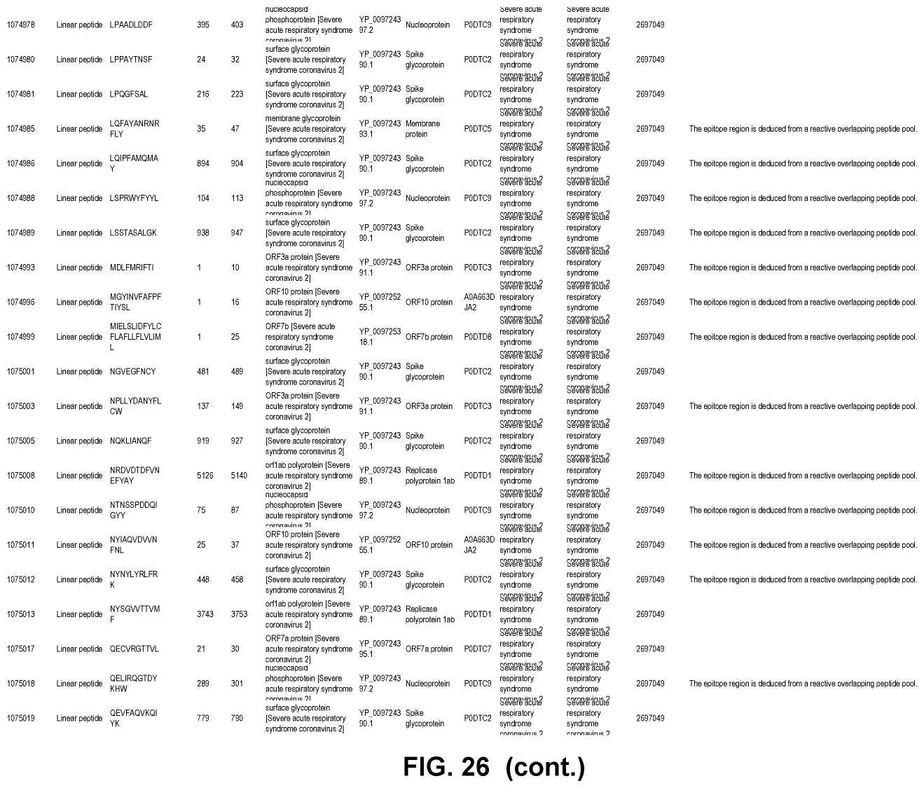

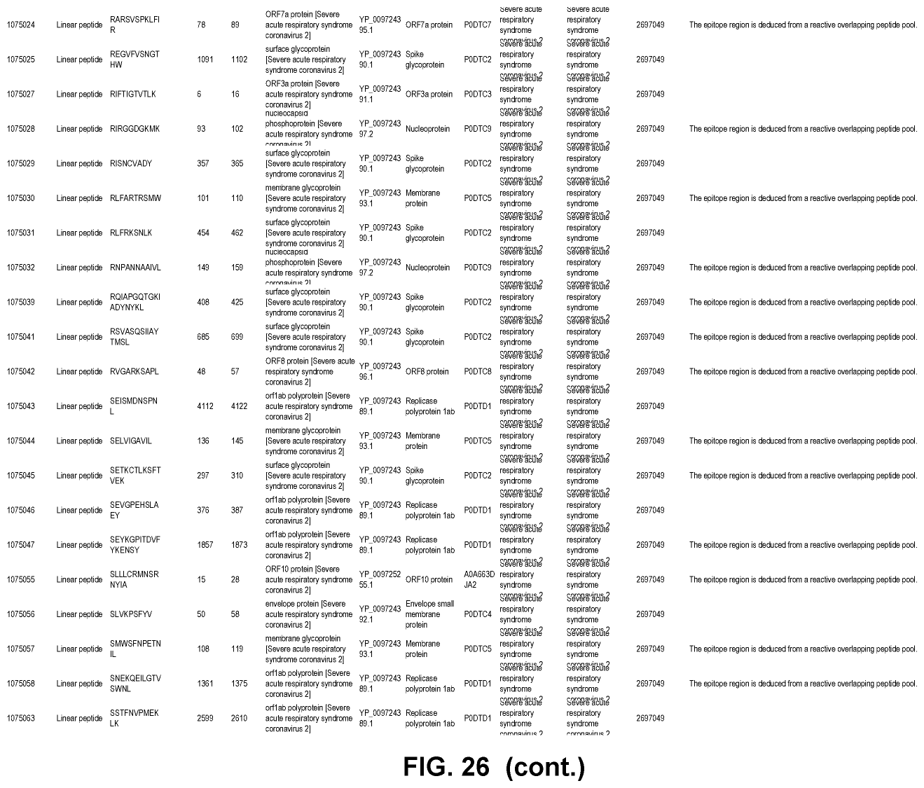

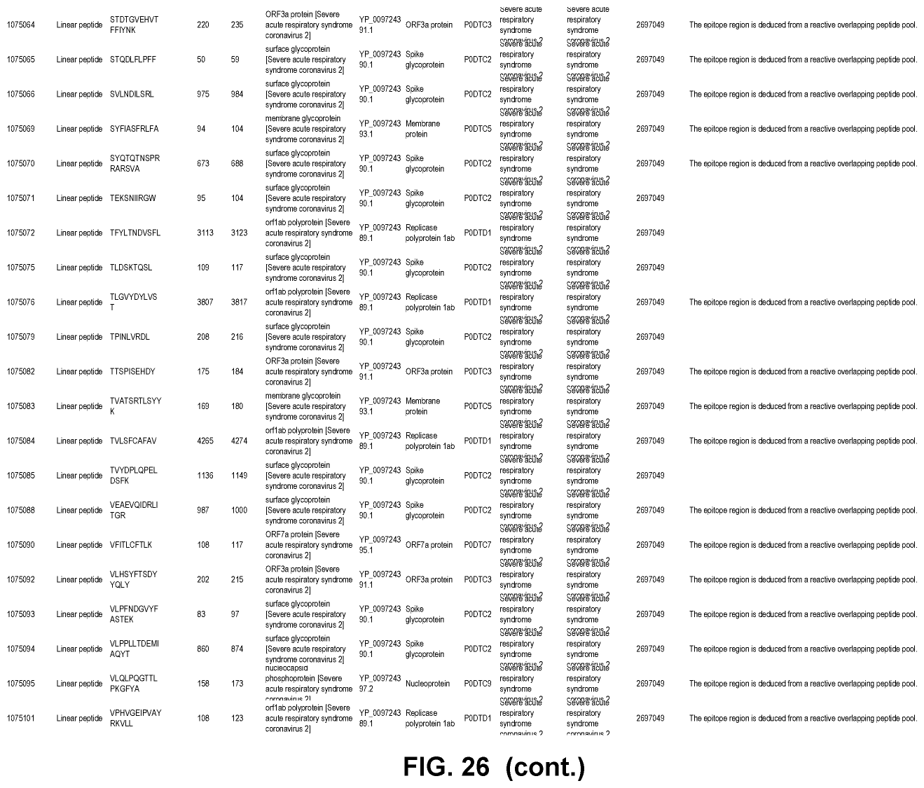

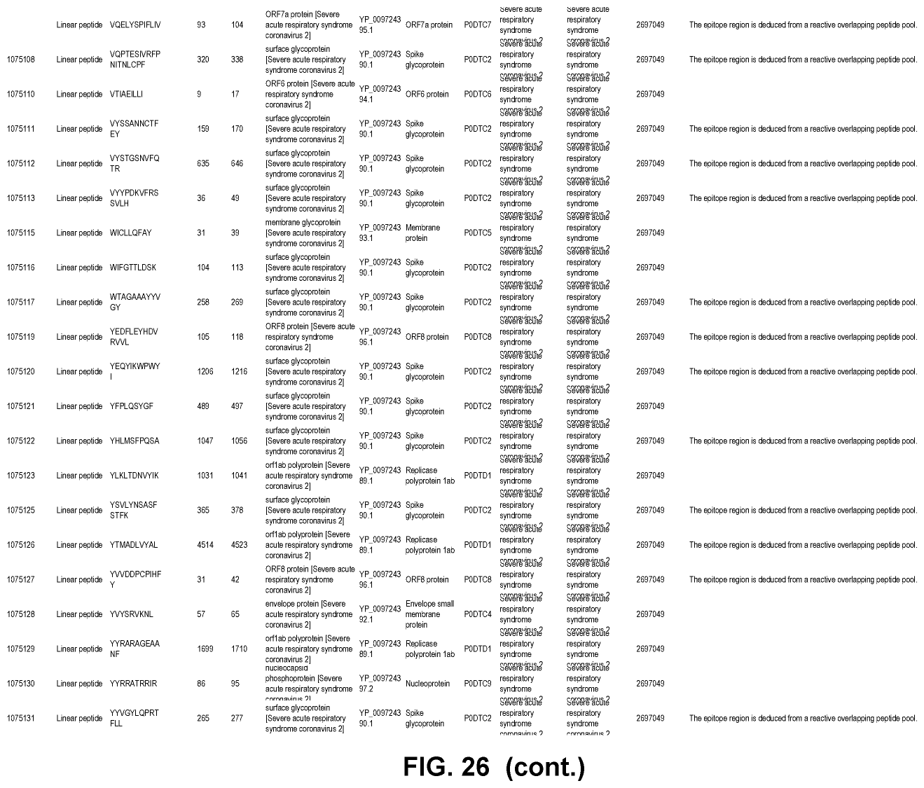

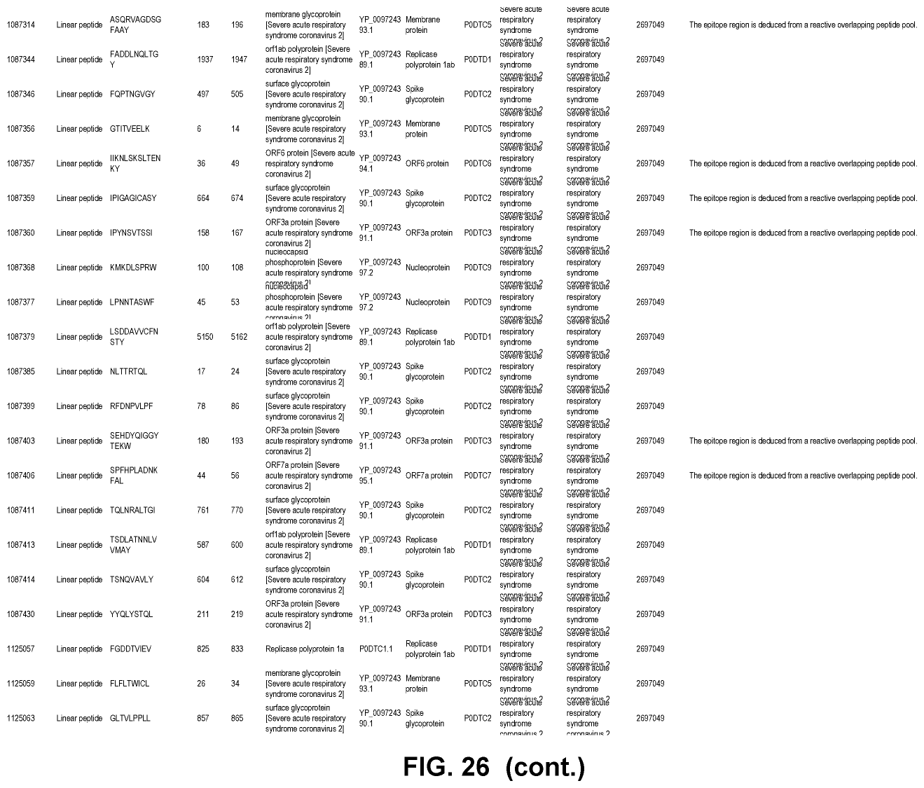

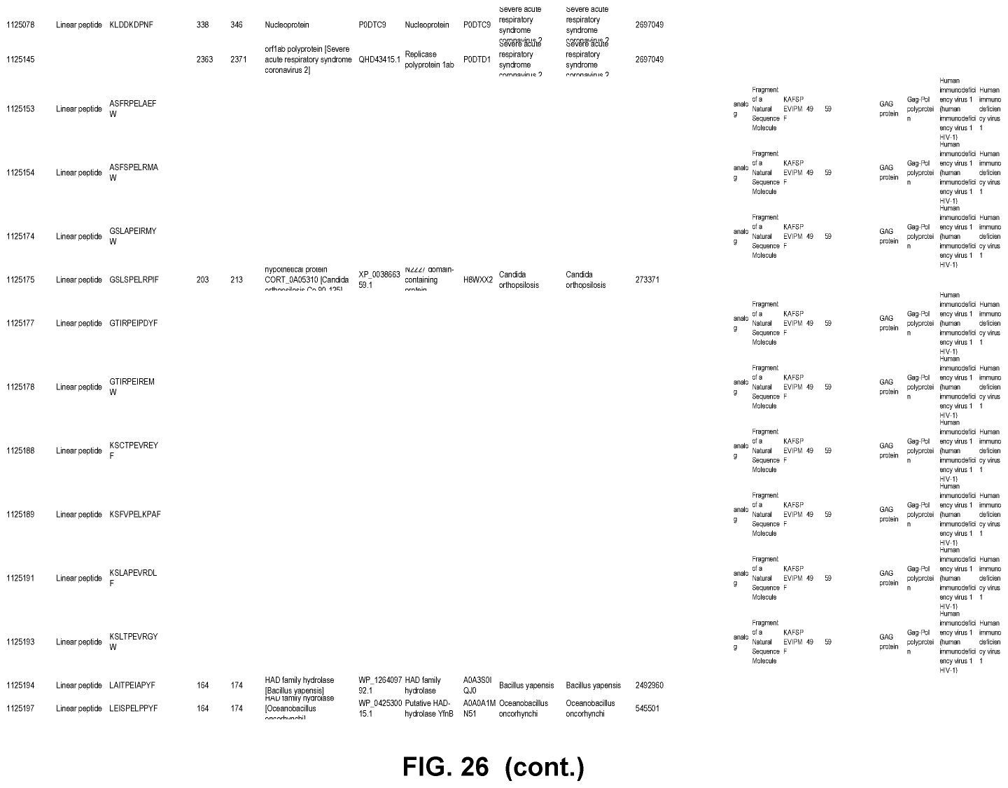

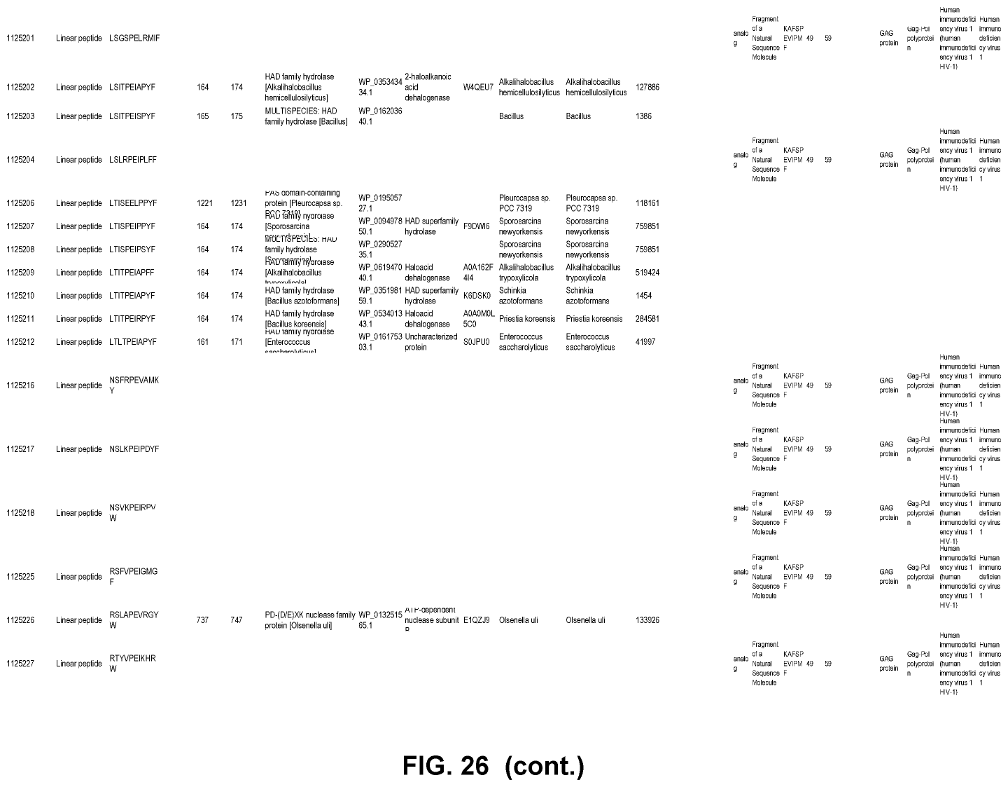

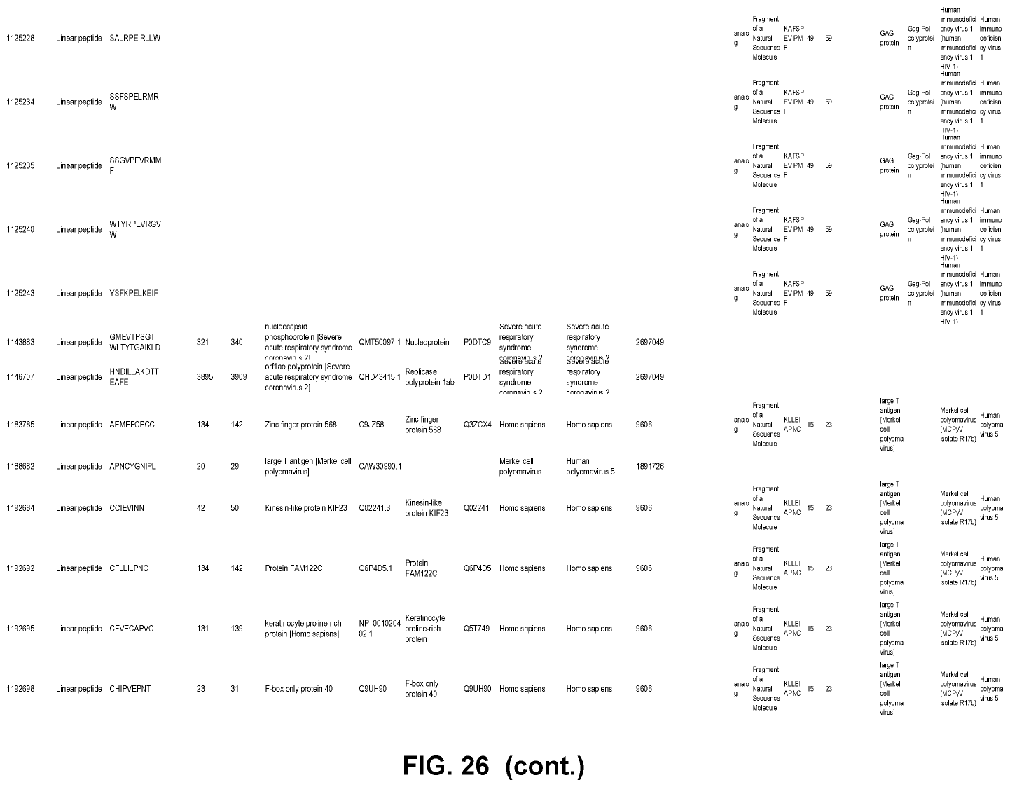

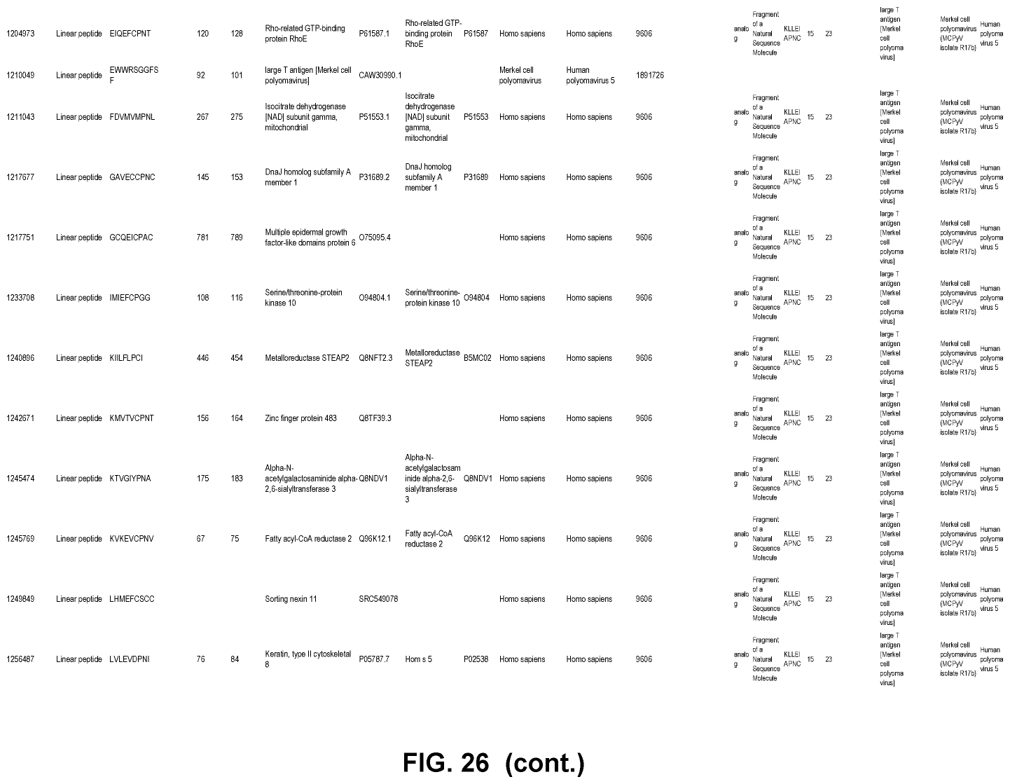

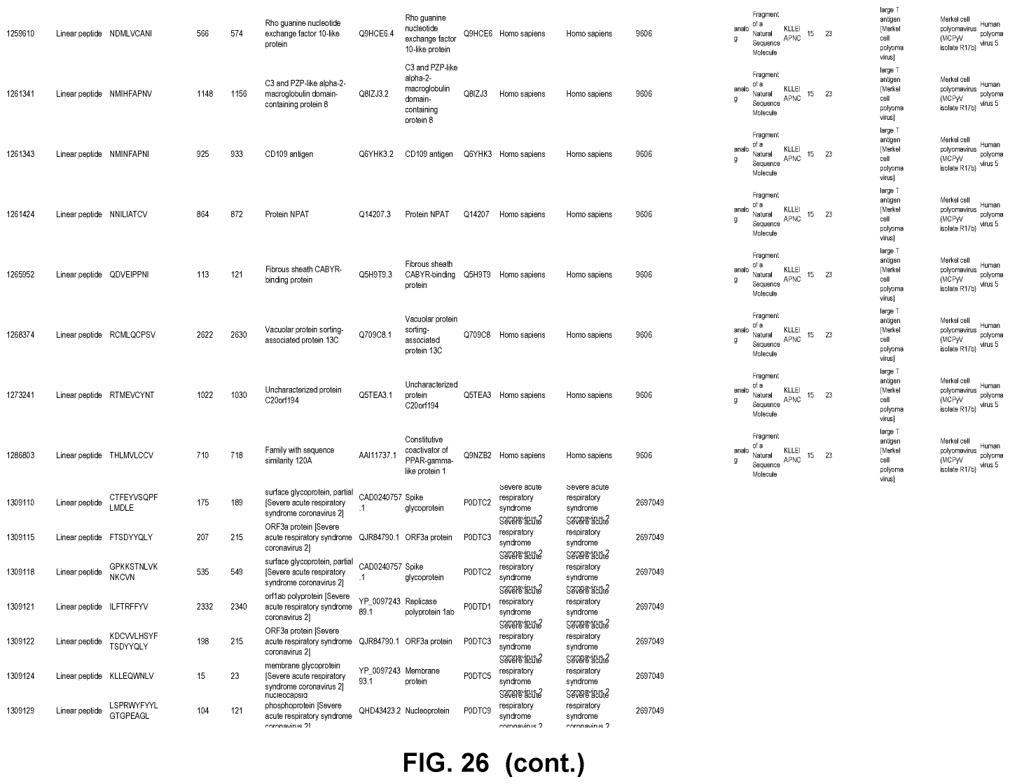

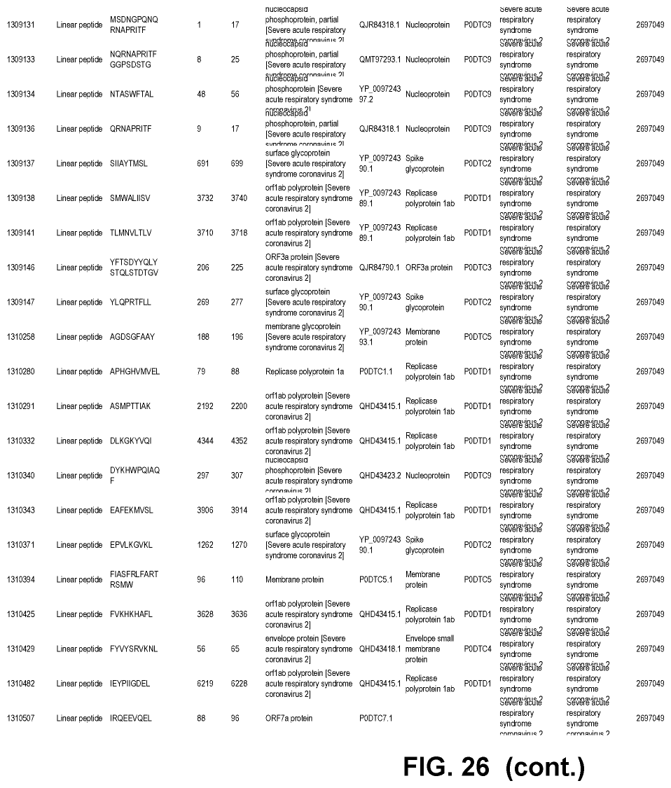

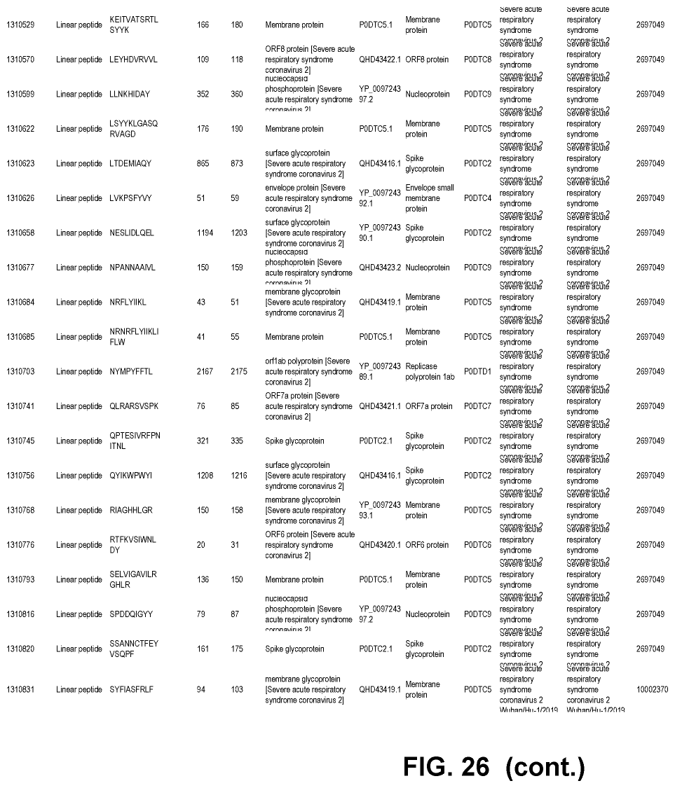

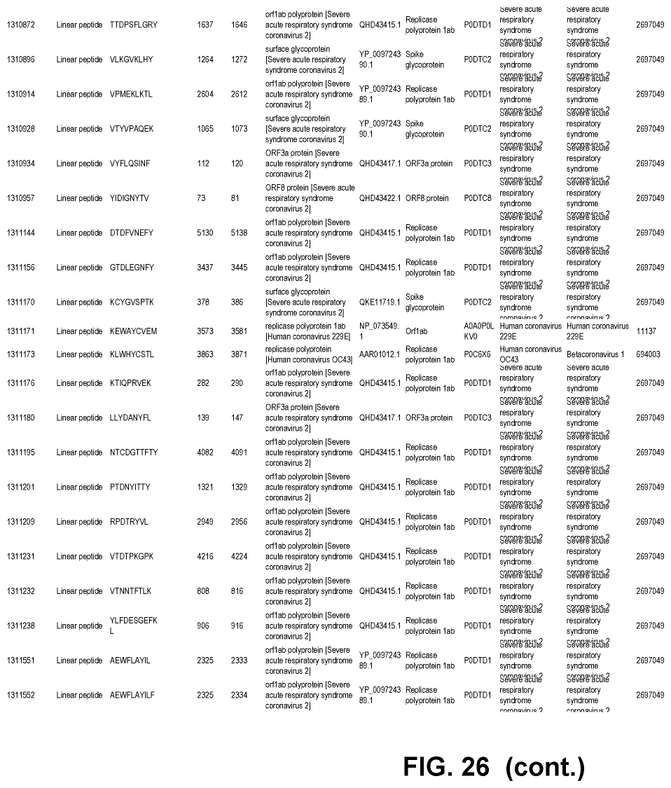

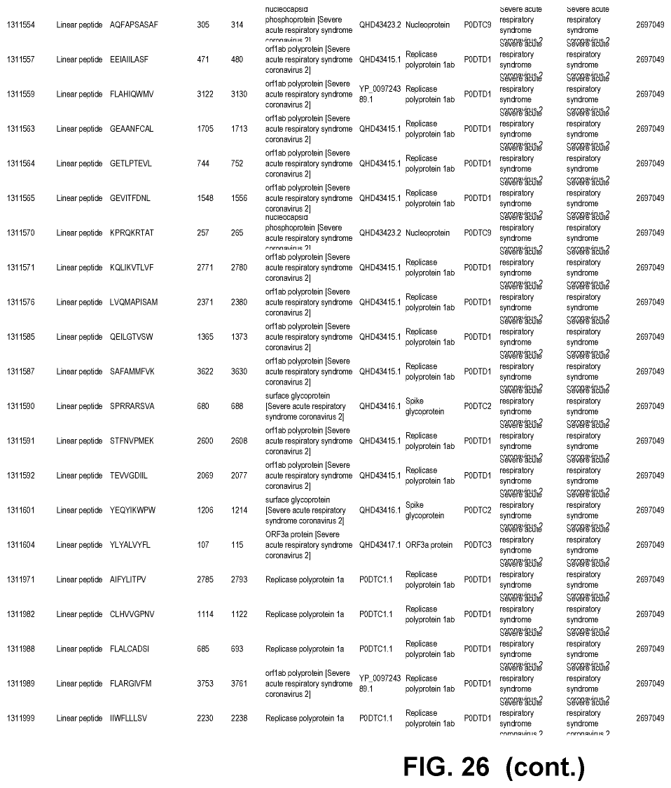

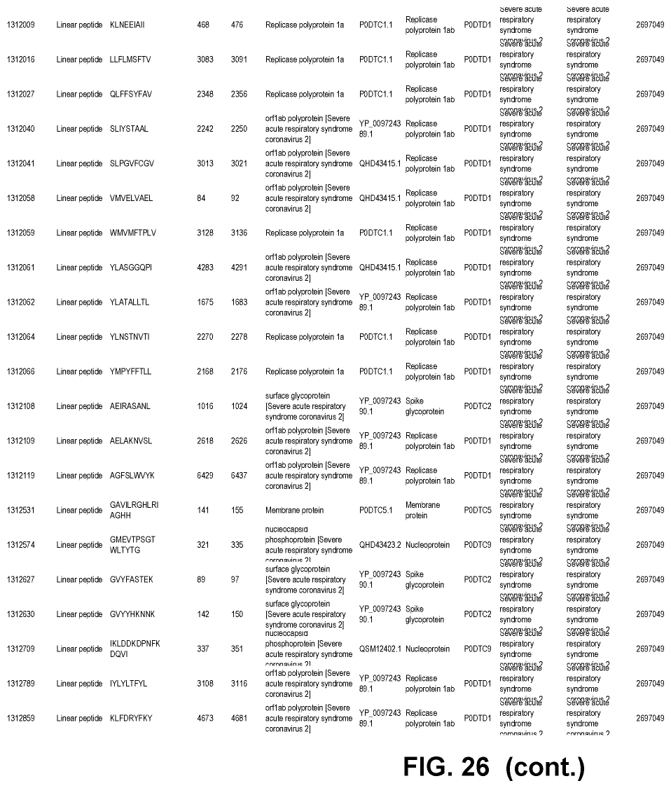

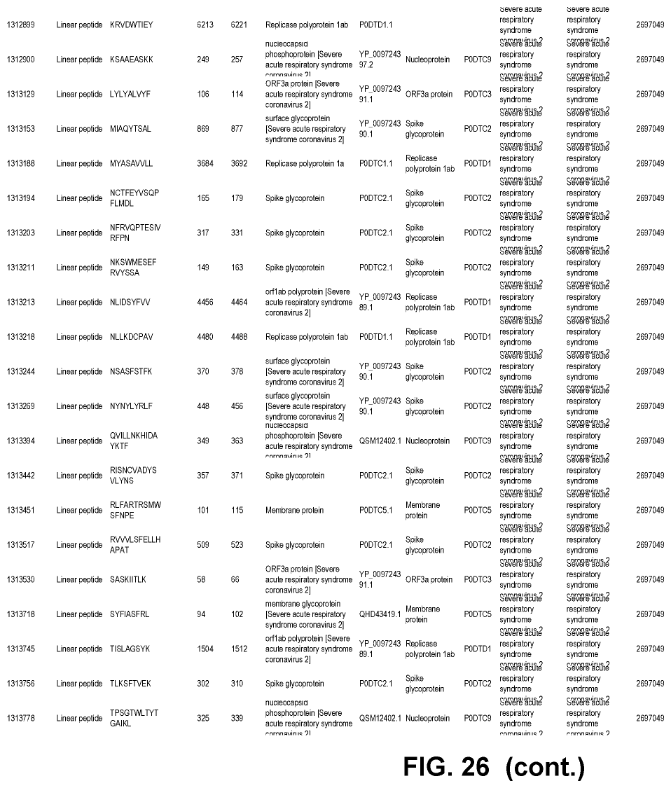

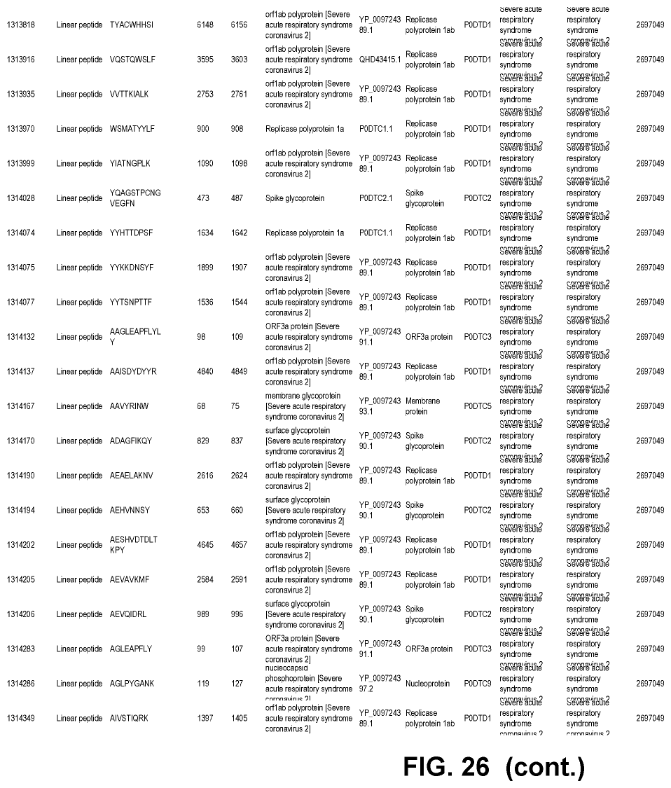

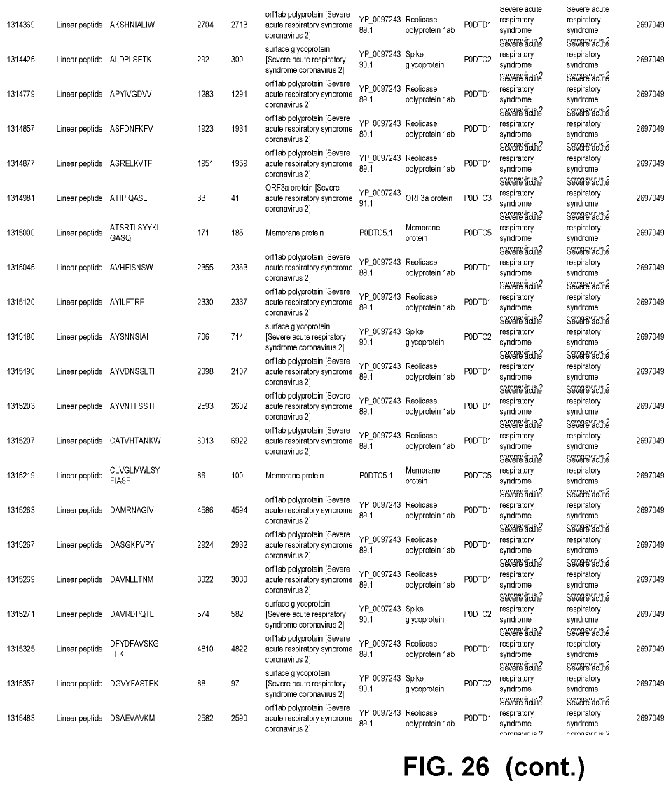

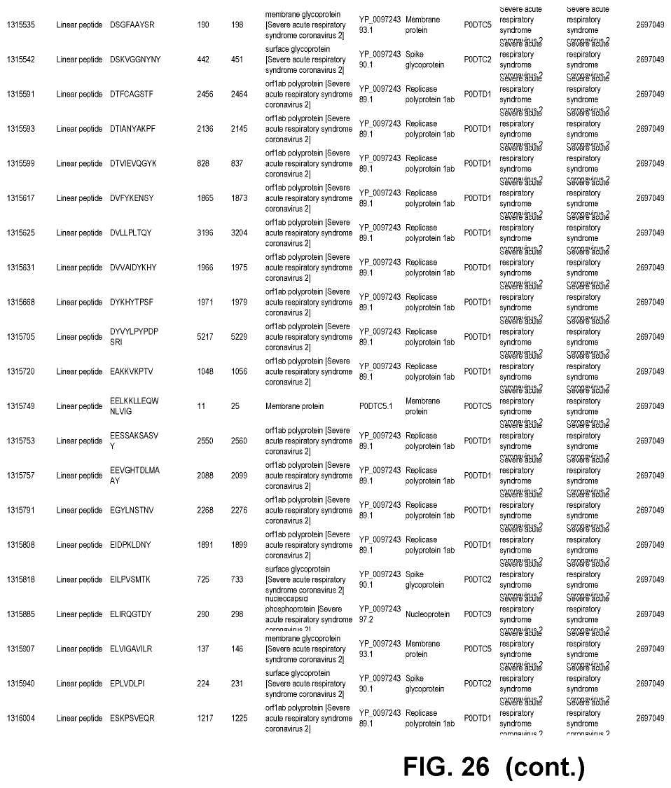

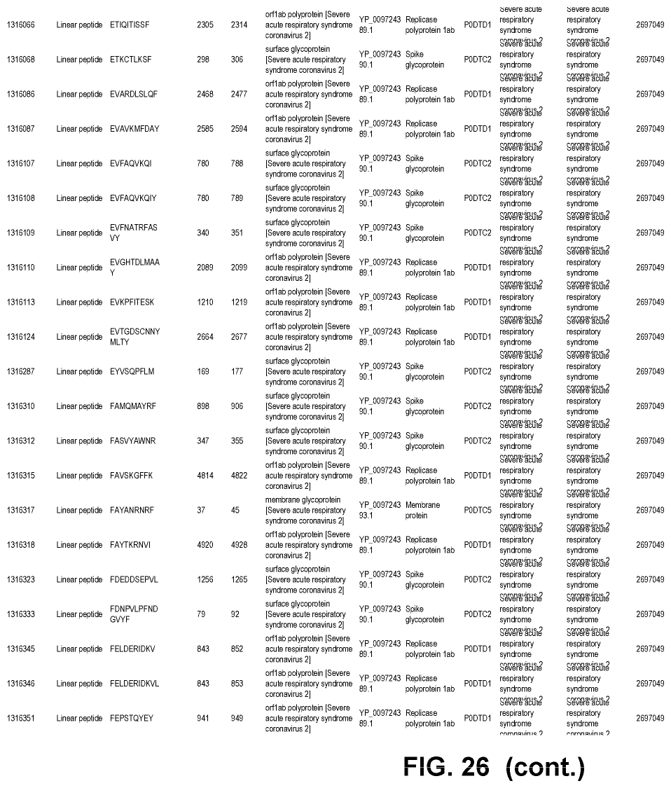

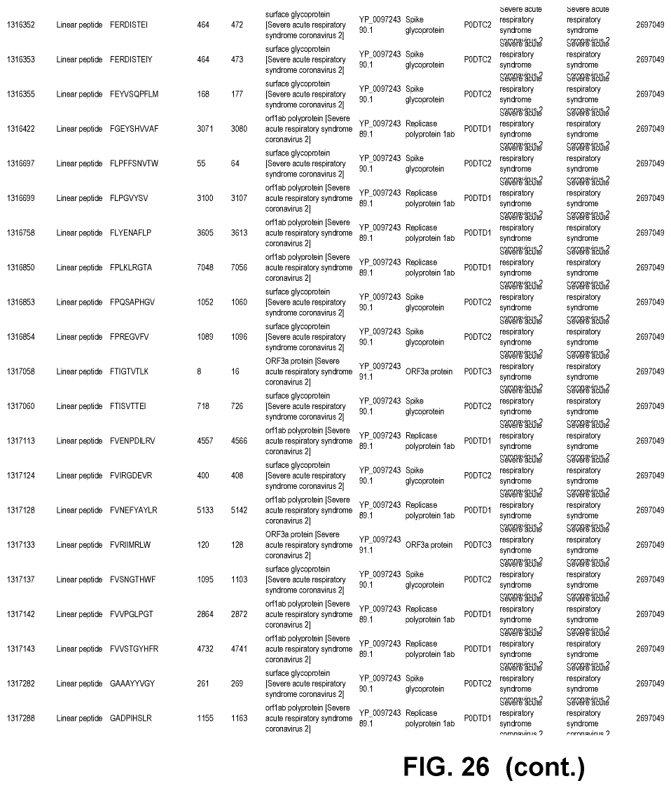

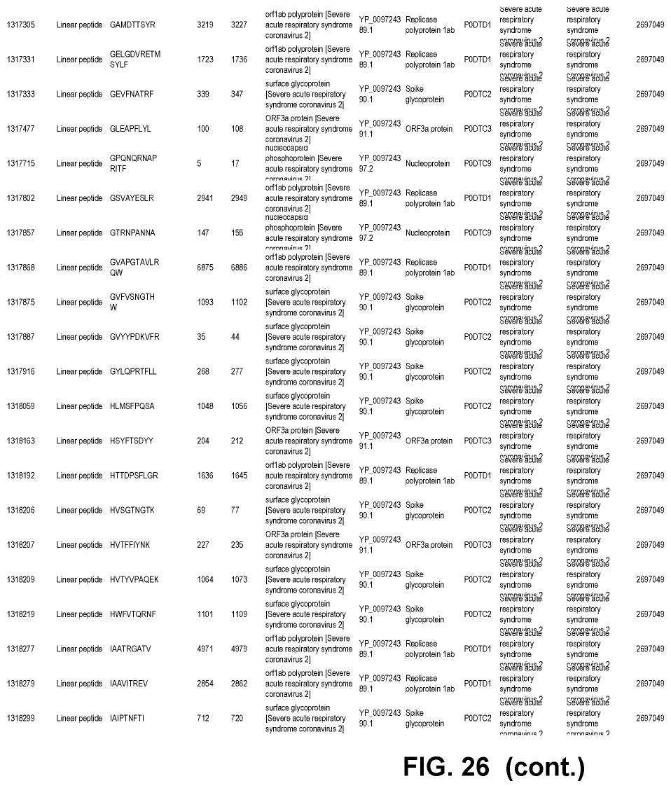

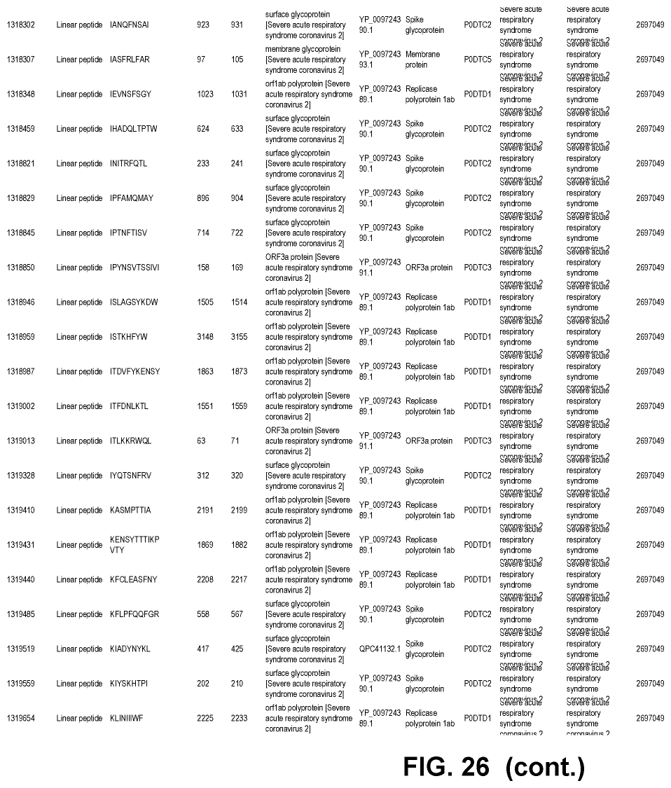

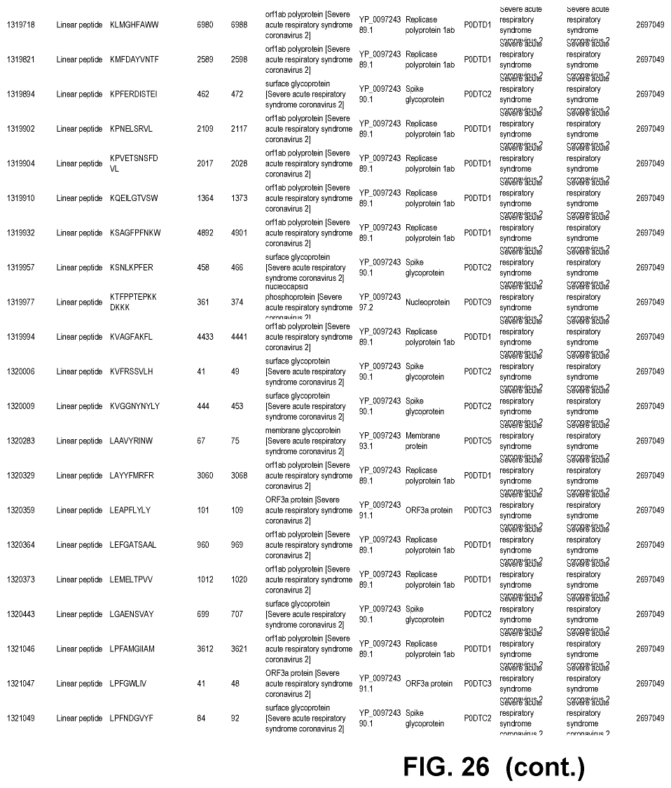

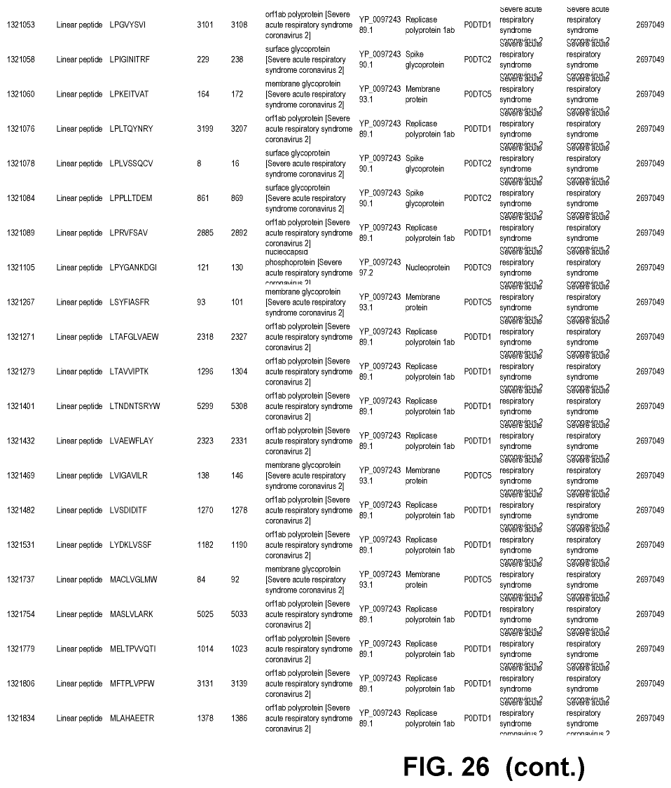

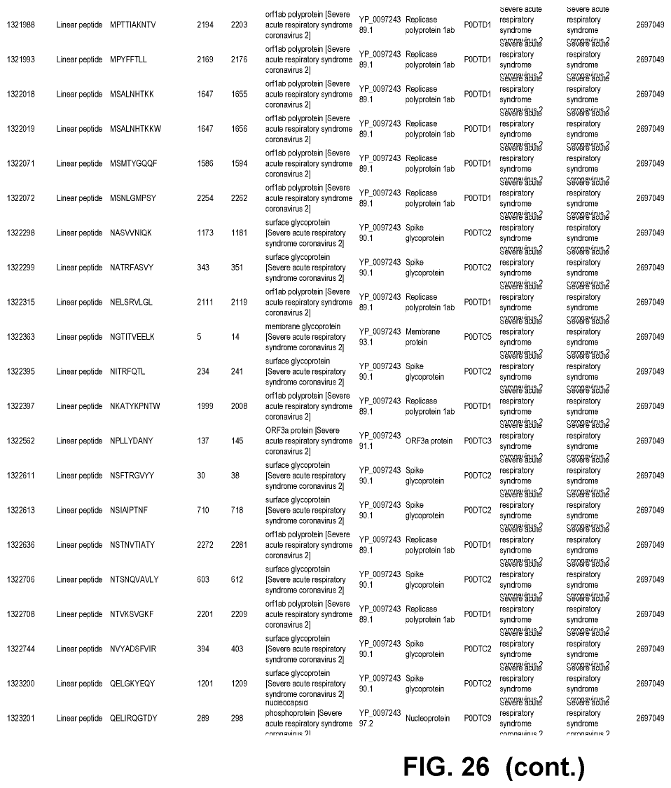

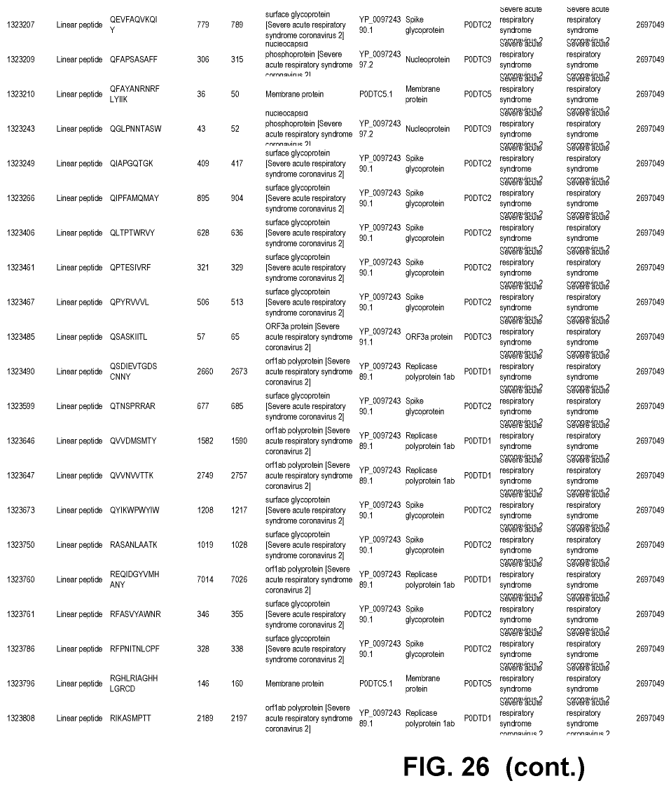

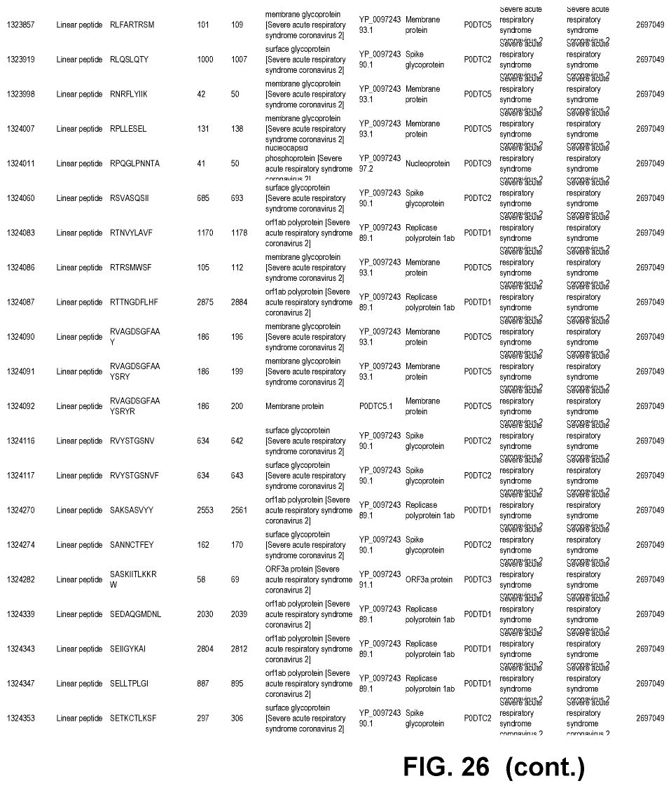

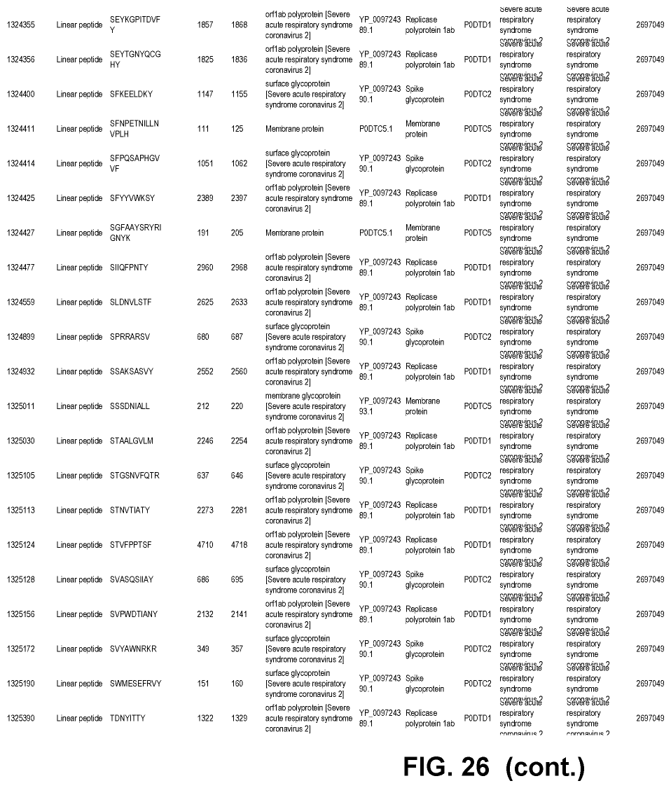

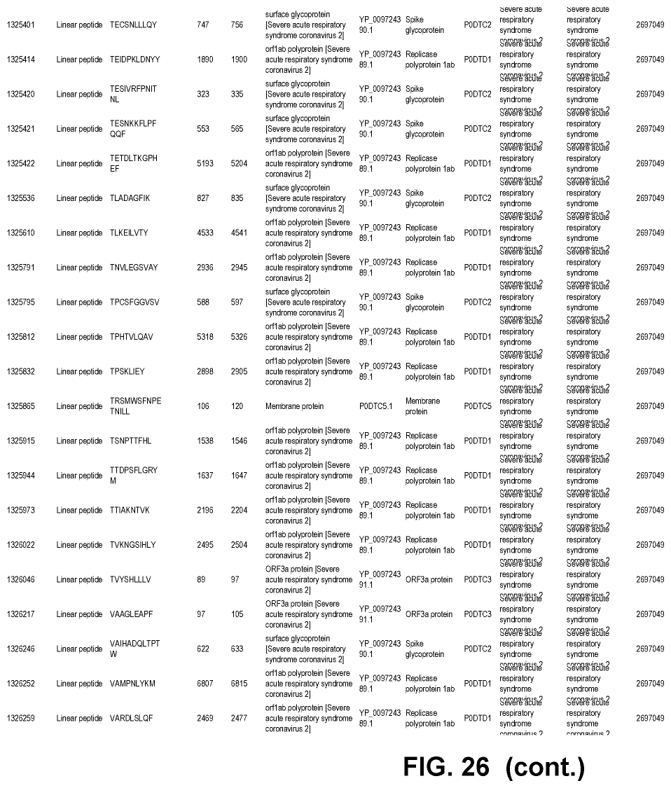

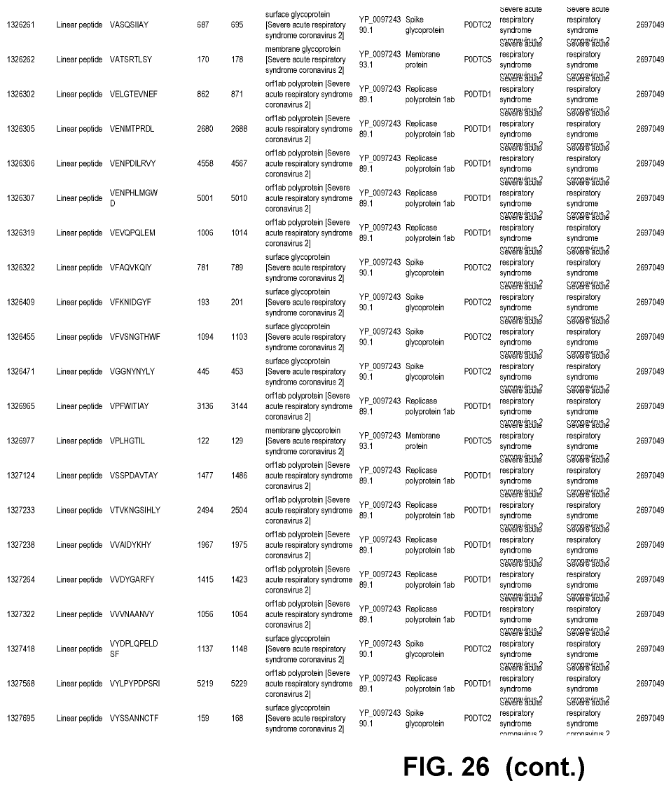

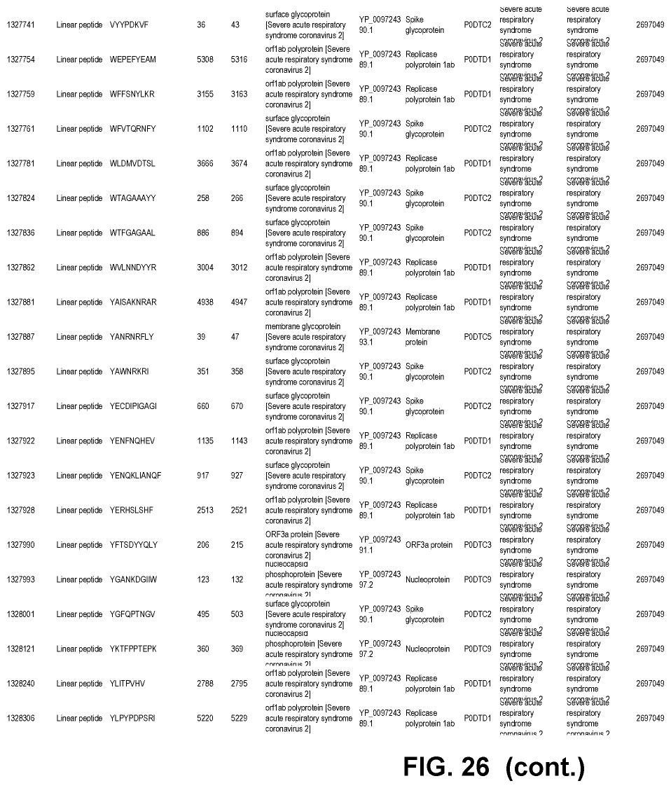

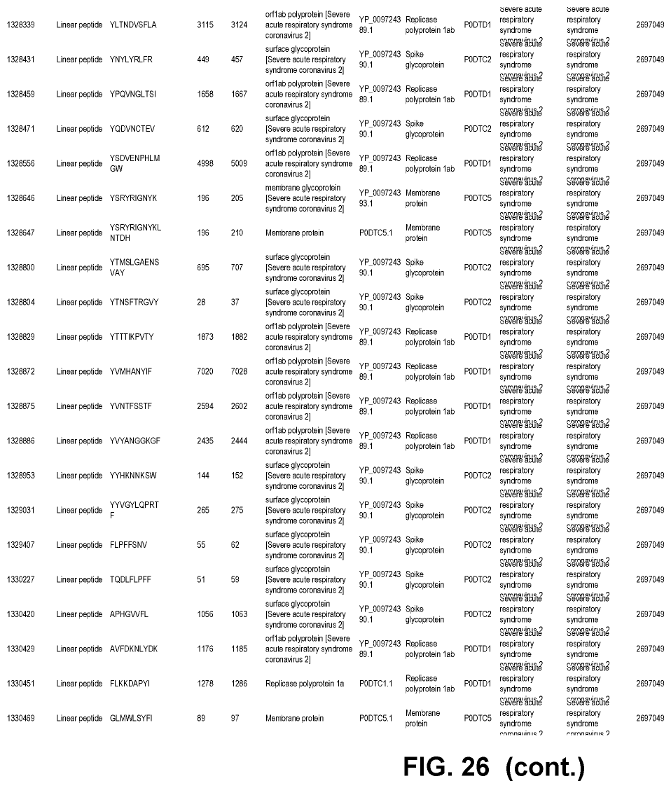

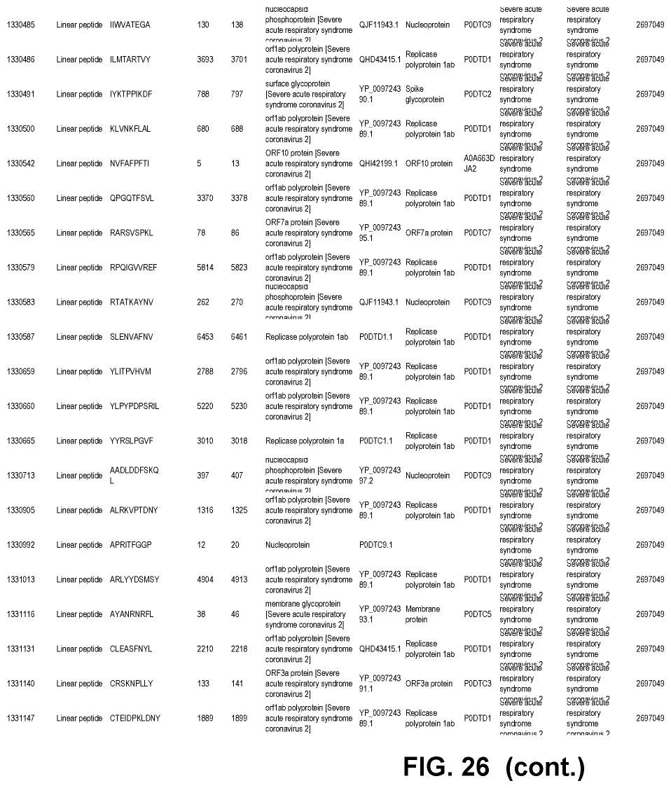

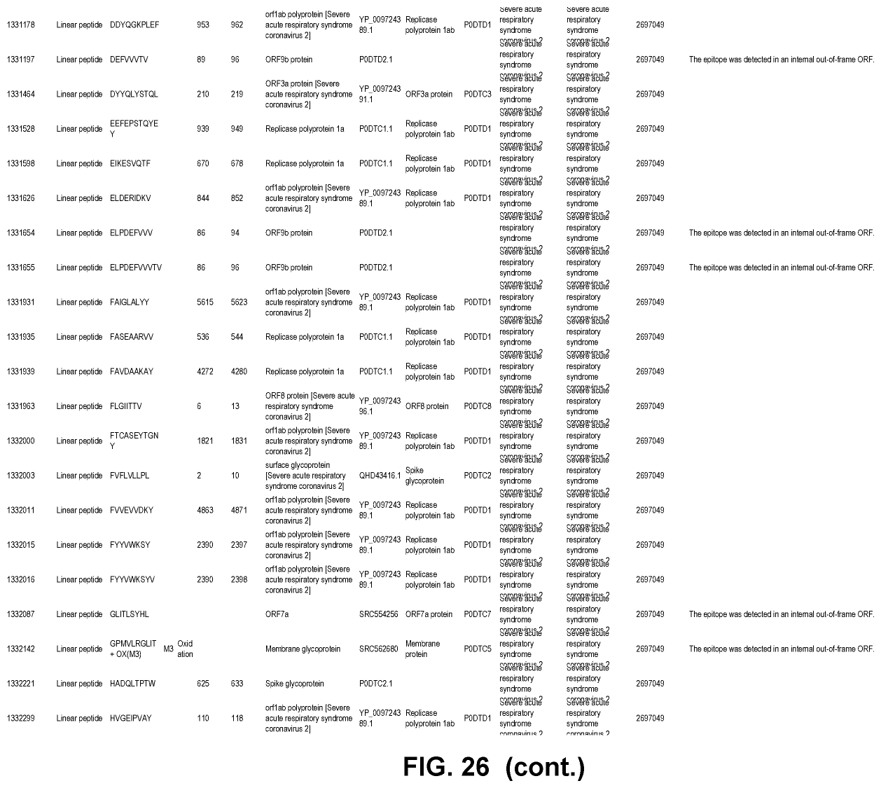

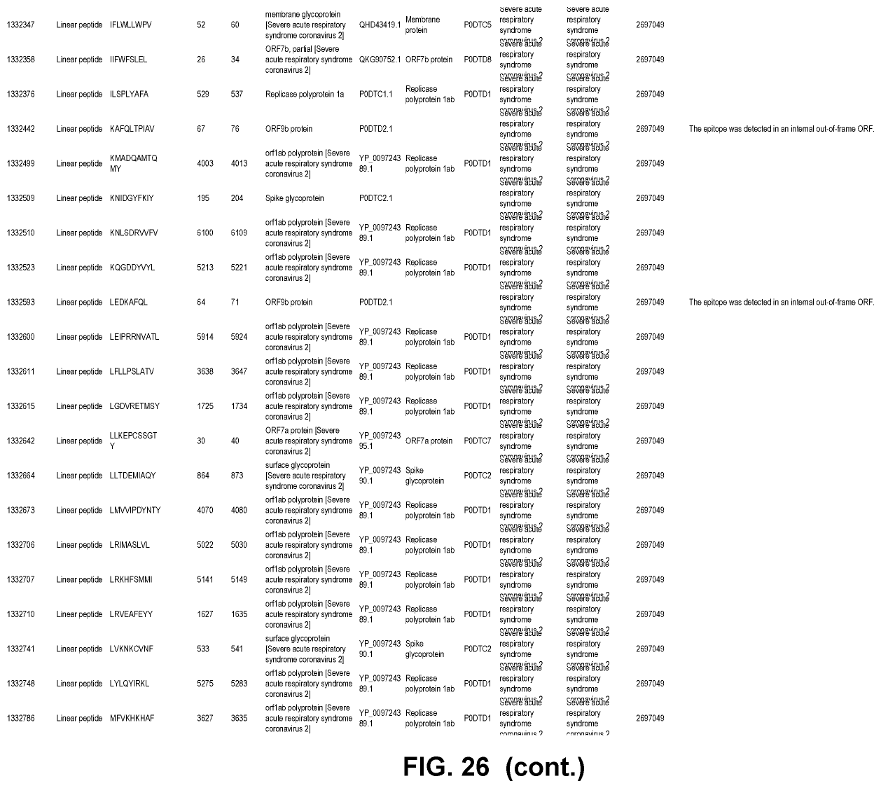

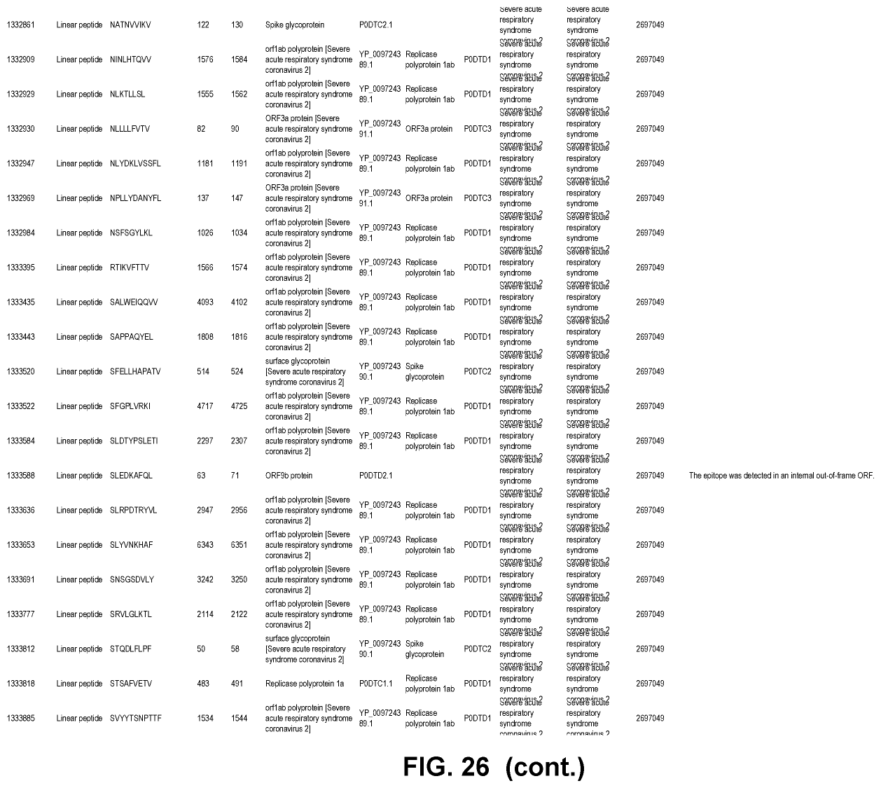

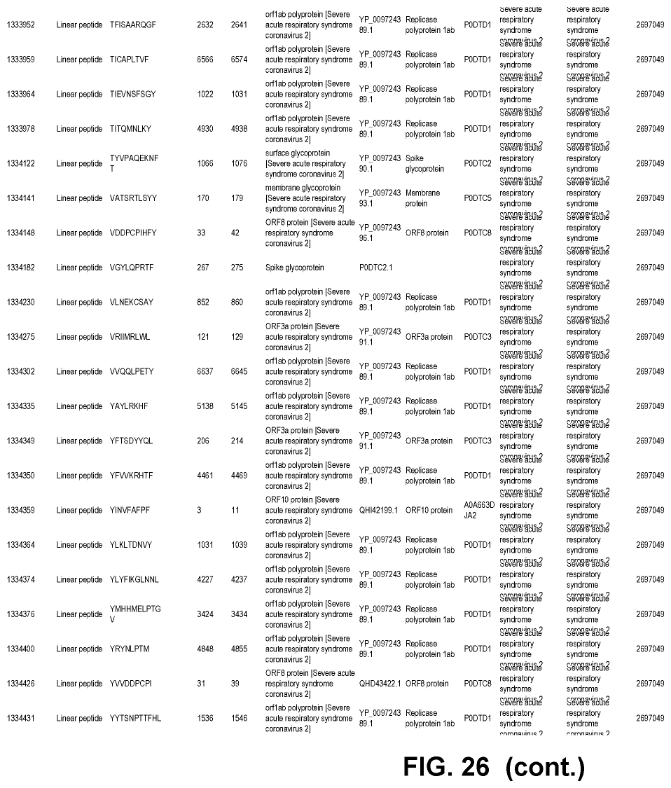

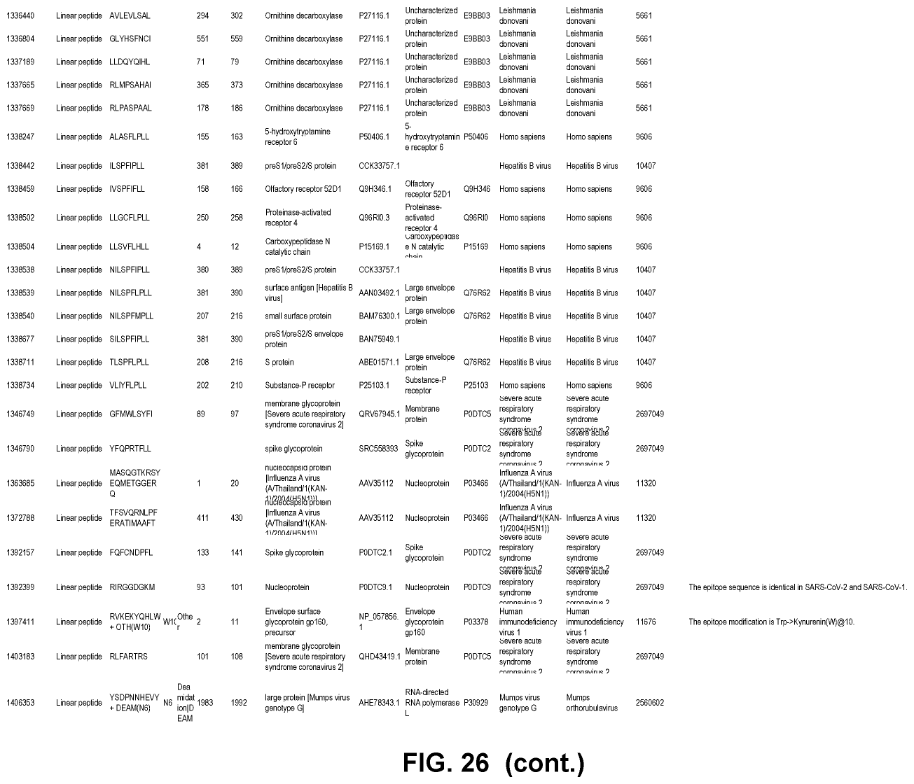

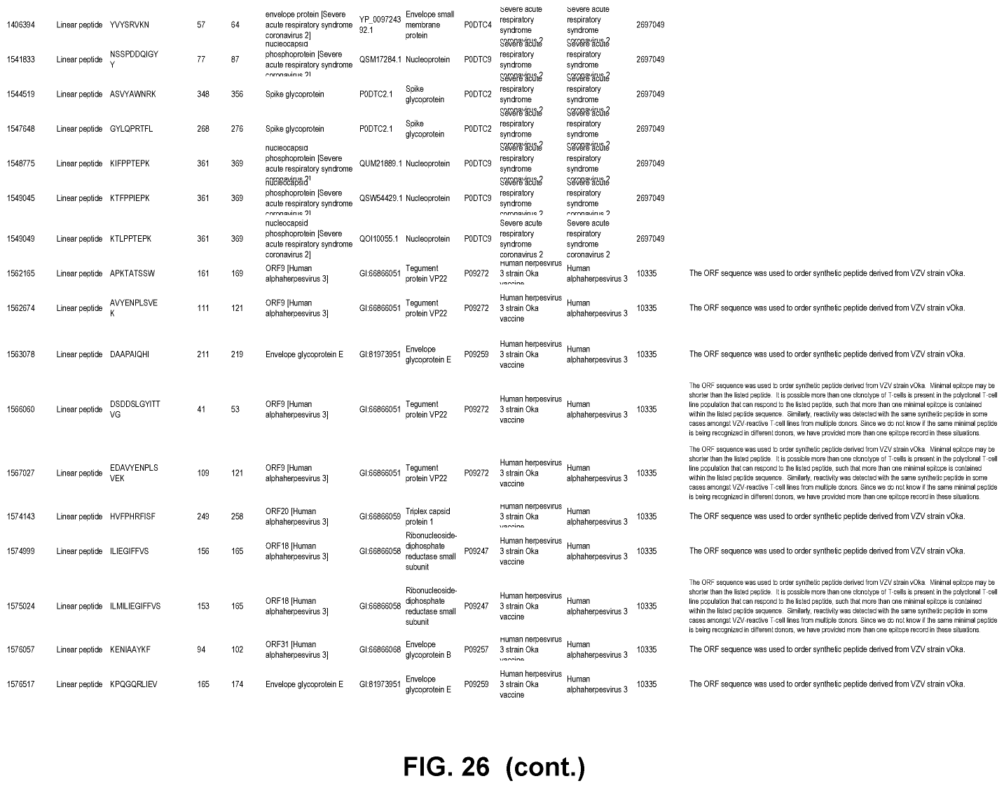

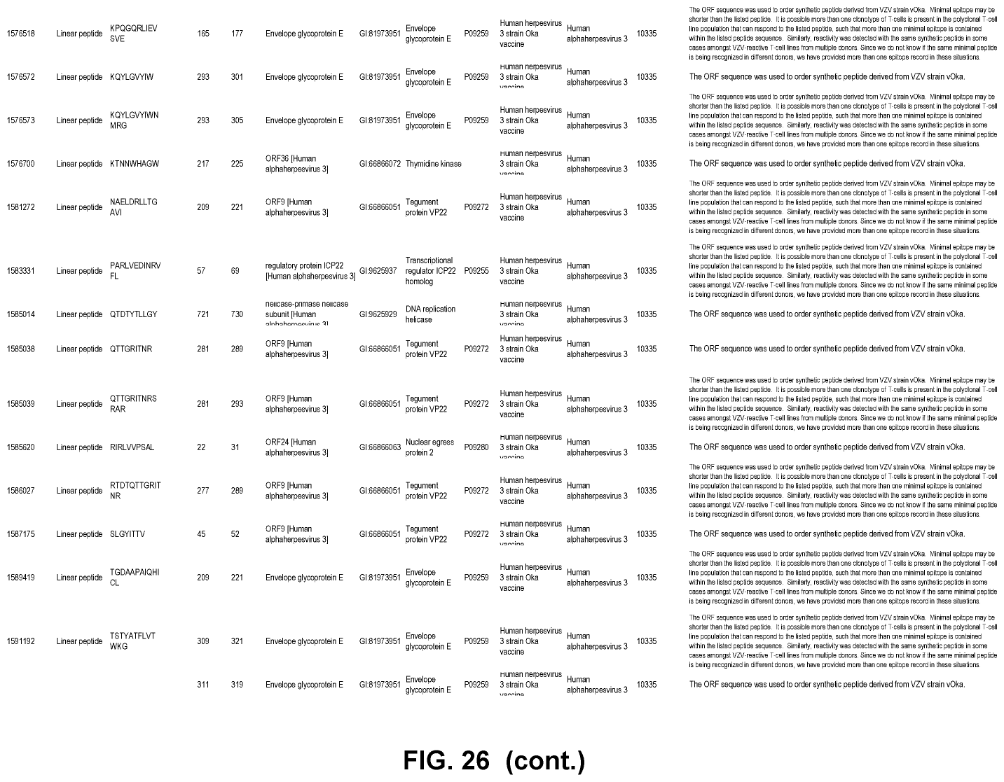

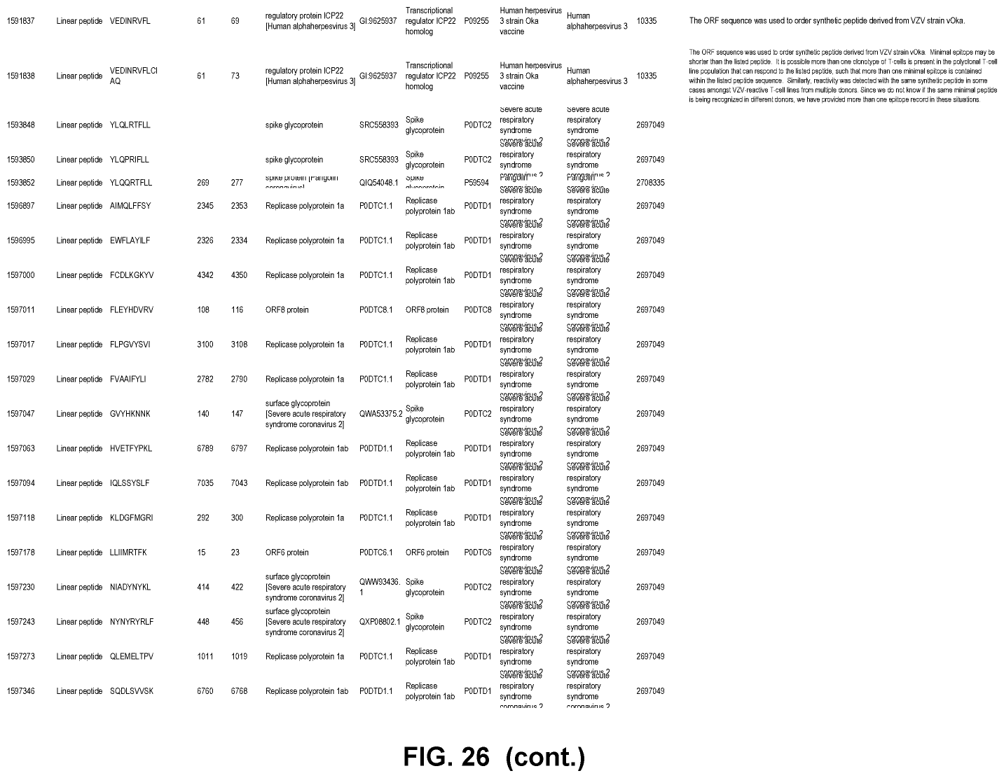

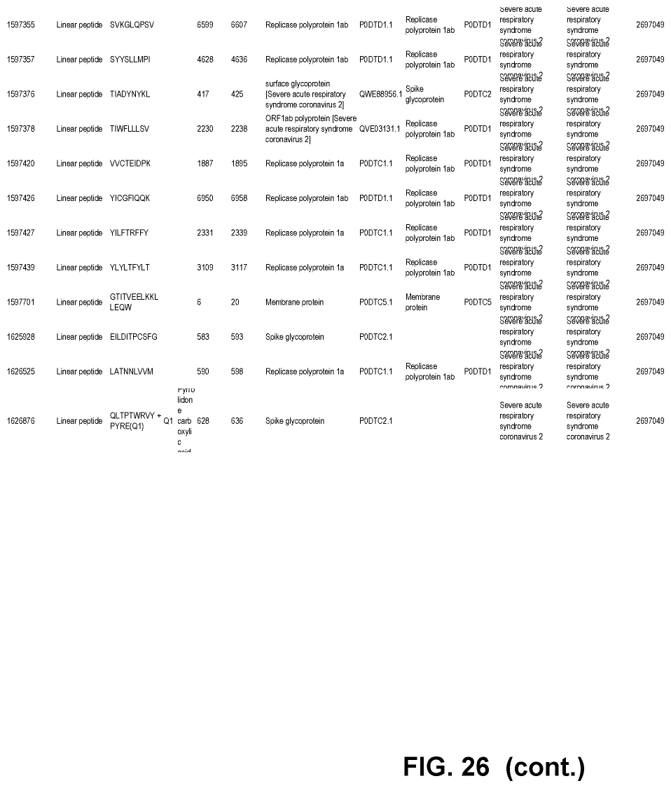

To estimate the “non-selfness” of a peptide we calculate the similarity of a given peptide to epitopes which have been previously recognized as non-self. To do so we estimate a recognition probability R of a peptide with sequence p based on its sequence similarity to a dataset of recognizable epitopes e. Here similarity is measured using a gapless alignment with BLOSUM62, though this can be easily generalized. We use a thermodynamically motivated model,^{4,5}

$$R ( p ) ≡ R ( p ; a ; k ) = Z {( p ; a ; k )}^{- 1} {\sum }_{e} exp ( - k ( a - \vert p , e \vert ) ) , \qquad 2 )$$

where Z(p; a; k)=1+Σₑ exp(−k(a−|p, e|)) is the normalization constant, |p, e| is a local alignment score between p and e, and free parameters a and k represent the horizontal displacement of the binding curve and the slope of the curve. In our case, recognizable epitopes come from the Immune Epitope Database (IEDB),⁴⁹ and we restrict our search to all human infectious disease class-I restricted targets with positive immune assays. As the peptides in IEDB can change over time, we use the current version of IEDB and list the positive epitopes used (FIG. 26). The parameters of the model are set to optimize the separation of survival curves (detailed description of parameter training is included in later sections). To find the set of IEDB epitope sequences with sequence similarity to neoantigens in our cohort, we used the blastp algorithm with the BLOSUM62 matrix (gap opening penalty=−11, gap extention penalty=−1). We calculated alignment scores with the Biopython Bio.pairwise2 package (http://biopython.org) for all alignments identified with blastp.

### Cross-Reactivity Distance: C

To model a cross-reactivity distance we measure and analyze TCR-pMHC avidity curves, by which we mean activation of a monoclonal T cell population as a function of pulsed exogenous peptide concentration. We define the cross-reactivity distance C between the two peptides as ratio of the EC₅₀ of the two avidity curves to a T cell clone that is specific to at least one of the peptides. If the EC₅₀ shift between the two avidity curves is small this reflects that the TCR is specific and highly cross-reactive to both peptides; consequently the peptides have a low cross-reactivity distance C. The reverse, a large shift in the EC₅₀, indicates a lack in reactivity against one of the peptides, low cross-reactivity, and thus a large cross-reactivity distance C. Formally, this quantity could depend on the TCR and the HLA allele, however we fit a minimal model for peptides that are one amino acid substitution from each other with the intention of extracting coarse grain features that are sufficiently robust for our application.

### Fitting Avidity Curves

To fit our model we measured avidity curves (FIG. 3, panel C, FIG. 7, panels C-E) corresponding to seven different peptide-TCR combinations: 3 TCRs specific to a CMV epitope (NLVPMVATV), 3 TCRs specific to a gp100 epitope (IMDQVPFSV), and 1 TCR specific to a neoantigen arising from a mutation in the RHBDF2 gene (GRLKALCQR). For each peptide-TCR combination we measured the avidity curves for the wildtype peptide along with all 171 peptides one amino acid substitution away from the wildtype peptide (1204 total TCR-pMHC combinations)(FIG. 8, panels A-C). For each peptide we extract the EC₅₀ from the TCR-pMHC reactivity curve by fitting a generalize Hill function:

$$V ( c ) = \frac{{V}_{∞}}{1 + {( \frac{{EC}_{50}}{c} )}^{n}} . \qquad ( 3 )$$

This function has 3 parameters: the maximum amplitude V., the cooperativity n, and, the term to be inferred, the EC₅₀. As we have 3 concentration points for each peptide, regularization is key to a robust fit of these curves. To motivate the regularization, we use the priors that V_{∞}≈1 (i.e. at infinite peptide concentration, TCR reactivity approaches 100%) and n≈1 (cooperativity of 1 is for the case of simple binding reactions of 2 molecules). Finally, we enforce a slight regularization on the EC₅₀ if it extends outside of the measured concentration region. We use an L2 cost and regularization to fit, yielding a cost function to minimize:

$${\sum }_{c} {( V ( c ) - {V}_{meas} ( c ) )}^{2} + {r}_{V} {( 1 - {V}_{∞} )}^{2} + {r}_{n} {( n - 1 )}^{2} + {r}_{{EC}_{50}} d {( {EC}_{50} , [ 0.01 , 100 ] )}^{2} . \qquad ( 4 )$$

Where d(EC₅₀, [0.01, 100]) indicates the log distance to the measured concentration range of [0.01 μg/mL, 100 μg/mL] (i.e. d(EC₅₀, [0.01, 100])=max(0, log(EC₅₀)−log(100), log(0.01)−log(EC₅₀)). The regularization constants used were r_{V}=0.01, rₙ=0.01, and r_{EC}_{₅₀}₌_{_{0.001}}. Parameters were then fit using standard least squares. The inferred EC₅₀'s were further clipped to the range of 10⁻⁴ μg/mL to 10⁴ μg/mL.

### Cross-Reactivity Distance Model

To model the effect of a single amino acid substitution on an avidity curve's EC₅₀, we assume that there is a position independent amino acid substitution matrix M that is rescaled by a position dependent factor dᵢ. Together this yields a model of the following form for the cross-reactivity distance C between two peptides, p^{A} and p^{B}, which differ only by a single mismatched amino acid in position i.

$$\vert log ( C ( {p}^{A} , {p}^{B} ) ) \vert = \vert log ( \frac{{EC}_{50} ( {p}^{B} )}{{EC}_{50} ( {p}^{A} )} ) \vert = {d}_{𝒾} ℳ ( {p}_{i}^{A} , {p}_{i}^{B} ) \qquad ( 5 )$$

This form of the model for C has more parameters than can reliably be inferred from our experimentally measured TCR-pMHC avidity curves—the distance weight dᵢ has 9 parameters, and the substitution matrix M has 380 free parameters (190 if we assume a symmetric matrix).

To ameliorate this problem, we implement two modifications to reduce the effective number of parameters—first, we embed the 20 amino acids into a bounded 2D region (a 20×20 square) and define the values of the substitution matrix M as the Euclidean distance between the positions of each embedded amino acid. This reduces the number of free parameters for M from 190 to 40 and allows for clear visualization of amino acid clustering.

Second, we introduce the BLOSUM62 substitution matrix as a prior (we find a model inference performed without this assumption shows that the inferred substitution matrix correlates significantly to BLOSUM62). We define a cost function that includes not only the differences between the measured and modeled distances but also a regularization term that reflects how well a linear transformation of the BLOSUM62 matrix matches the inferred substitution matrix (we exclude the diagonal terms from this fit as those terms are not fit under the model). The full expression is:

$$\frac{1}{\vert \{ {p}^{A} , {p}^{B} \} \vert} {\sum }_{\{ {p}^{A} , {p}^{B} \}} {( \vert log ( C ( {p}^{A} , {p}^{B} ) ) \vert - \vert log ( {C}_{meas} ( {p}^{A} , {p}^{B} ) ) \vert )}^{2} + {r}_{b 162} {RSS}_{b 162} \qquad ( 6 )$$

where we sum over pairs of measured peptides {p^{A}, p^{B}} and RSS_{bl62} is the sum of the square residuals of the optimal linear regression between M and BLOSUM62.

We used a value of r_{bl62}=0.01 for the constant that controls the relative weighting of the fit to the measured data or the fit to BLOSUM62. We then use the dual annealing method to minimize the cost function and fit the model parameters.

This model is inferred using the measured log distance between the EC₅₀ of two peptides to the same TCR. We restrict the peptide pairs we consider in our inference by requiring both that the peptides are a single amino acid substitution away from each other and that there is some minimal reactivity to at least one of the peptides to the associated TCR. We set this reactivity threshold as the criteria that the EC50 of at least one of the peptides must be less than 0.1 μg/mL. This criteria will include all pairs that include one of the wildtype peptides (NLVPMVATV, IMDQVPFSV, or GRLKALCQR), but may also include pairs of mutants that have substitutions in the same position in the wildtype (e.g. NLVMMVATV and NLVKMVATV). Including these additional combinations allows us to more accurately resolve amino acid substitutions not observed from the original 3 wildtype peptides.

### Cross-Reactivity Model Validation

To validate the cross-reactivity model C, we inferred a model using peptide pairs only from the NLV and gp100 TCRs (6 TCRs in total) (FIG. 10, panels A and B) and predicted on the remaining RHBDF2 neoantigen peptide pairs (with the same minimum reactivity restrictions as described above for the inference). The NLV and gp100 peptides are presented on HLA-A02:01 whereas the RHBDF2 neoantigen is predicted to be presented on HLA-B27:05, so the validation dataset stems from not only a different wildtype peptide-TCR combination, but also a wholly different HLA allele. We find that the model learned on the NLV and gp100 TCRs provides highly significant predictive power for the peptide pairs from the RHBDF2 neoantigen (FIG. 3, panel F).

### Self Discrimination: D

The self discrimination component quantifies how easily p^{MT} and p^{WT} can be distinguished from each other as a result of negative selection, and is a sum of terms relating to both the MHC presentation and our experimentally derived cross-reactivity distance C. For a given HLA allele h, we calculate the peptide-MHC-I dissociation constants, 50 K_{d}^{MT}≡K_{d}(p^{WT}, h) and K_{d}^{WT} ≡K_{d}(p^{WT}, h) for both peptides. We consider the relative MHC dissociation constants between p^{MT} and its p^{WT} counterpart, as the ratio of their inferred MHC-I binding affinities.

We define the combined self discrimination D of a neoantigen as:

$$D ( {p}^{MT} , {p}^{WT} , h ) = ( 1 - w ) log \frac{{K}_{d} ( {p}^{WT} , h )}{{K}_{d} ( {p}^{MT} , h )} + w log \frac{E {C}_{50} ( {p}^{MT} )}{E {C}_{50} ( {p}^{WT} )} . \qquad ( 7 )$$

Each term represents an affinity difference, or discrimination energy, between p^{MT} and p^{WT} either for MHC presentation or for T cell activation. The self discrimination D therefore increases if either the underlying mutation leads to an increased presentation probability, or if it results in a peptide not cross-reactive with the wildtype and thus recognized by a collection of TCRs distinct from those that recognize the wildtype peptide. Parameter w∈[0, 1], sets the relative weight between the two terms: MHC presentation and T cell activation.

### Amino Acid Clustering and Subsequent Ordering

The dendogram and the amino acid ordering in FIG. 3, panel G were computed by unsupervised agglomerative clustering using the sklearn package with 0 distance thresholding (sklearn.cluster.AgglomerativeClustering). The distances used for the clustering were the Euclidean distances arising from the 2D embedding of the amino acids in FIG. 3, panel G.

### Mutation and Neoantigen Distributions

The substitution frequency scatter plots (FIG. 3, panel H) are generated by determining all nonsynonymous mutations. We determine the corresponding amino acid substitution frequencies by binning the mutation substitutions (e.g. leucine to isoleucine, L→I) for each nonsynonymous mutation and then normalizing by the total number. Each substitution has a particular score from our inferred amino acid substitution matrix M, which we use as the x-axis. The linear fits are done using least squares regression and Pearson correlations are computed. Unseen amino acid substitutions (generally arising from requiring at least 2 nucleotide mutations) are excluded from the analysis.

The cumulative probability distributions (FIG. 3, panel I) are computed by determining the total fraction of neoantigens in the defined cohort that have a C or D larger than and or equal to the value on the x-axis.

### Clonal Structure of Tumors

Tumor clones are reconstructed using the PhyloWGS algorithm.²⁸ We use multisample reconstruction, combining all primary and recurrent samples from a given patient.^{j=1} The algorithm returns a family of 10000 trees, each associated with a likelihood, (Tᵢ, Lᵢ). When appropriate, our tree-based statistics for a tumor sample will be reported as averages over the top scoring trees, with weight of the i tree defined as wᵢ=Lᵢ/Σⱼ₌₁⁵ Lⱼ, the averaging operator will be denoted as [symbol] · [symbol] _{τ}. We empirically checked that the full set of results are consistent with those that use only the 5 top scoring trees.

A given tree T provides a common clone topology for all samples in the patient, with clone definitions informed by clustering of mutations across all the samples. For a given clone α∈T, the algorithm estimates the maximum likelihood clone frequency, X^{α}≡X^{α}( [symbol] ) X^{α}≡X_{α}( [symbol] )ⁱ, which is equivalent to the cellular cancer fraction (CCF) associated with that clone in that sample. We refer to these frequencies as the inclusive clone frequencies. Based on the clone definitions, we additionally define the exclusive clone frequencies,

$${𝓍}^{a} = {𝒳}^{a} - {\sum }_{β ∈ 𝒟 ( α )} {𝒳}^{β} . \qquad ( 8 )$$

Here D(α) is the set of clones that are direct descendants of clone α, as defined by the tree T. By this definition, x^{a} is a probability distribution, with Σ_{α}x^{α}=1. We denote by x the ensemble of cluster size distributions for each of the phylogenies for a given tumor sample.

### Genetic Heterogeneity of Tumor Samples.

We compute the heterogeneity of a tumor sample as the entropy of the clone frequency distribution,

$$S = {〈 - {\sum }_{α ∈ 𝒯} {x}^{a} log {x}^{a} 〉}_{𝒯} \qquad ( 9 )$$

A higher entropy indicates a more diverse and less clonal tumor composition.

### Distance Between Time Points.

The amount of evolution between the paired primary and recurrent tumor samples can be computed as the Kullback-Leibler divergence, which quantifies the amount of changes between the clones sizes between time points,

$$\vert {D}_{K L} ( { 𝓍}_{rec} ‖ {𝓍}_{prim} ) = {〈 {\sum }_{α ∈ 𝒯} {𝓍}_{rec}^{a} log \frac{{𝓍}_{rec}^{a}}{{𝓍}_{prim}^{a}} 〉}_{𝒯} \qquad ( 10 )$$

To account for predictable evolution, i.e. concerning the fate of the clones present in the primary tumor, we disregard all clones α with inclusive clone frequency X_{α}<0.03; we observed that such clones are more likely to contain mutations with unobserved reads in the primary tumor and are introduced to the topology by the reconstruction algorithm by support of mutations in the recurrent tumors.

We define the clone frequencies of these shared clones shared between primary and recurrent tumors as:

$${\tilde{𝓍}}^{α} = \{ \begin{matrix}{𝓍}^{α} / \tilde{Z ,} & if {𝒳}_{prim}^{α} \geq 0 \text{.03} \\ 0 , & otherwise\end{matrix} , \qquad ( 11 )$$

$${\tilde{χ}}^{α} = \{ \begin{matrix}{χ}^{α} / \tilde{Z ,} & if {X}_{prim}^{α} \geq 0 \text{.03} \\ 0 , & otherwise\end{matrix} \qquad ( 11 )$$

Where \tilde{Z}= [symbol] ≥0.03^{X}^{_{α}} is the normalization constant.

### Fitness Model for Tumor Clones

A fitness model is used to quantify the growth rates of clones. Here we provide a two-component model, which accounts for balancing selective pressures.

Immune fitness component. We quantify the negative selection on tumor clones imposed by the T cell recognition based on the neoantigen quality model as defined in eq. (1)

$${ℱ}^{α} = {max}_{( {p}^{MT} , h ) ∈ 𝒩 ( a )} 𝒬 ( {p}^{MT} ) , h \qquad ( 12 )$$

where N(α) is the set of neoantigens in clone α and their associated HLA alleles.

### Driver Gene Component.

We quantify selective advantage due to mutations in the recognized PDAC oncogenes, O={KRAS, TP53, CDKN2A, SMAD4} by awarding each mutation from one of these genes,

$${ℱ}_{p}^{a} = \vert 𝒢 ( a ) ⋂ 𝒪 \vert , \qquad ( 13 )$$

where G(α) is the set of genes mutated in clone α (including genes mutated in clones ancestral to α).

Combined fitness model. To account for negative selection due to immune recognition and positive selection of mutated oncogenes, we define an additive combined fitness model as

$${ℱ}^{a} ( {I}_{} , 𝓅 ) = {ℱ}_{0} - {I}_{} {ℱ}_{I}^{a} + {P}_{} {ℱ}_{p}^{a} , \qquad ( 14 )$$

where σ_{I}≥0 and σ_{P}≥0 are weights assigning the amplitude to their respective fitness components; they also determine the total amplitude of selection described by the fitness model. Constant F₀≡F₀(T, σ_{I}, σ_{P}) is a tree specific and clone independent constant determined by the normalization of clone frequencies for a given tumor sample and tree T,

$${\sum }_{α ∈ 𝒯} {𝓍}^{a} exp ( {ℱ}^{α} ( {I}_{} , 𝓅 ) ) = 1 , \qquad ( 15 )$$

Giving F₀=−(Σ_{α∈τ}χ^{α}exp(−σ_{I}F_{I}^{α}+σ_{P}F_{P}^{α})).

### Tumor Immune Cost

For a given tumor, we compute its total immune fitness cost as the negative average of fitness, over clones in a given tree, and over the possible reconstructed trees,

$${\overline{F}}_{I} = {〈 {\sum }_{α ∈ 𝒯} {𝓍}^{a} {ℱ}_{I}^{a} 〉}_{𝒯} \qquad ( 16 )$$

To evaluate the fitness of the new clones in the recurrent tumors, we compute an analogous average only over the clones that were not present in the primary tumor. As before, we use a 3% threshold on clone frequency,

$${\overline{F}}_{I}^{new} = {〈 \frac{{\sum }_{α ∈ 𝒯 : {𝒳}_{prim}^{α} < {0.03}^{{χ}_{r e c}^{α}} {ℱ}_{I}^{α}}}{{\sum }_{α ∈ 𝒯 : {𝒳}_{prim}^{α} < {0.03}^{{χ}_{r e c}^{α}}}} 〉}_{𝒯} \qquad ( 17 )$$

### Recurrent Tumor Clone Composition Predictions

For each primary and recurrent tumor pair, we predict the distribution of clone sizes in the recurrent tumor by fitness model projections from the primary tumor. In our model we combine the probability that a given clone in the primary tumor seeds a recurrence, together with a selective pressure as given by the fitness model. For a given clone α with a fitness F^{α}, the predicted exclusive clone frequency is

$${\hat{𝒳}}_{rec}^{a} ( {σ}_{I} , {σ}_{P} ) = {\tilde{𝒳}}_{prim}^{α} exp ( {F}^{α} ( {σ}_{I} , {σ}_{P} ) ) , \qquad ( 18 )$$

And the inclusive frequency is

$${\hat{𝒳}}_{rec}^{a} ( {σ}_{I} , {σ}_{P} ) = {\sum }_{β ∈ 𝒯α} {\tilde{𝒳}}_{prim}^{β} exp ( {F}^{β} ( {σ}_{I} , {σ}_{P} ) ) , \qquad ( 19 )$$

where β iterates over all subclones of α (T_{α} being a subtree of the tumor clone tree, rooted at clone α). We restrict predictions to clones that have been observed in the primary tumor, and we will use the shared clone frequency distributions as defined in eq. (11), both for the primary and recurrent tumors.

### Neoantigen Quality Model Fitting and Model Selection

The free parameters of the neoantigen quality model, Θ={a, k, w}, are trained on an independent cohort of 58 pancreatic cancer patients,⁵ to optimize survival analysis log-rank score.^{4,5} This cohort comprises samples from short and long term survivors and we have previously shown that the long-term survivors are likely to have increased immune activity in their tumors. We use our fitness model for tumor clones (14) to predict tumor growth in the pancreatic cancer patients. For each patient sample in the cohort we compute

$$\hat{𝓃} ( {σ}_{I} , {σ}_{P} , Θ ) = {\sum }_{α} {x}_{prim}^{a} exp ( {F}_{a} ( {σ}_{I} , {σ}_{P} ) ) , \qquad ( 20 )$$

the predicted tumor population size. To limit the number of parameters, we fixed the slope parameter of the R component, k=1.⁵ The survival analysis is performed by splitting the patient cohort by the median value of n(σ_{I}, σ_{P}, Θ) into high and low fitness groups and evaluation of the log-rank score, S(σ_{I}, σ_{P}, Θ) (multivariate log rank test from python package lifelines.statistics).

We computed the optimal parameters \hat{σ}_{I}, \hat{σ}_{P}, \hat{Θ} as an average ((σ_{I}, σ_{P}, Θ)), over ρ, the probability distribution defined by the log-rank test score landscape for the cohort,

$$ρ ( {σ}_{I} , C , Θ ) = {𝒵}_{ρ}^{- 1} exp ( S ( {σ}_{I} , {σ}_{P} , Θ ) ) \qquad ( 21 )$$

with Z_{ρ} the normalization constant assuring p is a probability distribution over the parameters with significant score, {(σ_{I}, σ_{P}, Θ):p(S(σ_{I}, σ_{P}, Θ))<0.01}(p-values are computed with χ² test). This smoothing procedure is applied to select optimal parameters while preventing over-fitting on a potentially rugged score landscape. If no choice of parameters meets the significance threshold, we average the parameters that have the maximum observed value of the score.

The optimal value of parameter a, the midpoint of the logistic binding function R, is at a=22.9 and the relative weight parameter for the two terms in component D eq. (7) is w=0.22 (FIG. 13). These are the parameter values we use to compute neoantigen qualities in the recurrent tumors cohort used in this study.

### Model Selection

Along with parameter training we performed a model selection effort to justify that all components of the fitness and neoantigen quality models are informative. We considered a variety of partial models and repeated the parameter training procedure via maximization of the log-rank test score. We considered clone fitness model of single components only, namely the driver gene component- and the immune component-only,

$${F}^{a} ( {σ}_{P} ) = {F}_{0} + {σ}_{P} {F}_{P}^{a} , \qquad ( 22 )$$

$${F}^{a} ( {σ}_{I} ) = {F}_{0} - {σ}_{I} {F}_{I}^{a} . \qquad ( 23 )$$

Further we decomposed the immune fitness component by considering various variants of the neoantigen quality model:

$$𝒬 ( {p}^{MT} , h ) = D ( {p}^{MT} , {p}^{MT} , h ) , \qquad ( 24 )$$

$$𝒬 ( {p}^{MT} , h ) = R ( {p}^{MT} ) . \qquad ( 25 )$$

To compare the performance of the models of different complexities (number of fitted parameters), we computed the BIC³⁴ and AIC values (FIG. 13). According to these criteria, the best performing model is our full clone fitness model with both the driver gene- and immune components, and the full neoantigen quality model.

### Fitness Model Fitting and Model Selection

For a given pair of primary-recurrent tumor samples from a given patient, we fit the fitness model parameters, σ_{I}, σ_{P}, to minimize the Kullback-Leibler divergence between the predicted clone composition and the observed clone composition of the recurrent tumor sample,

$${D}_{KL} ( {\tilde{x}}_{rec} ‖ {\hat{x}}_{rec} ) = {min}_{{I}_{} , 𝓅 , \geq 0} {〈 {\sum }_{α ∈ 𝒯} {\tilde{x}}_{rec}^{a} log \frac{{\tilde{x}}_{rec}^{a}}{{\hat{x}}_{rec ( {I}_{} , {P}_{} )}^{a}} 〉}_{𝒯} \qquad ( 26 )$$

The likelihood that the observed distributions are samples of populations with the predicted clone frequencies takes the form (by Sanov's theorem⁵¹)

$${L}_{0} ∼ exp ( - 𝓃 {𝒟}_{KL} ( {\tilde{x}}_{rec} ‖ {\hat{x}}_{rec} ) ) , \qquad ( 27 )$$

where n is a factor standing for the effective population size of the cells in the recurrent tumor sample, from which the clone frequencies were inferred. The effective population size reflects the sampling error and our ability to correctly estimate clone frequencies from the bulk sequencing data. It depends on multiple factors, such as the sequencing depth, the purity of the sample, and the phylogeny reconstruction algorithm. We estimate the effective population size for each sample, as described in a following section.

We evaluate the likelihood L₀ under the null model of neutral evolution, which assigns fitness values F_{N}^{α}=0 to all clones and predicts clade frequencies \hat{X}_{rec}=\hat{X}ₚᵣᵢₘ. The likelihood of the data under this model is, amorously to (27), given by

$${L}_{0} ∼ exp ( - 𝓃 {𝒟}_{KL} ( {\tilde{x}}_{prim} ‖ {\overline{x}}_{rec} ) ) . \qquad ( 28 )$$

To compare models of varying complexity, we compute the Bayesian Information Criterion (BIC),³⁴

$$BIC ( L ) = \vert Θ \vert log ( 𝓃 ) - 2 log ( L ) , \qquad ( 29 )$$

where Θ is the set of optimized parameters, |Θ|=2, arriving at an adjusted log-likelihood,

$$log ( {L}^{adj} ) ∼ - BIC ( L ) / 2 = log ( L ) - log ( 𝓃 ) . \qquad ( 30 )$$

To assess the predictive power of individual fitness model components, we consider partial models and their corresponding optimized likelihoods: the immune component only model F_{I}^{∝}( [symbol] _{I})≡(F^{∝})( [symbol] _{I}, [symbol] p, =0) with likelihood L_{I}; and the driver gene component only model F_{P}^{∝}( [symbol] _{P})≡(F^{∝})( [symbol] _{I}=0, [symbol] _{P}) with likelihood LP. Each of these models has one free parameter, we apply the BIC-based correction (30) to compute the adjusted log-likelihoods

$$log ( {L}_{I}^{adj} ) = log ( {L}_{I} ) - log ( 𝓃 ) / 2 and log ( {L}_{P}^{adj} ) = log ( {L}_{P} ) - log ( 𝓃 ) / 2.$$

In general, to compare fitting alternative models F₁ and F₂ on a cohort S, for each sample s in the cohort we compute the log-likelihood score, We also evaluate the aggregated score over samples Δ [symbol] (s, F₁, F₂)=log(L₁^{adj}(s))−log(L₂^{adj}(s)). We also evaluate the aggregated score over samples in cohort S,

$${Δℒ}^{s} ( {F}_{1} , {F}_{2} ) = {\sum }_{s ∈ s} Δℓ ( s , {F}_{1} , {F}_{2} ) \qquad ( 31 )$$

where s iterates over samples in the cohort. Positive scores favor model L₁ over L₂.

### Effective Cancer Cell Population Size n

To account for sampling error which effects clone frequency inference, we estimate the error of mutation frequencies for each of the tumor samples in our data. We evaluate frequencies for each mutation m in a given sample s, with the frequencies from the individual trees T, given by x(m)=X^{α} where m originates in clone α∈T. The variance is computed over the 5 trees reconstructed for that sample, σ²(xₘ)= [symbol] (m)² [symbol] _{τ}−(x(m))_{τ}². The effective size n for a given sample scales proportionally with the inverse of variance, giving our estimate of n as

$$𝓃 ( s ) ∼ \frac{1}{〈 {2}^{} ( {x}_{𝓂} ) {〉}_{𝓂 ∈ s}} \qquad ( 32 )$$

For the patients with multiple samples the variance of mutation frequencies from tree reconstruction is reduced due to information from other samples; to account for this effect we divide n(s) by the total number of additional samples. The estimated effective cancer population sizes vary from 79.79 to 1189.60, with a mean of 187.23 and median 244.6.

### Clone Fitness Model Selection

We compute the log-likelihood score for the alternative models for each recurrent tumor sample (FIG. 13). In particular, we observe that 19 out of 22 LTS samples (86%) and 17 out of 33 (52%) are better described by the model with selection, F, rather than the null model, F_{N} ≡0 (giving Δ [symbol] (s, F, F_{N})>0ⁱ). Evaluating the aggregated log-likelihood score on the LTS and STS cohorts, we observe evidence for the model with selection in both cohorts, Δ [symbol] ^{LTS}(F, F_{N})=1241 nats and Δ [symbol] ^{STS}(F, F_{N})=198 nats, with a mean of 56.42 nats and 6.01 nats respectively. The fit, and therefore the predictive power of model F, is relatively stronger in the LTS cohort.

We assess that the oncogenic selection, described by the driver gene component model F_{P}, provides predictive signal on its own, with 14 out of 22 LTS samples (64%) and 13 out of 33 STS samples (39%) having positive log-likelihood score, Δ [symbol] (s, F_{P}, F_{N})>0ⁱ. Evaluating the aggregated log-likelihood score on the LTS and STS cohorts, we observe evidence for the model with oncogenic selection in both cohorts, Δ [symbol] ^{LTS}(F_{P}, F_{N})=1041 nats and Δ [symbol] ^{STS}(F_{P}, F_{N})=223 nats, with a mean of 47.34 nats and 6.77 nats respectively. Again, the fit of the partial model is stronger in the LTS cohort. This effect could be explained indirectly by the negative immune selection, which reduces tumor heterogeneity and facilitates clonal composition predictions in the LTS cohort.

The immune component F_{I} on its own has less predictive power, with positive log-likelihood score for Δ [symbol] (s, F_{I}, F_{N}) for 11 out of 22 LTS samples (50%) and only 4 out of 33 STS samples (12%). The aggregated log-likelihood score values are Δ [symbol] ^{LTS}(F_{I}, F_{N})=579 nats (mean of 26.31 nats) and Δ [symbol] ^{STS}(F_{I}, F_{N})=91 nats (mean of 2.75 nats).

The full model with both components provides an improvement to the driver-gene component-only model for 11 out of 22 LTS samples (50%) but only 5 out of 33 STS samples (15%), as quantified by Δ [symbol] (s, F, F_{P})>0. The aggregated log-likelihood score on the LTS and STS cohorts are Δ [symbol] (F, F_{P})=200 nats and Δ [symbol] ^{STS}(F, F_{P})=−25 nats respectively. This results means that inclusion of the immune component directly improves prediction of clone dynamics in the LTS cohort, but it does not for the STS cohort. All these results are reported in FIG. 13 and in FIG. 12.

### Accuracy of Clone Growth Fitting

We consider the observed and model fitted clone frequency changes, X_{rec}^{α}/Xₚᵣᵢₘ^{α} and \hat{X}_{rec}^{α}/Xₚᵣᵢₘ^{α} across all clones in tumors in the cohorts. For a given cohort we define the accuracy model as the fraction of clones for which the direction of change is the same, i.e. (X_{rec}^{α}/Xₚᵣᵢₘ^{α}>1 and \hat{X}_{rec}^{α}/Xₚᵣᵢₘ^{α}>1) or (X_{rec}^{α}/Xₚᵣᵢₘ^{α}<1 and \hat{X}_{rec}^{α}/Xₚᵣᵢₘ^{α}<1) or (X_{rec}^{α}/Xₚᵣᵢₘ^{α}=1 and \hat{X}_{rec}^{α}/Xₚᵣᵢₘ^{α}=1). We consider all clones with frequency larger than 0.03 in the primary tumor, from the top scoring trees for each patient. We obtained accuracy of 71% over 243 clones in the LTS cohort, and 58% over 389 clones in the STS cohort. The Pearson correlation coefficients are r^{LTS}=0.57 and r^{STS}=0.35 (as computed on log-transformed frequency changes, log X_{rec}^{α}/Xₚᵣᵢₘ^{α} and log \hat{X}_{rec}^{α}/Xₚᵣᵢₘ^{α}) and Spearman rank coefficients are p^{LTS}=0.65 and p^{STS}=0.28 (FIG. 13, panel B).

### TCR Beta (TCRB) Sequencing

We extracted genomic DNA from n=23 primary and recurrent STS and LTS PDACs according to the manufacturer's instructions (QIAsymphony, Qiagen). We verified the quantity and quality of extracted DNA before sequencing. We then used a standard quantity of input DNA, amplified and sequenced the CDR3 [symbol] regions using the survey multiplexed PCR ImmunoSeq assay (Adaptive Biotechnologies). The ImmunoSeq platform combines multiplex PCR with high-throughput sequencing to selectively amplify the rearranged complementarity-determining region 3 (CDR3 [symbol] ) of the TCR, producing fragments sufficiently long to identify the VDJ region spanning each unique CDR3 [symbol] After correcting for sequencing coverage, PCR bias, primer bias, and sequencing errors, we define a T cell clone as a T cell with a unique TCRB CDR3 [symbol] amino acid sequence.

### Dissimilarity Index to Estimate Antigen-Specificity in a T Cell Repertoire

To estimate the antigen-specificity of a T cell repertoire, for each repertoire, we apply a sequence based probabilistic model called a Restricted Boltzmann Machine (RBM).¹⁸ The RBM model assigns a probabilistic score of an antigen specific response to each T cell clone in a sample, based on the frequency and the CDR3 [symbol] sequence similarity of the top 25 ranking clones. Based on these RBM scores for each clone, we estimate for each repertoire, a TCR dissimilarity index

$$DI = \frac{1}{f} . \qquad ( 33 )$$

where:

$$f = \frac{1}{T} {\sum }_{𝒾 < j} {e}^{- ( \frac{d ( {σ}_{𝒾} , {σ}_{j} )}{δ} ) 2} \qquad ( 33 )$$

where T is the total number of terms in the sum (T=M (M−1)/2), M=25 (the top 25 clones in the repertoire with the highest RBM scores), and d(σᵢ, σⱼ) is a distance obtained from the global pairwise alignment score between the CDR3 [symbol] amino acid sequences σᵢ and σⱼ. This score is computed using the BLOSUM62 matrix corrected with an offset such that all its weights are positive, −S(A, B)+max_{A,B}(S(A,B))≥0, where S(A, B) are the usual BLOSUM62 matrix elements. The parameter δ represents a typical scale of the BLOSUM-weighted distance d and it is set to δ=9.37, the average distance d between reported epitope-specific CDR3 [symbol] sequences (we use an influenza-specific repertoire²¹). As a control, we calculate this TCR dissimilarity index between the top 25 clones in the repertoire based on clone size (TCR dissimilarity index−clone size), and not the RBM computed probability (FIG. 4, panel C).

To verify that the difference in the TCR dissimilarity index between LTSs and STSs is robust, we randomly subsample the repertoire down to a few hundred clones and repeat the RBM training, score assignment, and TCR dissimilarity index estimation 10 times (FIG. 4, panel B).

### Statistics

Survival curves were compared using log-rank test (Mantel-Cox). Comparison between two groups was performed using unpaired two-tailed Mann-Whitney test, or Wald's test for gene expression analyses to correct for multiple comparison testing. Correlation between two variables was performed using two-tailed Pearson correlation. Categorical variables were compared using chi-square test. Probability distributions were compared using two-sided Kolmogorov-Smirnov (KS) test. All comparison groups had equivalent variances. P<0.05 was considered to be statistically significant. Data analysis was performed using statistical software (Prism 7.0, GraphPad Software v.9.1.0 and Python v.3.4).

### REFERENCES CITED IN THIS EXAMPLE

1. Shankaran, V. et al. Pillars article: Ifngamma and lymphocytes prevent primary tumour development and shape tumour immunogenicity. nature. 2001. 410: 1107-1111. J Immunol 201, 827-831 (2018). URL https://www.ncbi.nlm.nih.gov/pubmed/30038035.
2. Hanahan, D. & Weinberg, R. A. Hallmarks of cancer: the next generation. Cell 144, 646-74 (2011).
3. Matsushita, H. et al. Cancer exome analysis reveals a t-cell-dependent mechanism of cancer immunoediting. Nature 482, 400-4 (2012).
4. Luksza, M. et al. A neoantigen fitness model predicts tumour response to checkpoint blockade immunotherapy. Nature 551, 517-520 (2017).
5. Balachandran, V. P. et al. Identification of unique neoantigen qualities in long-term survivors of pancreatic cancer. Nature 551, 512-516 (2017).
6. Burnet, F. M. The concept of immunological surveillance. Prog Exp Tumor Res 13, 1-27 (1970).
7. Dunn, G. P., Bruce, A. T., Ikeda, H., Old, L. J. & Schreiber, R. D. Cancer immunoediting: from immunosurveillance to tumor escape. Nat Immunol 3, 991-8 (2002).
8. Schumacher, T. N. & Schreiber, R. D. Neoantigens in cancer immunotherapy. Science 348, 69-74 (2015).
9. Rosenthal, R. et al. Neoantigen-directed immune escape in lung cancer evolution. Nature 567, 479-485 (2019).
10. Zhang, A. W. et al. Interfaces of malignant and immunologic clonal dynamics in ovarian cancer. Cell 173, 1755-1769 e22 (2018).
11. Jimenez-Sanchez, A. et al. Heterogeneous tumor-immune microenvironments among differentially growing metastases in an ovarian cancer patient. Cell 170, 927-938 e20 (2017).
12. Balli, D., Rech, A. J., Stanger, B. Z. & Vonderheide, R. H. Immune cytolytic activity stratifies molecular subsets of human pancreatic cancer. Clin Cancer Res 23, 3129-3138 (2017).
13. Rizvi, N. A. et al. Cancer immunology. mutational landscape determines sensitivity to pd-1 blockade in non-small cell lung cancer. Science 348, 124-8 (2015).
14. Van Allen, E. M. et al. Genomic correlates of response to ctla-4 blockade in metastatic melanoma. Science 350, 207-211 (2015).
15. Yachida, S. et al. Distant metastasis occurs late during the genetic evolution of pancreatic cancer. Nature 467, 1114-7 (2010).
16. Ino, Y. et al. Immune cell infiltration as an indicator of the immune microenvironment of pancreatic cancer. British Journal of Cancer 108, 914-923 (2013).
17. Riquelme, E. et al. Tumor microbiome diversity and composition influence pancreatic cancer outcomes. Cell 178, 795-806 e 12 (2019).
18. Bravi, B. et al. Probing t-cell response by sequence-based probabilistic modeling. PLoS Comput Biol 17, e1009297 (2021).
19. Sakamoto, H. et al. The evolutionary origins of recurrent pancreatic cancer. Cancer Discov 10, 792-805 (2020).
20. Dyall, R. et al. Heteroclitic immunization induces tumor immunity. J Exp Med 188, 1553-61 (1998).
21. Dash, P. et al. Quantifiable predictive features define epitope-specific t cell receptor repertoires. Nature 547, 89-93 (2017).
22. Glanville, J. et al. Identifying specificity groups in the t cell receptor repertoire. Nature 547, 94-98 (2017).
23. Birnbaum, M. E. et al. Deconstructing the peptide-mhc specificity of t cell recognition. Cell 157, 1073-87 (2014).
24. Solache, A. et al. Identification of three hla-a*0201-restricted cytotoxic t cell epitopes in the cytomegalovirus protein pp65 that are conserved between eight strains of the virus. J Immunol 163, 5512-8 (1999).
25. Kawakami, Y. et al. Recognition of multiple epitopes in the human melanoma antigen gp100 by tumor-infiltrating t lymphocytes associated with in vivo tumor regression. J Immunol 154, 3961-8 (1995).
26. Parkhurst, M. R. et al. Improved induction of melanoma-reactive ctl with peptides from the melanoma antigen gp100 modified at hla-a*0201-binding residues. J Immunol 157, 2539-48 (1996).
27. Capietto, A. H. et al. Mutation position is an important determinant for predicting cancer neoantigens. J Exp Med 217 (2020)
28. Deshwar, A. G. et al. Phylowgs: reconstructing subclonal composition and evolution from whole-genome sequencing of tumors. Genome Biol 16, 35 (2015).
29. Evans, R. A. et al. Lack of immunoediting in murine pancreatic cancer reversed with neoantigen. JCI Insight 1 (2016).
30. Freed-Pastor, W. A. et al. Abstract prl4: Preclinical models to dissect immune escape in pancreatic cancer. Cancer Research 79, PR14-PR14 (2019).
31. Barthel, F. P. et al. Longitudinal molecular trajectories of diffuse glioma in adults. Nature 576, 112-120 (2019).
32. Zaretsky, J. M. et al. Mutations associated with acquired resistance to pd-1 blockade in melanoma. N Engl J Med 375, 819-29 (2016).
33. Cancer Genome Atlas Research Network. Electronic address, a. a. d. h. e. & Cancer Genome Atlas Research, N. Integrated genomic characterization of pancreatic ductal adenocarcinoma. Cancer Cell32, 185-203 e13 (2017).
34. Schwarz, G. Estimating the dimension of a model. The Annals of Statistics 6, 461-464 (1978).
35. Cibulskis, K. et al. Sensitive detection of somatic point mutations in impure and heterogeneous cancer samples. Nat Biotechnol 31, 213-9 (2013).
36. Saunders, C. T. et al. Strelka: accurate somatic small-variant calling from sequenced tumor-normal sample pairs. Bioinformatics 28, 1811-7 (2012).
37. Tate, J. G. et al. Cosmic: the catalogue of somatic mutations in cancer. Nucleic Acids Res 47, D941-D947 (2019).
38. Robinson, J. T., Thorvaldsdottir, H., Wenger, A. M., Zehir, A. & Mesirov, J. P. Variant review with the integrative genomics viewer. Cancer Res 77, e31-e34 (2017).
39. Grossman, R. L. et al. Toward a shared vision for cancer genomic data. N Engl J Med 375, 1109-12(2016).
40. Szolek, A. et al. Optitype: precision hla typing from next-generation sequencing data. Bioinformatics 30, 3310-6 (2014).
41. Cingolani, P. et al. A program for annotating and predicting the effects of single nucleotide polymorphisms, snpeff: Snps in the genome of Drosophila melanogaster strain w 1118; iso-2; iso-3. Fly 6, 80-92 (2012).
42. Nielsen, M. et al. Reliable prediction of t-cell epitopes using neural networks with novel sequence representations. Protein Sci 12, 1007-17 (2003).
43. Lundegaard, C. et al. Netmhc-3.0: accurate web accessible predictions of human, mouse and monkey mhc class i affinities for peptides of length 8-11. Nucleic Acids Res 36, W509-12 (2008).
44. Gallardo, H. F., Tan, C., Ory, D. & Sadelain, M. Recombinant retroviruses pseudotyped with the vesicular stomatitis virus g glycoprotein mediate both stable gene transfer and pseudotransduction in human peripheral blood lymphocytes. Blood 90, 952-7 (1997).
45. Ghani, K. et al. Efficient human hematopoietic cell transduction using rd114- and galv-pseudotyped retroviral vectors produced in suspension and serum-free media. Hum Gene Ther 20, 966-74 (2009).
46. Gros, A. et al. Prospective identification of neoantigen-specific lymphocytes in the peripheral blood of melanoma patients. Nat Med 22, 433-8 (2016).
47. Cohen, C. J., Zhao, Y., Zheng, Z., Rosenberg, S. A. & Morgan, R. A. Enhanced antitumor activity of murine-human hybrid t-cell receptor (ter) in human lymphocytes is associated with improved pairing and tcr/cd3 stability. Cancer Res 66, 8878-86 (2006).
48. Riviere, I., Brose, K. & Mulligan, R. C. Effects of retroviral vector design on expression of human adenosine deaminase in murine bone marrow transplant recipients engrafted with genetically modified cells. Proc Natl Acad Sci USA 92, 6733-7 (1995).
49. Vita, R. et al. The immune epitope database (iedb) 3.0. Nucleic acids research 43, D405-D412 (2014).
50. Andreatta, M. & Nielsen, M. Gapped sequence alignment using artificial neural networks: application to the mhc class i system. Bioinformatics 32, 511-7 (2016).
51. Cover, T. M. & Thomas, J. A. Elements of Information Theory (Wiley Series in Telecommunications and Signal Processing) (Wiley-Interscience, USA, 20

### Example 3

Allen, E. M. V. et al. Genomic correlates of response to CTLA-4 blockade in metastatic melanoma. Science 350, 207-211 (2015).
Balachandran, V. P. et al. Identification of unique neoantigen qualities in long-term survivors of pancreatic cancer. Nature 551, 512-516 (2017).
Balli, D., et al. Immune Cytolytic Activity Stratifies Molecular Subsets of Human Pancreatic Cancer. Clin Cancer Res 23, 3129-3138 (2017).
Birnbaum, M. E. et al. Deconstructing the Peptide-MHC Specificity of T Cell Recognition. Cell 157, 1073-1087 (2014).
Bravi, B. et al. Probing T-cell response by sequence-based probabilistic modeling. Plos Comput Biol 17, e1009297 (2021).
Capietto, A.-H. et al. Mutation position is an important determinant for predicting cancer neoantigens. J Exp Med 217, e20190179 (2020).
Dash, P. et al. Quantifiable predictive features define epitope-specific T cell receptor repertoires. Nature 547, 89-93 (2017).
Dyall, R. et al. Heteroclitic Immunization Induces Tumor Immunity. J Exp Medicine 188, 1553-1561 (1998).
Glanville, J. et al. Identifying specificity groups in the T cell receptor repertoire. Nature 547, 94-98 (2017).
Ino, Y. et al. Immune cell infiltration as an indicator of the immune microenvironment of pancreatic cancer. Brit J Cancer 108, 914-923 (2013).
Kawakami, Y. et al. Recognition of multiple epitopes in the human melanoma antigen gp100 by tumor-infiltrating T lymphocytes associated with in vivo tumor regression. J Immunol 154, 3961-8 (1995).
Luksza, M. & Lassig, M. A predictive fitness model for influenza. Nature 507, 57-61 (2014).
Luksza, M. et al. A neoantigen fitness model predicts tumour response to checkpoint blockade immunotherapy. Nature 551, 517-520 (2017).
Parkhurst, M. R. et al. Improved induction of melanoma-reactive CTL with peptides from the melanoma antigen gp100 modified at HLA-A*0201-binding residues. J Immunol 157, 2539-48 (1996).
Riquelme, E. et al. Tumor Microbiome Diversity and Composition Influence Pancreatic Cancer Outcomes. Cell 178, 795-806.e12 (2019).
Rizvi, N. A. et al. Mutational landscape determines sensitivity to PD-1 blockade in non-small cell lung cancer. Science 348, 124-128 (2015).
Sakamoto, H. et al. The Evolutionary Origins of Recurrent Pancreatic Cancer. Cancer Discov 10, 792-805 (2020).
Solache, A. et a. Identification of three HLA-A*0201-restricted cytotoxic T cell epitopes in the cytomegalovirus protein pp65 that are conserved between eight strains of the virus. J Immunol 163, 5512-8 (1999).
Yachida, S. et al. Distant metastasis occurs late during the genetic evolution of pancreatic cancer. Nature 467, 1114-1117 (2010).

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A method for determining within a computing system a likelihood that a human subject afflicted with a cancer will be responsive to a treatment regimen that comprises administering a checkpoint blockade immunotherapy directed to the cancer of the subject, the computing system having one or more programmable processors, memory, and a plurality of instructions stored on the memory that are executable by the one or programmable processors, the method comprising:
a) obtaining a plurality of sequencing reads from one or more samples from the human cancer subject that is representative of the cancer;
b) determining a human leukocyte antigen (HLA) type of the human subject;
c) computing for a plurality of clones, and for each respective clone a in the plurality of clones, an initial frequency Xₐ of the respective clone a in the one or more samples;
d) for each respective clone a in the plurality of clones, computing a corresponding clone fitness score of the respective clone, thereby computing a plurality of clone fitness scores, each corresponding clone fitness score computed for a respective clone a by a first procedure comprising:
i. identifying a plurality of neoantigens in the respective clone a;
ii. computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and
iii. determining the corresponding clone fitness score of the respective clone a as an aggregate of the neoantigen recognition potentials across the plurality of neoantigens in the respective clone a; and
e) computing a total fitness for the one or more samples as a sum of the clone fitness scores across the plurality of clones, wherein each clone fitness score is weighted by the initial frequency Xₐ of the corresponding clone a, the total fitness quantifies the likelihood that the human subject afflicted with the cancer will be responsive to the treatment regimen,
optionally wherein the initial frequency Xₐ of the respective clone a in the one or more samples is determined using the plurality of sequencing reads from the one or more samples from the human subject or optionally wherein the cancer is a carcinoma, a melanoma, a lymphoma/leukemia, a sarcoma, a neuro-glial tumor, lung cancer, pancreatic cancer, colon cancer, stomach cancer, esophagus cancer, breast cancer, ovary cancer, prostate cancer, or liver cancer.

2. The method of claim 1, further comprising administering to the human subject the checkpoint blockade immunotherapy if the human subject is likely to be responsive to the treatment regimen, optionally wherein the checkpoint blockade immunotherapy comprises an antibody, fragment, or derivative thereof, wherein the antibody is specific for CTLA4, PD1, PD-L1, LAG3, TIM-3, GITR, OX40, CD40, TIGIT, 4-1BB, B7-H3, B7-H4, or BTLA or the checkpoint blockade immunotherapy comprises ipilimumab or tremelimumab.

3. (canceled)

4. (canceled)

5. (canceled)

6. (canceled)

7. (canceled)

8. The method of claim 1, wherein
each clone a in the plurality of clones is uniquely defined by a unique set of somatic mutations, and the plurality of clones is determined by a variant allele frequency of each respective somatic mutation in a plurality of somatic mutations determined from the plurality of sequencing reads, optionally wherein the somatic mutation is a single nucleotide variant or an indel.

9. (canceled)

10. (canceled)

11. The method of claim 1, wherein the plurality of clones is determined by identifying a plurality of inferred copy number variations using the plurality of sequencing reads, wherein
each clone a in the plurality of clones is uniquely defined by a unique set of somatic mutations, and
the plurality of clones is determined by a combination of (i) a variant allele frequency of each respective somatic mutation in the plurality of somatic mutations determined from the plurality of sequencing reads and (ii) an identification of a plurality of inferred copy number variations using the whole-genome sequencing data.

12. The method of claim 1, wherein the plurality of sequencing reads exhibits an average read depth of less than 40 or between 25 and 60 or wherein each neoantigen in the plurality of neoantigens of a clone in the plurality of clones is a nonamer peptide or a peptide comprising eight, nine, ten, or eleven residues in length.

13. (canceled)

14. (canceled)

15. (canceled)

16. The method of claim 1, wherein the HLA type of the human subject is determined from the plurality of sequencing reads or using a polymerase chain reaction using a biological sample from the human subject.

17. (canceled)

18. The method of claim 1, wherein the plurality of clones comprises two clones, between two clones and ten clones, or greater than ten clones.

19. (canceled)

20. (canceled)

21. (canceled)

22. (canceled)

23. A system for determining a likelihood that a human subject afflicted with a cancer will be responsive to a treatment regimen that comprises administering a checkpoint blockade immunotherapy directed to the cancer to the subject, comprising
memory;
one or more processors; and
one or more modules stored in memory and configured for execution by the one or more processors, the modules comprising instructions for carrying out the method of claim 1.

24. (canceled)

25. A non-transitory computer readable storage medium for determining a likelihood that a human subject afflicted with a cancer will be responsive to a treatment regimen that comprises administering a checkpoint blockade immunotherapy directed to the cancer to the subject, the non-transitory computer readable storage medium storing one or more programs for execution by one or more processors of a computer system, the one or more computer programs comprising instructions for carrying out the method of claim 1.

26. A method for identifying within a computing system a neoantigen to target as an immunotherapy for a cancer, the computing system having one or more programmable processors, memory, and a plurality of instructions stored on the memory that are executable by the one or programmable processors, the method comprising:
a) obtaining a plurality of sequencing reads from one or more samples from a human cancer subject that is representative of the cancer;
b) determining a human leukocyte antigen (HLA) type of the human subject from the plurality of sequencing reads;
c) determining a plurality of clones, and for each respective clone a in the plurality of clones, an initial frequency Xₐ of the respective clone a in the one or more samples from the plurality of sequencing reads; for each respective clone a in the plurality of clones, computing a corresponding clone fitness score of the respective clone, thereby computing a plurality of clone fitness scores, each corresponding clone fitness score computed for a respective clone a by a first procedure comprising:
i. identifying a plurality of neoantigens in the respective clone a;
ii. computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and
iii. determining the corresponding clone fitness score of the respective clone a as an aggregate of the neoantigen recognition potentials across the plurality of neoantigens in the respective clone a; and
d) selecting at least a first neoantigen from a plurality of neoantigens for a respective clone a in the plurality of respective clones based upon the recognition potential of the first neoantigen to target as an immunotherapy for the cancer
optionally wherein the cancer is a carcinoma, a melanoma, a lymphoma/leukemia, a sarcoma, a neuro-glial tumor, lung cancer, pancreatic cancer, colon cancer, stomach cancer, esophagus cancer, breast cancer, ovary cancer, prostate cancer, or liver cancer or optionally wherein the initial frequency Xa of the respective clone a in the one or more samples is determined using the plurality of sequencing reads from the one or more samples from the human subject.

27. The method of claim 26, wherein the obtaining (a), the determining (b), the determining (c), and the computing (d) are repeated for a plurality of human subjects across a plurality of HLA types, optionally wherein the first neoantigen is selected on the basis of the recognition potential of the first neoantigen across the plurality of HLA types.

28. (canceled)

29. The method of claim 26, wherein the obtaining (a), the determining (b), the determining (c), and the computing (d) are repeated for a plurality of human subjects, optionally wherein the first neoantigen is selected on the basis of the recognition potential of the first neoantigen across the plurality of human subjects.

30. (canceled)

31. (canceled)

32. (canceled)

33. The method of claim 26, wherein each clone a in the plurality of clones is uniquely defined by a unique set of somatic mutations, and wherein the plurality of clones is determined by a variant allele frequency of each respective somatic mutation in a plurality of somatic mutations determined from the whole-genome sequencing data, optionally wherein the somatic mutation is a single nucleotide variant or an indel: or
wherein the plurality of clones is determined by identifying a plurality of inferred copy number variations using the whole-genome sequencing data: or
wherein the plurality of clones comprises two clones or between two clones and ten clones.

34. (canceled)

35. (canceled)

36. The method of claim 26, wherein
each clone a in the plurality of clones is uniquely defined by a unique set of somatic mutations, and
the plurality of clones is determined by a combination of (i) a variant allele frequency of each respective somatic mutation in the plurality of somatic mutations determined from the whole-genome sequencing data and (ii) an identification of a plurality of inferred copy number variations using the whole-genome sequencing data.

37. The method of claim 26, wherein the plurality of sequencing reads exhibits an average read depth of less than 40, or between 25 and 60 or wherein each neoantigen in the plurality of neoantigens of a clone in the plurality of clones is a nonamer peptide or is a peptide comprising eight, nine, ten, or eleven residues in length.

38. (canceled)

39. (canceled)

40. (canceled)

41. The method of claim 26, wherein the determining the HLA type of the human subject is determined from the plurality of sequencing reads or wherein the determining the HLA type of the human subject is determined using a polymerase chain reaction using a biological sample from the cancer subject.

42. (canceled)

43. (canceled)

44. (canceled)

45. (canceled)

46. (canceled)

47. A system for identifying an immunotherapy for a cancer, comprising:
memory;
one or more processors; and
one or more modules stored in memory and configured for execution by the one or more processors, the modules comprising instructions for executing the method of claim 26.

48. (canceled)

49. A non-transitory computer readable storage medium for identifying an immunotherapy for a cancer, the non-transitory computer readable storage medium storing one or more programs for execution by one or more processors of a computer system, the one or more computer programs comprising instructions for executing the method of claim 26.

50. A method of treating a human subject afflicted with a cancer with a checkpoint blockade immunotherapy directed to the cancer, wherein the method comprises:
(1) determining within a computing system that the human subject is likely to be responsive to the checkpoint blockade immunotherapy, wherein the computing system has one or more programmable processors, memory, and a plurality of instructions stored on the memory that are executable by the one or programmable processors; and
(2) administering the checkpoint block immunotherapy when it is determined that the human subject is likely to be responsive to the checkpoint blockade immunotherapy;
wherein the determination that the human subject will be responsive to the checkpoint blockade immunotherapy comprises:
(A) obtaining a plurality of sequencing reads from one or more samples from the human cancer subject that is representative of the cancer;
(B) determining a human leukocyte antigen (HLA) type of the human subject;
(C) determining a plurality of clones, and for each respective clone a in the plurality of clones, an initial frequency Xₐ of the respective clone a in the one or more samples;
(D) for each respective clone a in the plurality of clones, computing a corresponding clone fitness score of the respective clone, thereby computing a plurality of clone fitness scores, each corresponding clone fitness score computed for a respective clone a by a first procedure comprising:
(a) identifying a plurality of neoantigens in the respective clone a;
(b) computing a recognition potential of each respective neoantigen in the plurality of neoantigens in the respective clone a by a second procedure, wherein the second procedure comprises computing a T-cell cross-reactivity distance C between the respective neoantigen and the wildtype counterpart as a function of the half-maximal effective concentration for T cell activation of the wildtype counterpart of the respective neoantigen relative to the half-maximal effective concentration for T cell activation of the respective neoantigen; and
(c) determining the corresponding clone fitness score of the respective clone a as an aggregate of the neoantigen recognition potentials across the plurality of neoantigens in the respective clone a; and
(E) computing a total fitness for the one or more samples as a sum of the clone fitness scores across the plurality of clones, wherein each clone fitness score is weighted by the initial frequency Xₐ of the corresponding clone a, and the total fitness quantifies the likelihood that the human subject afflicted with the cancer will be responsive to the checkpoint blockade immunotherapy,
optionally wherein the cancer is a carcinoma, a melanoma, a lymphoma/leukemia, a sarcoma, a neuro-glial tumor, lung cancer, pancreatic cancer, colon cancer, stomach cancer, esophagus cancer, breast cancer, ovary cancer, prostate cancer, or liver cancer.

51. The method of claim 50, wherein the checkpoint blockade immunotherapy comprises an antibody, fragment, or derivative thereof, optionally wherein the antibody is specific for CTLA4, PD1, PD-L1, LAG3, TIM-3, GITR, OX40, CD40, TIGIT, 4-1BB, B7-H3, B7-H4, or BTLA or wherein the checkpoint blockade immunotherapy comprises ipilimumab or tremelimumab.

52. (canceled)

53. (canceled)

54. (canceled)

55. (canceled)
